SEQUENCE LISTING (112 sequences)

SEQ ID NO: 1 - protein, 17 aa, Homo Sapiens
TVFRSSREKRRSADIFI

SEQ ID NO: 2 - protein, 17 aa, Artificial Sequence
  Synthetic
AVFRSSREKRRSADIFI

SEQ ID NO: 3 - protein, 17 aa, Artificial Sequence
  Synthetic
TAFRSSREKRRSADIFI

SEQ ID NO: 4 - protein, 17 aa, Artificial Sequence
  Synthetic
TVARSSREKRRSADIFI

SEQ ID NO: 5 - protein, 17 aa, Artificial Sequence
  Synthetic
TVFASSREKRRSADIFI

SEQ ID NO: 6 - protein, 17 aa, Artificial Sequence
  Synthetic
TVFRASREKRRSADIFI

SEQ ID NO: 7 - protein, 17 aa, Artificial Sequence
  Synthetic
TVFRSAREKRRSADIFI

SEQ ID NO: 8 - protein, 17 aa, Artificial Sequence
  Synthetic
TVFRSSAEKRRSADIFI

SEQ ID NO: 9 - protein, 17 aa, Artificial Sequence
  Synthetic
TVFRSSRAKRRSADIFI

SEQ ID NO: 10 - protein, 17 aa, Artificial Sequence
  Synthetic
TVFRSSREARRSADIFI

SEQ ID NO: 11 - protein, 17 aa, Artificial Sequence
  Synthetic
TVFRSSREKARDADIFI

SEQ ID NO: 12 - protein, 17 aa, Artificial Sequence
  Synthetic
TVFRSSREKRASADIFI

SEQ ID NO: 13 - protein, 17 aa, Artificial Sequence
  Synthetic
TVFRSSREKRRAADIFI

SEQ ID NO: 14 - protein, 17 aa, Artificial Sequence
  Synthetic
TVFRSSREKRRSAAIFI

SEQ ID NO: 15 - protein, 17 aa, Artificial Sequence
  Synthetic
TVFRSSREKRRSADAFI

SEQ ID NO: 16 - protein, 17 aa, Artificial Sequence
  Synthetic
TVFRSSREKRRSADIAI

SEQ ID NO: 17 - protein, 17 aa, Artificial Sequence
  Synthetic
TVFRSSREKRRSADIFA

SEQ ID NO: 18 - protein, 17 aa, Artificial Sequence
  Synthetic
XVFRSSREKRRSADIFI

SEQ ID NO: 19 - protein, 17 aa, Artificial Sequence
  Synthetic
TXFRSSREKRRSADIFI

SEQ ID NO: 20 - protein, 17 aa, Artificial Sequence
  Synthetic
TVXRSSREKRRSADIFI

SEQ ID NO: 21 - protein, 17 aa, Artificial Sequence
  Synthetic
TVFXSSREKRRSADIFI

SEQ ID NO: 22 - protein, 17 aa, Artificial Sequence
  Synthetic
TVFRXSREKRRSADIFI

SEQ ID NO: 23 - protein, 17 aa, Artificial Sequence
  Synthetic
TVFRSXREKRRSADIFI

SEQ ID NO: 24 - protein, 17 aa, Artificial Sequence
  Synthetic
TVFRSSXEKRRSADIFI

SEQ ID NO: 25 - protein, 17 aa, Artificial Sequence
  Synthetic
TVFRSSRXKRRSADIFI

SEQ ID NO: 26 - protein, 17 aa, Artificial Sequence
  Synthetic
TVFRSSREKXRSADIFI

SEQ ID NO: 27 - protein, 17 aa, Artificial Sequence
  Synthetic
TVFRSSREKRXSADIFI

SEQ ID NO: 28 - protein, 17 aa, Artificial Sequence
  Synthetic
TVFRSSREKRRSADXFI

SEQ ID NO: 29 - protein, 17 aa, Artificial Sequence
  Synthetic
TVFRSSREKRRSADIFX

SEQ ID NO: 30 - protein, 17 aa, Artificial Sequence
  Synthetic
TVFRSSREKRRXADIFI

SEQ ID NO: 31 - protein, 17 aa, Artificial Sequence
  Synthetic
TVFRSSREKRRSXDIFI

SEQ ID NO: 32 - protein, 17 aa, Artificial Sequence
  Synthetic
TVFRSSREKRRSAXIFI

SEQ ID NO: 33 - protein, 17 aa, Artificial Sequence
  Synthetic
TVFRSSREXRRSADIFI

SEQ ID NO: 34 - protein, 17 aa, Artificial Sequence
  Synthetic
TVFWSSREKRRSADIFI

SEQ ID NO: 35 - protein, 16 aa, Artificial Sequence
  Synthetic
VFRSSREKRRSADIFI

SEQ ID NO: 36 - protein, 15 aa, Artificial Sequence
  Synthetic
FRSSREKRRSADIFI

SEQ ID NO: 37 - protein, 15 aa, Artificial Sequence
  Synthetic
SSREKRRSADIFIAS

SEQ ID NO: 38 - protein, 14 aa, Artificial Sequence
  Synthetic
RSSREKRRSADIFI

SEQ ID NO: 39 - protein, 14 aa, Artificial Sequence
  Synthetic
FRSSREKRRSADIA

SEQ ID NO: 40 - protein, 14 aa, Artificial Sequence
  Synthetic
FRSSREKRRSADIV

SEQ ID NO: 41 - protein, 14 aa, Artificial Sequence
  Synthetic
TVFRSSREKRRSAD

SEQ ID NO: 42 - protein, 14 aa, Artificial Sequence
  Synthetic
SSREKRRSADIFIA

SEQ ID NO: 43 - protein, 14 aa, Artificial Sequence
  Synthetic
VSSREKRRSADIFI

SEQ ID NO: 44 - protein, 13 aa, Artificial Sequence
  Synthetic
SSREKRRSADIFI

SEQ ID NO: 45 - protein, 13 aa, Artificial Sequence
  Synthetic
FRSSREKRRSADI

SEQ ID NO: 46 - protein, 12 aa, Artificial Sequence
  Synthetic
FRSSREKRRSAD

SEQ ID NO: 47 - protein, 12 aa, Artificial Sequence
  Synthetic
SREKRRSADIFI

SEQ ID NO: 48 - protein, 11 aa, Artificial Sequence
  Synthetic
REKRRSADIFI

SEQ ID NO: 49 - protein, 11 aa, Artificial Sequence
  Synthetic
RSSREKRRSAD

SEQ ID NO: 50 - protein, 10 aa, Artificial Sequence
  Synthetic
REKRRSADIF

SEQ ID NO: 51 - protein, 10 aa, Artificial Sequence
  Synthetic
SSREKRRSAD

SEQ ID NO: 52 - protein, 9 aa, Artificial Sequence
  Synthetic
REKRRSADI

SEQ ID NO: 53 - protein, 9 aa, Artificial Sequence
  Synthetic
SREKRRSAD

SEQ ID NO: 54 - protein, 9 aa, Artificial Sequence
  Synthetic
EREKRRSAD

SEQ ID NO: 55 - protein, 8 aa, Artificial Sequence
  Synthetic
REKRRSAD

SEQ ID NO: 56 - protein, 22 aa, Homo Sapiens
DRYLAIVRPVANARLRLRVSGA

SEQ ID NO: 57 - protein, 18 aa, Artificial Sequence
  Synthetic
LAIVRPVANARLRLRVSG

SEQ ID NO: 58 - protein, 17 aa, Artificial Sequence
  Synthetic
AIVRPVANARLRLRVSG

SEQ ID NO: 59 - protein, 16 aa, Artificial Sequence
  Synthetic
IVRPVANARLRLRVSG

SEQ ID NO: 60 - protein, 15 aa, Artificial Sequence
  Synthetic
VRPVANARLRLRVSG

SEQ ID NO: 61 - protein, 14 aa, Artificial Sequence
  Synthetic
RPVANARLRLRVSG

SEQ ID NO: 62 - protein, 14 aa, Artificial Sequence
  Synthetic
VRPVANARLRLRVS

SEQ ID NO: 63 - protein, 13 aa, Artificial Sequence
  Synthetic
AIVRPVANARLRL

SEQ ID NO: 64 - protein, 13 aa, Artificial Sequence
  Synthetic
RPVANARLRLRVS

SEQ ID NO: 65 - protein, 13 aa, Artificial Sequence
  Synthetic
VRPVANARLRLLL

SEQ ID NO: 66 - protein, 13 aa, Artificial Sequence
  Synthetic
VRPVANARLRLRV

SEQ ID NO: 67 - protein, 12 aa, Artificial Sequence
  Synthetic
RPVANARLRLRV

SEQ ID NO: 68 - protein, 12 aa, Artificial Sequence
  Synthetic
VRPVANARLRLR

SEQ ID NO: 69 - protein, 11 aa, Artificial Sequence
  Synthetic
RPVANARLRLR

SEQ ID NO: 70 - protein, 11 aa, Artificial Sequence
  Synthetic
VRPVANARLRL

SEQ ID NO: 71 - protein, 10 aa, Artificial Sequence
  Synthetic
RPVANARLRL

SEQ ID NO: 72 - protein, 10 aa, Artificial Sequence
  Synthetic
VRPVANARLR

SEQ ID NO: 73 - protein, 9 aa, Artificial Sequence
  Synthetic
VRPVANARL

SEQ ID NO: 74 - protein, 30 aa, Homo Sapiens
IAQTIAGHFRKERIEGLRKRRRLLSIIVVL

SEQ ID NO: 75 - protein, 23 aa, Artificial Sequence
  Synthetic
QTIAGHFRKERIEGLRKRRRLLS

SEQ ID NO: 76 - protein, 23 aa, Artificial Sequence
  Synthetic
QTIAGHFRKERIEGLRKRRRLLA

SEQ ID NO: 77 - protein, 23 aa, Artificial Sequence
  Synthetic
QTIAGHFRKERIEGLRKRRRLLF

SEQ ID NO: 78 - protein, 23 aa, Artificial Sequence
  Synthetic
QTIAGHFRKERIEGLRKRRRLLW

SEQ ID NO: 79 - protein, 23 aa, Artificial Sequence
  Synthetic
GSGSGHFRKERIEGLRKRRRLLA

SEQ ID NO: 80 - protein, 22 aa, Artificial Sequence
  Synthetic
SGSGHFRKERIEGLRKRRRLLA

SEQ ID NO: 81 - protein, 21 aa, Artificial Sequence
  Synthetic
IAGHFRKERIEGLRKRRRLLS

SEQ ID NO: 82 - protein, 21 aa, Artificial Sequence
  Synthetic
QTIAGHFRKERIEGLRKRRRL

SEQ ID NO: 83 - protein, 21 aa, Artificial Sequence
  Synthetic
GSGHFRKERIEGLRKRRRLLA

SEQ ID NO: 84 - protein, 20 aa, Artificial Sequence
  Synthetic
SGHFRKERIEGLRKRRRLLA

SEQ ID NO: 85 - protein, 19 aa, Artificial Sequence
  Synthetic
GHFRKERIEGLRKRRRLLS

SEQ ID NO: 86 - protein, 19 aa, Artificial Sequence
  Synthetic
QTIAGHFRKERIEGLRKRR

SEQ ID NO: 87 - protein, 19 aa, Artificial Sequence
  Synthetic
GHFRKERIEGLRKRRRLLA

SEQ ID NO: 88 - protein, 18 aa, Artificial Sequence
  Synthetic
HFRKERIEGLRKRRRLLS

SEQ ID NO: 89 - protein, 17 aa, Artificial Sequence
  Synthetic
QTIAGHFRKERIEGLRK

SEQ ID NO: 90 - protein, 17 aa, Artificial Sequence
  Synthetic
FRKERIEGLRKRRRLLA

SEQ ID NO: 91 - protein, 16 aa, Artificial Sequence
  Synthetic
RKERIEGLRKRRRLLS

SEQ ID NO: 92 - protein, 16 aa, Artificial Sequence
  Synthetic
ERIEGLRKRRRLLSII

SEQ ID NO: 93 - protein, 16 aa, Artificial Sequence
  Synthetic
HFRKERIEGLRKRRRL

SEQ ID NO: 94 - protein, 15 aa, Artificial Sequence
  Synthetic
FRKERIGGKRRRLLA

SEQ ID NO: 95 - protein, 14 aa, Artificial Sequence
  Synthetic
ERIEGLRKRRRLLS

SEQ ID NO: 96 - protein, 14 aa, Artificial Sequence
  Synthetic
HFRKERIEGLRKRR

SEQ ID NO: 97 - protein, 12 aa, Artificial Sequence
  Synthetic
QTIAGHFRKERI

SEQ ID NO: 98 - protein, 11 aa, Artificial Sequence
  Synthetic
EGLRKRRRLLA

SEQ ID NO: 99 - protein, 11 aa, Artificial Sequence
  Synthetic
QTIAGHFRKER

SEQ ID NO: 100 - protein, 70 aa, Homo Sapiens
FFDPRFRQACTSMLCCGQSRCAGTSHSSSGEKSASYSSGHSQGPGPNMGKGGEQMHEKSIPYSQETLVVD

SEQ ID NO: 101 - protein, 14 aa, Artificial Sequence
  Synthetic
FFDPRFRQASTSML

SEQ ID NO: 102 - protein, 13 aa, Artificial Sequence
  Synthetic
FDPRFRQASTSML

SEQ ID NO: 103 - protein, 12 aa, Artificial Sequence
  Synthetic
DPRFRQASTSML

SEQ ID NO: 104 - protein, 18 aa, Artificial Sequence
  Synthetic
TVFRSSREKRRSADIFIA

SEQ ID NO: 105 - protein, 18 aa, Artificial Sequence
  Synthetic
TVFRSSREKRRSADIFIS

SEQ ID NO: 106 - protein, 19 aa, Artificial Sequence
  Synthetic
TVFRSSREKRRSADIFISA

SEQ ID NO: 107 - protein, 19 aa, Artificial Sequence
  Synthetic
TVFRSSREKRRSADIFIAS

SEQ ID NO: 108 - protein, 17 aa, Artificial Sequence
  Synthetic
TVFRSSREXRRSADIFI

SEQ ID NO: 109 - protein, 18 aa, Artificial Sequence
  Synthetic
TVFRSSREXKRRSADIFI

SEQ ID NO: 110 - protein, 18 aa, Artificial Sequence
  Synthetic
TVFRSSRXEKRRSADIFI

SEQ ID NO: 111 - protein, 17 aa, Artificial Sequence
  Synthetic
TVFRSSREXRRSADIFI

SEQ ID NO: 112 - protein, 17 aa, Artificial Sequence
  Synthetic
TVFRSSREXRRSADIFI

(19) United States
(12) Patent Application Publication
(10) Pub. No.: US 2012/0028888 A1
(43) Pub. Date: Feb. 2, 2012

(54) APJ RECEPTOR COMPOUNDS

(76) Inventors: Jay Janz, Natick, MA (US); Athan Kuliopulos, Winchester, MA (US); Thomas J. McMurry, Winchester, MA (US)

(21) Appl. No.: 13/127,428

(22) PCT Filed: Nov. 4, 2009
(86) PCT No.: PCT/US09/05974

§ 371 Date: Aug. 22, 2011

(30) Foreign Application Priority Data
Nov. 4, 2008 (US) 61198292

(51) Int. Cl. A61K 38/16; C07K 7/06; C07K 7/08; C07K 14/00; C07K 5/10; A61K 38/03; A61K 38/04; A61K 38/08; A61K 38/10; A61P 9/12; A61P 9/10; A61P 35/00; A61P 35/04; A61P 3/10; A61P 3/04; A61P 37/00; A61P 31/18; C07K 5/08

(52) U.S. Cl. 514/3.8; 530/331; 530/329; 530/326; 530/325; 530/330; 530/327; 530/328; 530/324; 514/19.3; 514/6.9; 514/4.8; 514/21.9; 514/21.8; 514/21.7; 514/21.6; 514/21.5; 514/21.4; 514/21.3; 514/16.4; 514/15.7; 514/19.8

(57) ABSTRACT

The invention relates generally to compounds which are allosteric modulators (e.g., negative and positive allosteric modulators, allosteric agonists, and ago-allosteric modulators) of the G protein coupled receptor apelin, also known as the APJ receptor. The APJ receptor compounds are derived from the intracellular loops and domains of the the APJ receptor. The invention also relates to the use of these APJ receptor compounds and pharmaceutical compositions comprising the APJ receptor compounds in the treatment of diseases and conditions associated with APJ receptor modulation, such as heart diseases (e.g., hypertension and tension and heart failure, such as congestive heart failure), cancer, diabetes, stem cell trafficking, fluid homeostasis, cell proliferation, immune function, obesity, metastatic disease, and HIV infection.

### RELATED APPLICATIONS

This application claims the benefit of U.S. Provisional Application No. 61/198,292, filed on Nov. 4, 2008. The entire teachings of the above application is incorporated herein by reference.

### BACKGROUND OF THE INVENTION

G protein coupled receptors (GPCRs) constitute one of the largest families of genes in the human genome. GPCRs are integral membrane signaling proteins. Hydrophobicity mapping of the amino acid sequences of G-protein coupled receptors has led to a model of the typical G-protein-coupled receptor as containing seven hydrophobic membrane-spanning regions with the amino terminal on the extracellular side of the membrane and the carboxyl terminal on the intracellular side of the membrane.

GPCRs mediate the transmission of intracellular signals (“signal transduction”) by activating guanine nucleotide-binding proteins (G proteins) to which the receptor is coupled. GPCRs are activated by a wide range of endogenous stimuli, including peptides, amino acids. hormones, light, and metal ions. The following reviews are incorporated by reference: Hill, British J. Pharm 147: s27 (2006); Palczeski, Ann Rev Biochemistry 75: 743-767 (2006); Dorsham & Gutkind, Nature Reviews 7: 79-94 (2007); Kobilka & Schertler, Trends Pharmacol Sci. 2: 79-83 (2008).

GPCRs are important targets for drug discovery as they are involved in a wide range of cellular signaling pathways and are implicated in many pathological conditions (e.g., cardiovascular and mental disorders, cancer, AIDS). In fact, GPCRs are targeted by 40-50% of approved drugs, illustrating the critical importance of this class of pharmaceutical targets. Interestingly, this number represents only about 30 GPCRs, a small fraction of the total number of GPCRs thought to be relevant to human disease. Over 1000 GPCRs are known in the human genome, and GPCRs remain challenging targets from a research and development perspective in part because these amembrane bound receptors with complex pharmacology.

There remains a need for the development of new pharmaceuticals that are allosteric modulators of GPCRs (e.g., negative and positive allosteric modulators, allosteric agonists, and ago-allosteric modulators).

### SUMMARY OF THE INVENTION

The invention relates generally to compounds which are allosteric modulators (e.g., negative and positive allosteric modulators, allosteric agonists, and ago-allosteric modulators) of the G protein coupled receptor apelin, also known as the APJ receptor. The APJ receptor compounds are derived from the intracellular loops and domains of the the APJ receptor. The invention also relates to the use of these APJ receptor compounds and pharmaceutical compositions comprising the APJ receptor compounds in the treatment of diseases and conditions associated with APJ receptor modulation, such as heart diseases (e.g., hypertension and heart failure, such as congestive heart failure), cancer, diabetes, stem cell trafficking, fluid homeostasis, cell proliferation, immune function, obesity, metastatic disease, and HIV infection.

More specifically, the invention relates to compounds represented by Formula I:

TLP,

or pharmaceutically acceptable salts thereof, wherein:

P is a peptide comprising at least three contiguous amino-acid residues of an intracellular i1, i2, i3 loop or an intracellular i4 domain of the APJ receptor;

L is a linking moiety represented by C(O) and bonded to P at an N terminal nitrogen of an N-terminal amino-acid residue;

and T is a lipophilic tether moiety bonded to L, wherein the C-terminal amino acid residue of P is optionally functionalized.

The invention also relates to pharmaceutical compositions comprising one or more compounds of the invention and a carrier, and the use of the disclosed compounds and compositions in methods of treating diseases and conditions responsive to modulation (inhibition or activation) of the APJ receptor.

The invention also relates to pharmaceutical compositions comprising one or more compounds of the invention and a carrier, and the use of the disclosed compounds and compositions in methods of treating diseases and conditions responsive to modulation of the APJ receptor.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A is a graph showing the inhibition of NKH477 stimulated increase in cAMP in HEK cells stably expressing the APJ receptor upon treatment with apelin or Compound 51.

FIG. 1B. is a graph showing the inhibition of NKH477 stimulated increase in cAMP in HEK cells stably expressing the APJ receptor upon treatment with apelin or Compound 12.

FIG. 2A is a bar graph that describes the effect of apelin-13 on mouse heart function at 15 minutes. Apelin concentration is shown on the x-axis.

FIG. 2B is a bar graph that describes the effect of Compound 12 on mouse heart function at 15 minutes. Compound 12 concentration is shown on the x-axis.

### DETAILED DESCRIPTION OF THE INVENTION

A description of example embodiments of the invention follows.

### G Protein Coupled Receptors (GPCRs)

G protein coupled receptors (GPCRs) constitute one of the largest superfamilies of genes in the human genome; these transmembrane proteins enable the cell the respond to its environment by sensing extracellular stimuli and initiating intracellular signal transduction cascades. GPCRs mediate signal transduction through the binding and activation of guanine nucleotide-binding proteins (G proteins) to which the receptor is coupled. Wide arrays of ligands bind to these receptors, which in turn orchestrate signaling networks integral to many cellular functions. Diverse GPCR ligands include small proteins, peptides, amino acids, biogenic amines, lipids, ions, odorants and even photons of light. The following reviews are incorporated by reference: Hill, British J. Pharm 147: s27 (2006); Dorsham & Gutkind, Nature Reviews 7: 79-94 (2007).

In addition to modulating a diverse array of homeostatic processes, GPCR signaling pathways are integral components of many pathological conditions (e.g., cardiovascular and mental disorders, cancer, AIDS). In fact, GPCRs are targeted by 40-50% of approved drugs illustrating the critical importance of this class of pharmaceutical targets. Interestingly, this number represents only about 30 GPCRs, a small fraction of the total number of GPCRs thought to be relevant to human disease. GPCRs are membrane bound receptors that exhibit complex pharmacological properties and remain challenging targets from a research and development perspective. Given their importance in human health combined with their prevalence (over 1000 known GPCRs in the human genome) GPCRs represent an important target receptor class for drug discovery and design.

GPCRs are integral membrane proteins that mediate diverse signaling cascades through an evolutionarily conserved structural motif. All GPCRs are thought to consist of seven hydrophobic transmembrane spanning α-helices with the amino terminus on the extracellular side of the membrane and the carboxyl terminus on the intracellular side of the membrane. The transmembrane helices are linked together sequentially by extracellular (e1, e2, e3) and intracellular (cytoplasmic) loops (i1, i2, i3). The intracellular loops or domains are intimately involved in the coupling and turnover of G proteins and include: il, which connects TM1-TM2; i2, connecting TM3-TM4; i3, connecting TM5-TM6; and a portion of the C-terminal cytoplasmic tail (domain 4). Due in part to the topological homology of the 7TM domains and the recent high resolution crystal structures of several GPCRs (Palczewski et al., Science 289, 739-45 (2000), Rasmussen, S.G. et al., Nature 450, 383-7 (2007)) skilled modelers are now able to predict the general boundaries of GPCR loop domains through the alignment of several related receptors. These predictions are aided in part by a number of programs used by computational biologists, including EMBOSS, ClustalW2, Kalign, and MAFFT (Multiple Alignment using Fast

Fourier Transform). Importantly, many of these programs are publically available (see, for example, The European Bioinformatics Institute (EMBL-EBI) web site http://www.ebi.ac.uk/Tools/) and most have web-based interfaces.

GPCR mediated signal transduction is initiated by the binding of a ligand to its cognate receptor. In many instances GPCR ligand binding is believed to take place in a hydrophilic pocket generated by a cluster of helices near the extracellular domain. However, other ligands, such as large peptides, are thought to bind to the extracellular region of protein and hydrophobic ligands are postulated to intercalate into a receptor binding pocket through the membrane between gaps in the helices. The process of ligand binding induces conformational changes within the receptor. These changes involve the outward movement of helix 6, which in turn alters the conformations of the intracellular loops and ultimately results in a receptor form that is able to bind and activate a heterotrimeric G protein (Farrens, D., et al. Science 274, 768-770 (1996), Gether, U. and Kobilka, B., J. Biol. Chem. 273, 17979-17982 (1998)). Upon binding the receptor catalyzes the exchange of GTP for GDP in the alpha subunit of the heterotrimeric G protein, which results in a separation of the G protein from the receptor as well a dissociation of the alpha and beta/gamma subunits of the G protein itself. Notably, this process is catalytic and results in signal amplification in that activation of one receptor may elicit the activation and turnover of numerous G proteins, which in turn may regulate multiple second messenger systems. Signaling diversity is further achieved through the existence of numerous G protein types as well as differing isoforms of alpha, beta and gamma subunits. Typically, GPCRs interact with G proteins to regulate the synthesis or inhibition of intracellular second messengers such as cyclic AMP, inositol phosphates, diacylglycerol and calcium ions, thereby triggering a cascade of intracellular events that eventually leads to a biological response.

GPCR signaling may be modulated and attenuated through cellular machinery as well as pharmacological intervention. Signal transduction may be ‘switched off with relatively fast kinetics (seconds to minutes) by a process called rapid desensitization. For GPCRs, this is caused by a functional uncoupling of receptors from heterotrimeric G proteins, without a detectable change in the total number of receptors present in cells or tissues. This process involves the phosphorylation of the receptor C terminus, which enables the protein Arrestin to bind to the receptor and occulude further G protein coupling. Once bound by Arrestin the receptor may be internalized into the cell and either recycled back to the cell surface or degraded. The alpha subunit of the G protein possesses intrisic GTPase activity, which attenuates signaling and promotes re-association with the beta/gamma subunits and a return to the basal state. GPCR signaling may also be modulated pharmacologically. Agonist drugs act directly to activate the receptors, whereas antagonist drugs act indirectly to block receptor signaling by preventing agonist activity through their associating with the receptor.

GPCR binding and signaling can also be modified through allosteric modulation, that is by ligands that bind not at the orthosteric binding site but through binding at an allosteric site elsewhere in the receptors. Allosteric modulators can include both positive and negative modulators of orthosteric ligand mediated activity, allosteric agonists (that act in the absence of the orthosteric ligand), and ago-allosteric modulators (ligands that have agonist activity on their own but that can also modulate the activity of the orthosteric ligand).

The large superfamily of GPCRs may be divided into subclasses based on structural and functional similarities. GPCR families include Class A Rhodopsin like, Class B Secretin like, Class C Metabotropic glutamate/pheromone, Class D Fungal pheromone, Class E cAMP receptors (Dictyostelium), the Frizzled/Smoothened family, and various orphan GPCRs. In addition, putative families include Ocular albinism proteins, Insect odorant receptors, Plant Mlo receptors, Nematode chemoreceptors, Vomeronasal receptors (VIR & V3R) and taste receptors.

Class A GPCRs, also called family A or rhodopsin-like, are the largest class of receptors and characteristically have relatively small extracellular loops that form the basis for selectivity vs. endogenous agonists and small-molecule drugs. In addition, Class A receptors also have relatively small intracellular loops. Class A receptors include amine family members such as dopamine and serotonin, peptide members such as chemokine and opioid, the visual opsins, odorant receptors and an array of hormone receptors.

The apelin receptor (APJ) is a Class A receptor that has been implicated in conditions such as heart diseases, such as heart diseases (e.g., hypertension and heart failure, such as congestive heart failure), cancer, diabetes, stem cell trafficking, fluid homeostasis, cell proliferation, immune function, obesity, metastatic disease, and HIV infection.

### Peptides

As defined herein, P is a peptide comprising at least three contiguous amino-acid residues (e.g., at least 3, 4, 5, 6, 7, 8, 9, 10, 11, 12, 13, 14, 15, 16, or 17) of an intracellular i1, i2 or i3 loop or intracellular i4 domain of the apelin (APJ) receptor. It is understood that, the N-terminal nitrogen of the N-terminal amino acid residue of P to which the linking moiety—C(O) is bonded can be one of the at least three contiguous amino acid residues or it can be an amino acid residue distinct from the at least three contiguous amino acid residues.

Intracellular i1 loop as used herein refers to the loop which connects TM1 to TM2 and the corresponding transmembrane junctional residues.

Intracellular i2 loop as used herein refers to the loop which connects TM3 to TM4 and the corresponding transmembrane junctional residues.

Intracellular i3 loop as used herein refers to the loop which connects TM5 to TM6 and the corresponding transmembrane junctional residues.

Intracellular i4 domain as used herein refers to the C-terminal cytoplasmic tail and the transmembrane junctional residue.

In a specific embodiment, P comprises at least three, at least four, at least five, at least six, at least seven, at least eight, at least nine, at least ten, at least eleven, at least twelve, at least thirteen, at least fourteen, at least fifteen, at least sixteen or at least seventeen contiguous amino acid residues of the intracellular i1, i2 or i3 loop or intracellular i4 domain of the apelin receptor (APJ).

In a more specific embodiment, the at least three contiguous amino acids of P (e.g., at least 3, 4, 5, 6, 7, 8, 9, 10, 11, 12, 13, 14, 15, 16, or 17) are derived are from the intracellular il, i2 or i3 loop or intracellular i4 domain of the apelin receptor (APJ), wherein the amino acid sequence of each loop and the i4 domain is as described in Table 1.

TABLE 1

| Intracellular | |
|---|---|
| Loop Number | APJ Receptor Intercellular Loop |
| i1 | TVFRSSREKRRSADIFI SEQ ID NO: 1 |
| i2 | DRYLAIVRPVANARLRLRVSGA SEQ ID NO: 56 |
| i3 | IAQTIAGHFRKERIEGLRKRRRLLSIIVVL |
| | SEQ ID NO: 74 |
| i4 | FFDPRFRQACTSMLCCGQSRCAGTSHSSSGEKSAS |
| | YSSGHSQGPGPNMGKGGEQMHEKSIPYSQETLVVD |
| | SEQ ID NO: 100 |

It is understood that in addition to the amino acids shown in the sequences in Table 1, the intracellular loop for the i1 loop, i2 loop, i3 loop and i4 domain can also include the transmembrane junctional residues. For example, the i1 loop can include SEQ ID NO: 1 where one or more residues from the transmembrane junctional residues are included on either the C-terminus, the N-terminus or both. For example, SEQ ID NO: 1 can include either an Alanine residue, Serine residue or both in either order at the C-terminus. Such sequences can be identified as SEQ ID NO: 104, SEQ ID NO. 105, SEQ ID NO: 106, and SEQ ID NO: 107, respectively (i.e, TVFRSSREKRRSADIFIA, SEQ ID NO: 104; TVFRSSREKRRSADIFIS, SEQ ID NO: 105; TVFRSSREKRRSADIFISA, SEQ ID NO:

106; and TVFRSSREKRRSADIFIAS, SEQ ID NO: 107)

In another embodiment, P comprises at least three, at least four, at least five, at least six, at least seven, at least eight, at least nine, at least ten, at least eleven, at least twelve, at least thirteen, at least fourteen, at least fifteen, at least sixteen, or at least seventeen contiguous amino acid residues of the i1 intracellular loop of the APJ receptor.

It is understood that for the embodiments presented herein, that when the amino acid residues of P are represented by X, M, Y or Z that the C-terminal amino acid residue does not include the —OH of the amino acid and that the end group R₁ that is bonded to the C-terminal residue includes —OH as well as other moieties defined herein.

In one embodiment, P is derived from the i1 loop and is represented by the following structural formula or pharmaceutically acceptable salts thereof,

X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-R₁,

wherein
X₁ is absent or a threonine or alanine residue;
X₂ is absent or a valine or alanine residue;
X₃ is absent or a phenylalanine or alanine residue;
X₄ is absent or an arginine, tryptophan, valine or alanine residue;
X₅ is absent or a serine or alanine residue;
X₆ is absent or a serine, glutamic acid or alanine residue;
X₇ is absent or an arginine or alanine residue;
X₈ is absent or a glutamic acid, alanine or proline residue;
X₉ is absent or a lysine, alanine, proline, glutamic acid, D-2,3-diaminionpropionic acid, or D-ornithine;
X₁₀ is absent or an arginine, alanine or lysine residue;
X₁₁ is absent or an arginine or alanine residue;
X₁₂ is absent or a serine, aspartic acid, alanine or arginine residue;
X₁₃ is absent or an alanine or serine residue;
X₁₄ is absent or an aspartic acid or alanine residue;
X₁₅ is absent or an isoleucine, alanine or aspartic acid residue;
X₁₆ is absent or a phenylalanine, valine, alanine or isoleucine residue;
X₁₇ is absent or an isoleucine, alanine or phenylalanine residue;

X₁₈ is absent or an alanine or isoleucine residue;
X₁₉ is absent or a serine residue;
provided that at least five of X₁- X₁₉ are present;
R₁ is OR₂ or N(R₂)₂;
each R₂ is independently hydrogen or (C₁-C₁₀)alkyl; and
from 0 to 5 amino acid residues are present in the D configuration.

It is understood that when P is described as X₁- X₁₉ it is bonded to L as written. For example, X₁ is bonded to L. If X₁ is absent, then X₂ is bonded to L.

In a specific embodiment, at least four of X₇, X₈, X₉, X₁₀, X₁₁, X₁₂, X₁₃ and X₁₄ are present.

In another specific embodiment,
X₇ is an arginine or alanine;
X₈ is a glutamic acid, alanine or proline;
X₉ is a lysine, alanine, proline, glutamic acid, D-2,3-diaminionpropionic acid, or D-ornithine; and
X₁₀ is an arginine, alanine or lysine.

In a further specific embodiment, at least one of X₇, X₈, X₉, or X₁₀ is alanine.

In another specific embodiment,
X₁₁ is an arginine or alanine;
X₁₂ is a serine, aspartic acid, alanine or arginine;
X₁₃ is an alanine or serine; and
X₁₄ is an aspartic acid or alanine.

In a further specific embodiment, at least one of X₁₁, X₁₂, X₁₃ or X₁₄ is alanine.

In another specific embodiment,
X₇ is an arginine or alanine;
X₈ is a glutamic acid, alanine or proline;
X₉ is a lysine, alanine, proline, glutamic acid, D-2,3-diaminionpropionic acid, or D-ornithine;
X₁₀ is an arginine, alanine or lysine;
X₁₁ is an arginine or alanine;
X₁₂ is a serine, aspartic acid, alanine or arginine;
X₁₃ is an alanine or serine; and
X₁₄ is an aspartic acid or alanine.

In another specific embodiment, at least one of X₇, X₈, X₉, X₁₀, X₁₁, X₁₂, X₁₃ or X₁₄ is alanine.

In yet another specific embodiment,
X₇ is an arginine;
X₈ is a glutamic acid;
X₉ is a lysine; and
X₁₀ is an arginine.

In yet another specific embodiment,
X₁₁ is an arginine;
X₁₂ is a serine;
X₁₃ is an alanine; and
X₁₄ is an aspartic acid.

In an even more specific embodiment, P is selected from the group consisting of SEQ ID NOS: 1-55 as listed in Table 2a below:

TABLE 2a

| APJ | | SEQ ID |
|---|---|---|
| i-Loop | Sequence | NO: |
| i1 | TVFRSSREKRRSADIFI | 1 |
| i1 | AVFRSSREKRRSADIFI | 2 |
| i1 | TAFRSSREKRRSADIFI | 3 |
| i1 | TVARSSREKRRSADIFI | 4 |
| i1 | TVFASSREKRRSADIFI | 5 |
| i1 | TVFRASREKRRSADIFI | 6 |
| i1 | TVFRSAREKRRSADIFI | 7 |
| i1 | TVFRSSAEKRRSADIFI | 8 |
| i1 | TVFRSSRAKRRSADIFI | 9 |
| i1 | TVFRSSREARRSADIFI | 10 |
| i1 | TVFRSSREKARDADIFI | 11 |
| i1 | TVFRSSREKRASADIFI | 12 |
| i1 | TVFRSSREKRRAADIFI | 13 |
| i1 | TVFRSSREKRRSAAIFI | 14 |
| i1 | TVFRSSREKRRSADAFI | 15 |
| i1 | TVFRSSREKRRSADIAI | 16 |
| i1 | TVFRSSREKRRSADIFA | 17 |
| i1 | tVFRSSREKRRSADIFI | 18 |
| i1 | TvFRSSREKRRSADIFI | 19 |
| i1 | TVfRSSREKRRSADIFI | 20 |
| i1 | TVFrSSREKRRSADIFI | 21 |
| i1 | TVFRsSREKRRSADIFI | 22 |
| i1 | TVFRSsREKRRSADIFI | 23 |
| i1 | TVFRSSrEKRRSADIFI | 24 |
| i1 | TVFRSSReKRRSADIFI | 25 |
| i1 | TVFRSSREKrRSADIFI | 26 |
| 11 | TVFRSSREKRrSADIFI | 27 |
| i1 | TVFRSSREKRRSADiFI | 28 |
| i1 | TVFRSSREKRRSADIFi | 29 |
| i1 | TVFRSSREKRRsADIFI | 30 |
| i1 | TVFRSSREKRRSaDIFI | 31 |
| i1 | TVFRSSREKRRSAdIFI | 32 |
| i1 | TVFRSSREkRRSADIFI | 33 |
| i1 | TVFWSSREKRRSADIFI | 34 |
| i1 | VFRSSREKRRSADIFI | 35 |
| i1 | FRSSREKRRSADIFI | 36 |
| i1 | SSREKRRSADIFIAS | 37 |
| i1 | RSSREKRRSADIFI | 38 |
| i1 | FRSSREKRRSADIA | 39 |
| i1 | FRSSREKRRSADIV | 40 |
| i1 | TVFRSSREKRRSAD | 41 |
| i1 | SSREKRRSADIFIA | 42 |
| i1 | VSSREKRRSADIFI | 43 |
| i1 | SSREKRRSADIFI | 44 |
| i1 | FRSSREKRRSADI | 45 |
| i1 | FRSSREKRRSAD | 46 |
| 11 | SREKRRSADIFI | 47 |
| i1 | REKRRSADIFI | 48 |
| i1 | RSSREKRRSAD | 49 |
| i1 | REKRRSADIF | 50 |
| i1 | SSREKRRSAD | 51 |
| i1 | REKRRSADI | 52 |
| i1 | SREKRRSAD | 53 |
| i1 | EREKRRSAD | 54 |
| i1 | REKRRSAD | 55 |

In another even more specific embodiment, P is selected from the group consisting of SEQ ID NOS: 108-112 as listed in Table 2b below:

TABLE 2b

| APJ | | SEQ ID |
|---|---|---|
| i-Loop | Sequence | NO: |
| i1 | TVFRSSRE(D-Dap)RRSADIFI | 108 |
| i1 | TVFRSSREpKRRSADIFI | 109 |
| i1 | TVFRSSRpEKRRSADIFI | 110 |
| I1 | TVFRSSRE(D-Dab)RRSADIFI | 111 |
| i1 | TVFRSSRE(D-Orn)RRSADIFI | 112 |

In another specific embodiment, P comprises at least three, at least four, at least five, at least six, at least seven, at least eight, at least nine, at least ten, at least eleven, at least twelve, at least thirteen, at least fourteen, at least fifteen, at least sixteen, or at least seventeen, contiguous amino acid residues of the i2 intracellular loop of the apelin (APJ) receptor.

In one embodiment, P derived from the i2 loop and is represented by the following structural formula or a pharmaceutically acceptable salt thereof,

Y₁-Y₂-Y₃-Y₄-Y₅-Y₆-Y₇-Y₈-Y₉-Y₁₀-Y₁₁-Y₁₂-Y₁₃-Y₁₄-Y₁₅-Y₁₆-Y₁₇-Y₁₈-Y₁₉-Y₂₀-Y₂₁-Y₂₂-R₁ wherein:

Y₁ is absent or an aspartic acid residue;

Y₂ is absent or an arginine residue;

Y₃ is absent or a tyrosine residue;

Y₄ is absent or a leucine residue;

Y₅ is absent or an alanine residue;

Y₆ is absent or an isoleucine residue;

Y₇ is absent or a valine residue;

Y₈ is absent or an arginine residue;

Y₉ is absent or a proline residue;

Y₁₀ is absent or a valine residue;

Y₁₁ is absent or an alanine residue;

Y₁₂ is absent or an asparagine residue;

Y₁₃ is absent or an alanine residue;

Y₁₄ is absent or an arginine residue;

Y₁₅ is absent or a leucine residue;

Y₁₆ is absent or an arginine residue;

Y₁₇ is absent or a leucine residue;

Y₁₈ is absent or an arginine or leucine residue;

Y₁₉ is absent or a valine or leucine residue;

Y₂₀ is absent or a serine;

Y₂₁ is absent or a glycine residue; and

Y₂₂ is absent or an alanine, provided that at least five of Y₁-Y₂₂ are present;

R₁ is OR₂ or N(R₂)₂;

each R₂ is independently hydrogen or (C₁-C₁₀)alkyl; and

from 0 to 5 amino acid residues are present in the D configuration.

It is understood that when P is described as Y₁- Y₂₂ it is bonded to L as written. For example, Y₁ is bonded to L. If Y₁ is absent, then Y₂ is bonded to L.

In a specific embodiment, at least three of Y₈, Y₉, Y₁₀, Y₁₁, Y₁₂, Y₁₃, and Y₁₄ are present.

In a further specific embodiment,

Y₈ is an arginine residue;

Y₉ is a proline residue;

Y₁₀ is a valine residue; and

Y₁₁ is an alanine residue;

In another further specific embodiment,

Y₁₂ is an asparagine residue;

Y₁₃ is an alanine residue; and

Y₁₄ is an arginine residue; and

Y₁₅ is a leucine residue.

In a more specific embodiment, P is selected from the group consisting of SEQ ID NOS: 56-73 as listed in Table 3 below.

TABLE 3

| APJ | | SEQ ID |
|---|---|---|
| i-Loop | Sequence | NOS: |
| i2 | DRYLAIVRPVANARLRLRVSGA | 56 |
| i2 | LAIVRPVANARLRLRVSG | 57 |
| i2 | AIVRPVANARLRLRVSG | 58 |
| i2 | IVRPVANARLRLRVSG | 59 |
| i2 | VRPVANARLRLRVSG | 60 |
| i2 | RPVANARLRLRVSG | 61 |
| i2 | VRPVANARLRLRVS | 62 |
| i2 | AIVRPVANARLRL | 63 |
| i2 | RPVANARLRLRVS | 64 |
| i2 | VRPVANARLRLLL | 65 |
| i2 | VRPVANARLRLRV | 66 |
| i2 | RPVANARLRLRV | 67 |
| i2 | VRPVANARLRLR | 68 |
| i2 | RPVANARLRLR | 69 |
| i2 | VRPVANARLRL | 70 |
| i2 | RPVANARLRL | 71 |
| i2 | VRPVANARLR | 72 |
| i2 | VRPVANARL | 73 |

In yet another specific embodiment, P comprises at least three, at least four, at least five, at least six, at least seven, at least eight, at least nine, at least ten, at least eleven, at least twelve, at least thirteen, at least fourteen, at least fifteen, at least sixteen, or at least seventeen, contiguous amino acid residues of the i3 intracellular loop of the apelin (APJ) receptor.

In one embodiment, P derived from the i3 loop and is represented by the following structural formula or a pharmaceutically acceptable salt thereof,

P is Z₁-Z₂-Z₃-Z₄-Z₅-Z₆-Z₇-Z₈-Z₉-Z₁₀-Z₁₁-Z₁₂-Z₁₃-Z₁₄-Z₁₅-Z₁₆-Z₁₇-Z₁₈-Z₁₉-Z₂₀-Z₂₁-Z₂₂-Z₂₃-Z₂₄-Z₂₅-Z₂₆-Z₂₇-Z₂₈-Z₂₉-Z₃₀-R₁,

wherein :

Z₁ is absent or an isoleucine residue;

Z₂ is absent or an alanine residue;

Z₃ is absent or a glutamine or glycine residue;

Z₄ is absent or a threonine or serine residue;

Z₅ is absent or a isoleucine or glycine residue;

Z₆ is absent or an alanine or serine residue;

Z₇ is absent or a glycine residue;

Z₈ is absent or a histidine residue;

Z₉ is absent or a phenylalanine residue;

Z₁₀ is absent or an arginine residue;

Z₁₁ is absent or a lysine residue;

Z₁₂ is absent or a glutamic acid residue;

Z₁₃ is absent or an arginine residue;

Z₁₄ is absent or an isoleucine residue;

Z₁₅ is absent or a glutamic acid or glycine residue;

Z₁₆ is absent or a glycine residue;

Z₁₇ is absent or a leucine residue;

Z₁₈ is absent or an arginine or glycine residue;

Z₁₉ is absent or lysine residue;

Z₂₀ is absent or an arginine residue;

Z₂₁ is absent or an arginine residue;

Z₂₂ is absent or an arginine residue;

Z₂₃ is absent or a leucine residue;

Z₂₄ is absent or a leucine residue;

Z₂₅ is absent or a serine, alanine, phenylalanine or tryptophan residue;

Z₂₆ is absent or an isoleucine residue;

Z₂₇ is absent or an isoleucine residue;

Z₂₈ is absent or a valine residue;

Z₂₉ is absent or a valine residue; and

Z₃₀ is absent or a leucine residue; provided that at least five of Z₁-Z₃₀ are present; and

R₁ is OR₂ or N(R₂)₂;

each R₂ is independently hydrogen or (C₁-C₁₀)alkyl; and

from 0 to 5 amino acid residues are present in the D configuration.

It is understood that when P is described as Z₁-Z₃₀ it is bonded to L as written. For example, Z₁ is bonded to L. If Z₁ is absent, then Z₂ is bonded to L.

In a specific embodiment, at least four of Z₁₂, Z₁₃, Z₁₄, Z₁₅, Z₁₆, Z₁₇, Z₁₈, and Z₁₉ are present.

In a further specific embodiment,

Z₁₂ is a glutamic acid residue;

Z₁₃ is an arginine residue;

Z₁₄ is an isoleucine residue; and

Z₁₅ is a glutamic acid or glycine residue.

In a further specific embodiment,

Z₁₆ is a glycine residue;

Z₁₇ is a leucine residue;

Zᵢ₈ is a arginine residue or glycine residue; and

Z₁₉ is a lysine residue.

In another specific embodiment, at least four of Z₂₁, Z₂₂, Z₂₃, Z₂₄, and Z₂₅ are present.

In a further specific embodiment,

Z₂₁ is an arginine residue;

Z₂₂ is an arginine residue;

Z₂₃ is a leucine residue;

Z₂₄ is a leucine residue; and

Z₂₅ is a serine residue or an alanine , phenylalanine or tryptophan residue.

In another further specific embodiment, Z₂₅ is an alanine residue.

In another specific embodiment, Z₃, Z₄, Z₅, and Z₆ are present.

In a further specific embodiment,

Z₃ is a glutamine residue;

Z₄ is a threonine residue;

Z₅ is an isoleucine residue; and

Z₆ is an alanine residue.

In another further specific embodiment,

Z₃ is a glycine residue;

Z₄ is a serine residue;

Z₅ is a glycine residue; and

Z₆ is a serine residue.

In a more specific embodiment, P is selected from the group consisting of SEQ ID NOS: 74-99, as listed in Table 4 below:

TABLE 4

| APJ | | SEQ ID |
|---|---|---|
| i-Loop | Sequence | NO: |
| i3 | IAQTIAGHFRKERIEGLRKRRRLLSIIVVL | 74 |
| i3 | QTIAGHFRKERIEGLRKRRRLLS | 75 |
| i3 | QTIAGHFRKERIEGLRKRRRLLA | 76 |
| i3 | QTIAGHFRKERIEGLRKRRRLLF | 77 |
| i3 | QTIAGHFRKERIEGLRKRRRLLW | 78 |
| i3 | GSGSGHFRKERIEGLRKRRRLLA | 79 |
| i3 | SGSGHFRKERIEGLRKRRRLLA | 80 |
| i3 | IAGHFRKERIEGLRKRRRLLS | 81 |
| i3 | QTIAGHFRKERIEGLRKRRRL | 82 |
| i3 | GSGHFRKERIEGLRKRRRLLA | 83 |
| i3 | SGHFRKERIEGLRKRRRLLA | 84 |
| i3 | GHFRKERIEGLRKRRRLLS | 85 |
| i3 | QTIAGHFRKERIEGLRKRR | 86 |
| i3 | GHFRKERIEGLRKRRRLLA | 87 |
| i3 | HFRKERIEGLRKRRRLLS | 88 |
| i3 | QTIAGHFRKERIEGLRK | 89 |
| i3 | FRKERIEGLRKRRRLLA | 90 |
| i3 | RKERIEGLRKRRRLLS | 91 |
| i3 | ERIEGLRKRRRLLSII | 92 |
| i3 | HFRKERIEGLRKRRRL | 93 |
| i3 | FRKERI G GKRRRLLA | 94 |
| i3 | ERIEGLRKRRRLLS | 95 |
| i3 | HFRKERIEGLRKRR | 96 |
| i3 | QTIAGHFRKERI | 97 |
| i3 | EGLRKRRRLLA | 98 |
| i3 | QTIAGHFRKER | 99 |

In a further specific embodiment, P comprises at least three, at least four, at least five, at least six, at least seven, at least eight, at least nine, at least ten, at least eleven, at least twelve, at least thirteen, at least fourteen, at least fifteen, at least sixteen, or at least seventeen, contiguous amino acid residues of i4 intracellular domain of the apelin (APJ) receptor.

In one embodiment, P derived from the i4 domain and is represented by the following structural formula or a pharmaceutically acceptable salt thereof, M₁-M₂-M₃-M₄-M₅-M₆-M₇-M₈-M₉-M₁₀-M₁₁-M₁₂-M₁₃-M₁₄-R₁,

wherein:

M₁ is absent or a phenylalanine residue;

M₂ is absent or a phenylalanine residue;

M₃ is absent or an aspartic acid residue;

M₄ is absent or a proline residue;

M₅ is absent or an arginine residue;

M₆ is absent or a phenylalanine residue;

M₇ is absent or an arginine residue;

M₈ is absent or a glutamine residue;

M₉ is absent or an alanine residue;

M₁₀ is absent or a serine residue;

M₁₁ is absent or a threonine residue;

M₁₂ is absent or a serine residue;

M₁₃ is absent or a methionine residue; and

M₁₄ is absent or a leucine residue; provided that at least five of M₁-M₁₄ are present;

R₁ is OR₂ or N(R₂)₂;

each R₂ is independently hydrogen or (C₁-C₁₀)alkyl; and from 0 to 5 amino acid residues are present in the D configuration.

It is understood that when P is described as M₁- M₁₄ it is bonded to L as written. For example, M, is bonded to L. If M₁ is absent, then M₂ is bonded to L.

In a specific embodiment,

M₃ is an aspartic acid residue;

M₄ is a proline residue;

M₅ is an arginine residue; and

M₆ is a phenylalanine residue.

In a more specific embodiment, P is selected from the group consisting of SEQ ID NOS: 101-103, as listed in Table 5 below:

TABLE 5

| APJ | | SEQ ID |
|---|---|---|
| i-Loop | Sequence | NO: |
| i4 | FFDPRFRQASTSML | 101 |
| i4 | FDPRFRQASTSML | 102 |
| i4 | DPRFRQASTSML | 103 |

It is understood that the sequences presented in Tables 2-5 (2b inclusive) can be optionally functionalized at the C-terminus. Functionalized at the C-terminus means that the acid moiety present at the C-terminus is replaced by some other functional group. Suitable functional groups include —C(O)N(R₂)₂, —C(O)OR₃, or C(O)NHC(O)OR₂, where R₂ is hydrogen or a (C₁-C₁₀) alkyl group and R₃ is a (C₁-C₁₀) alkyl group.

It is understood that as long as P comprises the indicated number of contiguous amino acids residues from the apelin (APJ) intracellular loop (i1, i2 or i3) or domain (i4) from which it is derived, the remainder of the peptide, if present, can be selected from:

(a) any natural amino acid residue, unnatural amino acid residue or a combination thereof;

(b) a peptide sequence comprising natural amino acid residues, non-natural amino acid residues and combinations thereof;

(c) a peptide sequence according to (b) comprising one or more peptide backbone modifications;

(d) a peptide sequence according to (c) comprising one or more retro-inverso peptide linkages;

(e) a peptide sequence according to (c) wherein one or more peptide bonds are replace by

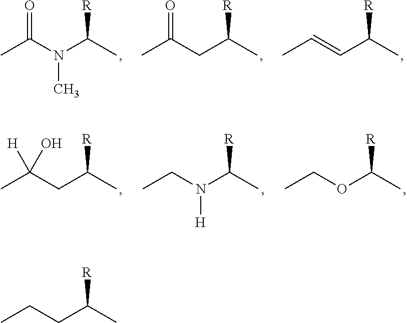

or a combination thereof;

(f) a peptide sequence according to (c) comprising one or more depsipeptide linkages, wherein the amide linkage is replaced with an ester linkage; and

(g) a peptide sequence according to (c) comprising one or more conformational restrictions; and

(h) a peptide sequence according to (c) comprising one or more of (d)-(g).

Furthermore, it is understood that even within the indicated number of contiguous amino acid residues derived from the GPCR intracellular loop (i1, i2 or i3) or domain (i4), there can be: peptide backbone modifications such as, but not limited to, those described in (e) above; retro-inverso peptide linkages; despsipeptide linkages; conformational restrictions; or a combination thereof.

It is noted that P of Formula I can be optionally functionalized at the C-terminus. Functionalized at the C-terminus means that the acid moiety present at the C-terminus is replaced by some other functional group. Suitable functional groups include —C(O)N(R₂)₂, —C(O)O R₃, or C(O)NHC(O)OR₂, where R₂ is hydrogen or a (C₁-C₁₀) alkyl group and R₃ is a (C₁-C₁₀) alkyl group . Functionalization of the C-terminus can result from the methods used to prepare.

Peptidomimetic as used herein refers to a compound comprising non-peptidic structural elements in place of a peptide sequence.

As used herein, the term “amino acid” includes both a naturally occurring amino acid and a non-natural amino acid.

As used herein, the term “naturally occurring amino acid” means a compound represented by the formula NH₂—CHR—COOH, wherein R is the side chain of a naturally occurring amino acids such as lysine, arginine, serine, tyrosine etc. as shown in the Table below.

Table of Common Naturally Occurring Amino Acids

| | Amino acid | Three letter code | One letter code |
|---|---|---|---|
| Non-polar; | alanine | Ala | A |
| neutral at | isoleucine | Ile | I |
| pH 7.4 | leucine | Leu | L |
| | methionine | Met | M |
| | phenylalanine | Phe | F |
| | proline | Pro | P |
| | tryptophan | Trp | W |
| | valine | Val | V |
| Polar, | asparagine | Asn | N |
| uncharged | cysteine | Cys | C |
| at pH 7.0 | glycine | Gly | G |
| | glutamine | Gln | Q |
| | serine | Ser | S |
| | threonine | Thr | T |
| | tyrosine | Tyr | Y |
| Polar; | glutamic acid | Glu | E |
| charged at | arginine | Arg | R |
| pH 7 | aspartic acid | Asp | D |
| | histidine | His | H |
| | lysine | Lys | K |

“Non-natural amino acid” means an amino acid for which there is no nucleic acid codon. Examples of non-natural amino acids include, for example, the D-isomers of the natural α-amino acids such as D-proline (D-P, D-Pro) as indicated above; natural α-amino acids with non-natural side chains (e.g.,

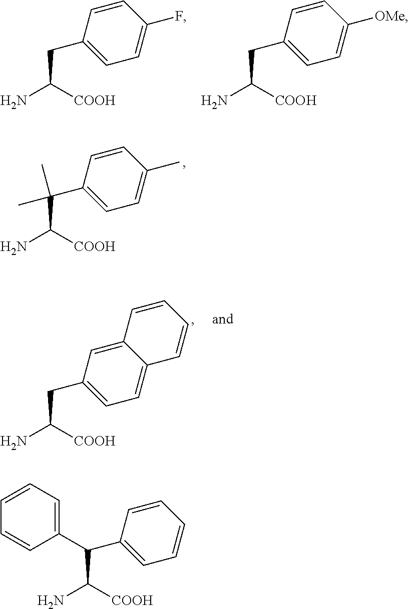

related to phenylalanine); Aib (aminobutyric acid), bAib (3-aminoisobutyric acid), Nva (norvaline), β-Ala, Aad (2-aminoadipic acid), bAad (3-aminoadipic acid), Abu (2-aminobutyric acid), Gaba (γ-aminobutyric acid), Acp (6-aminocaproic acid), Dbu (2,4-diaminobutryic acid), α-aminopimelic acid, TMSA (trimethylsilyl-Ala), aIle (allo-isoleucine), Nle (norleucine), tert-Leu, Cit (citrulline), Orn (ornithine, O), Dpm (2,2′-diaminopimelic acid), Dpr (2,3-diaminopropionic acid), α or β-Nal, Cha (cyclohexyl-Ala), hydroxyproline, Sar (sarcosine), Dap (2,3-diaminopropionic acid) and the like.

Unnatural amino acids also include cyclic amino acids; and amino acid analogs, for example, N^{α}-alkylated amino acids such as MeGly (N^{α}-methylglycine), EtGly (N^{α}-ethylglycine) and EtAsn (N^{α}-ethylasparagine); and amino acids in which the α-carbon bears two side-chain substituents. As with the natural amino acids, the residues of the unnatural amino acids are what are left behind when the unnatural amino acid becomes part of a peptide sequence as described herein.

Amino acid residues are amino acid structures as described above that lack a hydrogen atom of the amino group or the hydroxyl moiety of the carboxyl group or both resulting in the units of a peptide chain being amino-acid residues.

The D-isomers of the natural amino acids are designated herein with a lower case letter of the corresponding naturally occurring amino acid. For example, d-proline is designated “p” rather than “P” as is used for naturally occurring proline.

### Tethers (T)

T of Formula I is a lipohilic tether moiety which imparts lipophilicity to the APJ receptor compounds of the invention. The lipophilicity which T imparts, can promote penetration of the APJ receptor compounds into the cell membrane and tethering of the APJ receptor compounds to the cell membrane. As such, the lipophilicity imparted by T can facilitate interaction between the APJ receptor compounds of the invention and the cognate receptor.

The relative lipophilicity of compounds suitable for use as the lipophilic tether moiety of Formula I can be quantified by measuring the amount of the compound that partitions into an organic solvent layer (membrane-like) vs. an aqueous solvent layer (analogous to the extracellular or cytoplasmic environment). The partition coefficient in a mixed solvent composition, such as octanol/water or octanol/PBS, is the ratio of compound found at equilibrium in the octanol vs. the aqueous solvent (Partition coeff P =[compound]_{octanol}/[compound]_{aqueous}). Frequently, the partition coefficient is expressed in logarithmic form, as the log P. Compounds with greater lipophilicity have a more positive log P than more hydrophilic compounds and tend to interact more strongly with membrane bilayers.

Computational programs are also available for calculating the partition coefficient for compounds suitable for use as the lipophilic tether moiety (T). In situations where the chemical structure is being varied in a systematic manner, for example by adding additional methylene units (—CH₂-) onto to an existing alkyl group, the trend in log P can be calculated using, for example, ChemDraw (CambridgeSoft, Inc).

In one embodiment, T is an optionally substituted (C₆-C₃₀)alkyl, (C₆-C₃₀)alkenyl, (C₆-C₃₀)alkynyl wherein 0-3 carbon atoms are replaced with oxygen, sulfur, nitrogen or a combination thereof. In a specific embodiment, the (C₆-C₃₀)alkyl, (C₆-C₃₀)alkenyl, (C₆-C₃₀)alkynyl are substituted at one or more substitutable carbon atoms with halogen, —CN, —OH, —NH₂, NO₂, —NH(C₁-C₆)alkyl, —N((C₁-C₆)alkyl)₂, (C₁-C₆)alkyl, (C₁-C₆)haloalkyl, (C₁-C₆)haloalkoxy, aryloxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)N((C₁-C₆)alkyl)₂, —N(C₁-C₆)alkylC(S)N((C₁-C₆)alkyl)₂, —CONH(C₁-C₆)alkyl, —OCONH(C₁-C₆)alkyl, —CON(C₁-C₆)alkyl)₂, —C(S)(C₁-C₆)alkyl, —S(O)ₚ(C₁-C₆)alkyl, —S(O)ₚNH₂, —S(O)ₚNHC(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H; and p is 1 or 2.

In a specific embodiment, T is selected from the group consisting of: CH₃(CH₂)₉OPh-, CH₃(CH₂)₆C═C(CH₂)₆, CH₃(CH₂)₁₁O(CH₂)₃, CH₃(CH₂)₉O(CH₂)₂ and CH₃(CH₂)₁₃.

In a specific embodiment, T is selected from the group consisting of: CH₃(CH₂)₁₆, CH₃(CH₂)₁₅, CH₃(CH₂)₁₄, CH₃(CH₂)₁₃,CH₃(CH₂)₁₂, CH₃(CH₂)₁₁, CH₃(CH₂)₁₀, CH₃(CH₂)₉, CH₃(CH₂)₈, CH₃(CH₂)₉OPh-, CH₃(CH₂)₆C═C(CH₂)₆, CH₃(CH₂)₁₁O(CH₂)₃, and CH₃(CH₂)₉O(CH₂)₂ and CH₃(CH₂)₁₃.

It is understood that the lipophilic moiety (T) of Formula I can be derived from precursor liphophilic compounds (e.g., fatty acids and bile acids). As used herein, “derived from” with regard to T, means that T is derived from a precursor lipophilic compound and that reaction of the precursor lipophilic compound in preparing the APJ receptor compounds of Formula I, results in a lipophilic tether moiety represented by T in Formula I that is structurally modified in comparison to the precursor lipophilic compound. For example, the lipophilic tether moiety, T of Formula I, can be derived from a fatty acid or a bile acid. It is understood that in accordance with Formula I, when T is derived from a fatty acid (i.e., a fatty acid derivative) it is attached to L-P at the carbon atom alpha to the carbonyl carbon of the acid functional group in the fatty acid from which it is derived. For example, when T is derived from palmitic acid,

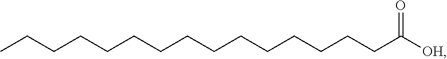

T of Formula 1 has the following structure:

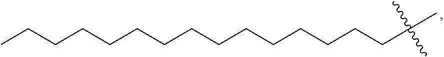

Similarly, when T is derived from stearic acid,

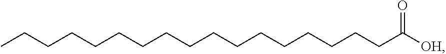

T of Formula 1 has the following structure

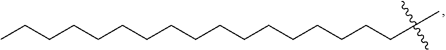

Similarly. when T is derived from 3-(dodecyloxy)propanoic acid,

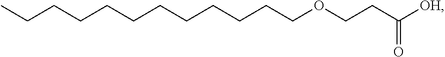

T of Formula I has the following structure:

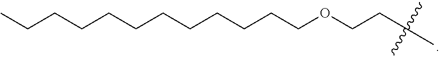

Similarly, when T is derived from 4-(undecyloxy)butanoic acid,

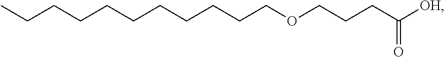

T of Formula I has the following structure:

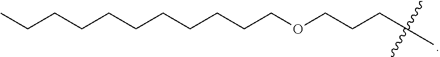

Similarly. when T is derived from elaidic acid,

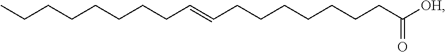

T of Formula I has the following structure:

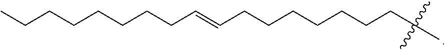

Similarly, when T is derived from oleic acid,

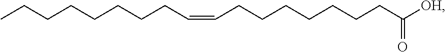

T of Formula I has the following structure:

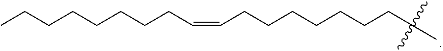

Similarly, when T is derived from 16-hydroxypalmitic acid,

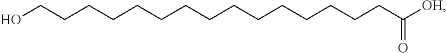

T of Formula I has the following structure:

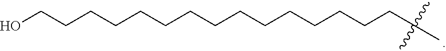

Similarly, when T is derived from 2-aminooctadecanoic acid

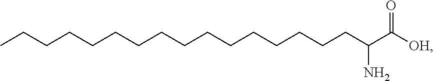

T of Formula I has the following structure

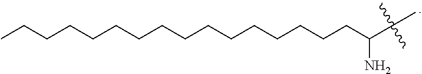

Similarly, when T is derived from 2-amino-4-(dodecyloxy)butanoic acid

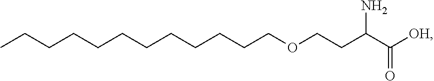

T of Formula 1 has the following structure:

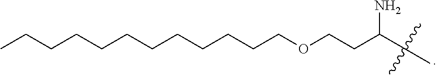

In a further embodiment, T is derived from a fatty acid. In a specific embodiment, T is derived from a fatty acid selected from the group consisting of: butyric acid, caproic acid, caprylic acid, capric acid, lauric acid, myristic acid, palmitic acid, stearic acid, arachidic acid, behenic acid, and lignoceric acid.

In another specific embodiment, T is derived from a fatty acid selected from the group consisting of: myristoleic acid, palmitoleic acid, oleic acid, linoleic acid, α-linolenic acid, arachidonic acid, eicosapentaenoic acid, erucic acid, docosahexaenoic acid

In another embodiment, T of Formula I can be derived from a bile acid. Similar to the embodiment where T is a fatty acid derivative, it is understood that in accordance with Formula I, when T is derived from a bile acid (i.e., a bile acid derivative) it is attached to L-P at the carbon atom alpha to the carbonyl carbon of the acid functional group in the bile acid from which it is derived. For example, when T is derived from lithocholic acid,

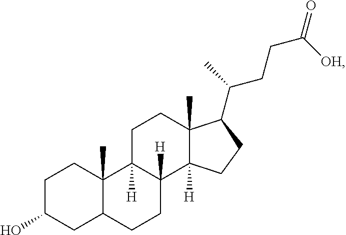

T of Formula I has the following structure:

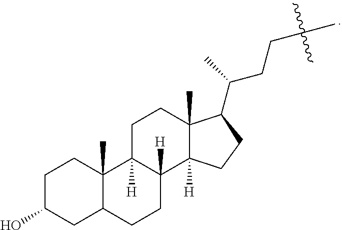

In a further embodiment, T is derived from a bile acid. In a specific embodiment, T is derived from a bile acid selected from the group consisting of: lithocholic acid, chenodeoxycholic acid, deoxycholic acid, cholanic acid, cholic acid, ursocholic acid, ursodeoxycholic acid, isoursodeoxycholic acid, lagodeoxycholic acid, dehydrocholic acid, hyocholic acid, hyodeoxycholic acid and the like.

For example, T is selected from:

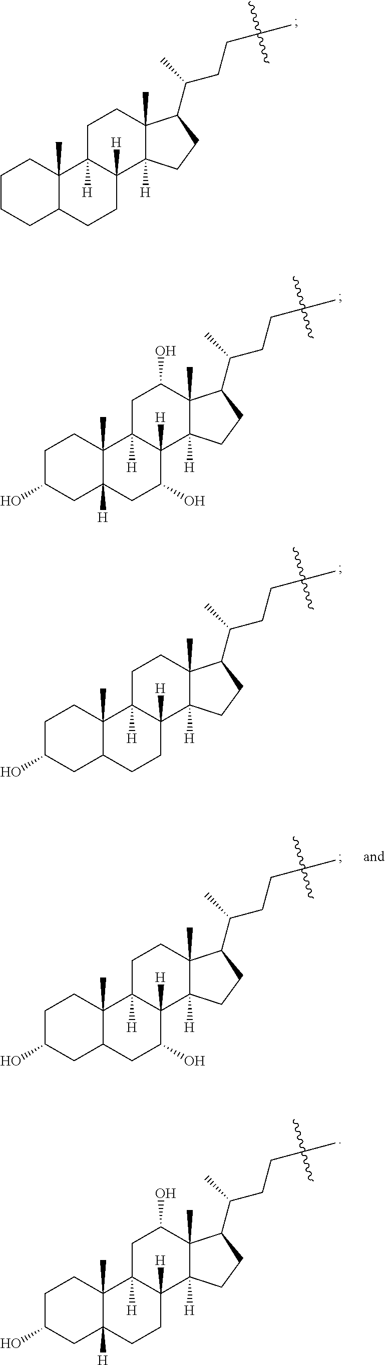

In another further embodiment, T is derived from a bile acid described above that has been modified at other than the acid functional group. For example, T can be derived from any of the bile acids described above, where the hydroxy position has been modified to form an ester or a halo ester. For example, T can be:

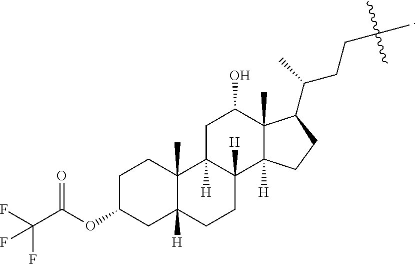

Other lipophilic moieties suitable for use as the lipophilic membrane tether, T, of Formula I, include but are not limited to steroids. Suitable steroids include, but are not limited to, sterols; progestagens; glucocorticoids; mineralcorticoids; androgens; and estrogens. Generally any steroid capable of attachment or which can be modified for incorporation into Formula I can be used. It is understood that the lipophilic membrane tether, T, may be slightly modified from the precursor lipophilic compound as a result of incorporation into Formula I.

Suitable sterols for use in the invention at T, include but are not limited to: cholestanol, coprostanol, cholesterol, epicholesterol, ergosterol, ergocalciferol, and the like. Preferred sterols are those that provide a balance of lipophilicity with water solubility.

Suitable progestagens include, but are not limited to progesterone. Suitable glucocorticoids include, but are not limited to cortisol. Suitable mineralcorticoids include, but are not limited to aldosterone. Suitable androgens include, but are not limited to testosterone and androstenedione. Suitable estrogens include, but are not limited to estrone and estradiol.

In another specific embodiment, T can be derived from 2-tetradecanamideooctadecanoid acid. Similar to the embodiment where T is a fatty acid derivative, it is understood that in accordance with Formula I, when T is derived from 2-tetradecanamideooctadecanoid acid it is attached to L-P at the carbon atom alpha to the carbonyl carbon of the acid functional group in the bile acid from which it is derived. For example, when T is derived from 2-tetradecanamideooctadecanoid acid, the tether is:

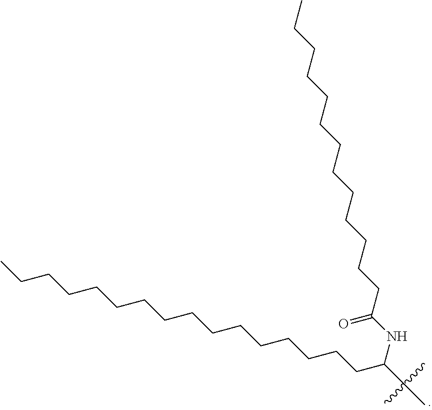

In another embodiment, T of Formula I can be derived from 2-(5-((3aS,4S,6aR)-2-oxohexahydro-1H-thieno[3,4-d]imidazol-4-yl)pentanamido)octadecanoic acid. For example, when T is derived from 2-(5)(3aS,4S,6aR)-2-oxohexahydro-1H-thieno[3,4-d]imidazol-4-yl)pentanamido)octadecanoic acid, the tether is:

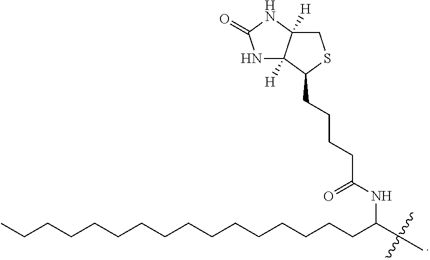

In yet another embodiment, T of Formula I can be:

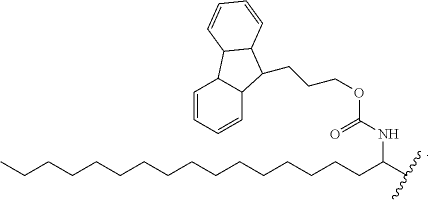

It is understood, that the compounds can contain one of more tether moieties. In certain aspects, the tether moieties are the same. In other embodiments, the tether moieties are different.

### Compounds (T-L-P)

In a first aspect, the GPCR Compound of the invention is represented by Formula I:

T-L-P,

or pharmaceutically acceptable salts thereof, wherein:

P is a peptide comprising at least three contiguous amino-acid residues of an intracellular il, i2, i3 loop or an intracellular i4 domain of the APJ receptor;

L is a linking moiety represented by C(O) and bonded to Pat an N terminal nitrogen of an N-terminal amino-acid residue;

and T is a lipophilic tether moiety bonded to L wherein, the C-terminal amino acid residue of P is optionally functionalized.

In a second aspect, P comprises at least six contiguous amino acid residues.

In a third aspect, P comprises at least 3 contiguous amino acids of the i1 loop.

In a specific embodiment of the third aspect, P is derived from the i1 loop and is represented by the following structural formula or pharmaceutically acceptable salts thereof,

X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-R₁,

wherein

X₁ is absent or a threonine or alanine residue;

X₂ is absent or a valine or alanine residue;

X₃ is absent or a phenylalanine or alanine residue;

X₄ is absent or an arginine, tryptophan, valine or alanine residue;

X₅ is absent or a serine or alanine residue;

X₆ is absent or a serine, glutamic acid or alanine residue;

X₇ is absent or an arginine or alanine residue;

X₈ is absent or a glutamic acid, alanine or proline residue;

X₉ is absent or a lysine, alanine, proline, glutamic acid, D-2,3-diaminionpropionic acid, or D-ornithine;

X₁₀ is absent or an arginine, alanine or lysine residue;

X₁₁ is absent or an arginine or alanine residue;

X₁₂ is absent or a serine, aspartic acid, alanine or arginine residue;

X₁₃ is absent or an alanine or serine residue;

X₁₄ is absent or an aspartic acid or alanine residue;

X₁₅ is absent or an isoleucine, alanine or aspartic acid residue;

X₁₆ is absent or a phenylalanine, valine, alanine or isoleucine residue;

X₁₇ is absent or an isoleucine, alanine or phenylalanine residue;

X₁₈ is absent or an alanine or isoleucine residue;

X₁₉ is absent or a serine residue;

provided that at least five of X₁- X₁₉ are present;

R₁ is OR₂ or N(R₂)₂;

each R₂ is independently hydrogen or (C₁-C₁₀)alkyl; and from 0 to 5 amino acid residues are present in the D configuration.

In a specific embodiment, at least four of X₇, X₈, X₉, X₁₀, X₁₁, X₁₂, X₁₃ and X₁₄ are present.

In another specific embodiment,

X₇ is an arginine or alanine;

X₈ is a glutamic acid, alanine or proline;

X₉ is a lysine, alanine, proline, glutamic acid, D-2,3-diaminionpropionic acid, or D-ornithine; and
X₁₀ is an arginine, alanine or lysine.

In a further specific embodiment, at least one of X₇, X₈, X₉, or X₁₀ is alanine.

In another specific embodiment,
X₁₁ is an arginine or alanine;
X₁₂ is a serine, aspartic acid, alanine or arginine;
X₁₃ is an alanine or serine; and
X₁₄ is an aspartic acid or alanine.

In a further specific embodiment, at least one of X₁₁, X₁₂, X₁₃ or X₁₄ is alanine.

In another specific embodiment,
X₇ is an arginine or alanine;
X₈ is a glutamic acid, alanine or proline;
X₉ is a lysine, alanine, proline, glutamic acid, D-2,3-diaminionpropionic acid, or D-ornithine;
X₁₀ is an arginine, alanine or lysine;
X₁₁ is an arginine or alanine;
X₁₂ is a serine, aspartic acid, alanine or arginine;
X₁₃ is an alanine or serine; and
X₁₄ is an aspartic acid or alanine.

In another specific embodiment, at least one of X₇, X₈, X₉, X₁₀, X₁₁, X₁₂, X₁₃ or X₁₄ is alanine.

In yet another specific embodiment,
X₇ is an arginine;
X₈ is a glutamic acid;
X₉ is a lysine; and
X₁₀ is an arginine.

In yet another specific embodiment,

X₁₁ is an arginine;

X₁₂ is a serine;

X₁₃ is an alanine; and

X₁₄ is an aspartic acid.

In another specific embodiment of the third aspect, the i1 loop of the APJ receptor from which P is derived has the following sequence: TVFRSSREKRRSADIFI (SEQ ID NO: 1).

In another embodiment of the third aspect, P is a sequence selected from:

| | |
|---|---|
| TVFRSSREKRRSADIFI; | (SEQ ID NO: 1) |
| AVFRSSREKRRSADIFI; | (SEQ ID NO: 2) |
| TAFRSSREKRRSADIFI; | (SEQ ID NO: 3) |
| TVARSSREKRRSADIFI; | (SEQ ID NO: 4) |
| TVFASSREKRRSADIFI; | (SEQ ID NO: 5) |
| TVFRASREKRRSADIFI; | (SEQ ID NO: 6) |
| TVFRSAREKRRSADIFI; | (SEQ ID NO: 7) |
| TVFRSSAEKRRSADIFI; | (SEQ ID NO: 8) |
| TVFRSSRAKRRSADIFI; | (SEQ ID NO: 9) |
| TVFRSSREARRSADIFI; | (SEQ ID NO: 10) |
| TVFRSSREKARDADIFI; | (SEQ ID NO: 11) |
| TVFRSSREKRASADIFI; | (SEQ ID NO: 12) |
| TVFRSSREKRRAADIFI; | (SEQ ID NO: 13) |
| TVFRSSREKRRSAAIFI; | (SEQ ID NO: 14) |
| TVFRSSREKRRSADAFI; | (SEQ ID NO: 15) |
| TVFRSSREKRRSADIAI; | (SEQ ID NO: 16) |
| TVFRSSREKRRSADIFA; | (SEQ ID NO: 17) |
| tVFRSSREKRRSADIFI; | (SEQ ID NO: 18) |
| TvFRSSREKRRSADIFI; | (SEQ ID NO: 19) |
| TVfRSSREKRRSADIFI; | (SEQ ID NO: 20) |
| TVFrSSREKRRSADIFI; | (SEQ ID NO: 21) |
| TVFRsSREKRRSADIFI; | (SEQ ID NO: 22) |
| TVFRSsREKRRSADIFI; | (SEQ ID NO: 23) |
| TVFRSSrEKRRSADIFI; | (SEQ ID NO: 24) |
| TVFRSSReKRRSADIFI; | (SEQ ID NO: 25) |
| TVFRSSREKrRSADIFI; | (SEQ ID NO: 26) |
| TVFRSSREKRrSADIFI; | (SEQ ID NO: 27) |
| TVFRSSREKRRSADiFI; | (SEQ ID NO: 28) |
| TVFRSSREKRRSADIFi; | (SEQ ID NO: 29) |
| TVFRSSREKRRsADIFI; | (SEQ ID NO: 30) |
| TVFRSSREKRRSaDIFI; | (SEQ ID NO: 31) |
| TVFRSSREKRRSAdIFI; | (SEQ ID NO: 32) |
| TVFRSSREkRRSADIFI; | (SEQ ID NO: 33) |
| TVFWSSREKRRSADIFI; | (SEQ ID NO: 34) |
| VFRSSREKRRSADIFI; | (SEQ ID NO: 35) |
| FRSSREKRRSADIFI; | (SEQ ID NO: 36) |
| SSREKRRSADIFIAS; | (SEQ ID NO: 37) |
| RSSREKRRSADIFI; | (SEQ ID NO: 38) |
| FRSSREKRRSADIA; | (SEQ ID NO: 39) |
| FRSSREKRRSADIV; | (SEQ ID NO: 40) |
| TVFRSSREKRRSAD; | (SEQ ID NO: 41) |
| SSREKRRSADIFIA; | (SEQ ID NO: 42) |
| VSSREKRRSADIFI; | (SEQ ID NO: 43) |
| SSREKRRSADIFI; | (SEQ ID NO: 44) |
| FRSSREKRRSADI; | (SEQ ID NO: 45) |
| FRSSREKRRSAD; | (SEQ ID NO: 46) |
| SREKRRSADIFI; | (SEQ ID NO: 47) |
| REKRRSADIFI; | (SEQ ID NO: 48) |
| RSSREKRRSAD; | (SEQ ID NO: 49) |
| REKRRSADIF; | (SEQ ID NO: 50) |
| SSREKRRSAD; | (SEQ ID NO: 51) |
| REKRRSADI; | (SEQ ID NO: 52) |
| SREKRRSAD; | (SEQ ID NO: 53) |
| EREKRRSAD; | (SEQ ID NO: 54) |
| and | |
| REKRRSAD. | (SEQ ID NO: 55) |

In another embodiment of the third aspect, P is a sequence selected from:

| | |
|---|---|
| TVFRSSRE(D-Dap)RRSADIFI; | (SEQ ID NO: 108) |
| TVFRSSREpKRRSADIFI; | (SEQ ID NO: 109) |
| TVFRSSRpEKRRSADIFI; | (SEQ ID NO: 110) |
| TVFRSSRE(D-Dab)RRSADIFI; | (SEQ ID NO: 111) |
| and | |
| TVFRSSRE(D-Orn)RRSADIFI. | (SEQ ID NO: 112) |

In a fourth aspect, P comprises at least 3 contiguous amino acids of the i2 loop. In a specific embodiment of the fourth aspect, P derived from the i2 loop and is represented by the following structural formula or a pharmaceutically acceptable salt thereof,

Y₁-Y₂-Y₃-Y₄-Y₅-Y₆-Y₇-Y₈-Y₉-Y₁₀-Y₁₁-Y₁₂-Y₁₃-Y₁₄-Y₁₅-Y₁₆-Y₁₇-Y₁₈-Y₁₉-Y₂₀-Y₂₁Y₂₂-R₁

wherein:

Y₁ is absent or an aspartic acid residue;

Y₂ is absent or an arginine residue;

Y₃ is absent or a tyrosine residue;

Y₄ is absent or a leucine residue;

Y₅ is absent or an alanine residue;

Y₆ is absent or an isoleucine residue;

Y₇ is absent or a valine residue;

Y₈ is absent or an arginine residue;

Y₉ is absent or a proline residue;

Y₁₀ is absent or a valine residue;

Y₁₁ is absent or an alanine residue;

Y₁₂ is absent or an asparagine residue;

Y₁₃ is absent or an alanine residue;

Y₁₄ is absent or an arginine residue;

Y₁₅ is absent or a leucine residue;

Y₁₆ is absent or an arginine residue;

Y₁₇ is absent or a leucine residue;

Y₁₈ is absent or an arginine or leucine residue;

Y₁₉ is absent or a valine or leucine residue;

Y₂₀ is absent or a serine;

Y₂₁ is absent or a glycine residue; and

Y₂₂ is absent or an alanine, provided that at least five of Y₁-Y₂₂ are present;

R₁ is OR₂ or N(R₂)₂;

each R₂ is independently hydrogen or (C₁-C₁₀)alkyl; and

from 0 to 5 amino acid residues are present in the D configuration.

It is understood that when P is described as Y₁- Y₂₂ it is bonded to L as written. For example, Y₁ is bonded to L. If Y₁ is absent, then Y₂ is bonded to L.

In a specific embodiment, at least three of Y₈, Y₉, Y₁₀, Y₁₁, Y₁₂, Y₁₃, and Y₁₄ are present.

In a further specific embodiment,

Y₈ is an arginine residue;

Y9 is a proline residue;

Y₁₀ is a valine residue; and

Y₁₁ is an alanine residue;

In another further specific embodiment,

Y₁₂ is an asparagine residue;

Y₁₃ is an alanine residue; and

Y₁₄ is an arginine residue; and

Y₁₅ is a leucine residue.

In another specific embodiment of the fourth aspect, the i2 loop of the APJ receptor from which P is derived has the following sequence: DRYLAIVRPVANARLRLRVSGA (SEQ ID NO: 56).

In another embodiment of the fourth aspect, P is a sequence selected from:

| | |
|---|---|
| DRYLAIVRPVANARLRLRVSGA; | (SEQ ID NO: 56) |
| LAIVRPVANARLRLRVSG; | (SEQ ID NO: 57) |
| AIVRPVANARLRLRVSG; | (SEQ ID NO: 58) |
| IVRPVANARLRLRVSG; | (SEQ ID NO: 59) |
| VRPVANARLRLRVSG; | (SEQ ID NO: 60) |
| RPVANARLRLRVSG; | (SEQ ID NO: 61) |
| VRPVANARLRLRVS; | (SEQ ID NO: 62) |
| AIVRPVANARLRL; | (SEQ ID NO: 63) |
| RPVANARLRLRVS; | (SEQ ID NO: 64) |
| VRPVANARLRLLL; | (SEQ ID NO: 65) |
| VRPVANARLRLRV; | (SEQ ID NO: 66) |
| RPVANARLRLRV; | (SEQ ID NO: 67) |
| VRPVANARLRLR; | (SEQ ID NO: 68) |
| RPVANARLRLR; | (SEQ ID NO: 69) |
| VRPVANARLRL; | (SEQ ID NO: 70) |
| RPVANARLRL; | (SEQ ID NO: 71) |
| VRPVANARLR; | (SEQ ID NO: 72) |
| and | |
| VRPVANARL. | (SEQ ID NO: 73) |

In a fifth aspect, P comprises at least 3 contiguous amino acids of the i3 loop.

In a specific embodiment of the fifth aspect, P derived from the i3 loop and is represented by the following structural formula or a pharmaceutically acceptable salt thereof,

P is Z₁-Z₂-Z₃-Z₄-Z₅-Z₆-Z₇-Z₈-Z₉-Z₁₀-Z₁₁-Z₁₂-Z₁₃-Z₁₄-Z₁₅-Z₁₆-Z₁₇-Z₁₈-Z₁₉-Z₂₀-Z₂₁-Z₂₂-Z₂₃-Z₂₄-Z₂₅-Z₂₆-Z₂₇-Z₂₈-Z₂₉-Z₃₀-R₁,

wherein :

Z₁ is absent or an isoleucine residue;

Z₂ is absent or an alanine residue;

Z₃ is absent or a glutamine or glycine residue;

Z₄ is absent or a threonine or serine residue;

Z₅ is absent or a isoleucine or glycine residue;

Z₆ is absent or an alanine or serine residue;

Z₇ is absent or a glycine residue;

Z₈ is absent or a histidine residue;

Z₉ is absent or a phenylalanine residue;

Z₁₁ is absent or an arginine residue;

Z₁₁ is absent or a lysine residue;

Z₁₂ is absent or a glutamic acid residue;

Z₁₃ is absent or an arginine residue;

Z₁₄ is absent or an isoleucine residue;

Z₁₅ is absent or a glutamic acid or glycine residue;

Z₁₆ is absent or a glycine residue;

Z₁₇ is absent or a leucine residue;

Z₁₈ is absent or an arginine or glycine residue;

Z₁₉ is absent or lysine residue;

Z₂₀ is absent or an arginine residue;

Z₂₁ is absent or an arginine residue;

Z₂₂ is absent or an arginine residue;

Z₂₃ is absent or a leucine residue;

Z₂₄ is absent or a leucine residue;

Z₂₅ is absent or a serine, alanine, phenylalanine or tryptophan residue;

Z₂₆ is absent or an isoleucine residue;

Z₂₇ is absent or an isoleucine residue;

Z₂₈ is absent or a valine residue;

Z₂₉ is absent or a valine residue; and

Z₃₀ is absent or a leucine residue; provided that at least five of Z₁-Z₃₀ are present; and

R₁ is OR₂ or N(R₂)₂;

each R₂ is independently hydrogen or (C₁-C₁₀)alkyl; and

from 0 to 5 amino acid residues are present in the D configuration.

In a specific embodiment, at least four of Z₁₂, Z₁₃, Z₁₄, Z₁₅, Z₁₆, Z₁₇, Z₁₈, and Z₁₉ are present.

In a further specific embodiment,

Z₁₂ is a glutamic acid residue;

Z₁₃ is an arginine residue;

Z₁₄ is an isoleucine residue; and

Z₁₅ is a glutamic acid or glycine residue.

In a further specific embodiment,

Z₁₆ is a glycine residue;

Z₁₇ is a leucine residue;

Z₁₈ is a arginine residue or glycine residue; and

Z₁₉ is a lysine residue.

In another specific embodiment, at least four of Z₂₁, Z₂₂, Z₂₃, Z₂₄, and Z₂₅ are present.

In a further specific embodiment,

Z₂₁ is an arginine residue;

Z₂₂ is an arginine residue;

Z₂₃ is a leucine residue;

Z₂₄ is a leucine residue; and

Z₂₅ is a serine residue or an alanine, phenylalanine or tryptophan residue.

In another further specific embodiment, Z₂₅ is an alanine residue.

In another specific embodiment, Z₃, Z₄, Z₅, and Z₆ are present.

In a further specific embodiment,

Z₃ is a glutamine residue;

Z₄ is a threonine residue;

Z₅ is an isoleucine residue; and

Z₆ is an alanine residue.

In another further specific embodiment,

Z₃ is a glycine residue;

Z₄ is a serine residue;

Z₅ is a glycine residue; and

Z₆ is a serine residue.

In a specific embodiment of the fifth aspect, the i3 loop of the APJ receptor from which P is derived has the following sequence: IAQTIAGHFRKERIEGLRKRRRLLSIIVVL (SEQ ID NO: 74).

In another embodiment of the fifth aspect, P is a sequence selected from:

| | |
|---|---|
| IAQTIAGHFRKERIEGLRKRRRLLSIIVVL; | (SEQ ID NO: 74) |
| QTIAGHFRKERIEGLRKRRRLLS; | (SEQ ID NO: 75) |
| QTIAGHFRKERIEGLRKRRRLLA; | (SEQ ID NO: 76) |
| QTIAGHFRKERIEGLRKRRRLLF; | (SEQ ID NO: 77) |
| QTIAGHFRKERIEGLRKRRRLLW; | (SEQ ID NO: 78) |
| GSGSGHFRKERIEGLRKRRRLLA; | (SEQ ID NO: 79) |
| SGSGHFRKERIEGLRKRRRLLA; | (SEQ ID NO: 80) |
| IAGHFRKERIEGLRKRRRLLS; | (SEQ ID NO: 81) |
| QTIAGHFRKERIEGLRKRRRL; | (SEQ ID NO: 82) |
| GSGHFRKERIEGLRKRRRLLA; | (SEQ ID NO: 83) |
| SGHFRKERIEGLRKRRRLLA; | (SEQ ID NO: 84) |
| GHFRKERIEGLRKRRRLLS; | (SEQ ID NO: 85) |
| QTIAGHFRKERIEGLRKRR; | (SEQ ID NO: 86) |
| GHFRKERIEGLRKRRRLLA; | (SEQ ID NO: 87) |
| HFRKERIEGLRKRRRLLS; | (SEQ ID NO: 88) |
| QTIAGHFRKERIEGLRK; | (SEQ ID NO: 89) |
| FRKERIEGLRKRRRLLA; | (SEQ ID NO: 90) |
| RKERIEGLRKRRRLLS; | (SEQ ID NO: 91) |
| ERIEGLRKRRRLLSII; | (SEQ ID NO: 92) |
| HFRKERIEGLRKRRRL; | (SEQ ID NO: 93) |
| FRKERIGGKRRRLLA; | (SEQ ID NO: 94) |
| ERIEGLRKRRRLLS; | (SEQ ID NO: 95) |
| HFRKERIEGLRKRR; | (SEQ ID NO: 96) |
| QTIAGHFRKERI; | (SEQ ID NO: 97) |
| EGLRKRRRLLA; | (SEQ ID NO: 98) |
| and | |
| QTIAGHFRKER. | (SEQ ID NO: 99) |

In a sixth aspect, P comprises at least 3 contiguous amino acids of the i4 domain.

In a specific embodiment of the sixth aspect, P derived from the i4 domain and is represented by the following structural formula or a pharmaceutically acceptable salt thereof,

M₁-M₂-M₃-M₄-M₅-M₆-M₇-M₈-M₉-M₁₀-M₁₁-M₁₂-M₁₃-M₁₄-R₁,

wherein:

M₁ is absent or a phenylalanine residue;

M₂ is absent or a phenylalanine residue;

M₃ is absent or an aspartic acid residue;

M₄ is absent or a proline residue;

M₅ is absent or an arginine residue;

M₆ is absent or a phenylalanine residue;

M₇ is absent or an arginine residue;

M₈ is absent or a glutamine residue;

M₉ is absent or an alanine residue;

M₁₀ is absent or a serine residue;

M₁₁ is absent or a threonine residue;

M₁₂ is absent or a serine residue;

M₁₃ is absent or a methionine residue; and

M₁₄ is absent or a leucine residue; provided that at least five of M₁-M₁₄ are present;

R₁ is OR₂ or N(R₂)₂;

each R₂ is independently hydrogen or (C₁-C₁₀)alkyl; and

from 0 to 5 amino acid residues are present in the D configuration.

It is understood that when P is described as M₁- M₁₄ it is bonded to L as written. For example, M₁ is bonded to L. If M₁ is absent, then M₂ is bonded to L.

In a specific embodiment,

M₃ is an aspartic acid residue;

M₄ is a proline residue;

M₅ is an arginine residue; and

M₆ is a phenylalanine residue.

In a specific embodiment of the sixth aspect, the i4 domain of the APJ receptor from which P is derived has the following sequence:

(SEQ ID NO: 100)
FFDPRFRQACTSMLCCGQSRCAGTSHSSSGEKSASYSSGHSQGPGP
NMGKGGEQMHEKSIPYSQETLVVD.

In another embodiment of the sixth aspect, P is a sequence selected from:

| | |
|---|---|
| FFDPRFRQASTSML; | (SEQ ID NO: 101) |
| FDPRFRQASTSML; | (SEQ ID NO: 102) |
| and | |
| DPRFRQASTSML. | (SEQ ID NO: 103) |

In a seventh aspect, T is an optionally substituted (C₆-C₃₀)alkyl, (C₆-C₃ₒ)alkenyl, (C₆-C₃₀)alkynyl, wherein 0-3 carbon atoms are replaced with oxygen, sulfur, nitrogen or a combination thereof. This value of T is applicable to the first, second, third, fourth, fifth and sixth aspects and the specific (i.e., specific, more specific and most specific) embodiments of same.

In a specific embodiment of the seventh aspect, T is selected from: CH₃(CH₂)₁₆, CH₃(CH₂)₁₅, CH₃(CH₂)₁₄, CH₃(CH₂)₁₃,CH₃(CH₂)₁₂, CH₃(CH₂)₁₁, CH₃(CH₂)₁₀, CH₃(CH₂)₉, CH₃(CH₂)₈, CH₃(CH₂)₉OPh-, CH₃(CH₂)₆C═C(CH₂)₆, CH₃(CH₁₁O(CH₂)₃, and CH₃(CH₂)₉O(CH₂)₂.

In another specific embodiment of the seventh aspect, T is a fatty acid derivative.

In a more specific embodiment of the seventh aspect, the fatty acid is selected from the group consisting of: butyric acid, caproic acid, caprylic acid, capric acid, lauric acid, myristic acid, palmitic acid, stearic acid, arachidic acid, behenic acid, lignoceric acid, myristoleic acid, palmitoleic acid, oleic acid, linoleic acid, α-linolenic acid, arachidonic acid, eicosapentaenoic acid, erucic acid, docosahexaenoic acid.

In an eighth aspect, T is a bile acid derivative. This value of T is applicable to the first, second, third, fourth, fifth and sixth aspects and the specific (i.e., specific, more specific and most specific) embodiments of same.

In a specific embodiment of the eighth aspect, the bile acid is selected from the group consisting of: lithocholic acid, chenodeoxycholic acid, deoxycholic acid, cholanic acid, cholic acid, ursocholic acid, ursodeoxycholic acid, isoursodeoxycholic acid, lagodeoxycholic acid, dehydrocholic acid, hyocholic acid, and hyodeoxycholic acid.

In a ninth aspect, T is selected from sterols; progestagens; glucocorticoids; mineralcorticoids; androgens; and estrogens. This value of T is applicable to the first, second, third, fourth, fifth and sixth aspects and the specific (i.e., specific, more specific and most specific) embodiments of same.

In a tenth aspect, T-L of Formula I is represented by a moiety selected from the group consisting of:

CH₃(CH₂)₁₅—C(O);

CH₃(CH₂)₁₃—C(O);

CH₃(CH₂)₉O(CH₂)₂C(O);

CH₃(CH₂)₁₀O(CH₂)₂C(O);

CH₃(CH₂)₆C═C(CH₂)₆—C(O);

LCA-C(O); and

CH₃(CH₂)₉OPh-C(O) wherein

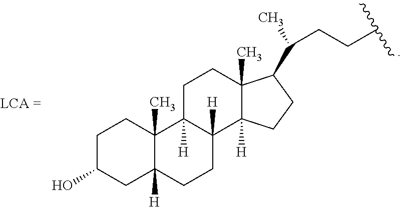

In an eleventh aspect, T of Formula I is represented by a moiety selected from the group consisting of:

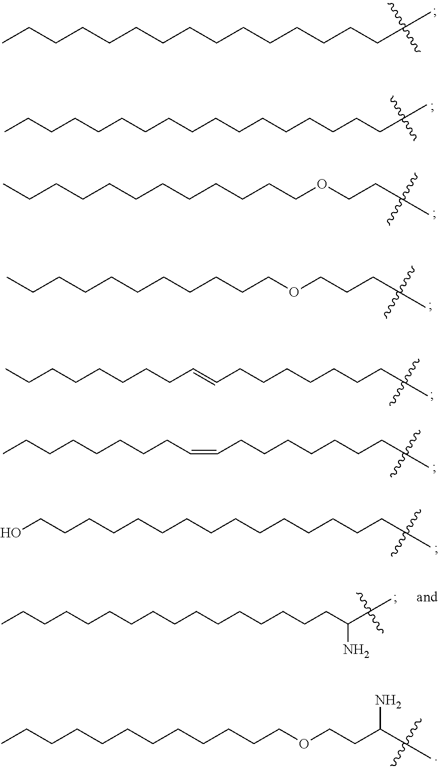

In yet another embodiment, a GPCR compound of the invention is selected from one of the following compounds or a pharmaceutically acceptable salt thereof:

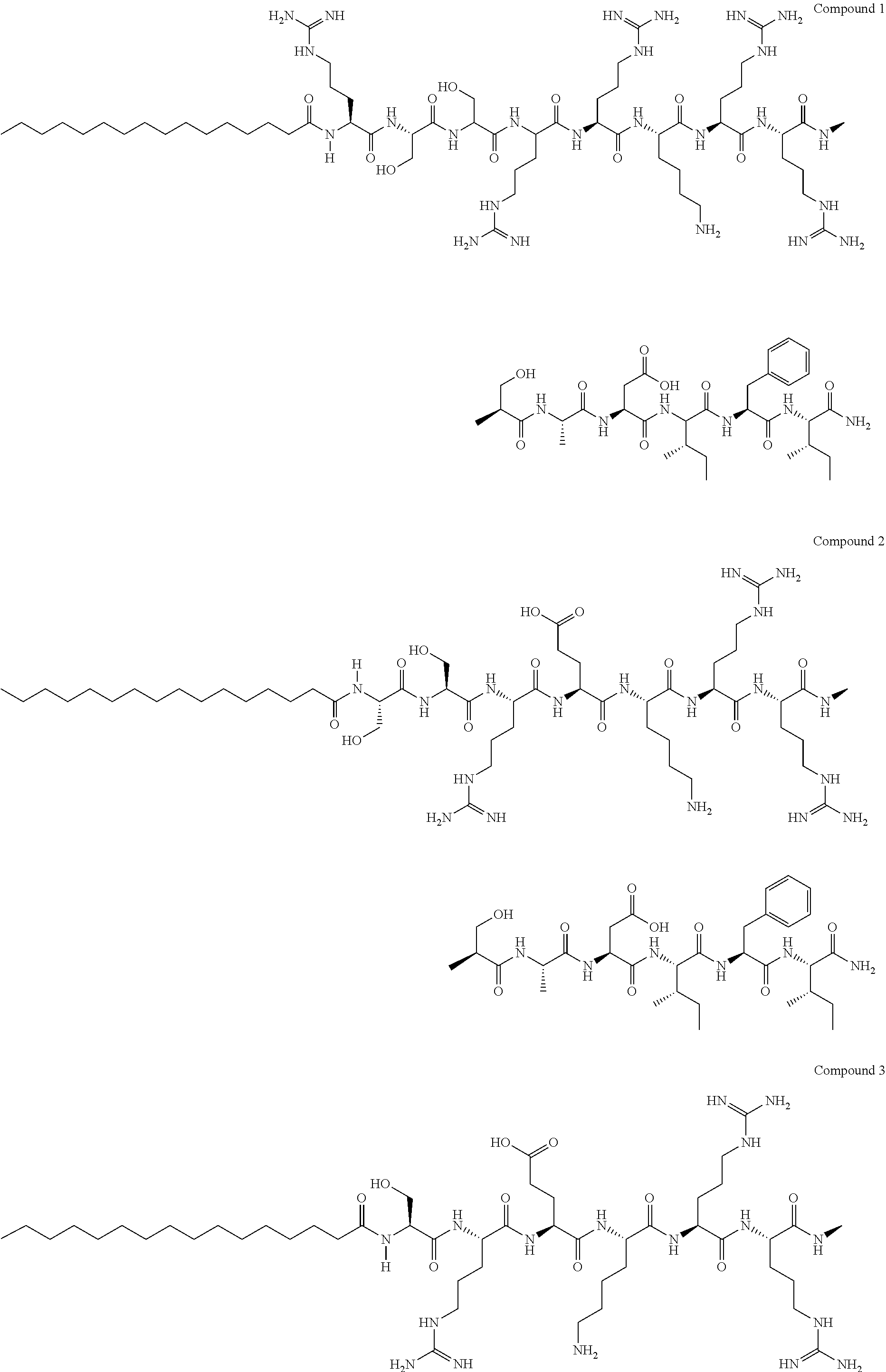

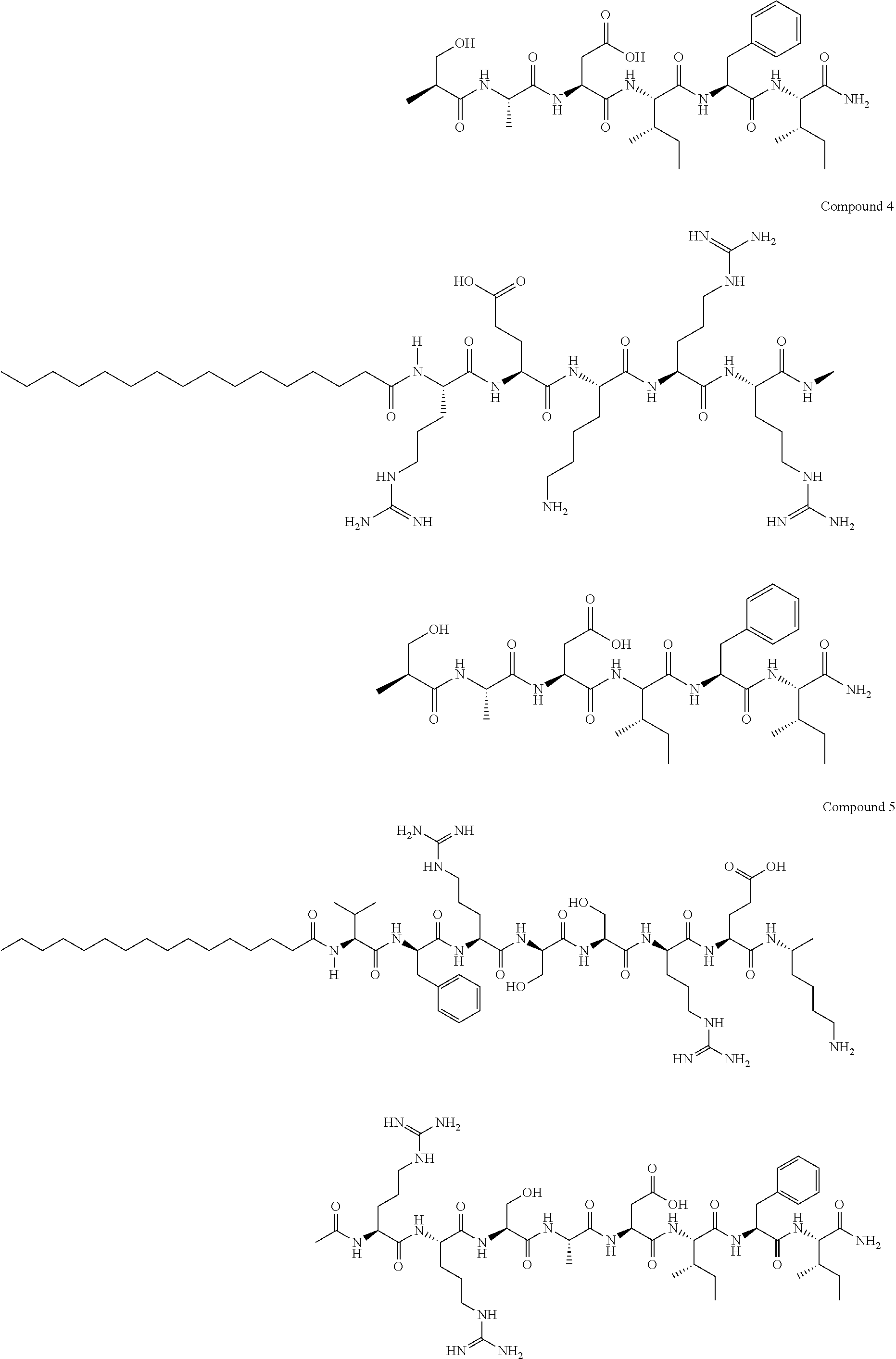

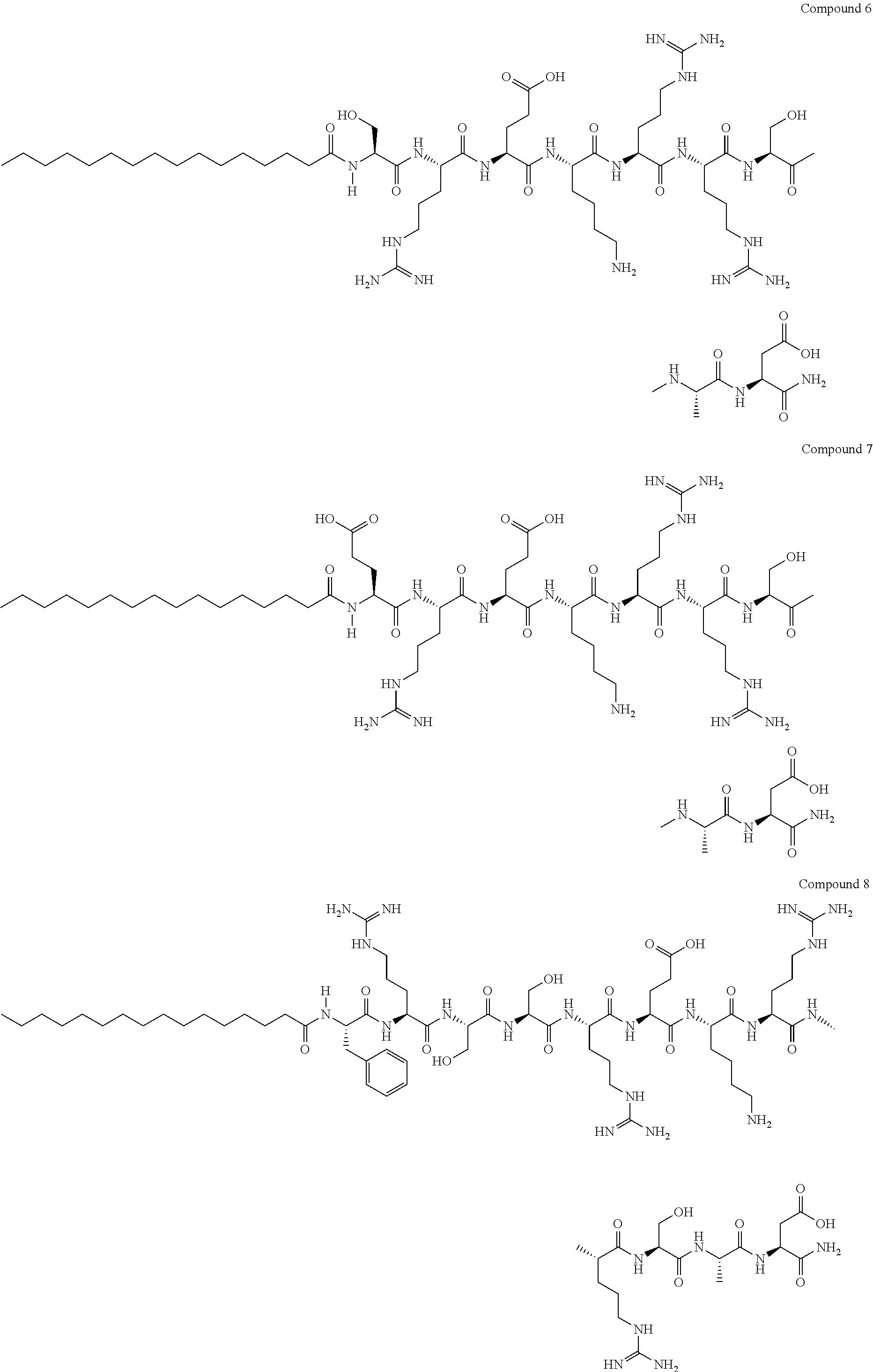

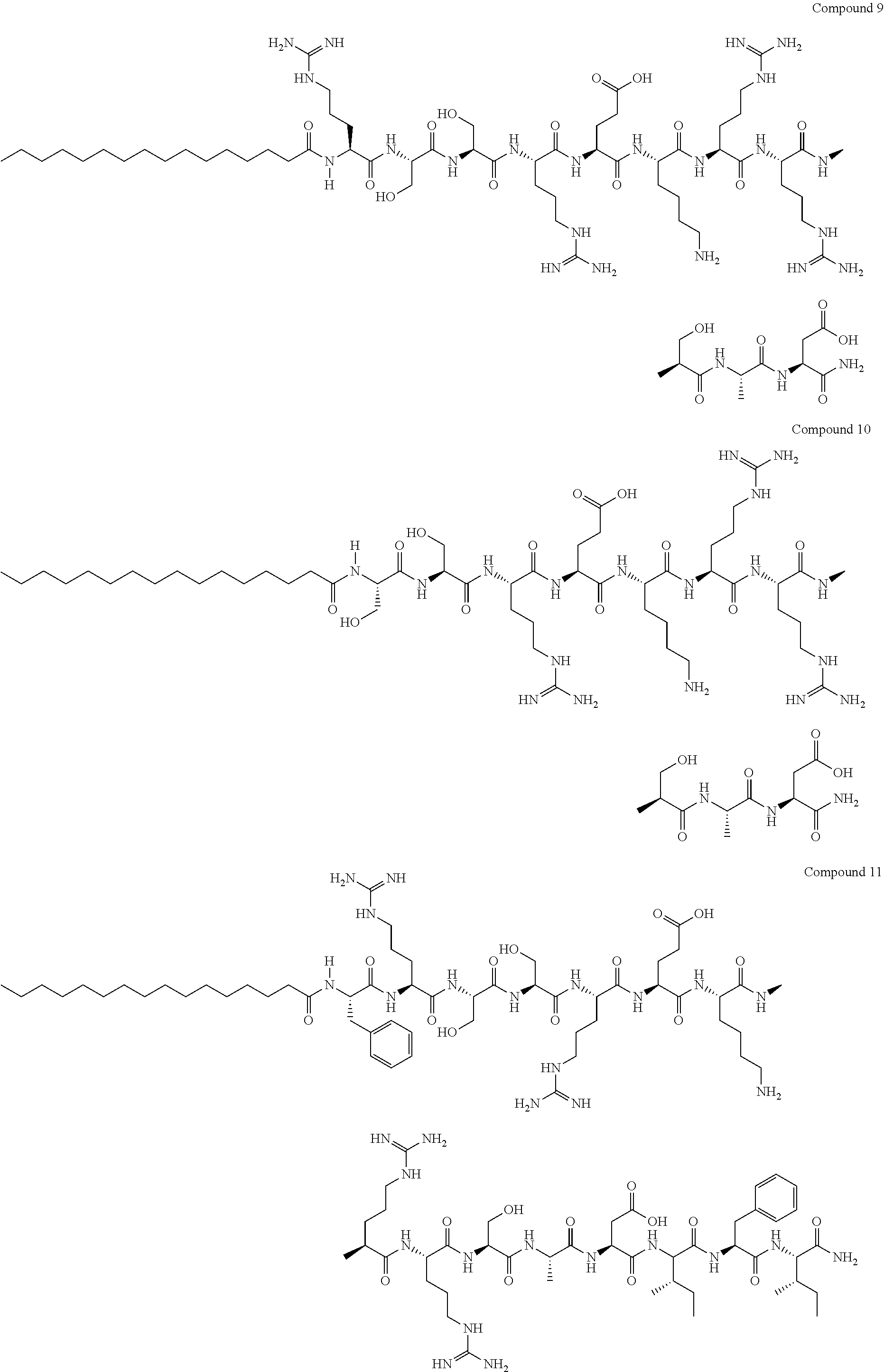

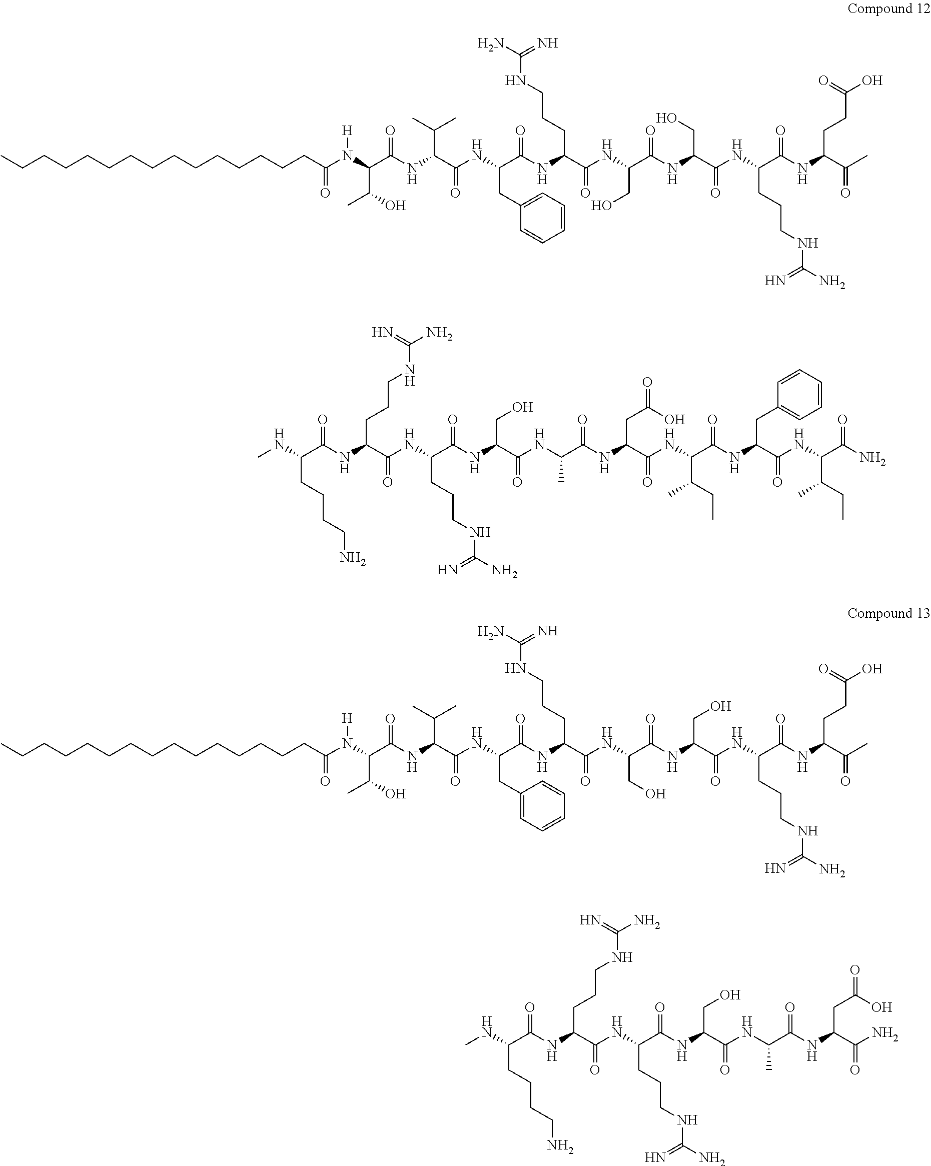

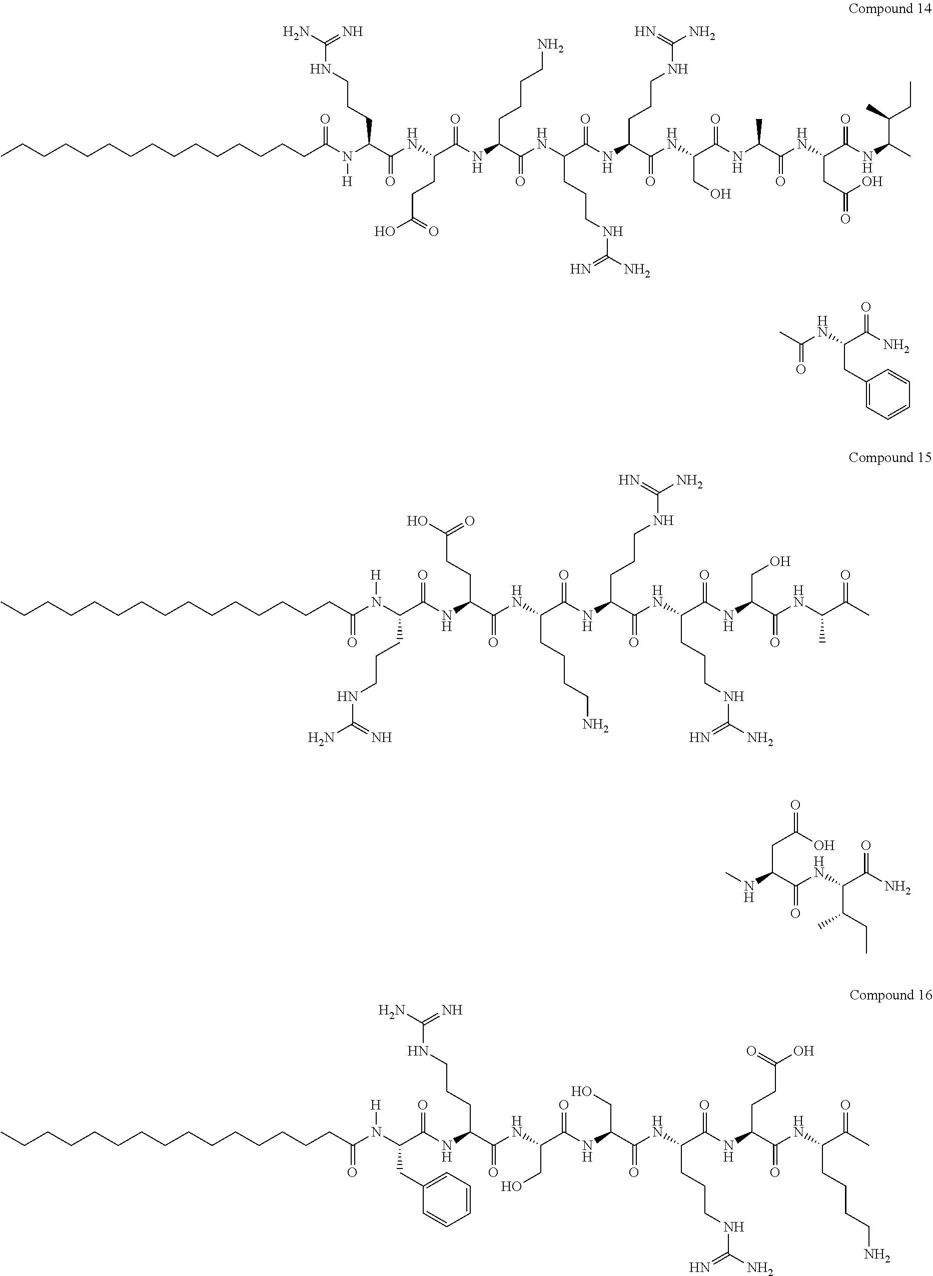

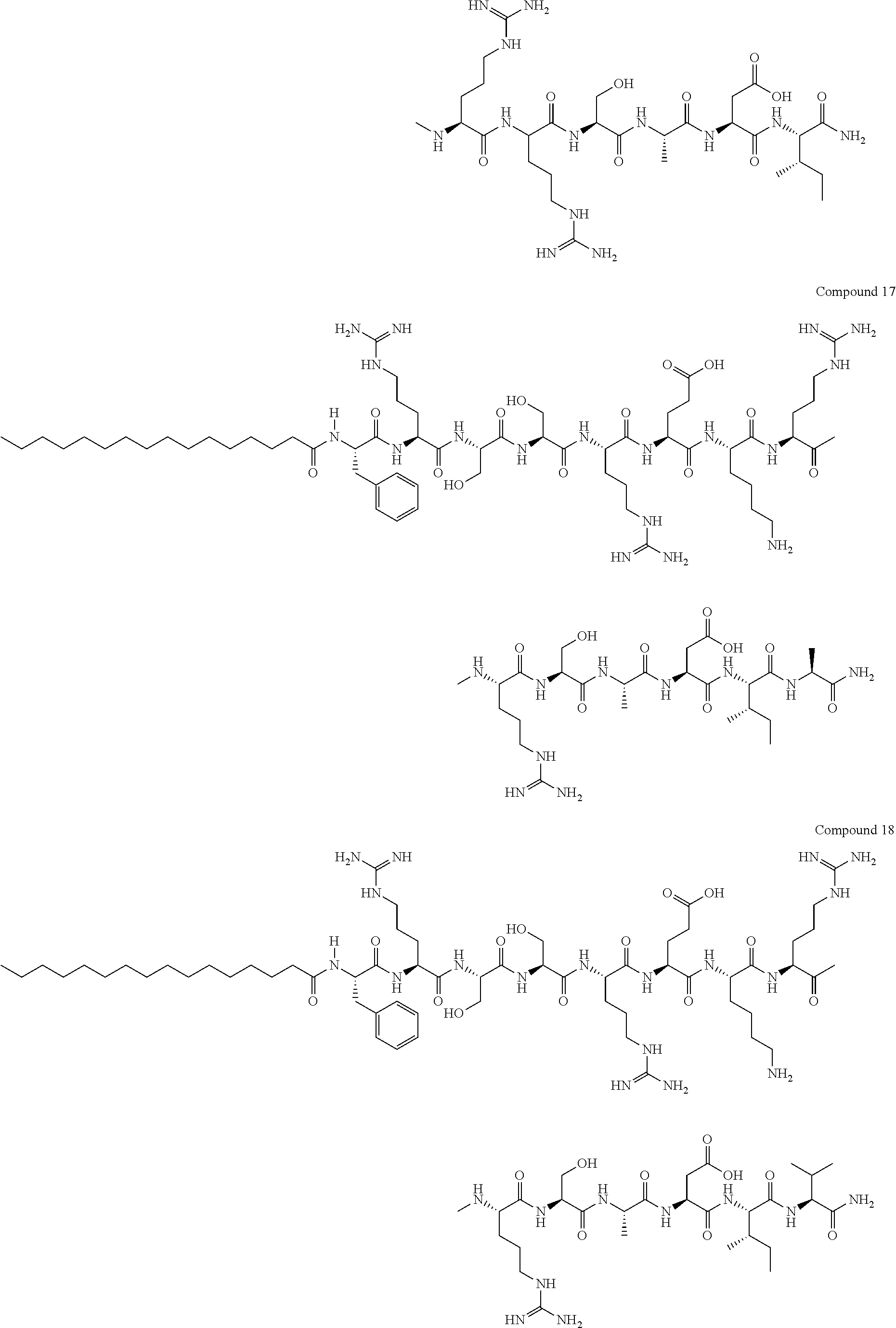

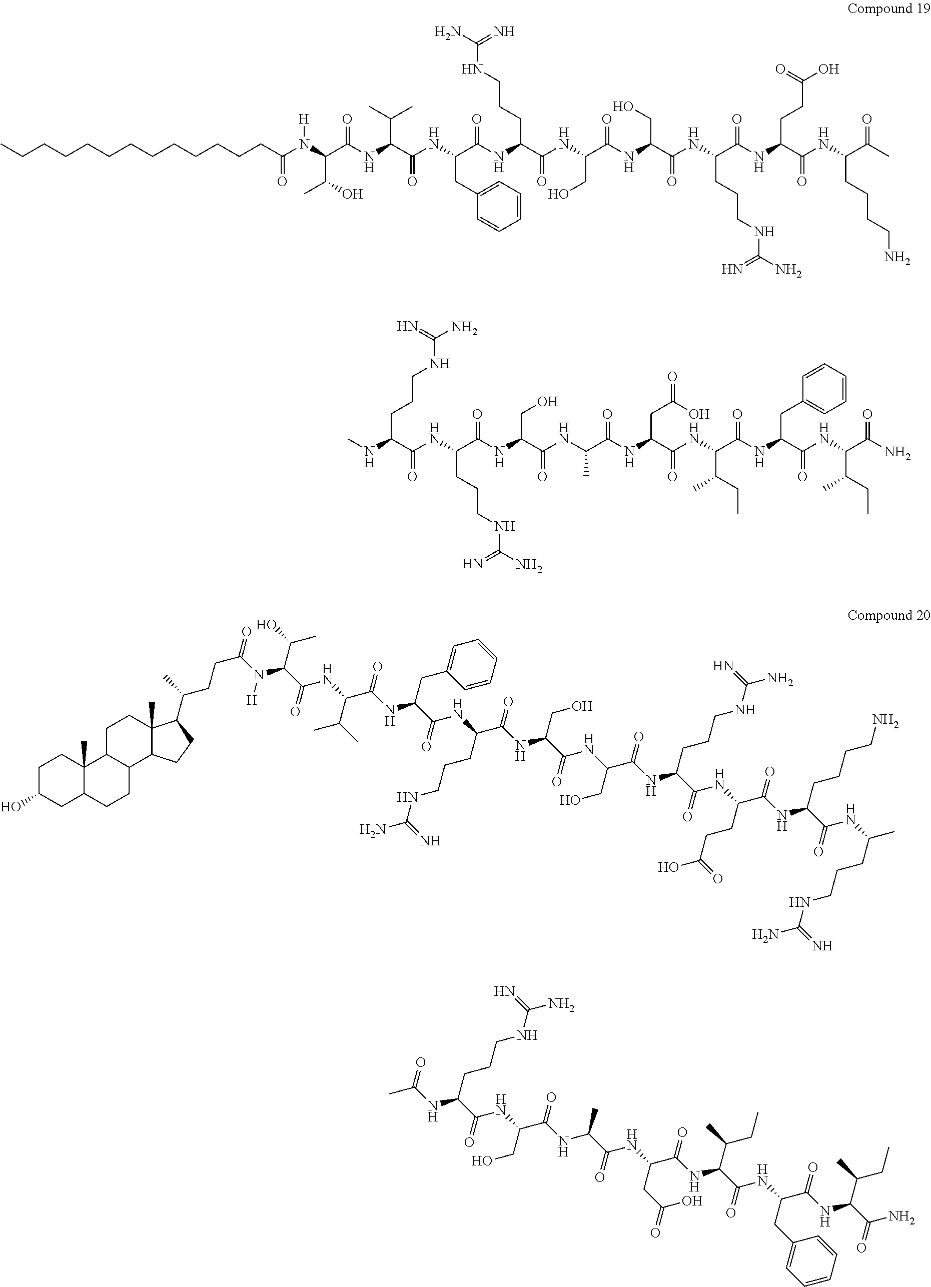

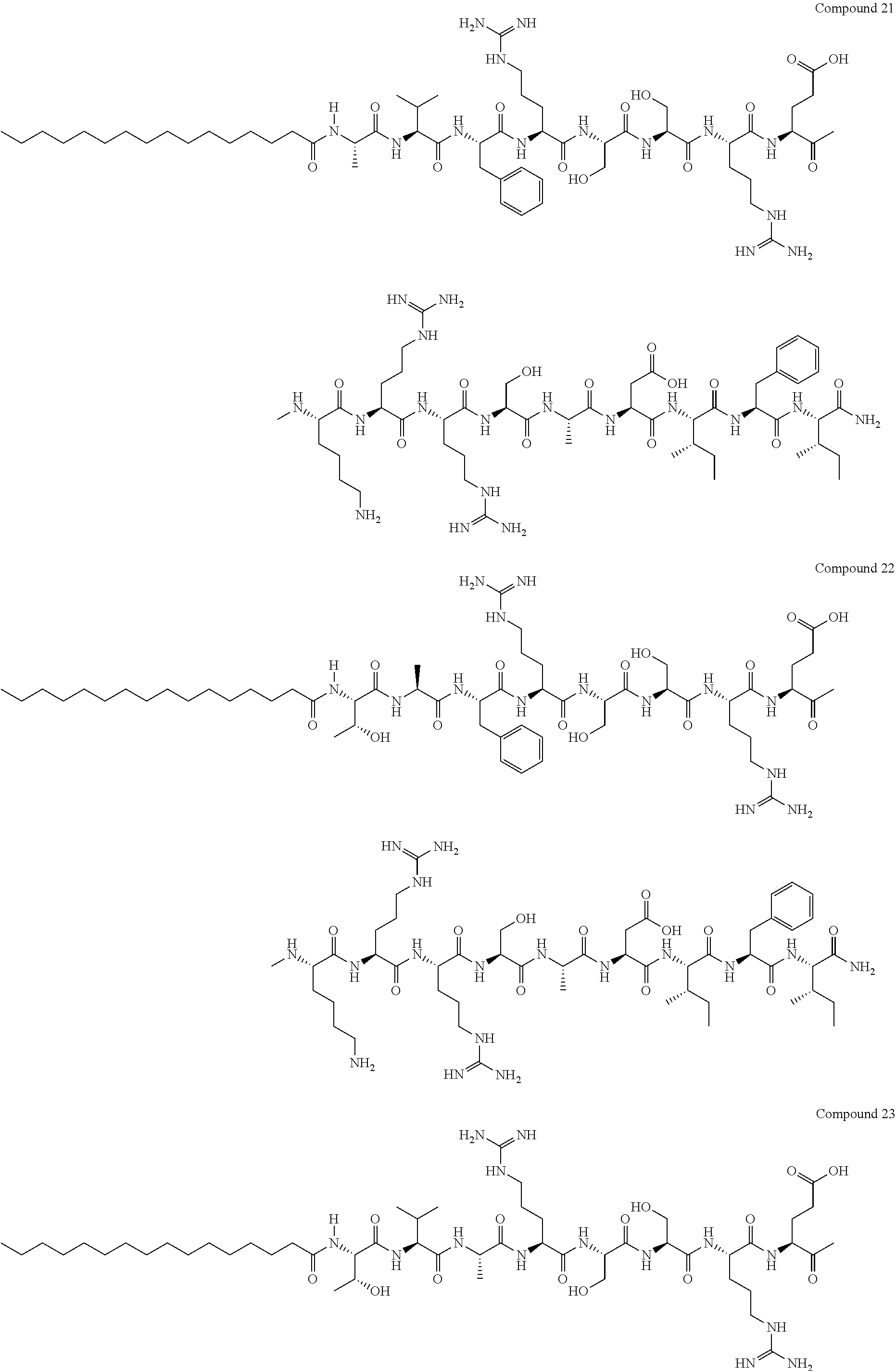

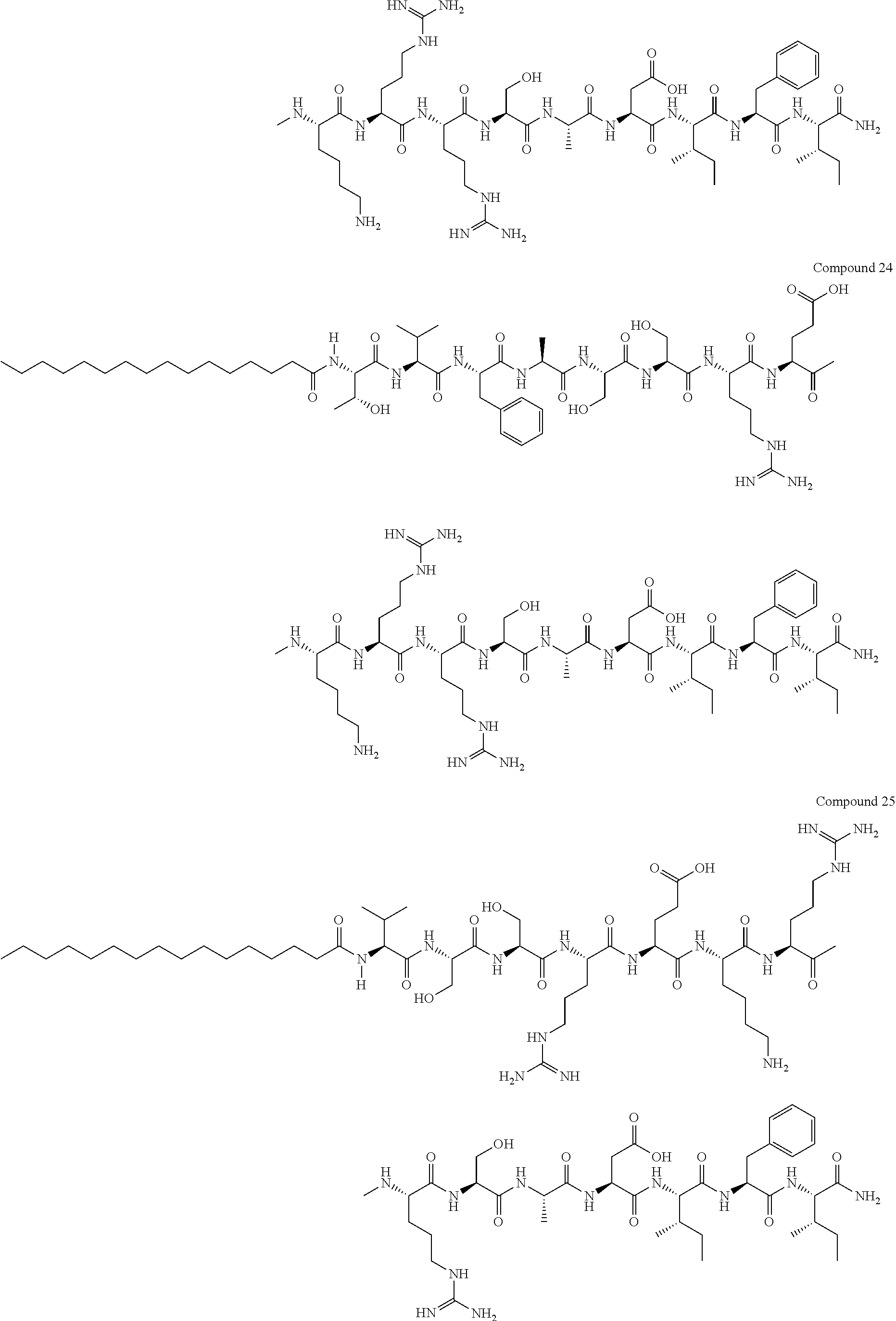

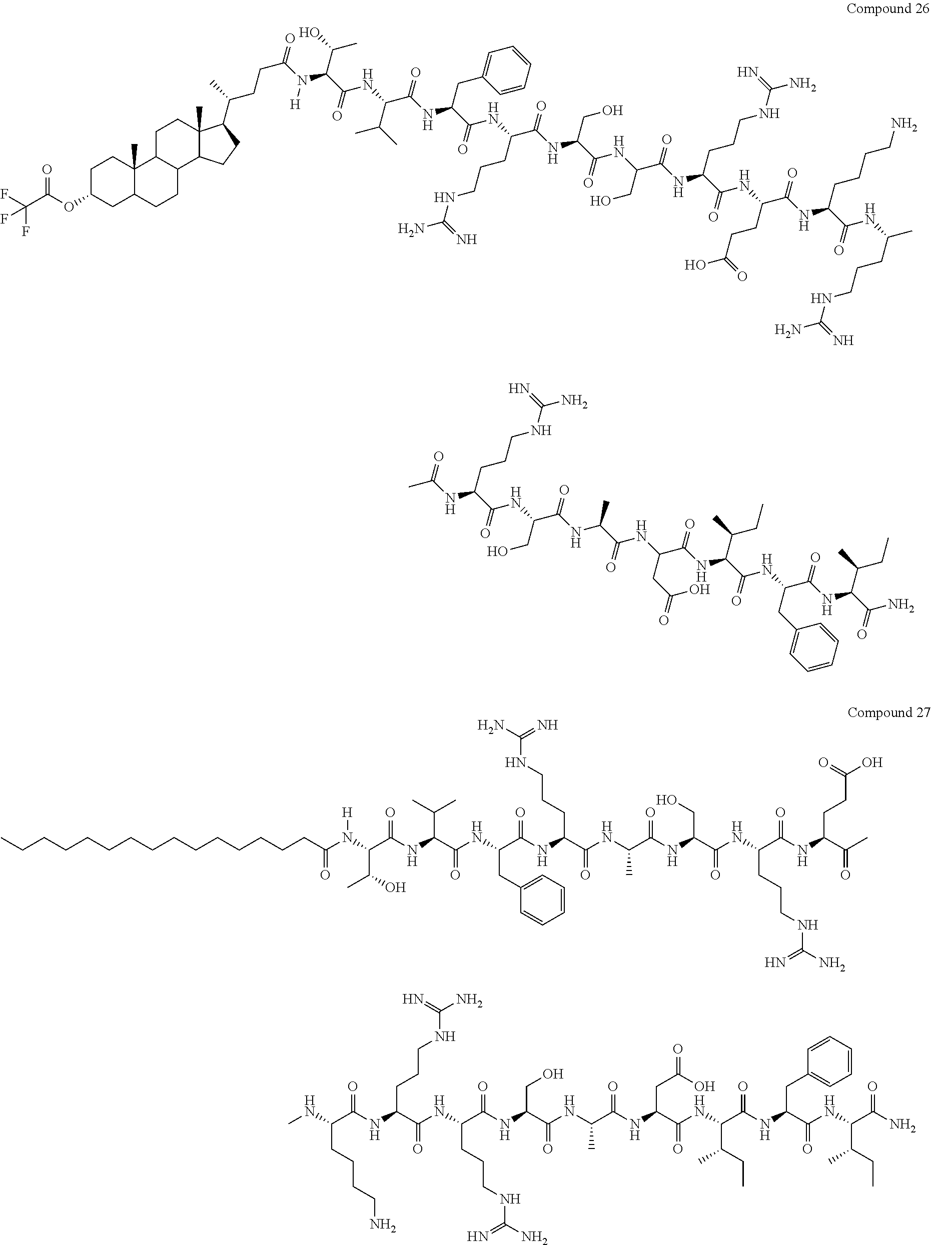

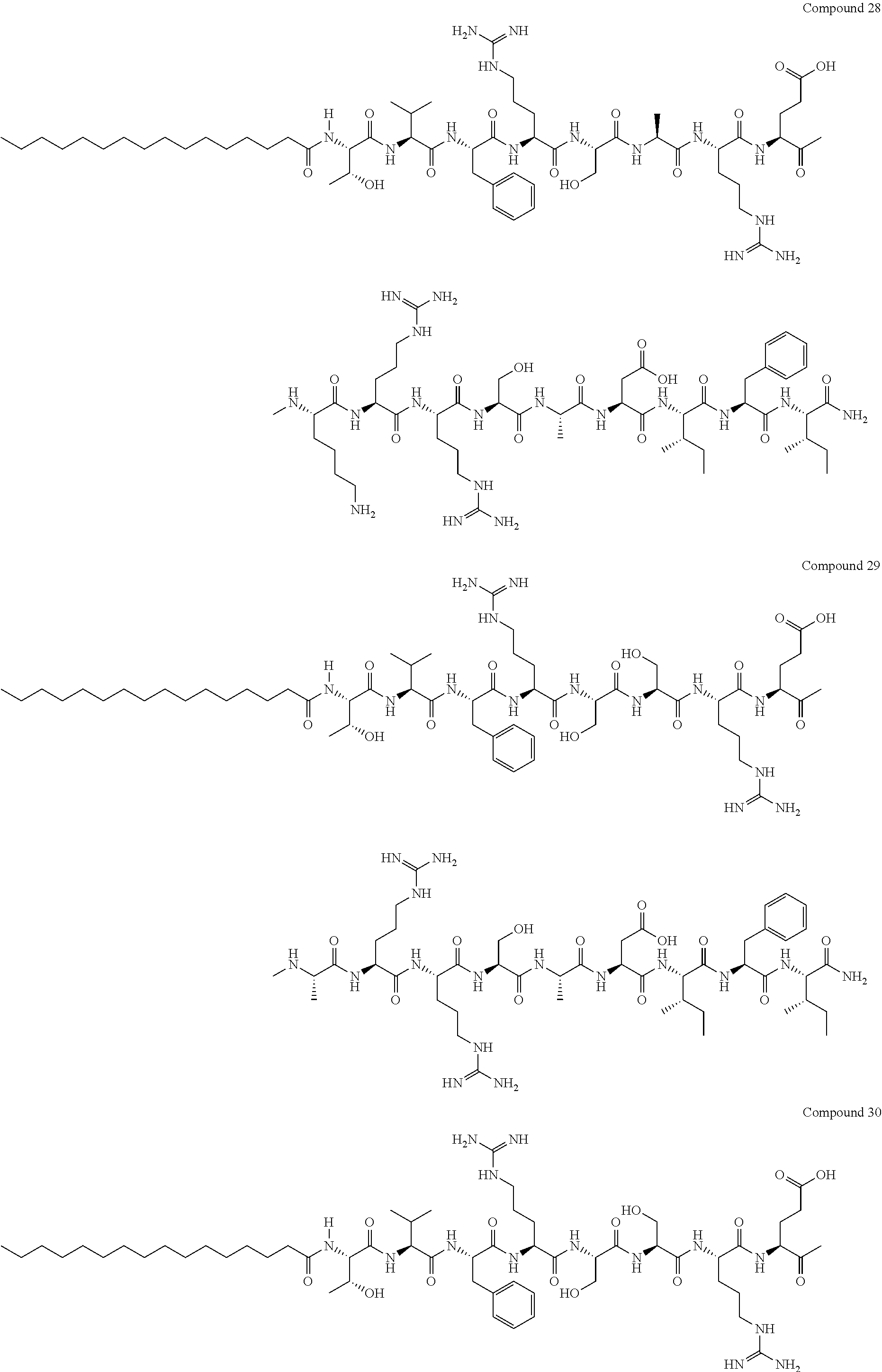

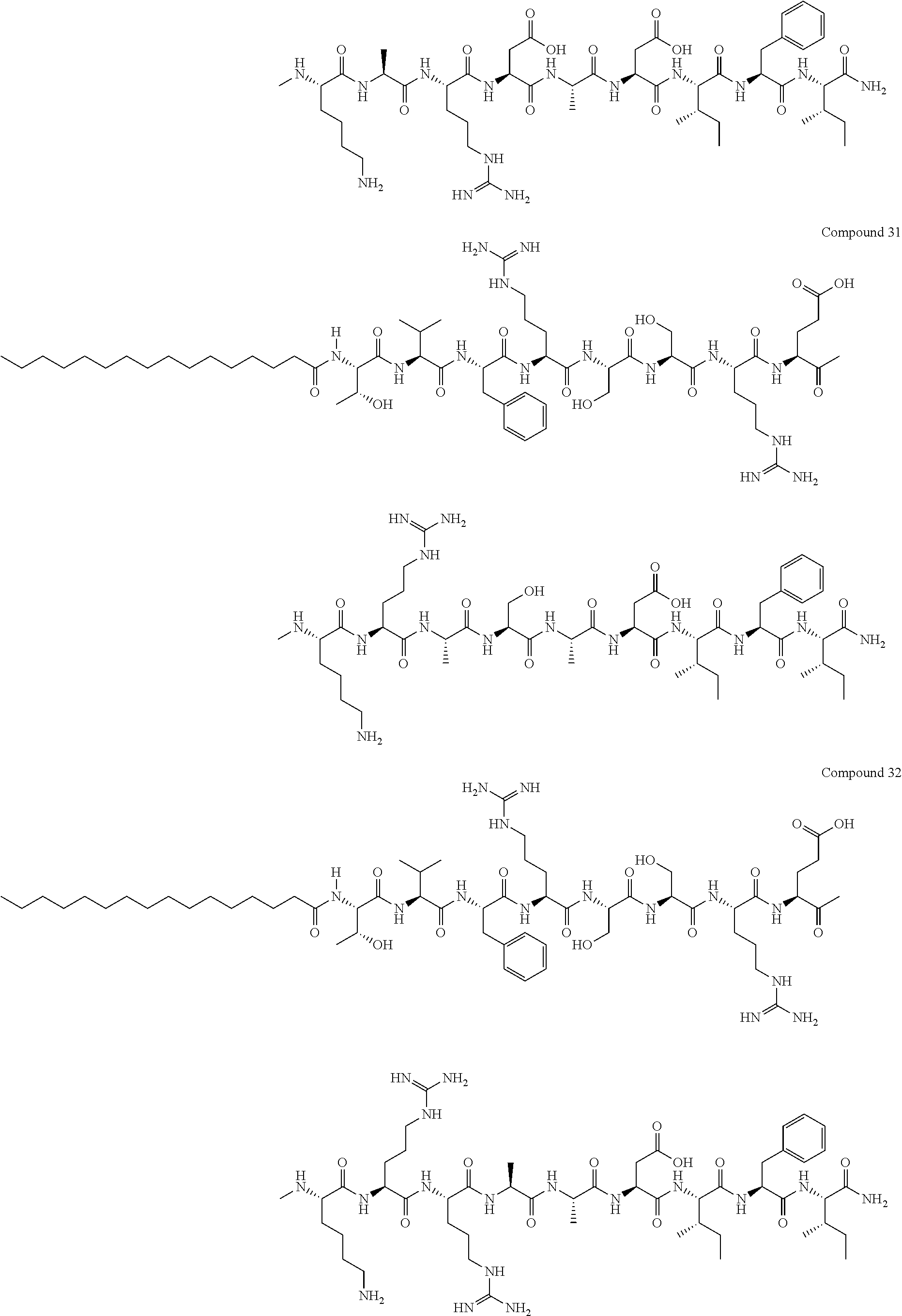

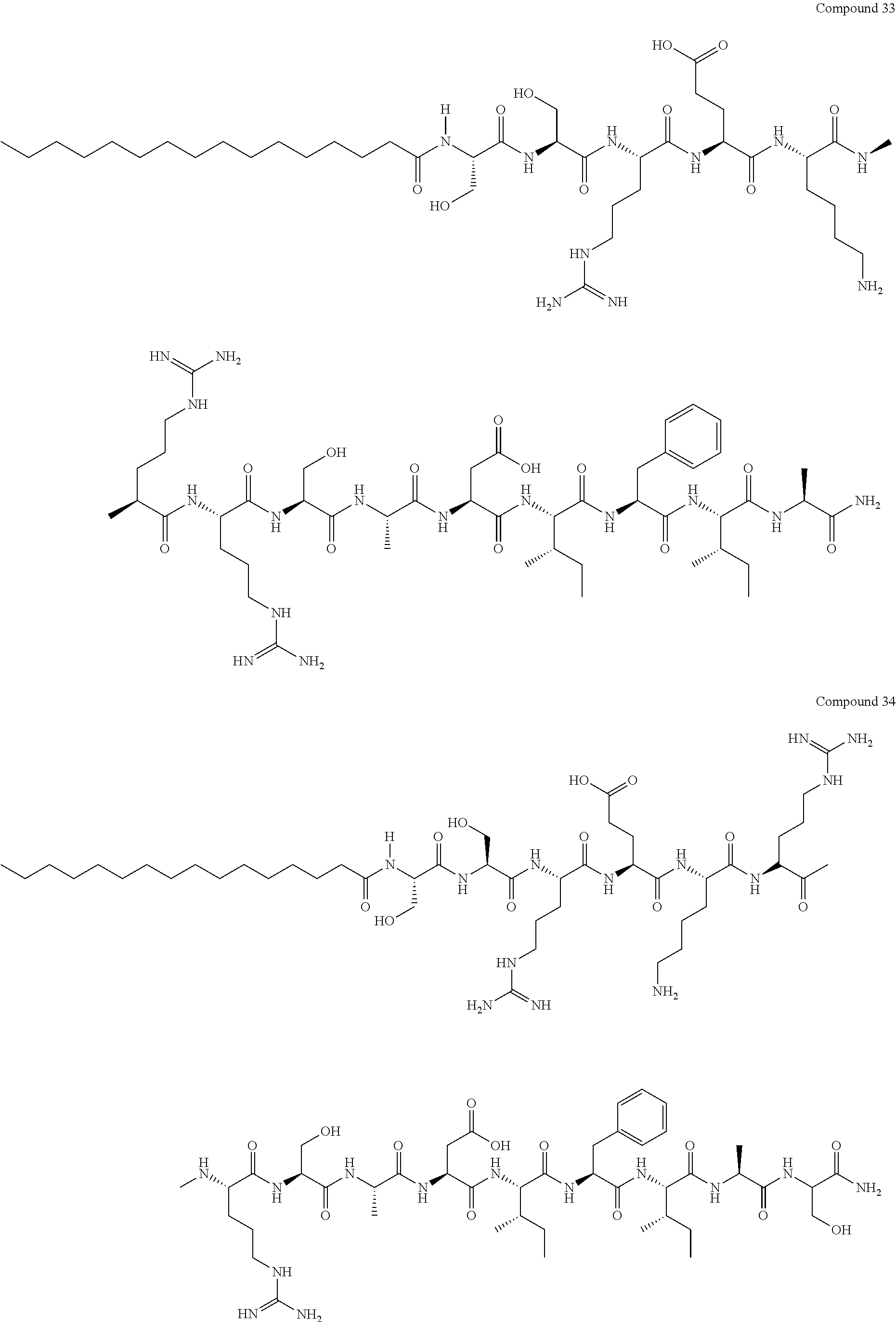

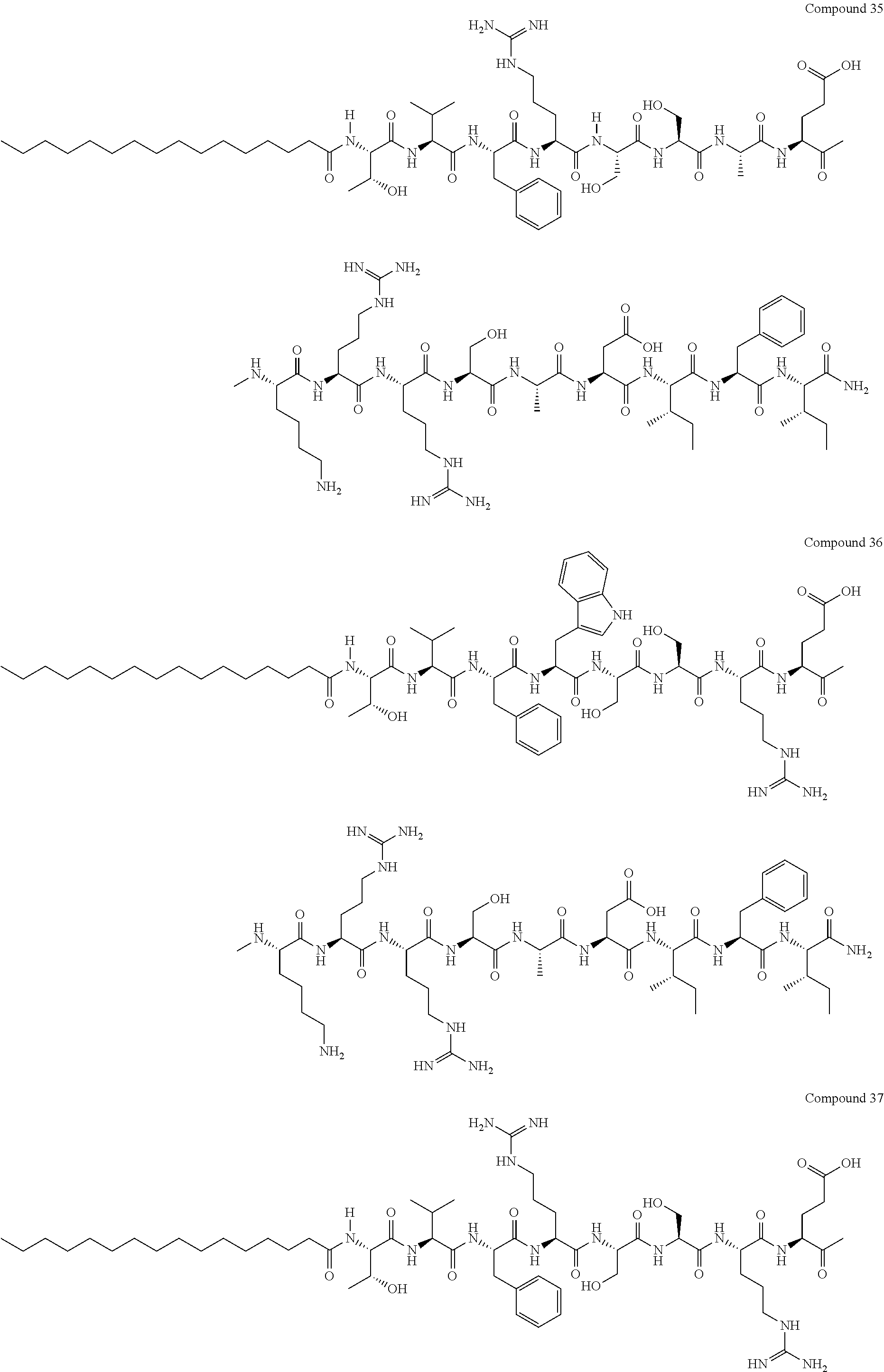

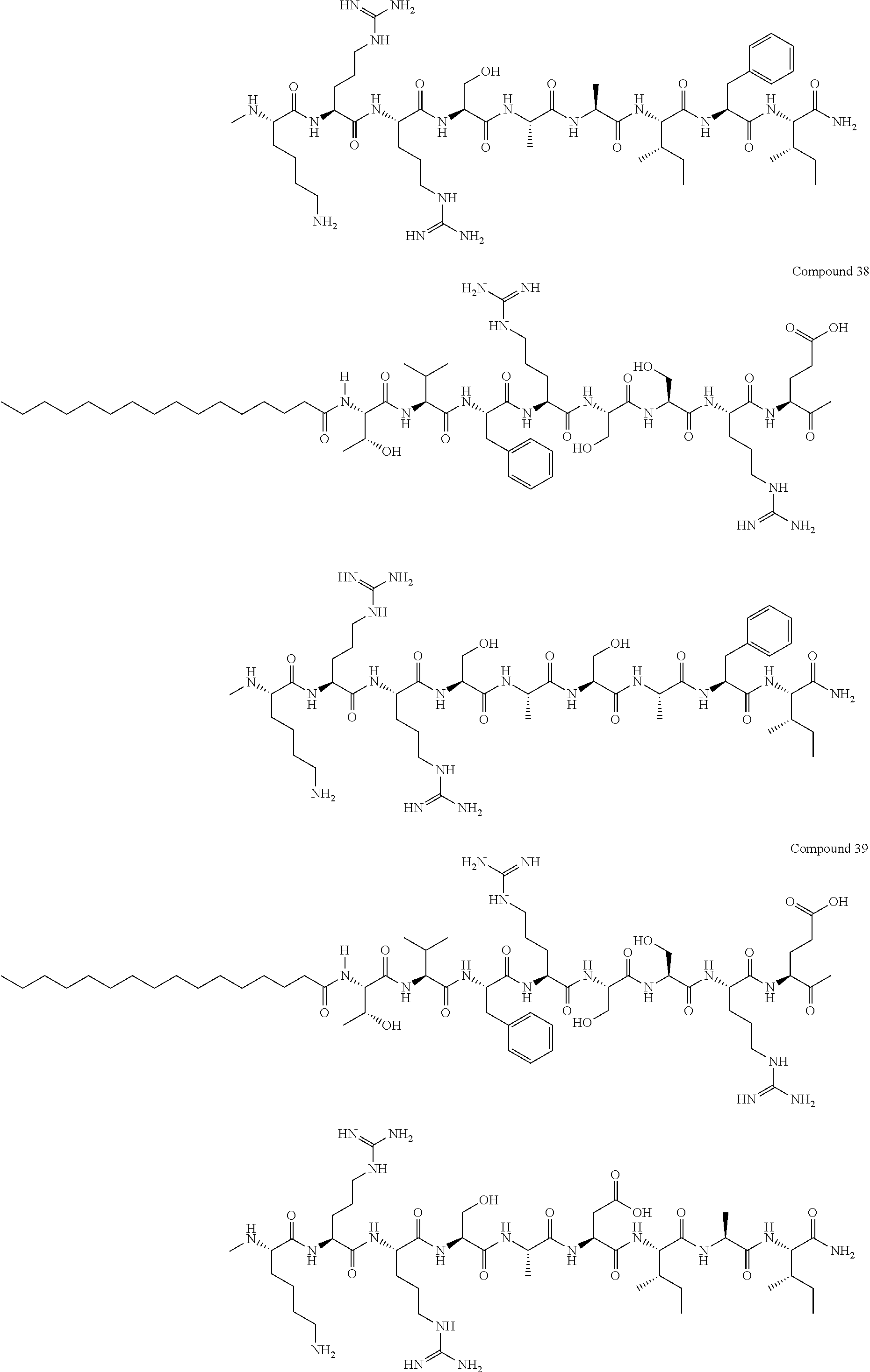

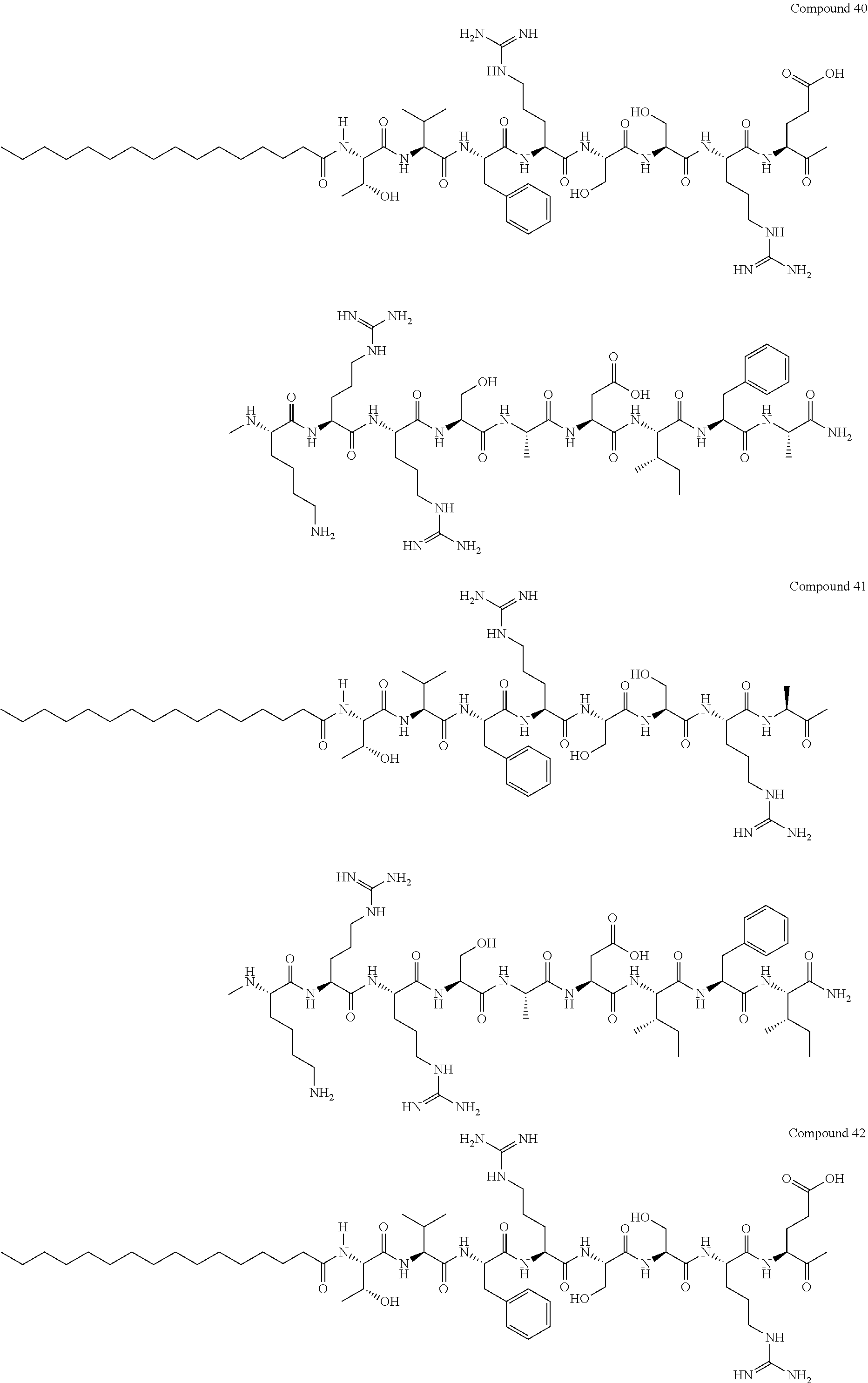

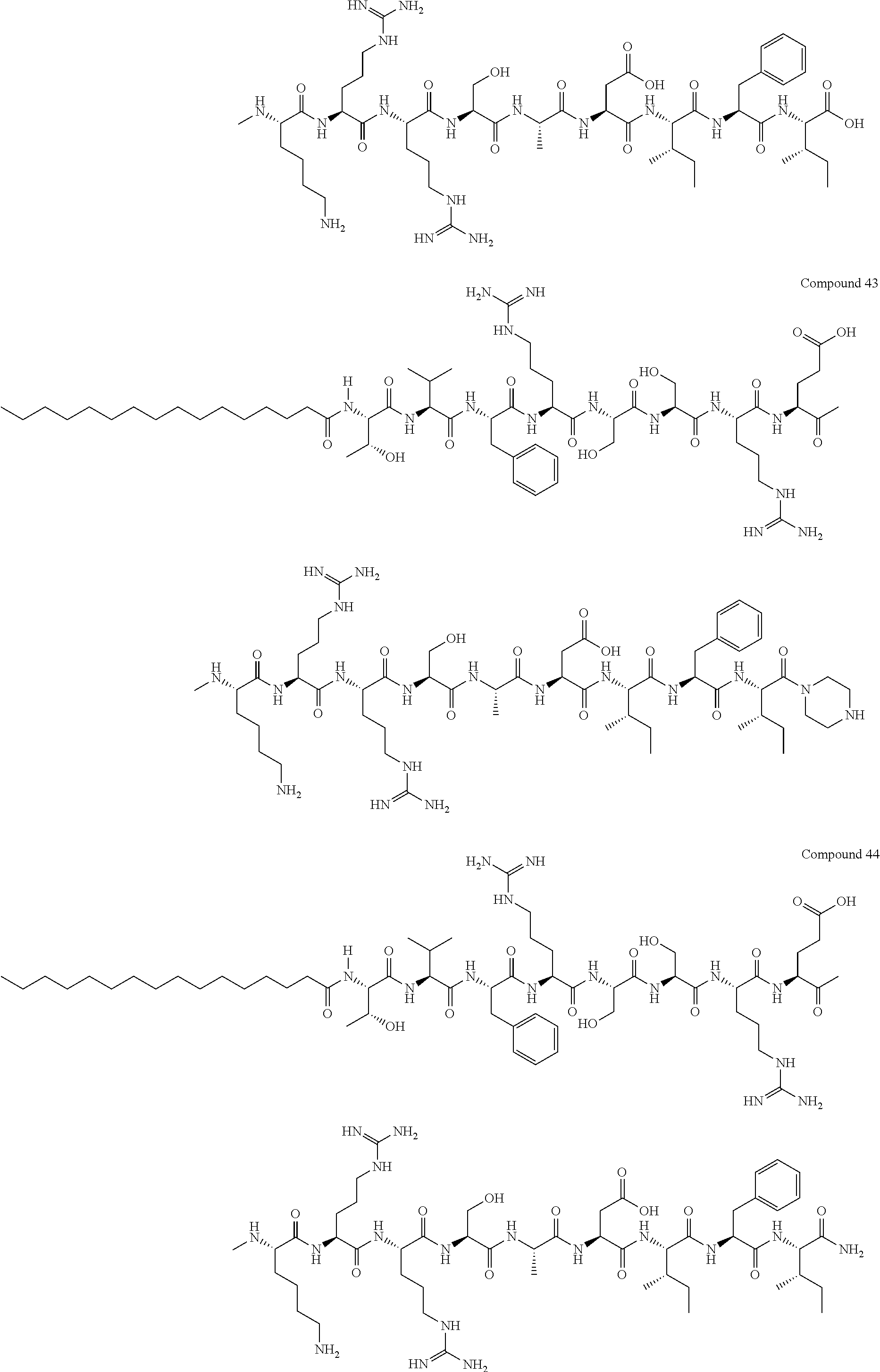

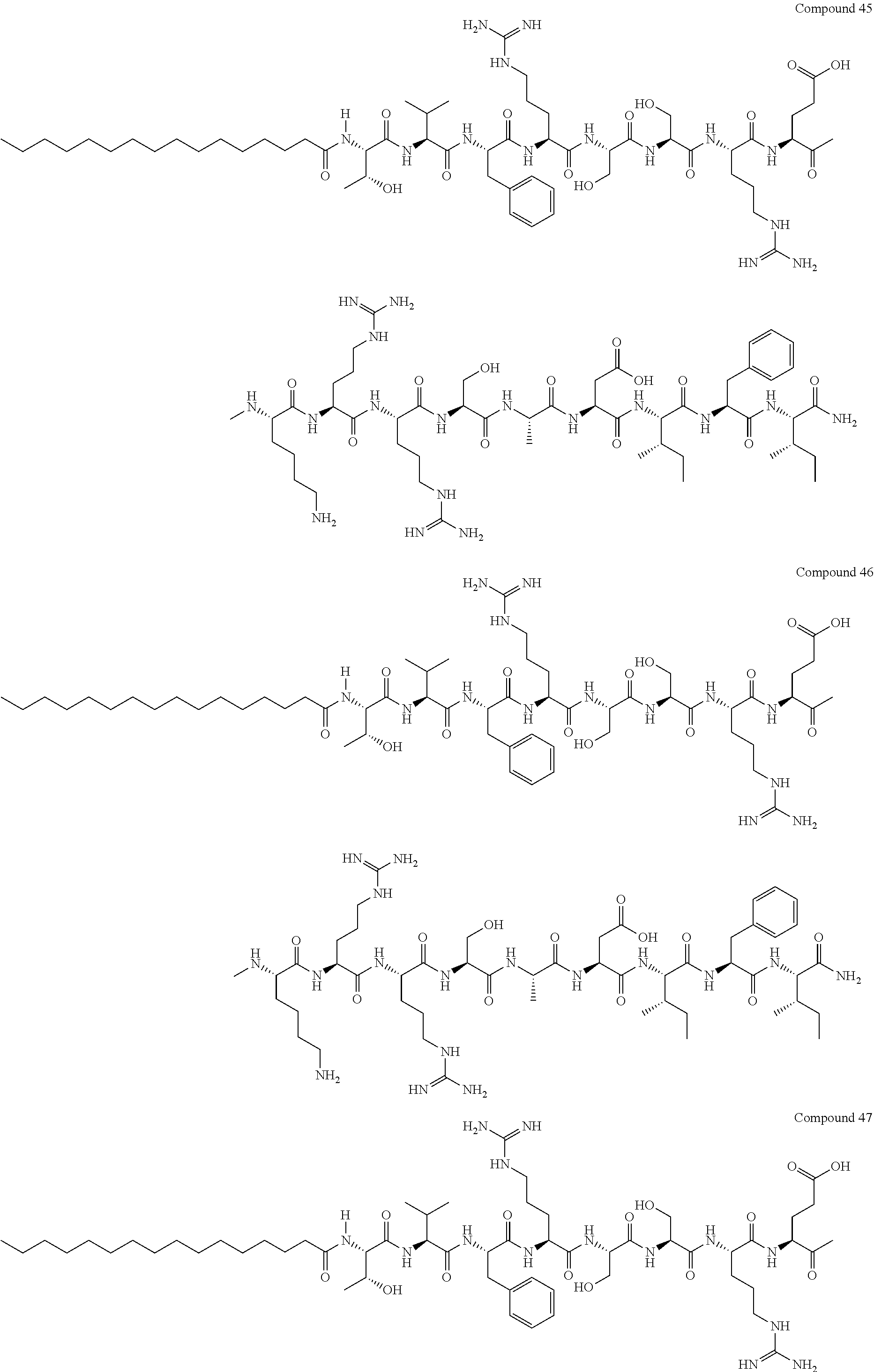

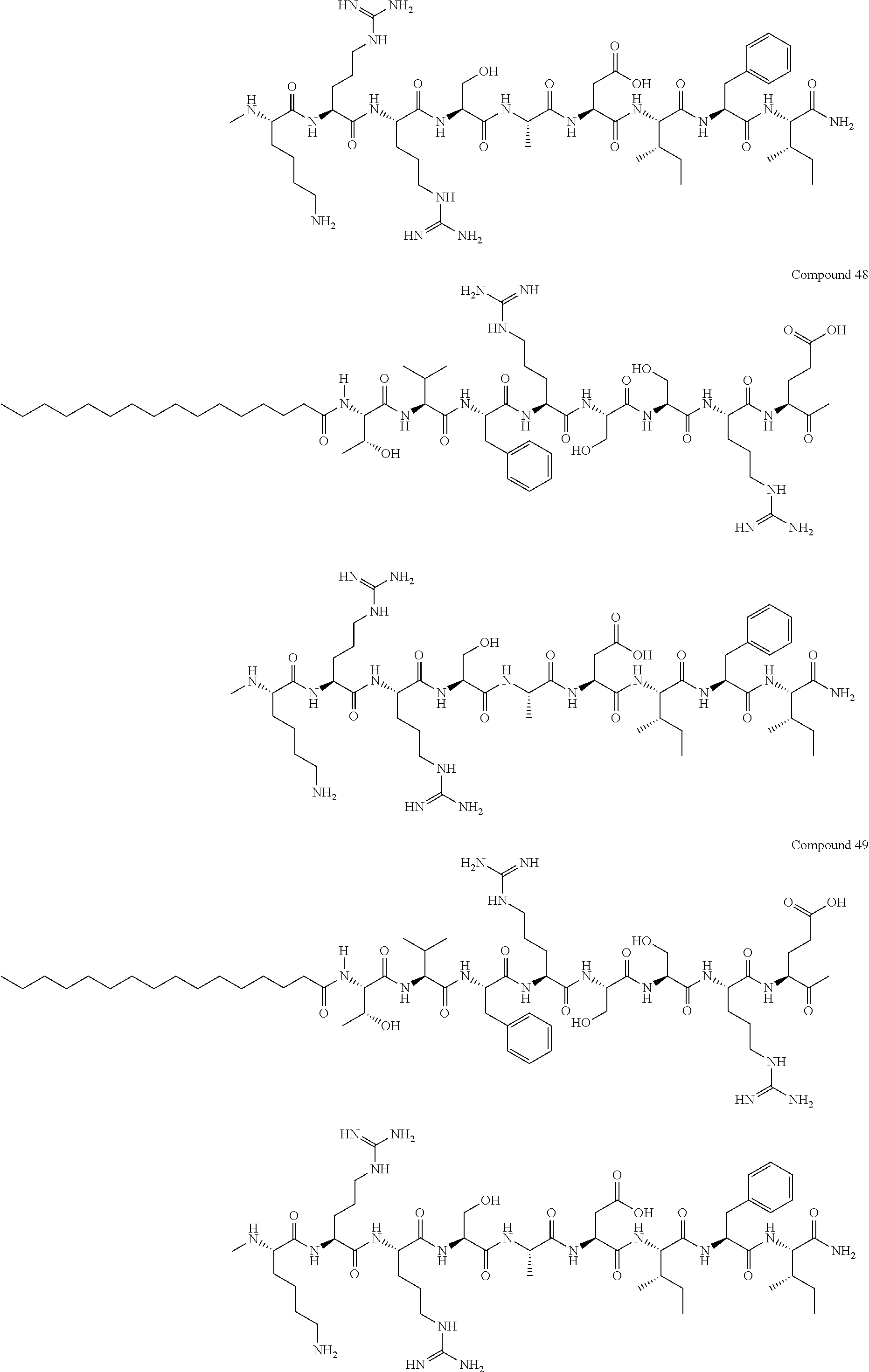

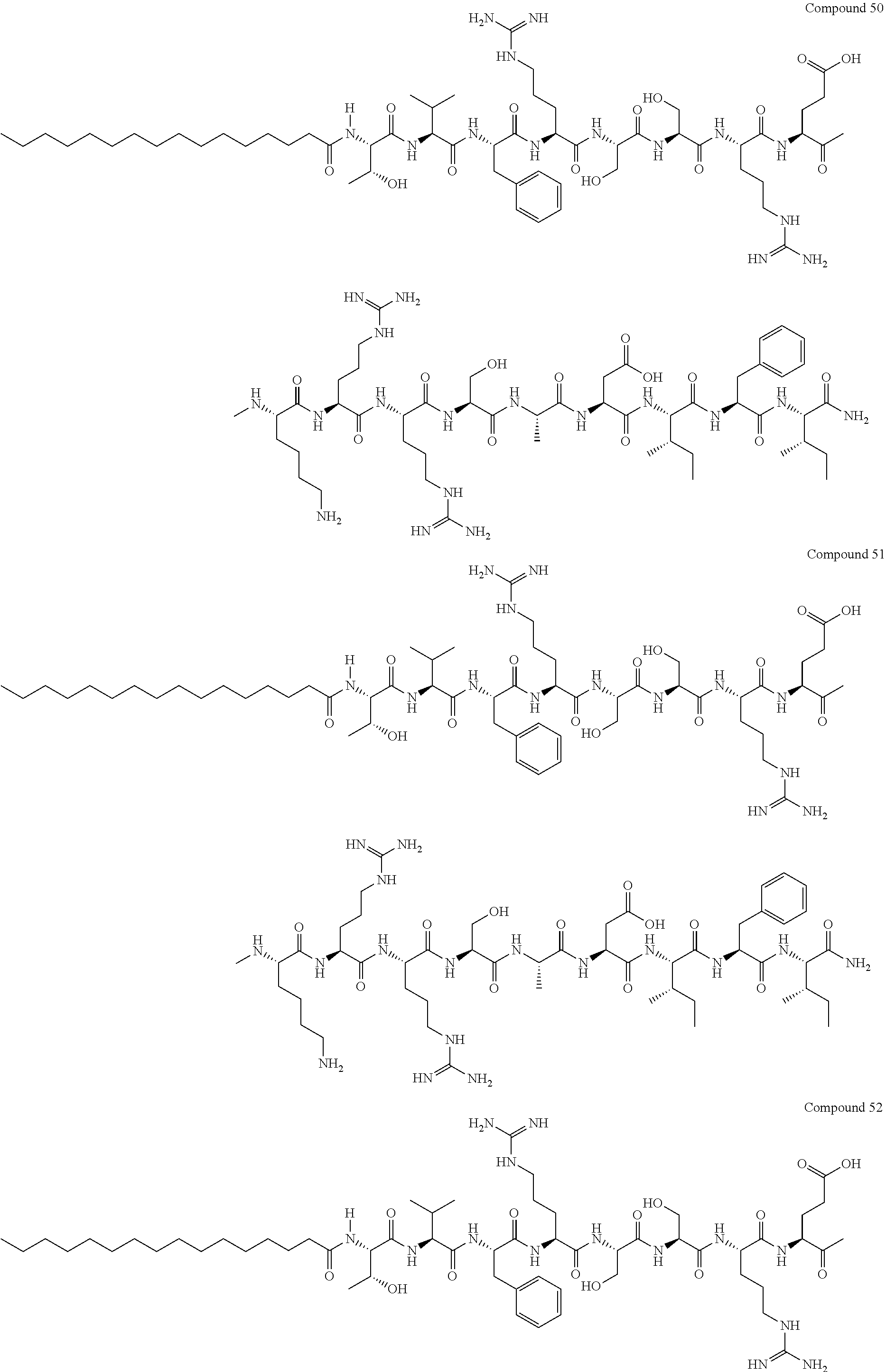

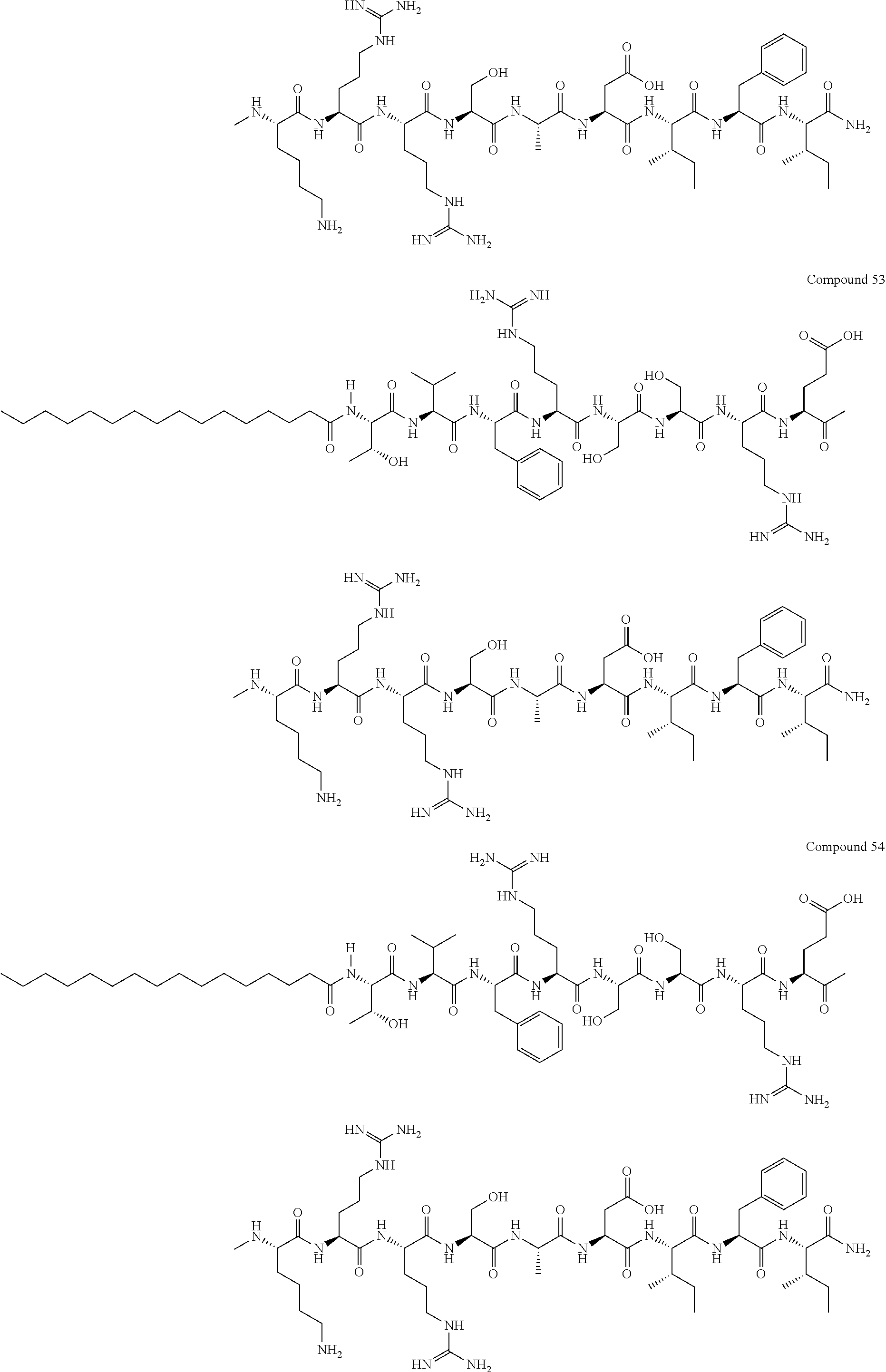

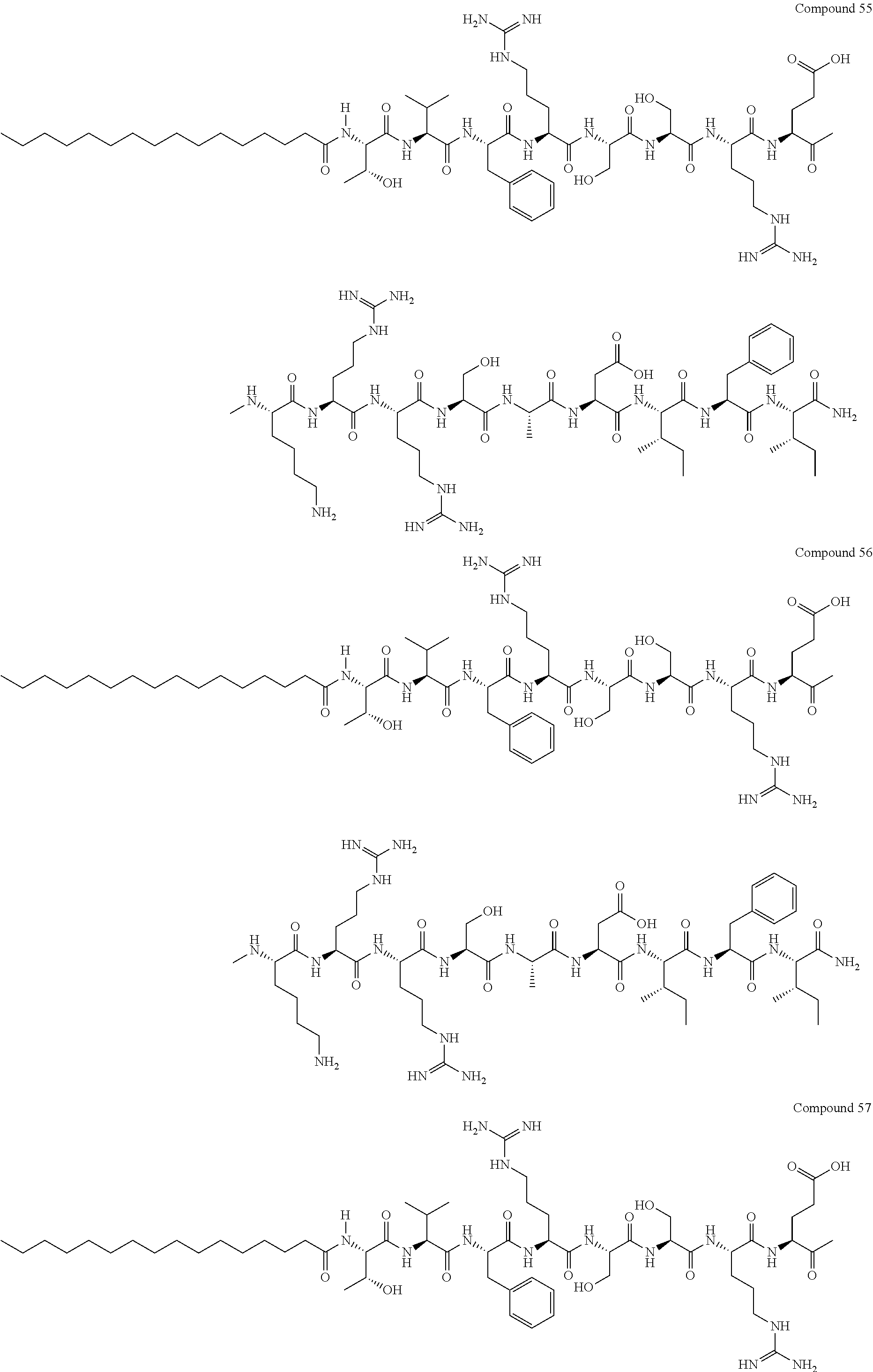

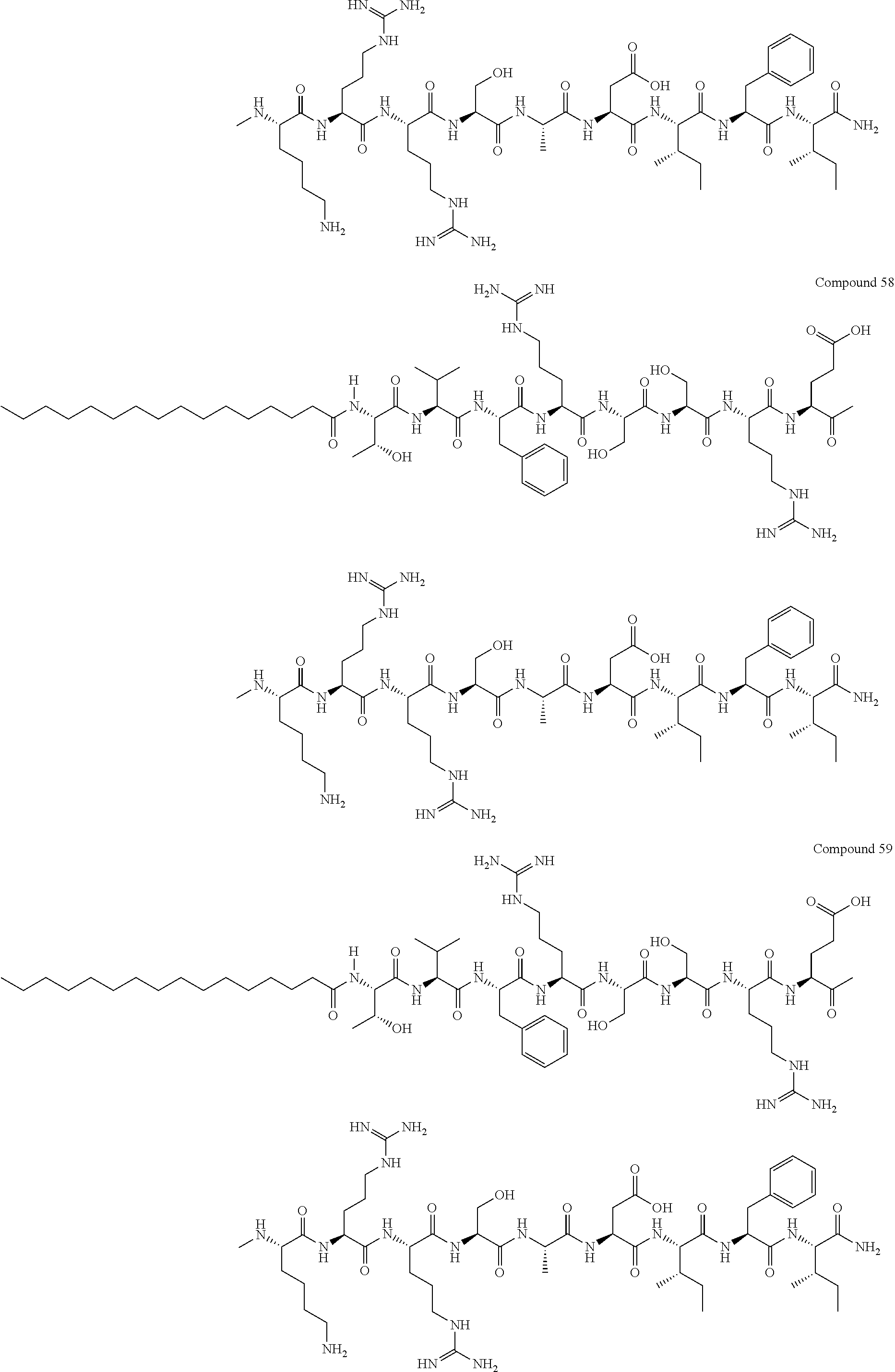

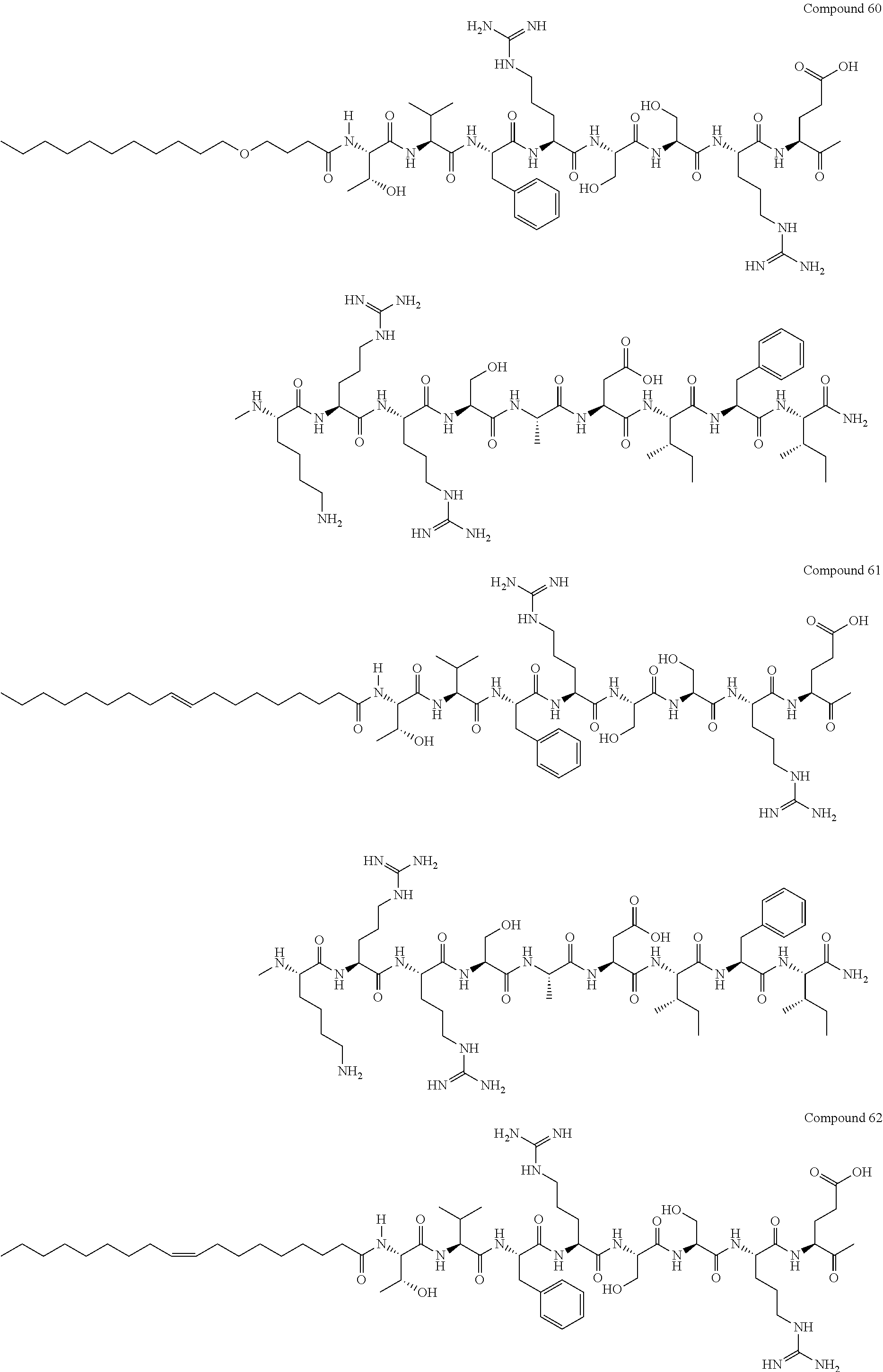

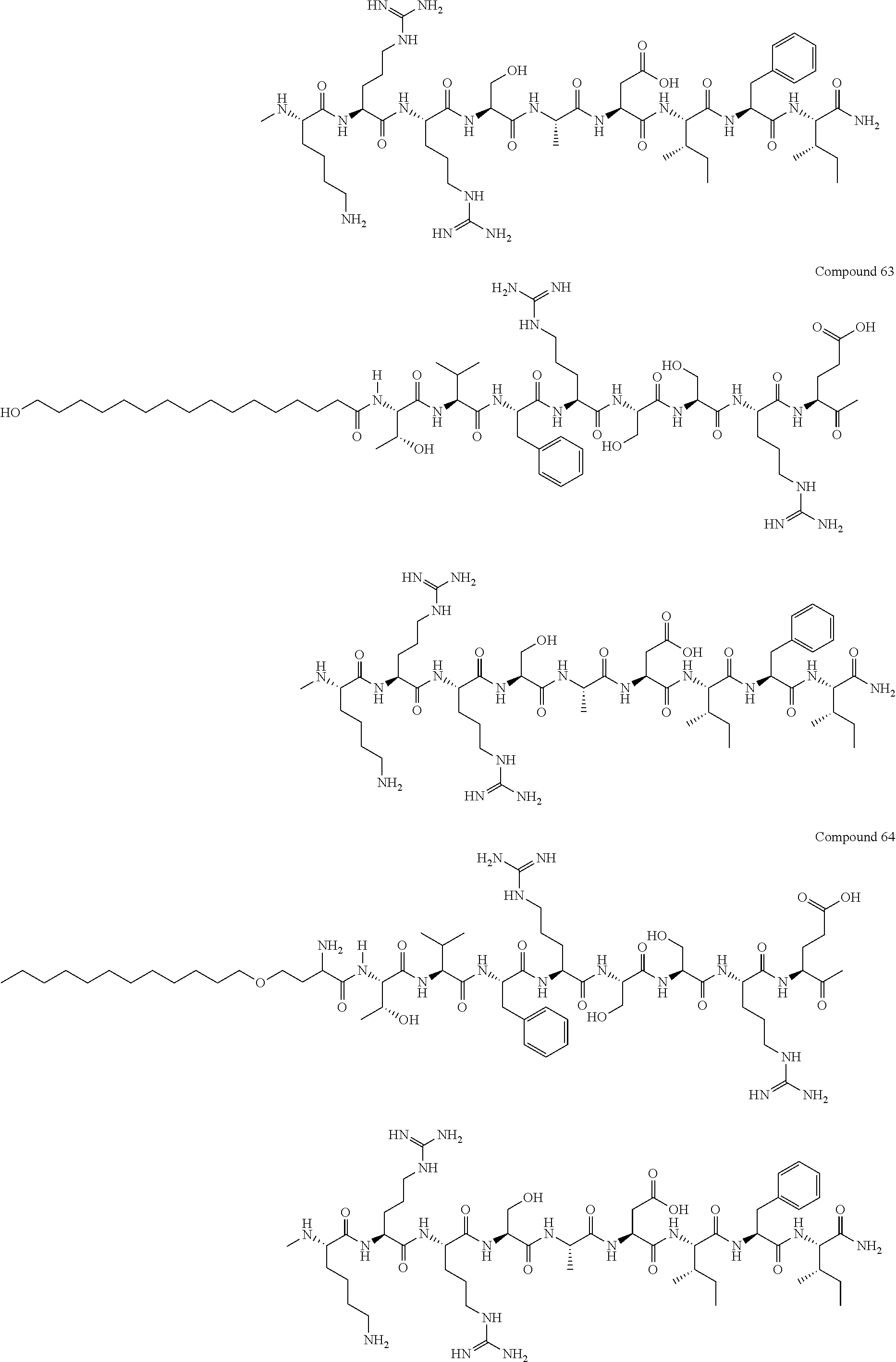

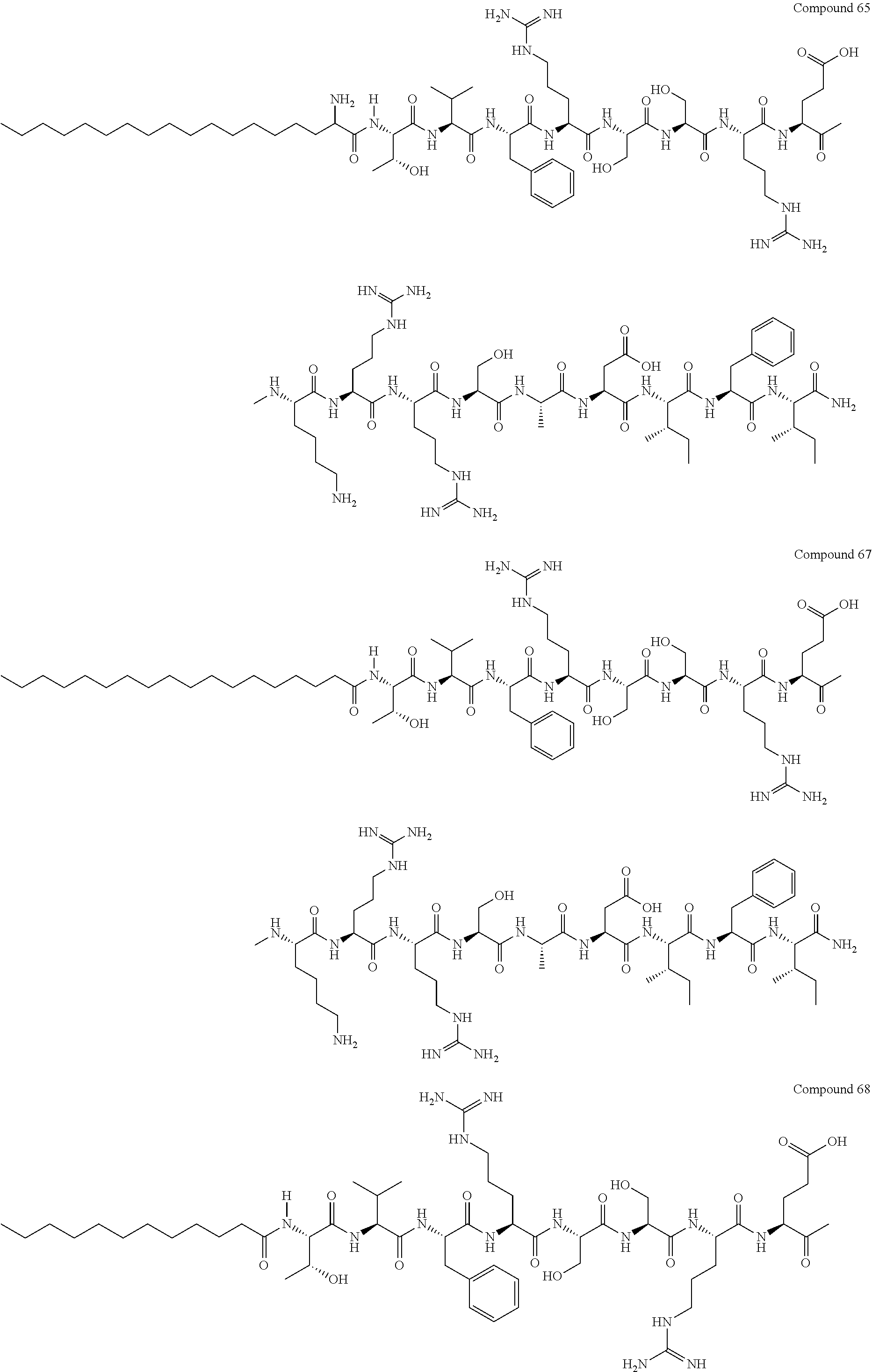

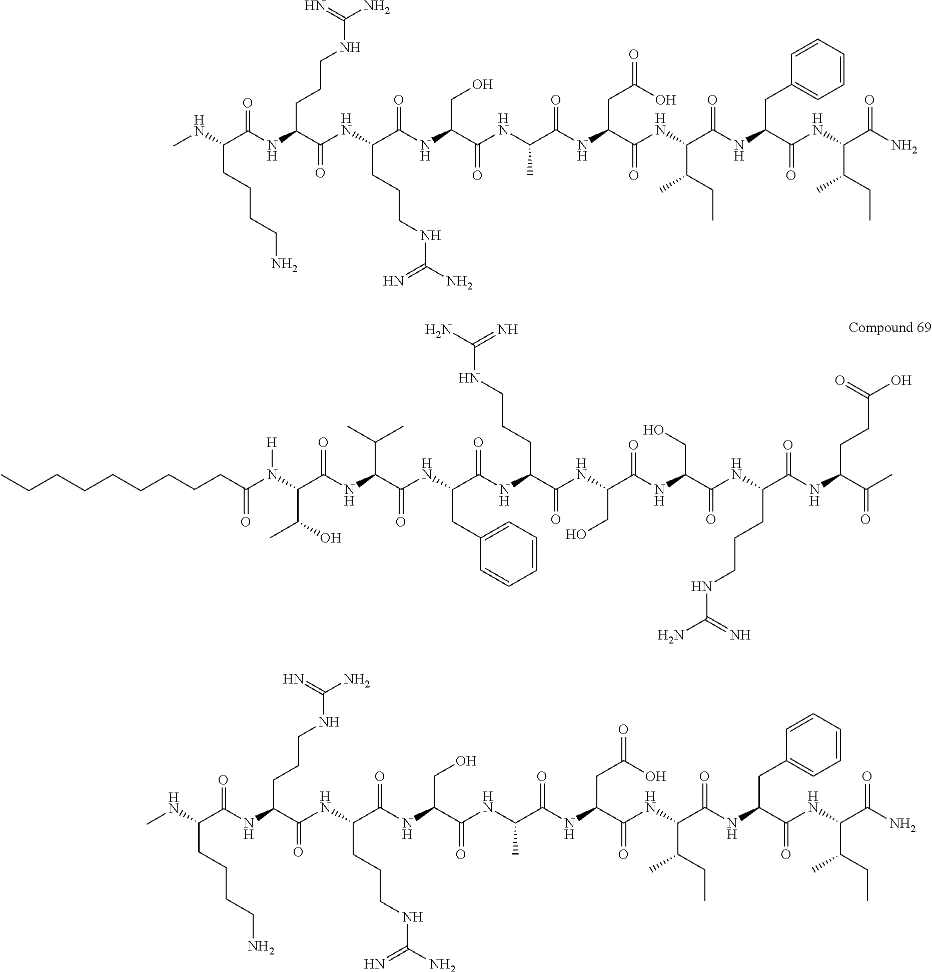

In the above listing of compounds, structures follow the compound number identifier.

In yet another embodiment, a GPCR compound of the invention is selected from one of the following compounds or a pharmaceutically acceptable salt thereof:

| | N- | | C- |
|---|---|---|---|
| Compound | terminus | Sequence | terminus |
| Compound 70 | Pal | TVFRSSRE(D-Dap)RRSADIFI | Amide |
| Compound 71 | Pal | TVFRSSREpKRRSADIFI | Amide |
| Compound 72 | Pal | TVFRSSRpEKRRSADIFI | Amide |
| Compound 73 | Pal | TVFRSSRE(D-Dab)RRSADIFI | Amide |
| Compound 74 | Pal | TVFRSSRE(D-Orn)RRSADIFI | Amide |

wherein Pal is C₁₅H₃₁C(O)—.

In yet another embodiment, a GPCR compound of the invention is selected from one of the following compounds or a pharmaceutically acceptable salt thereof:

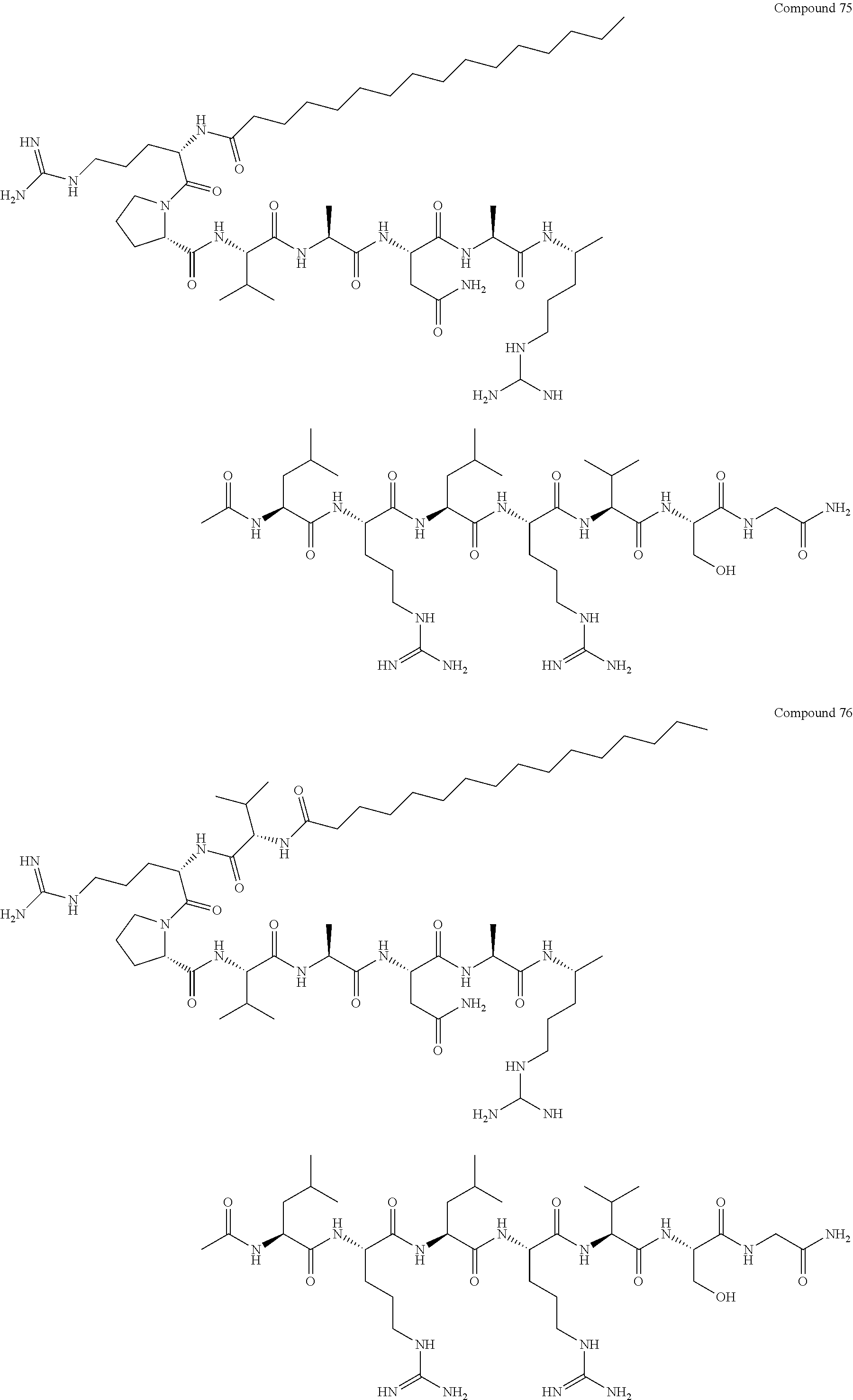

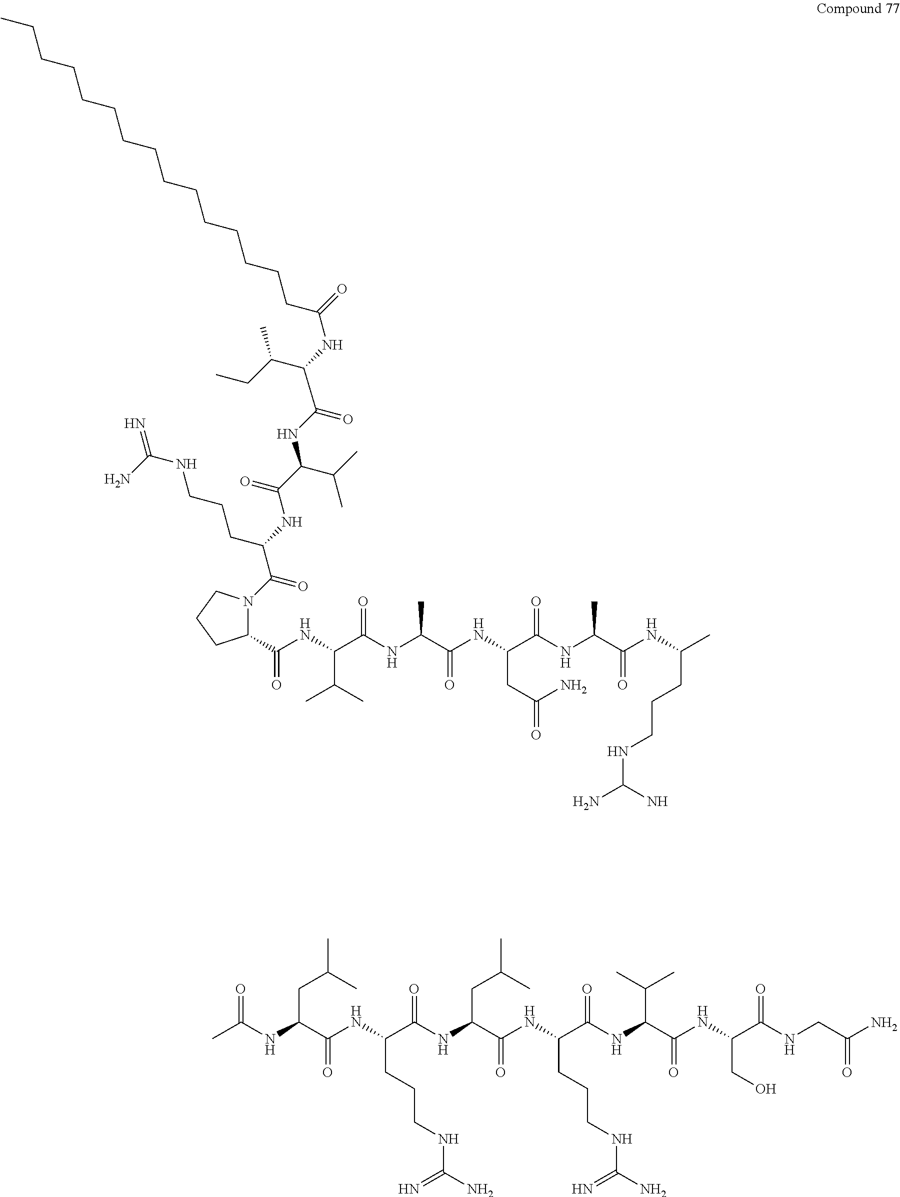

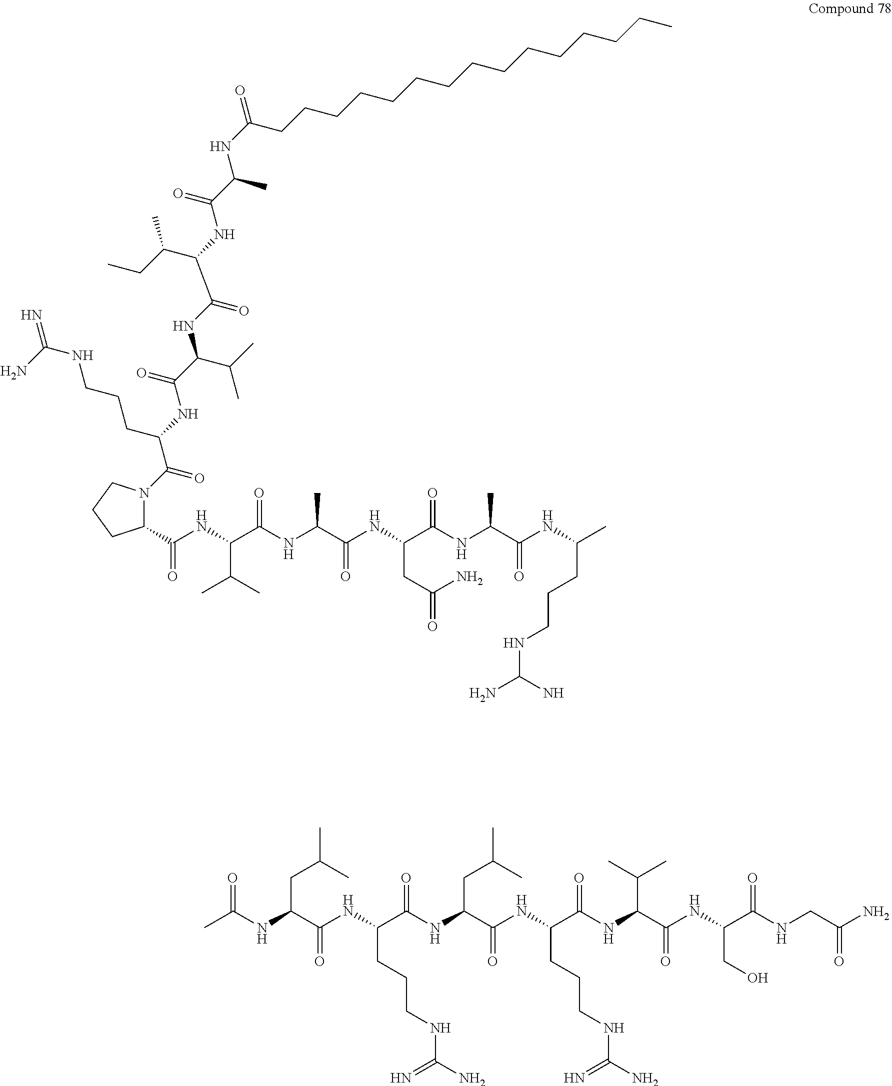

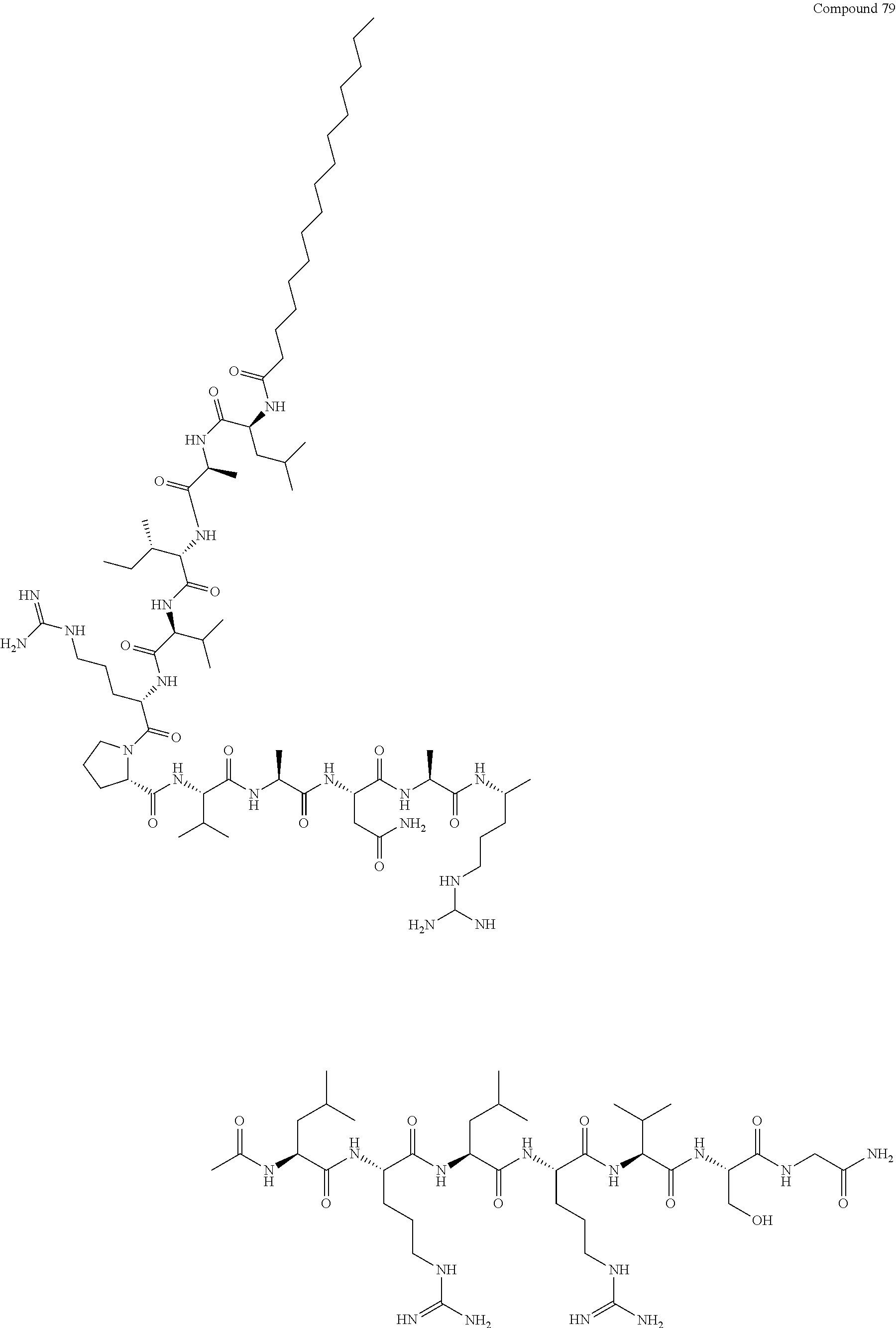

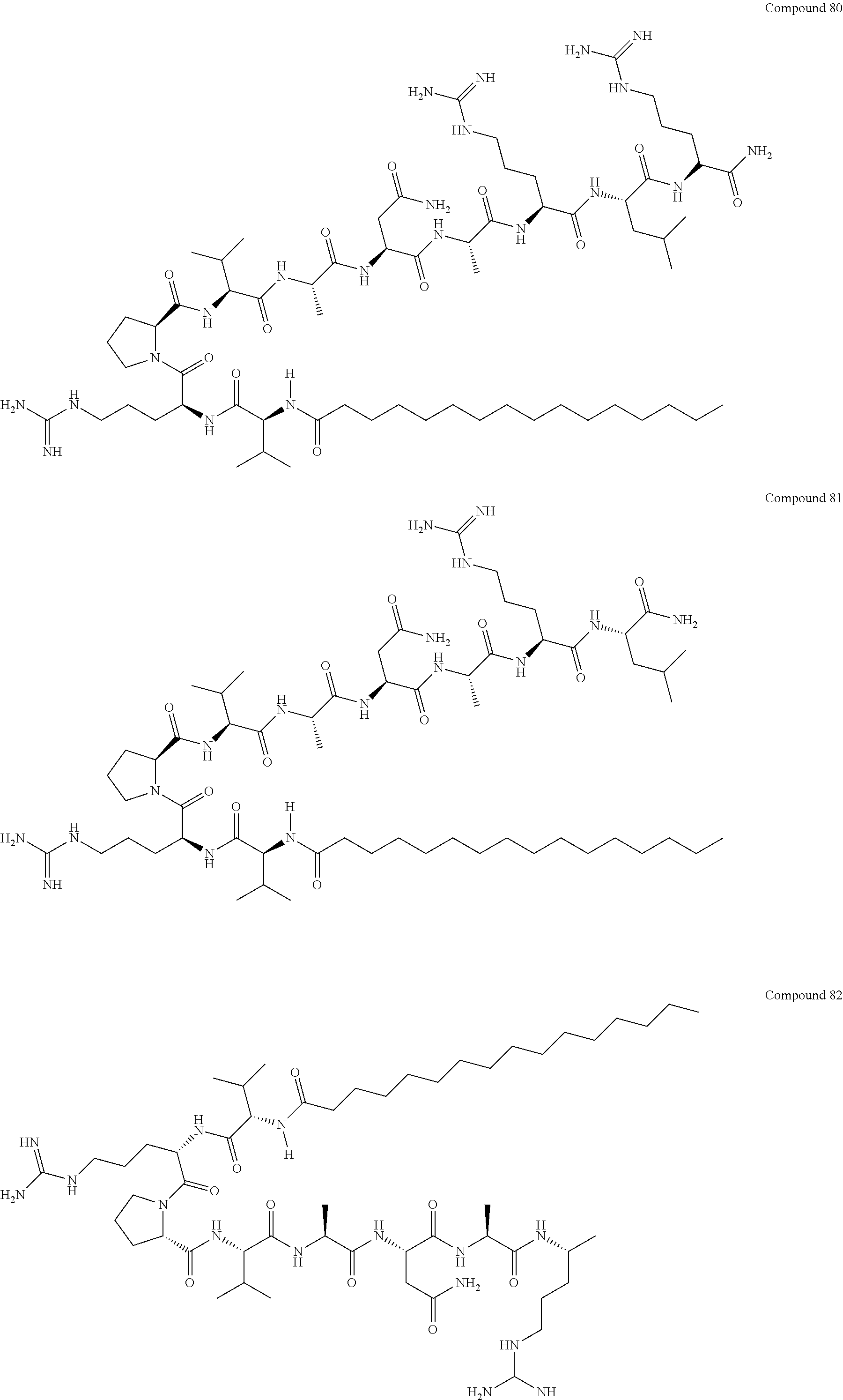

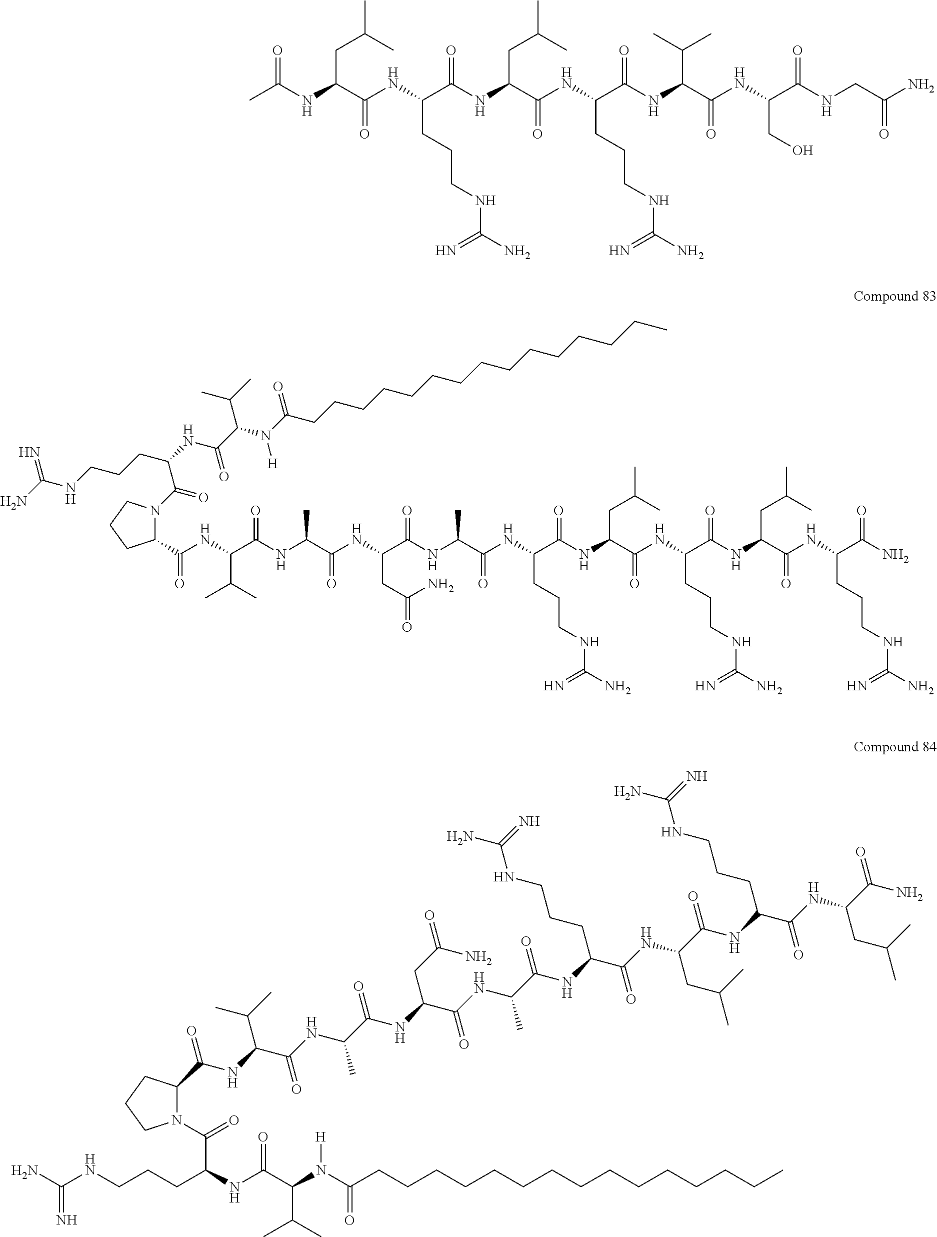

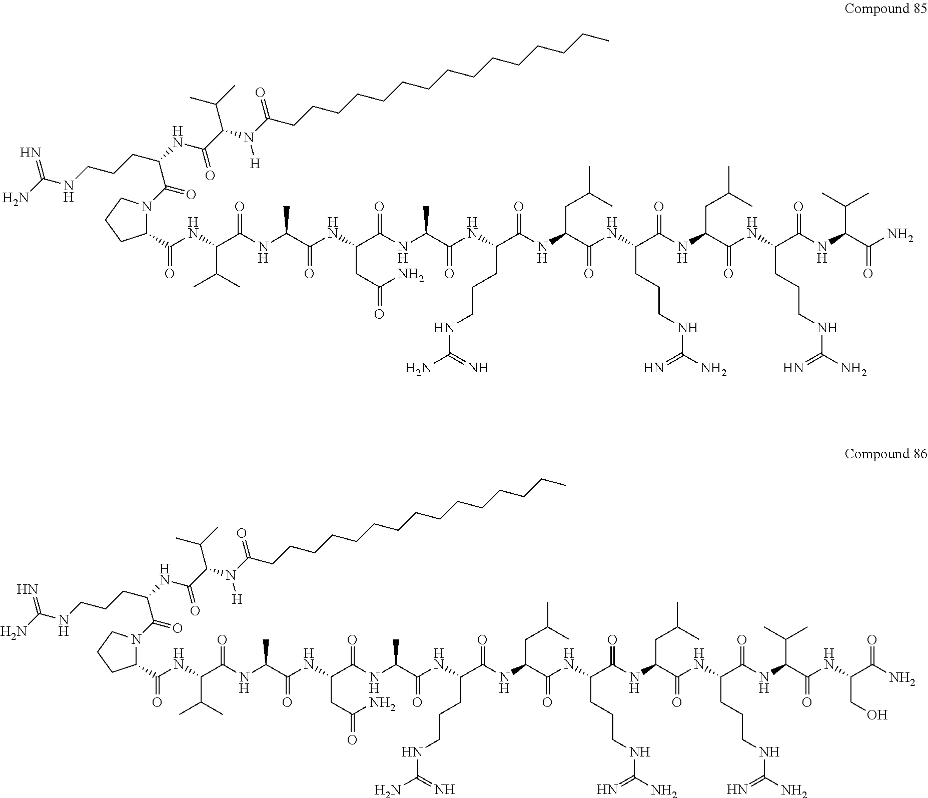

In yet another embodiment, a GPCR compound of the invention is selected from one of the following compounds or a pharmaceutically acceptable salt thereof:

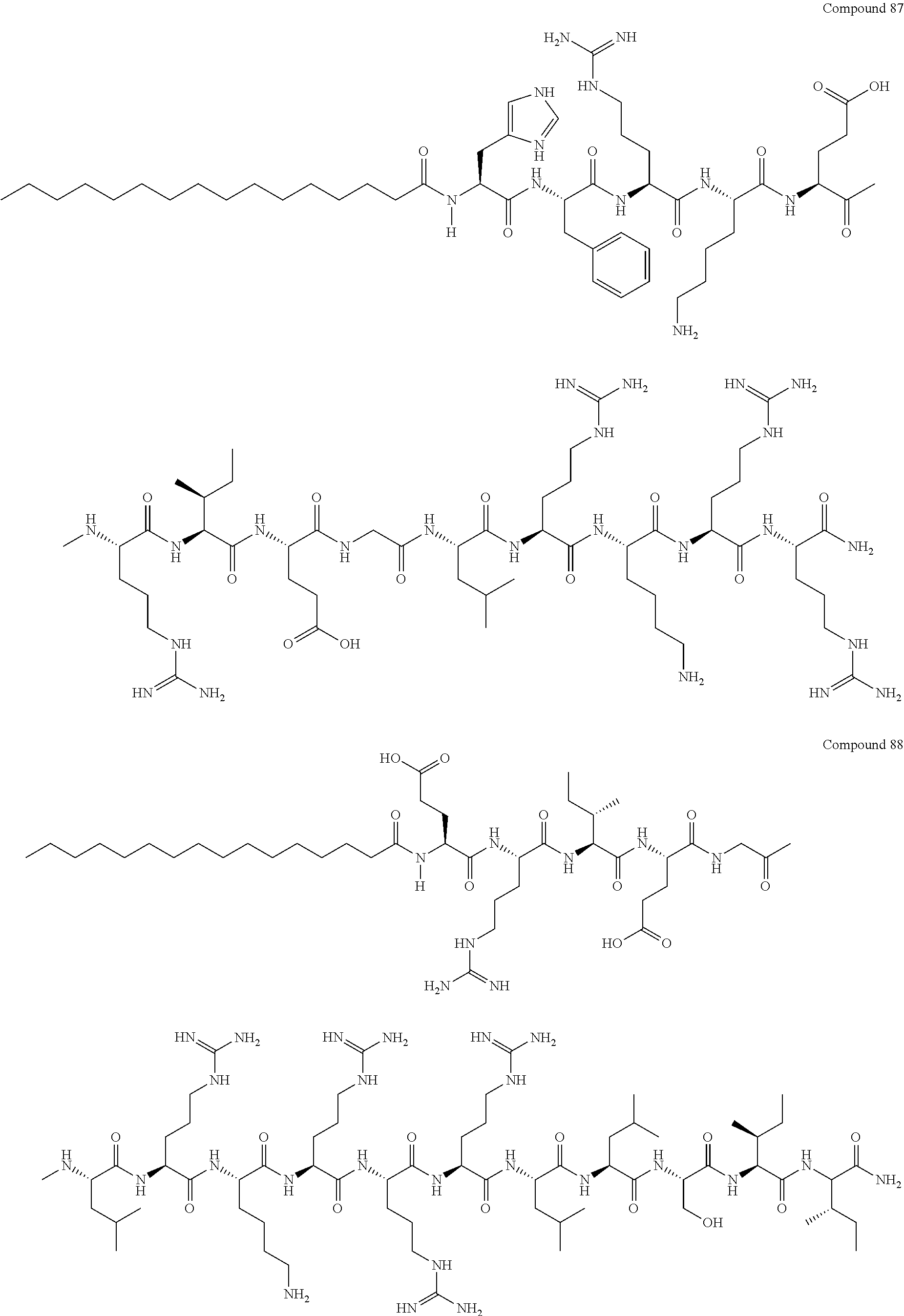

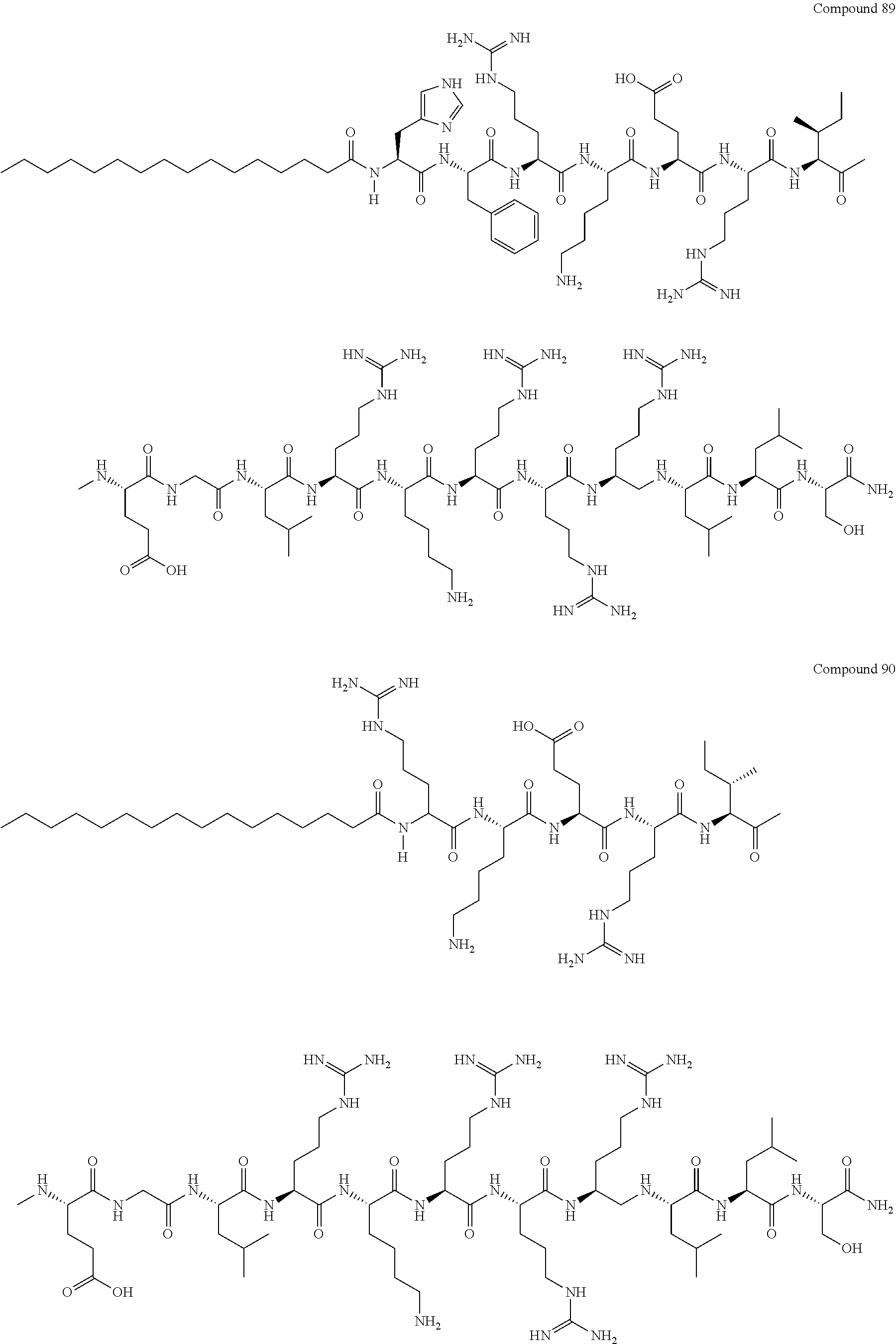

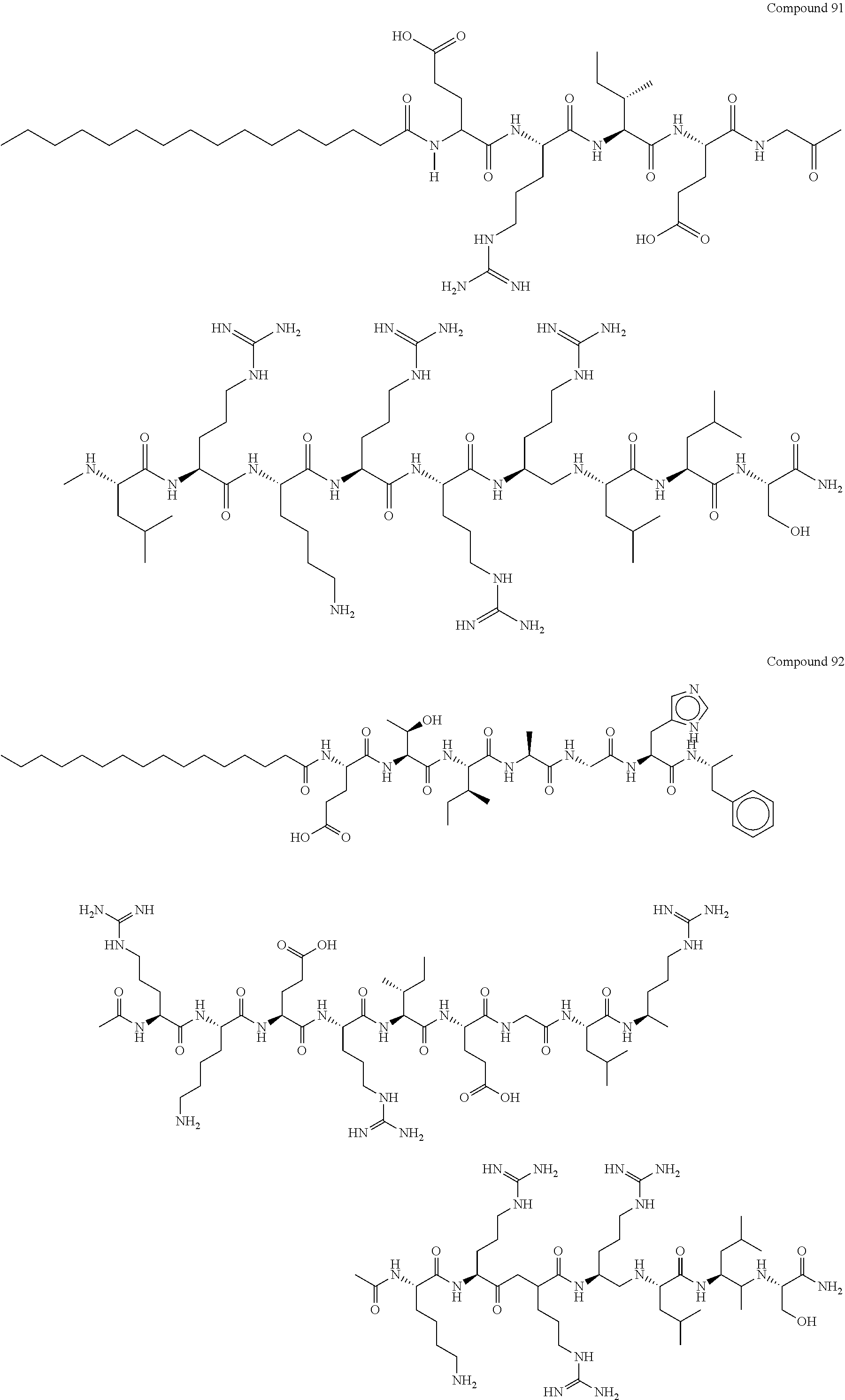

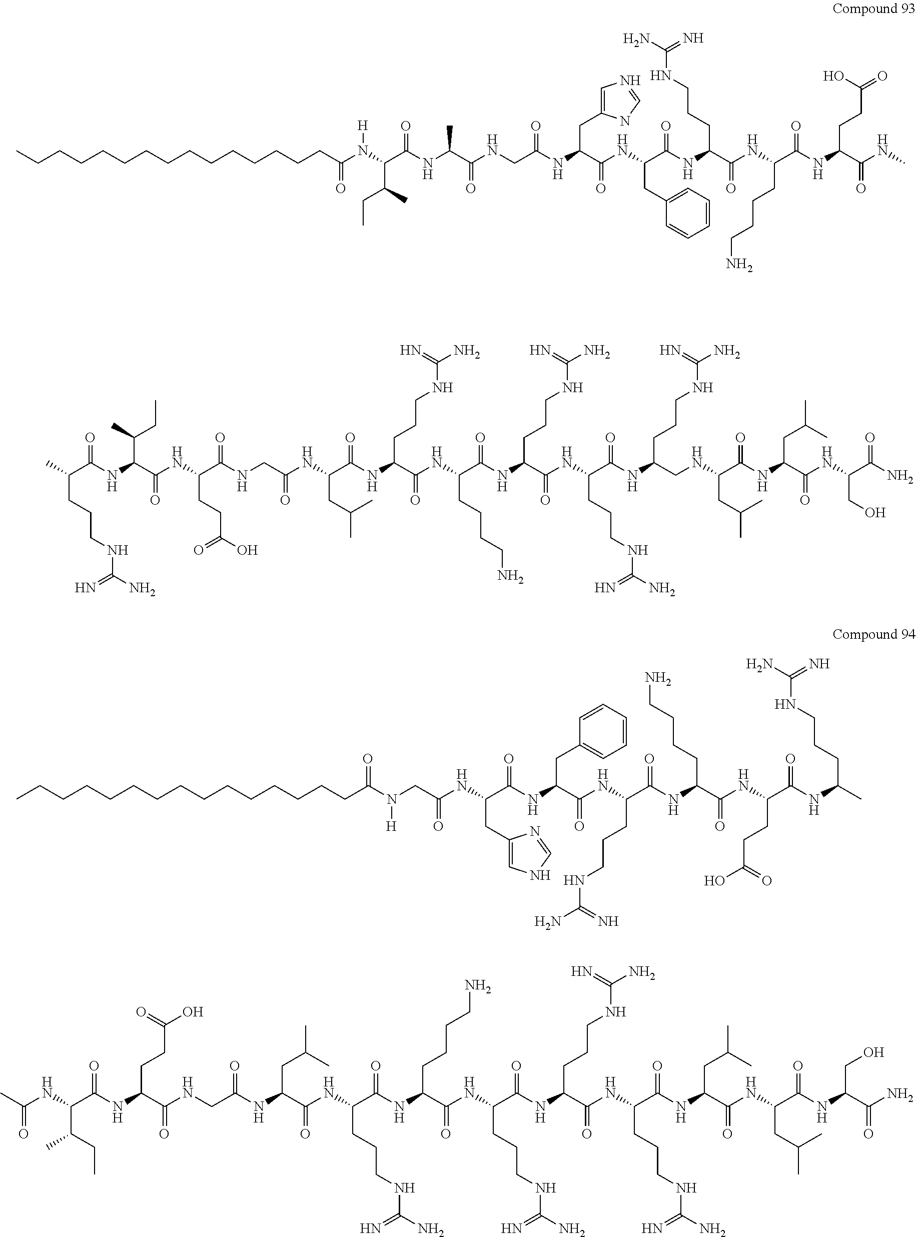

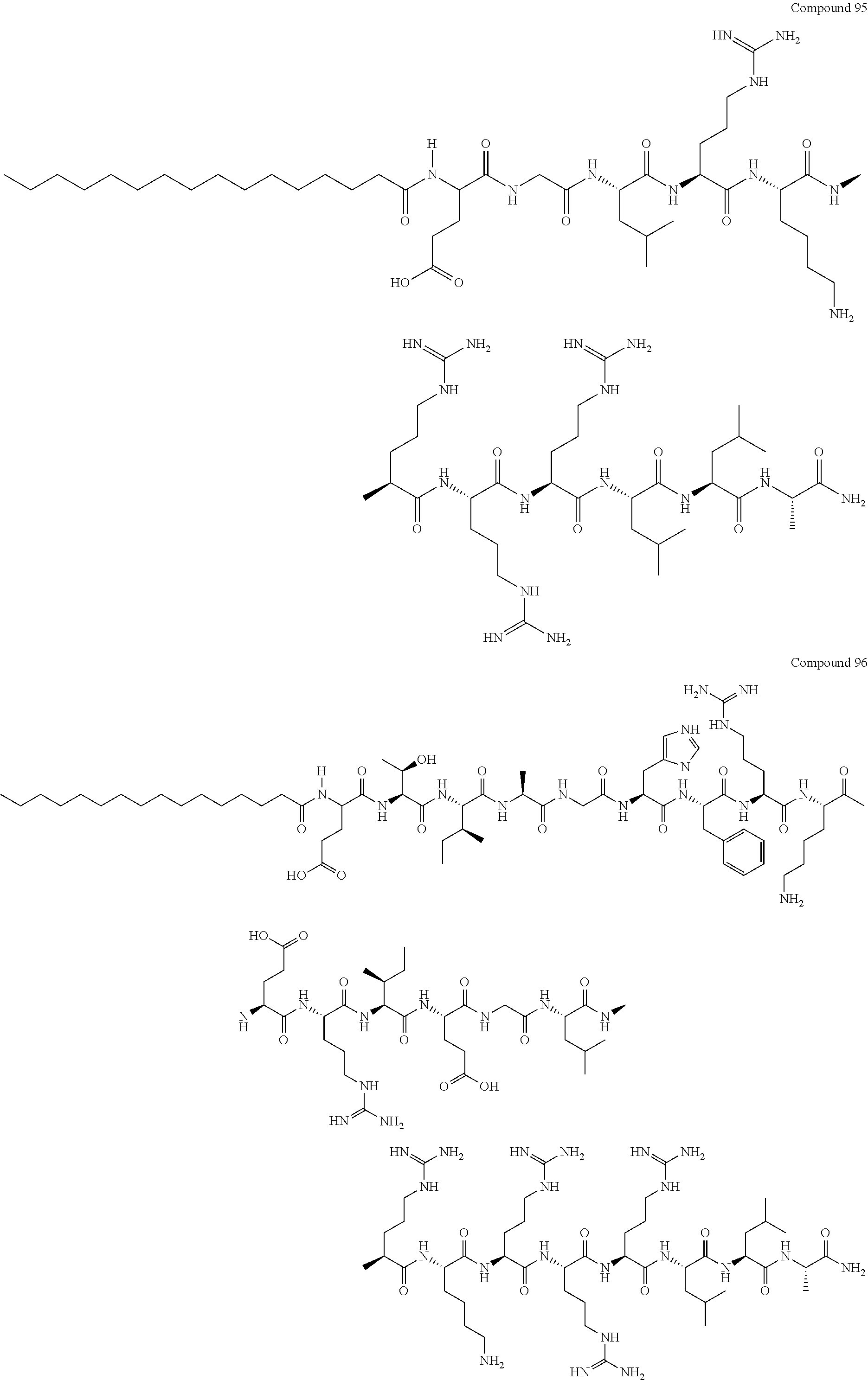

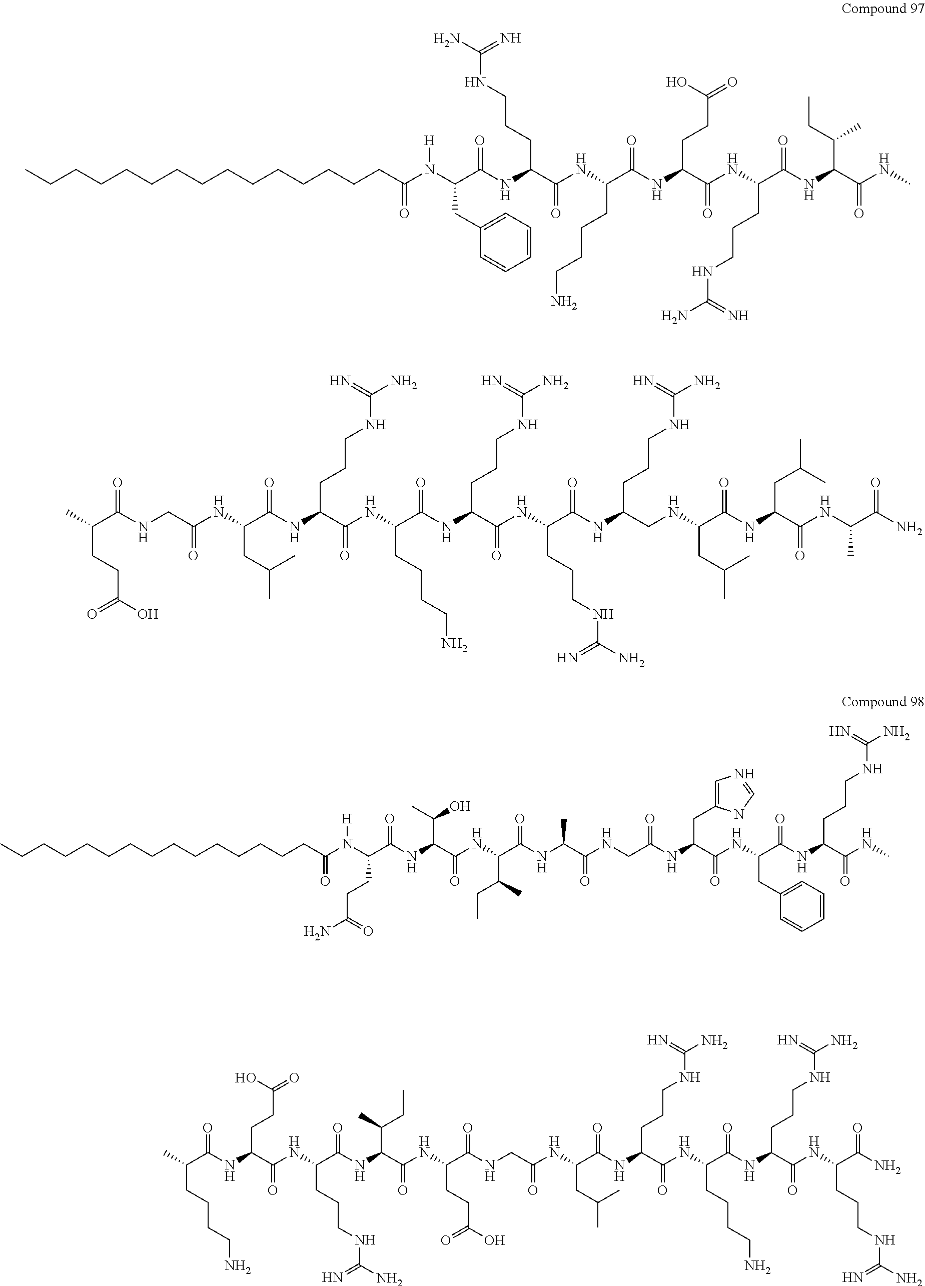

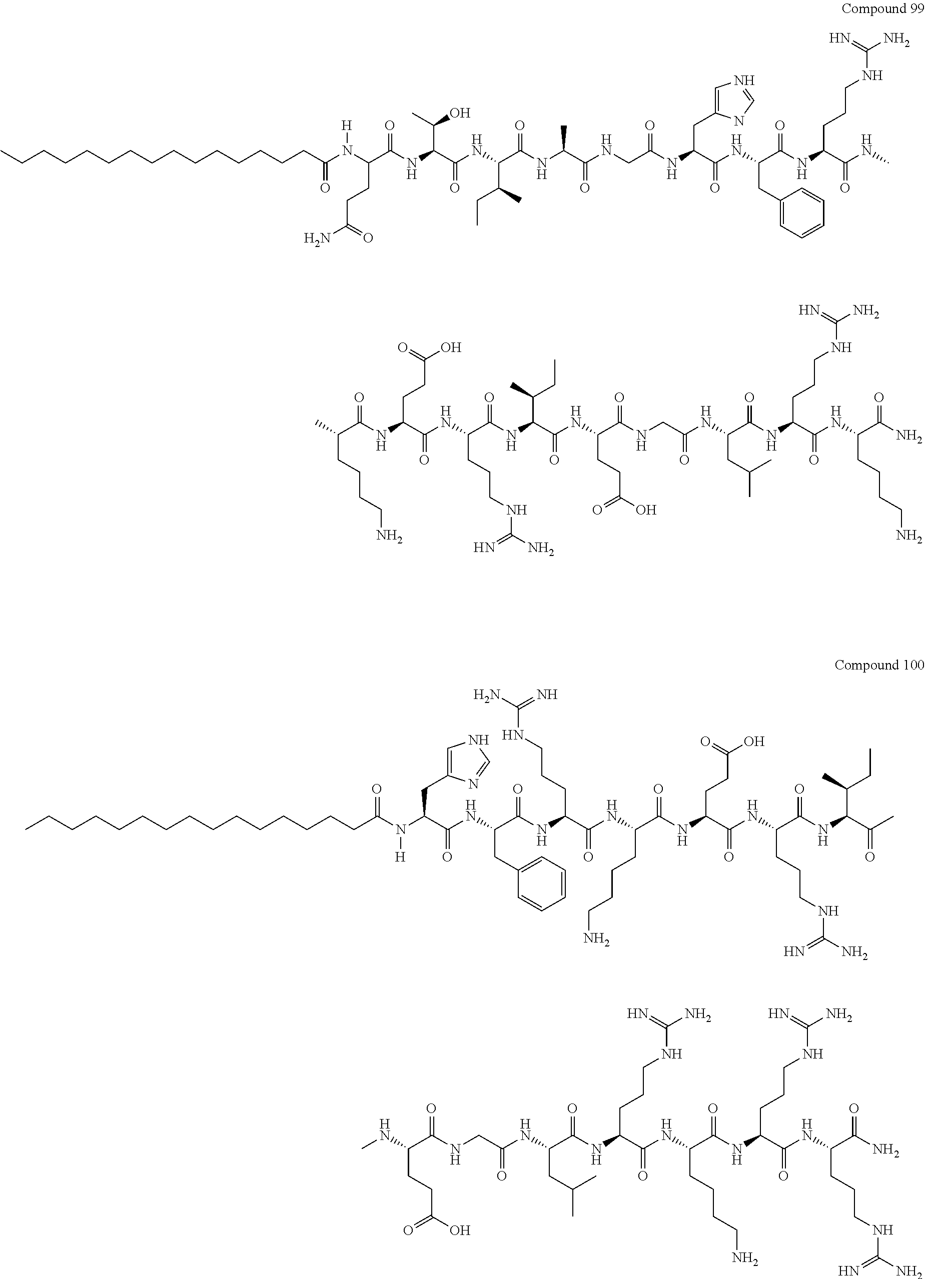

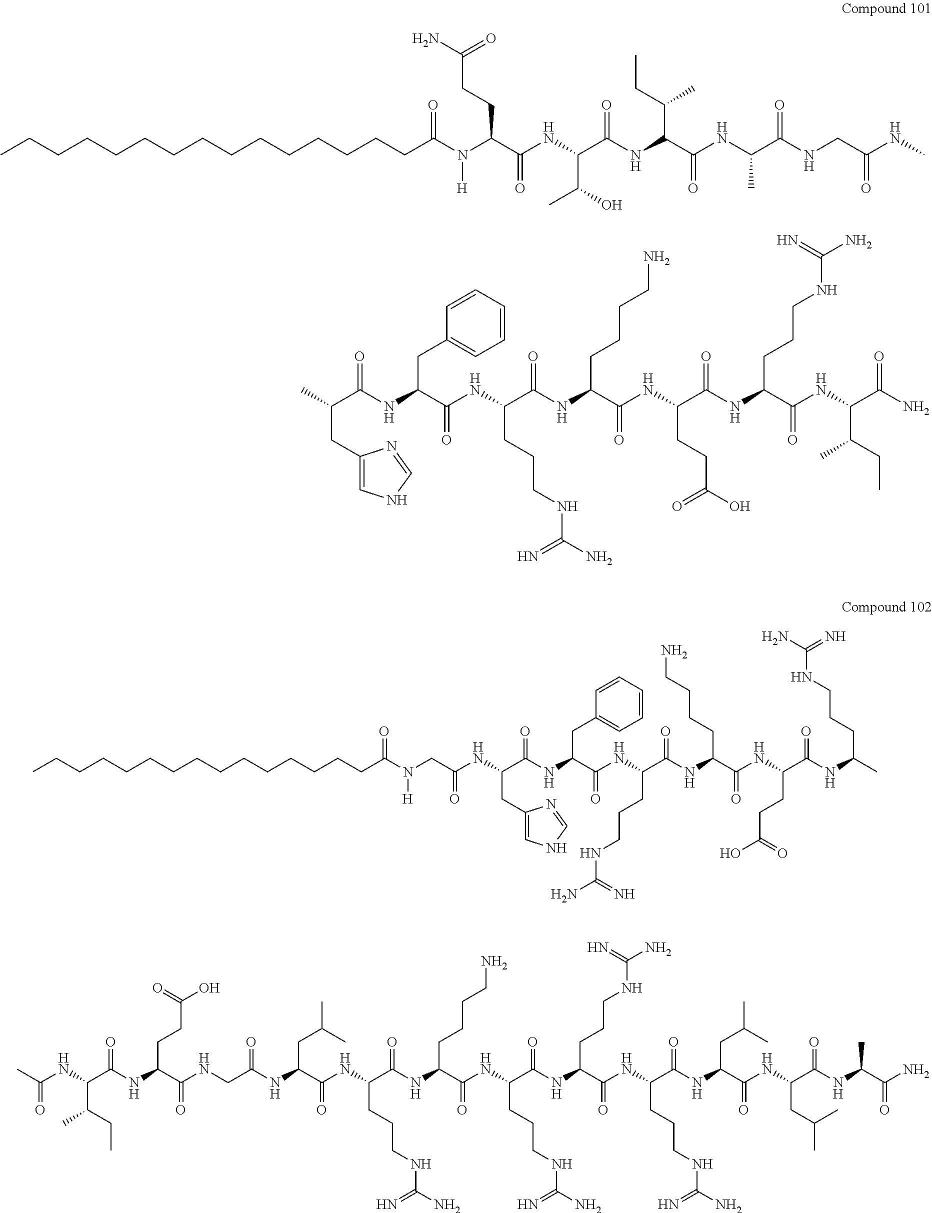

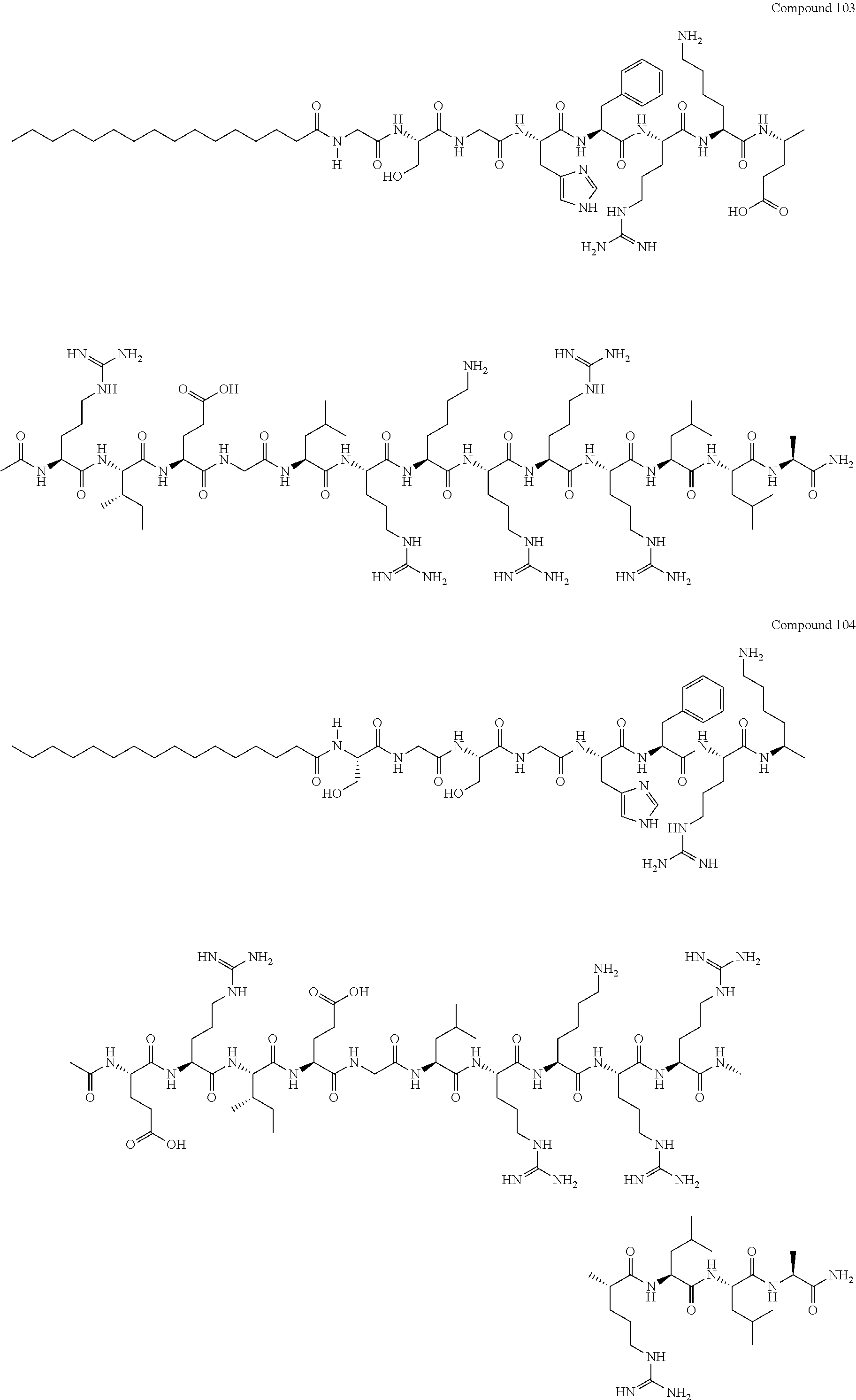

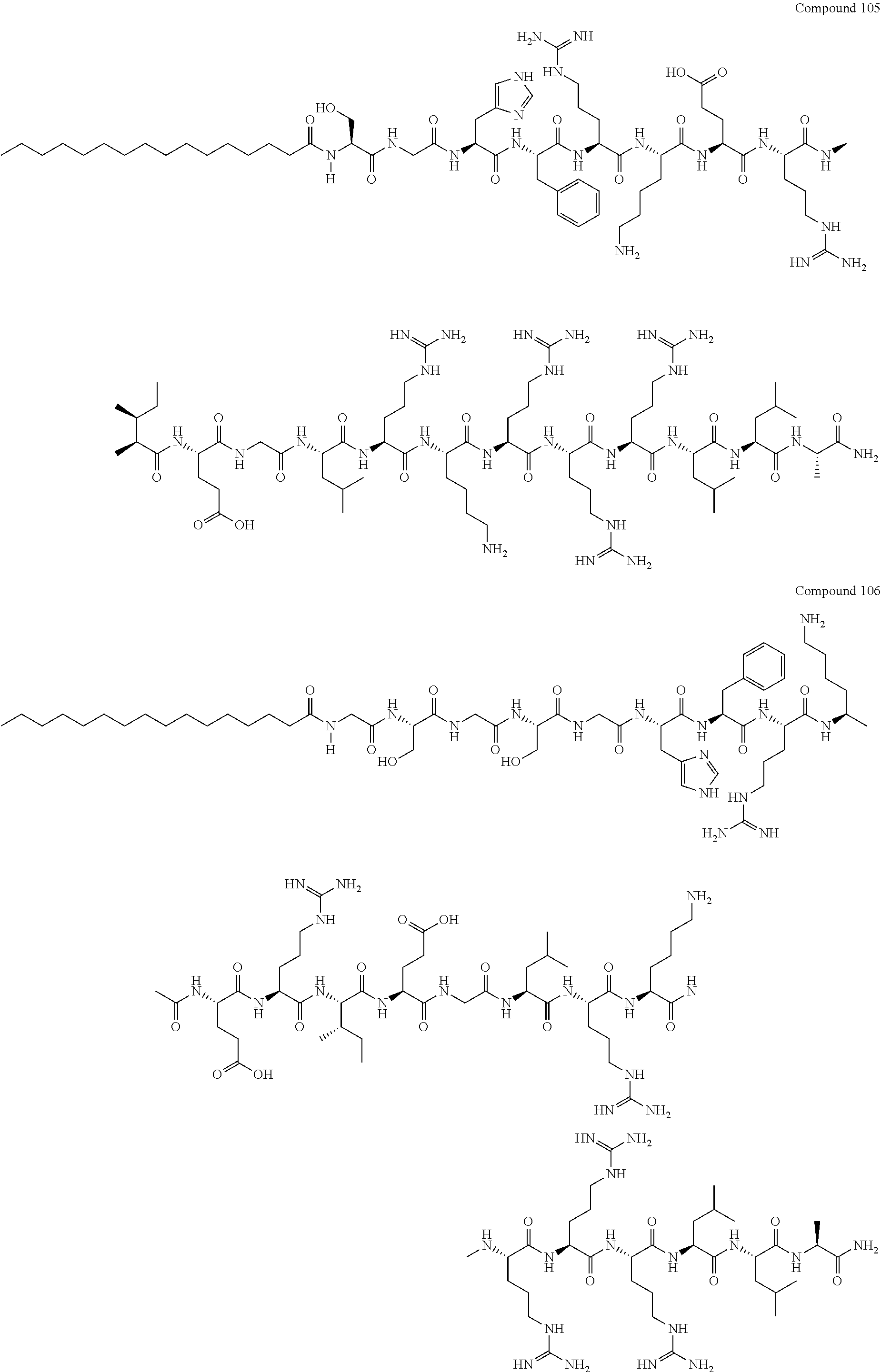

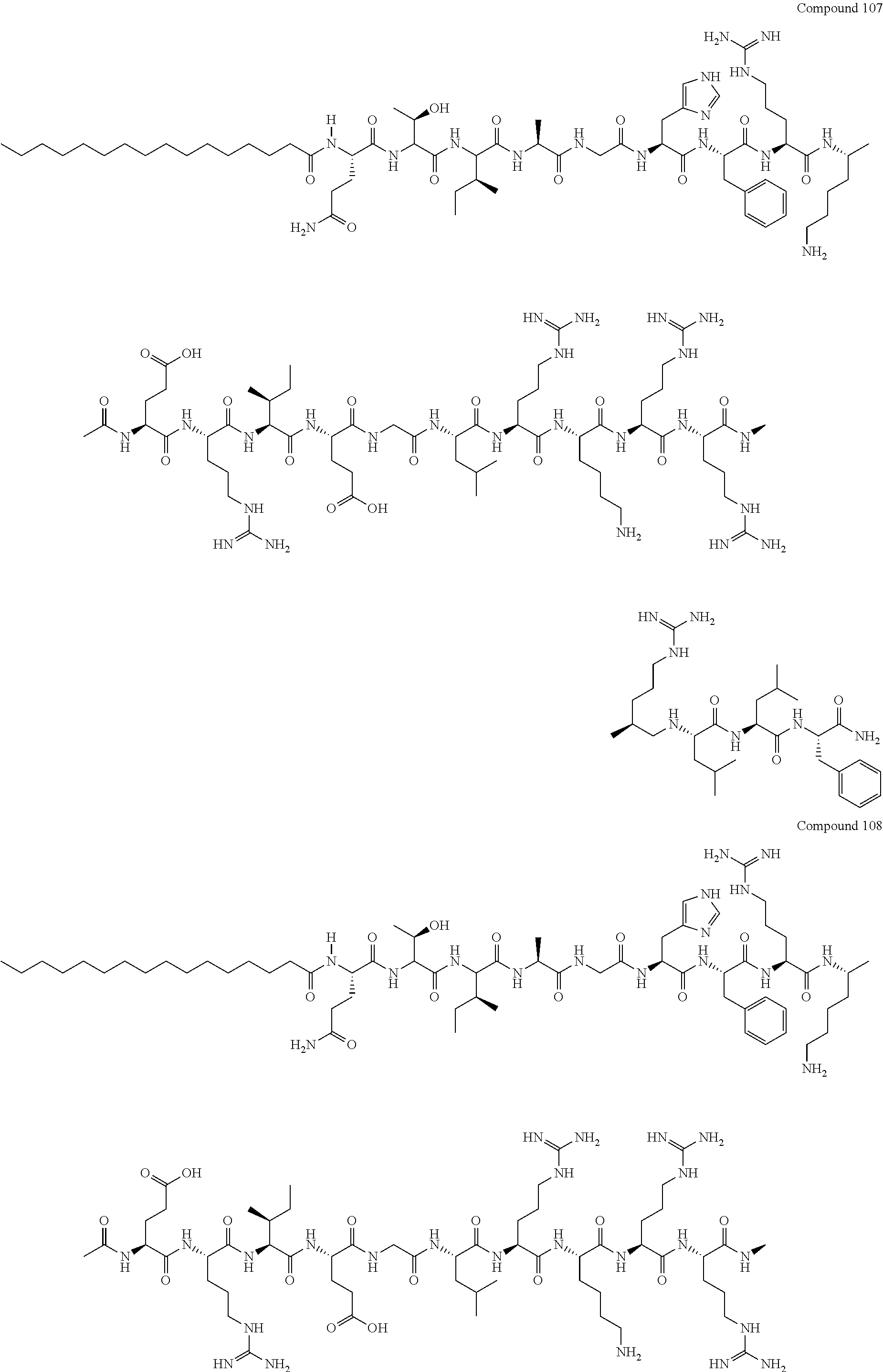

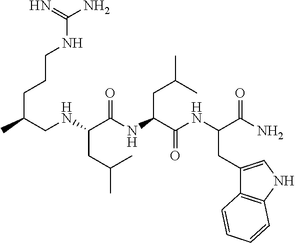

In the above listing of compounds, structures follow the compound number identifier.

In yet another embodiment, a GPCR compound of the invention is selected from one of the following compounds or a pharmaceutically acceptable salt thereof:

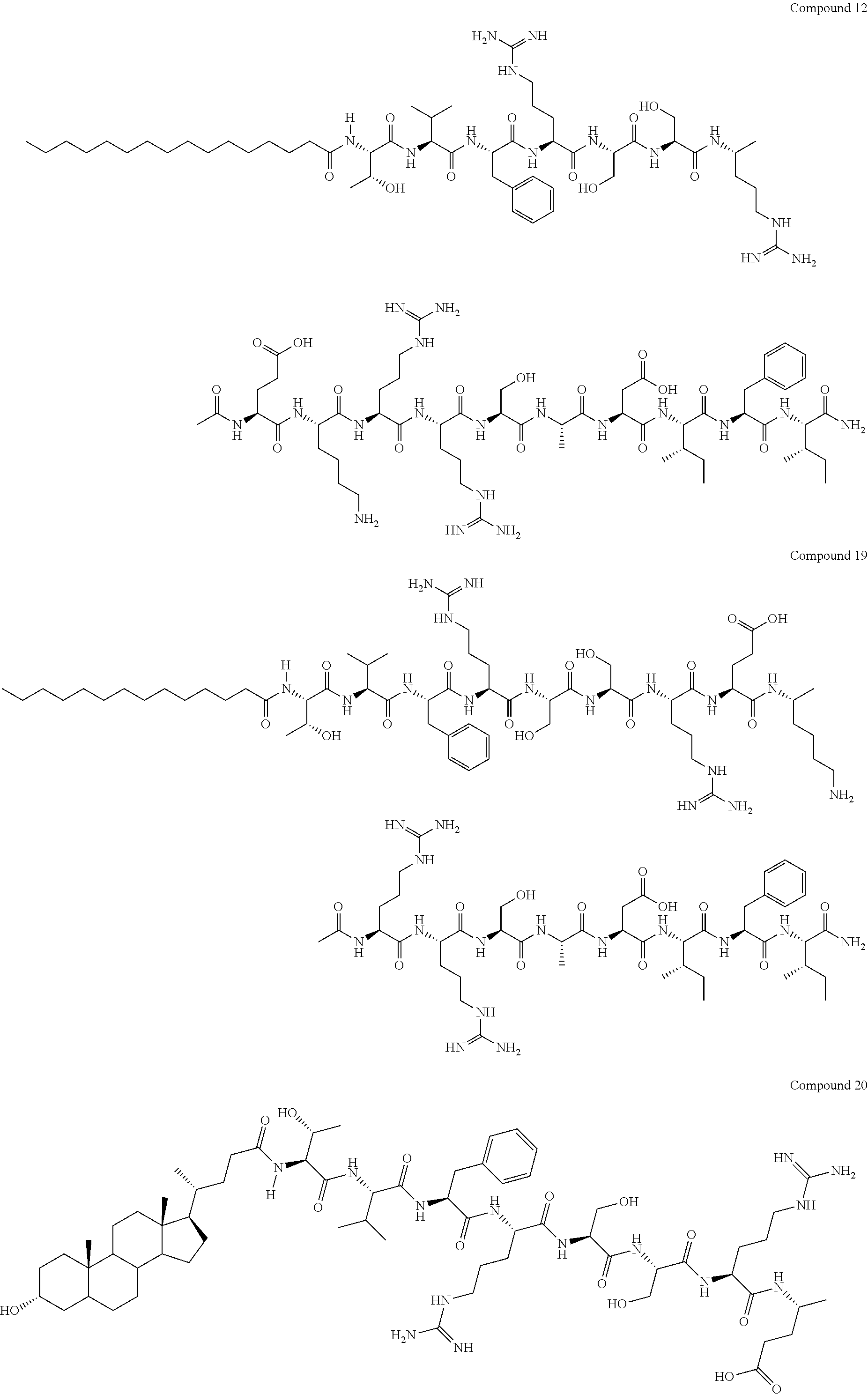

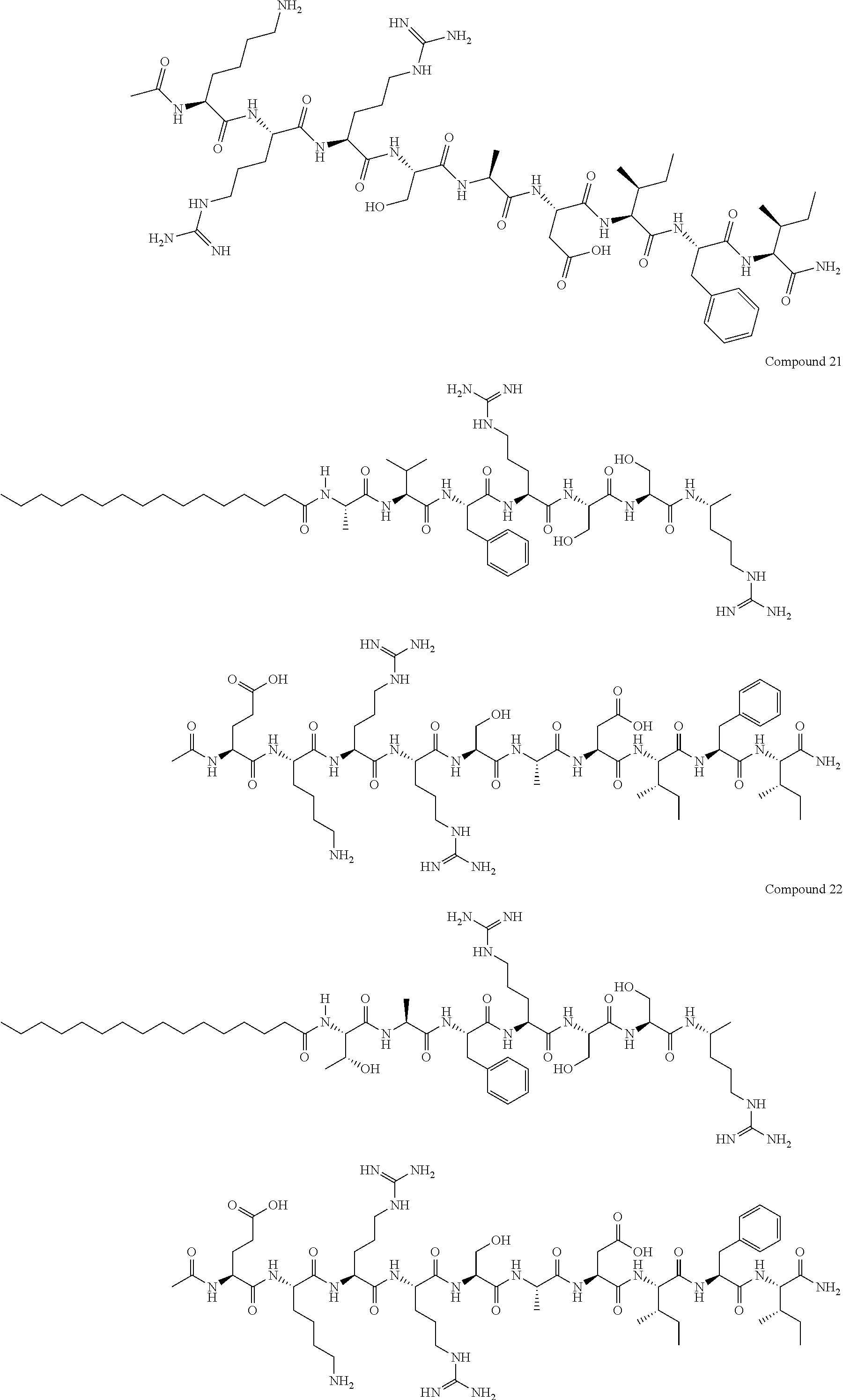

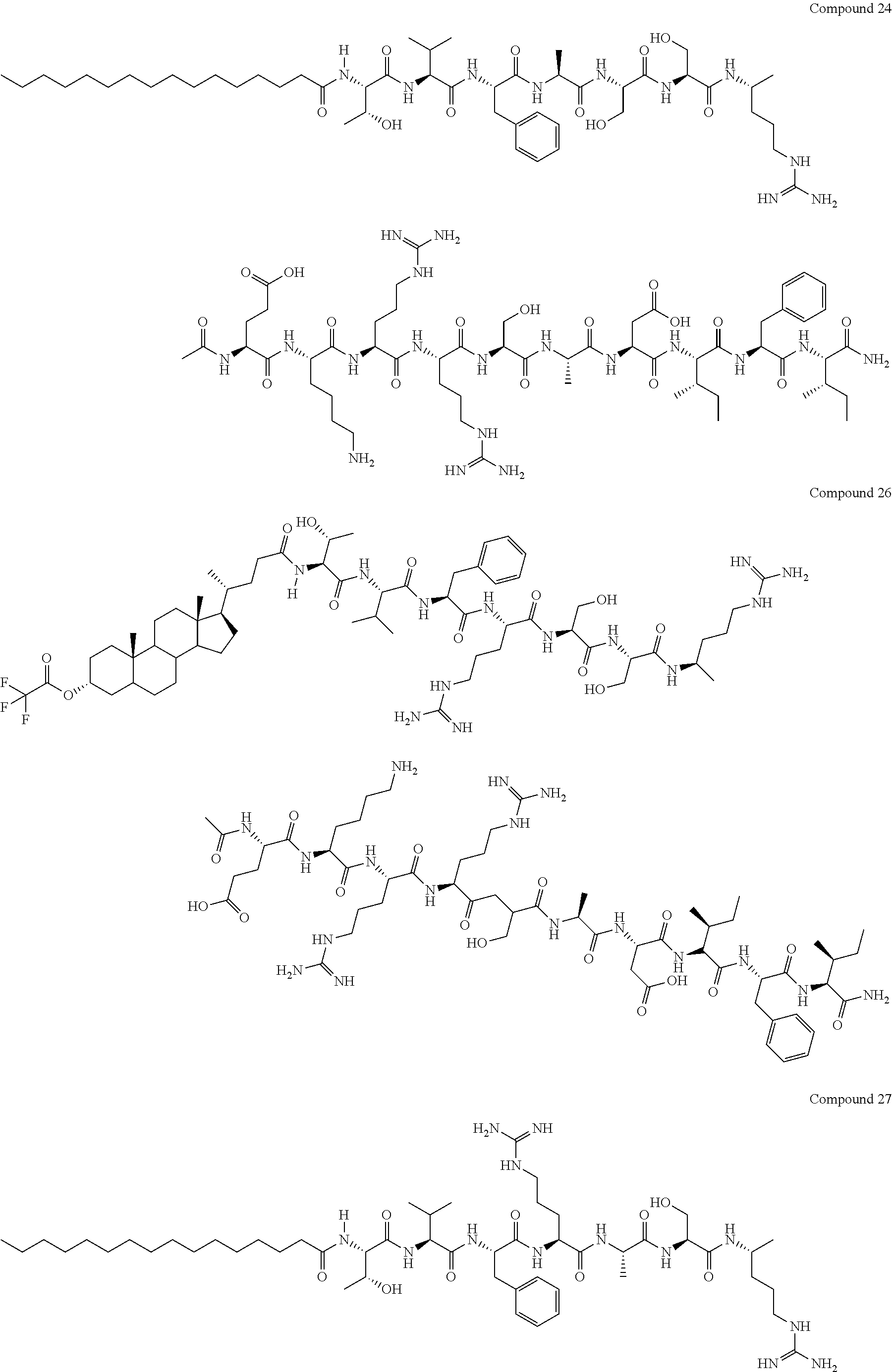

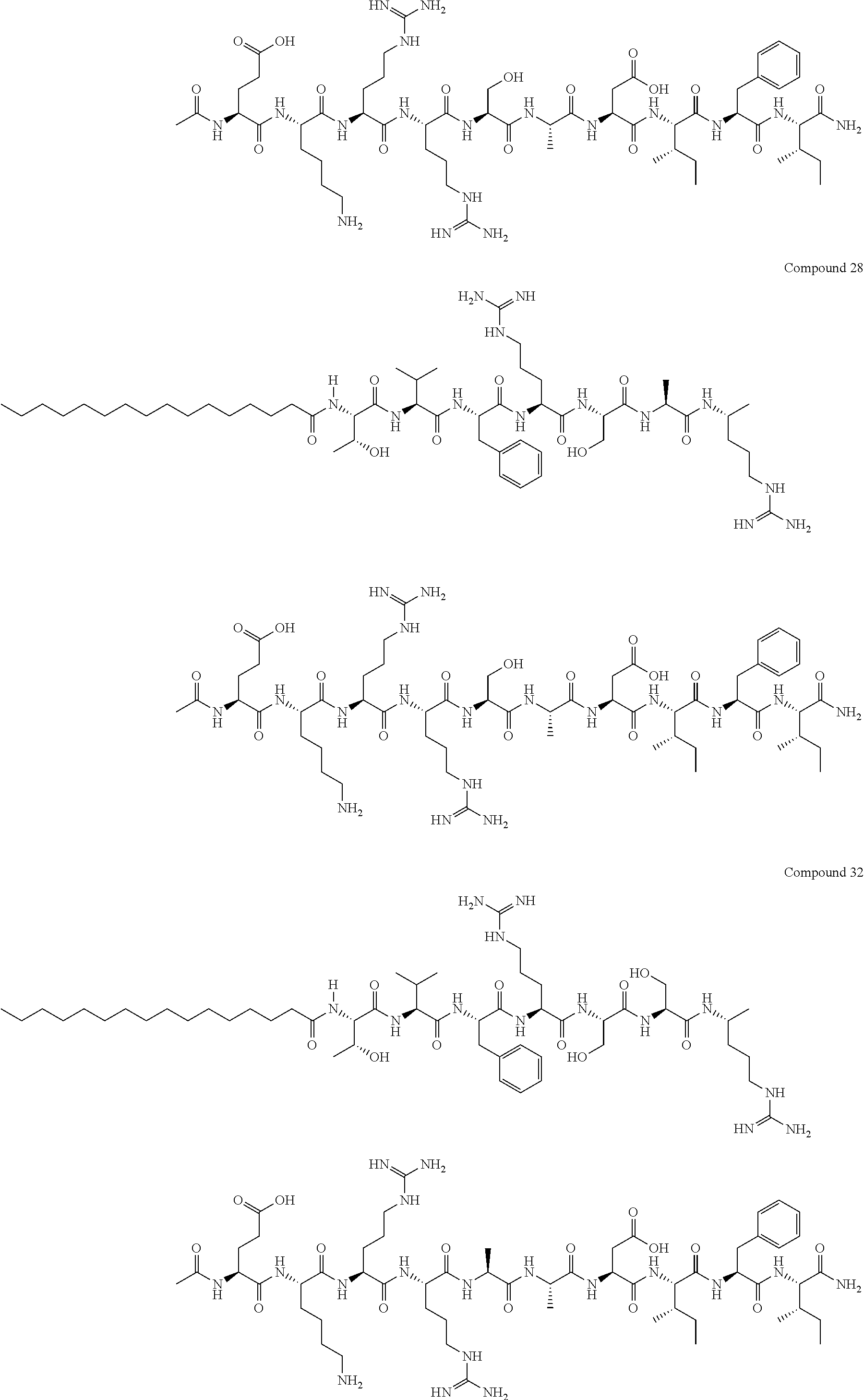

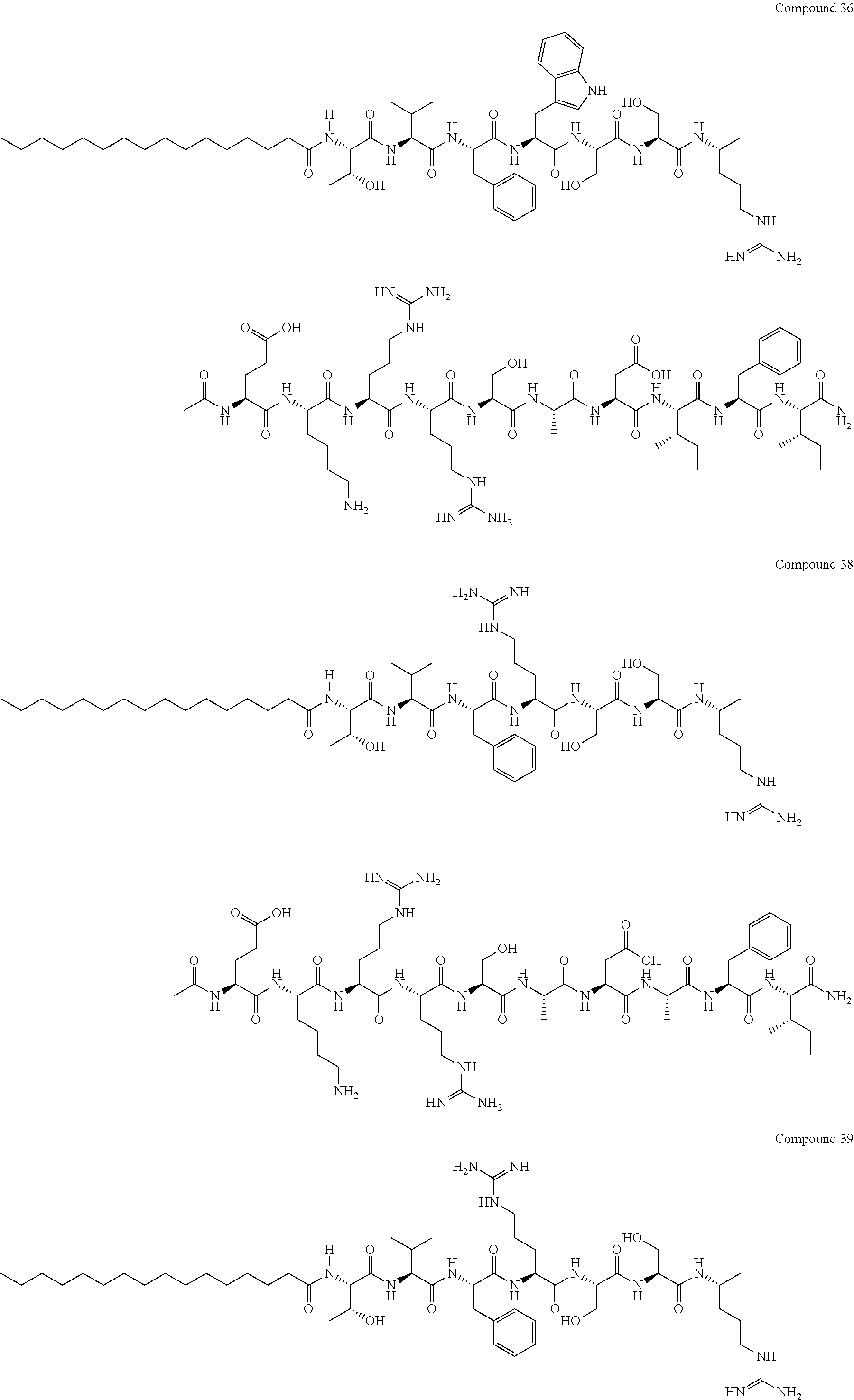

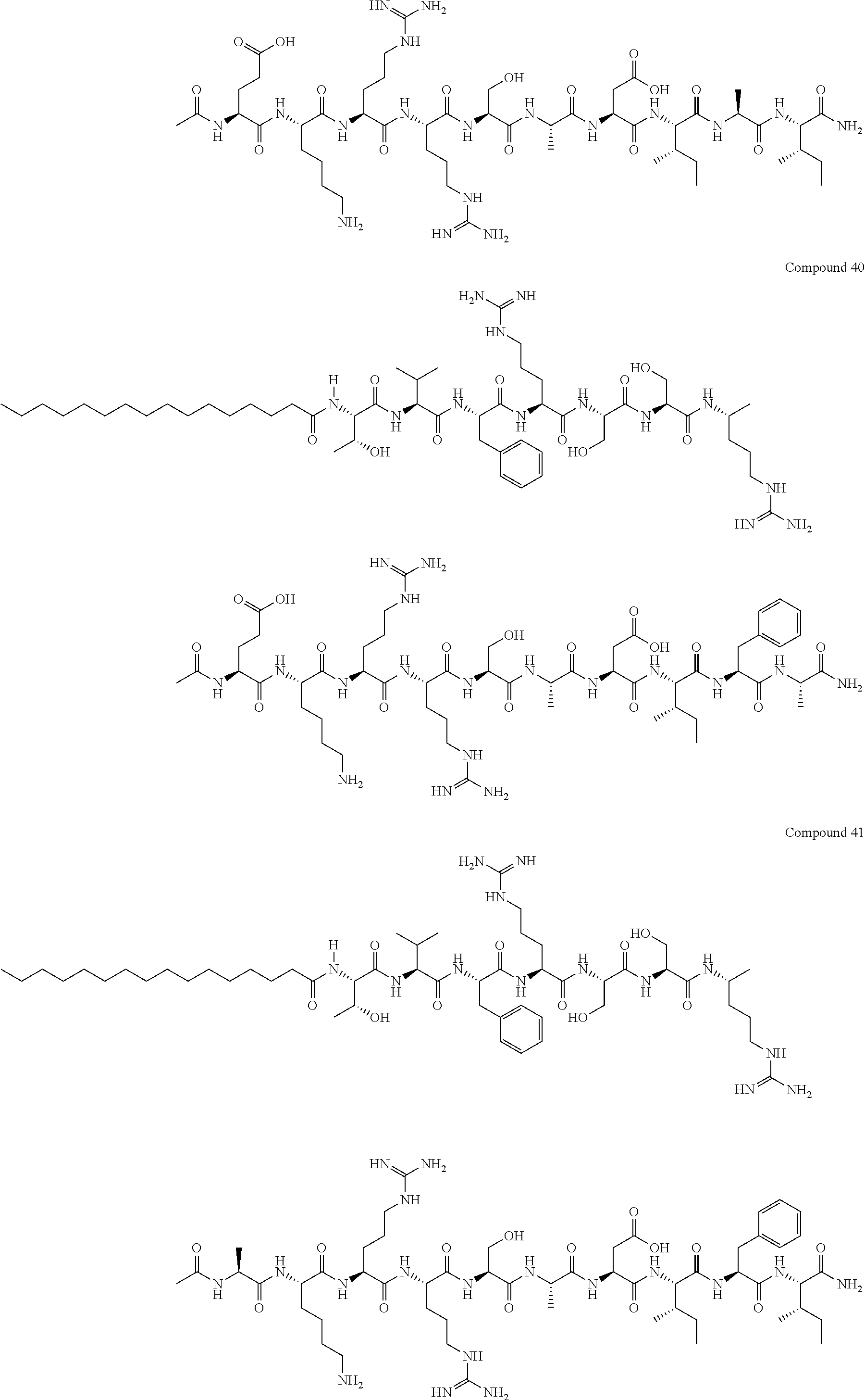

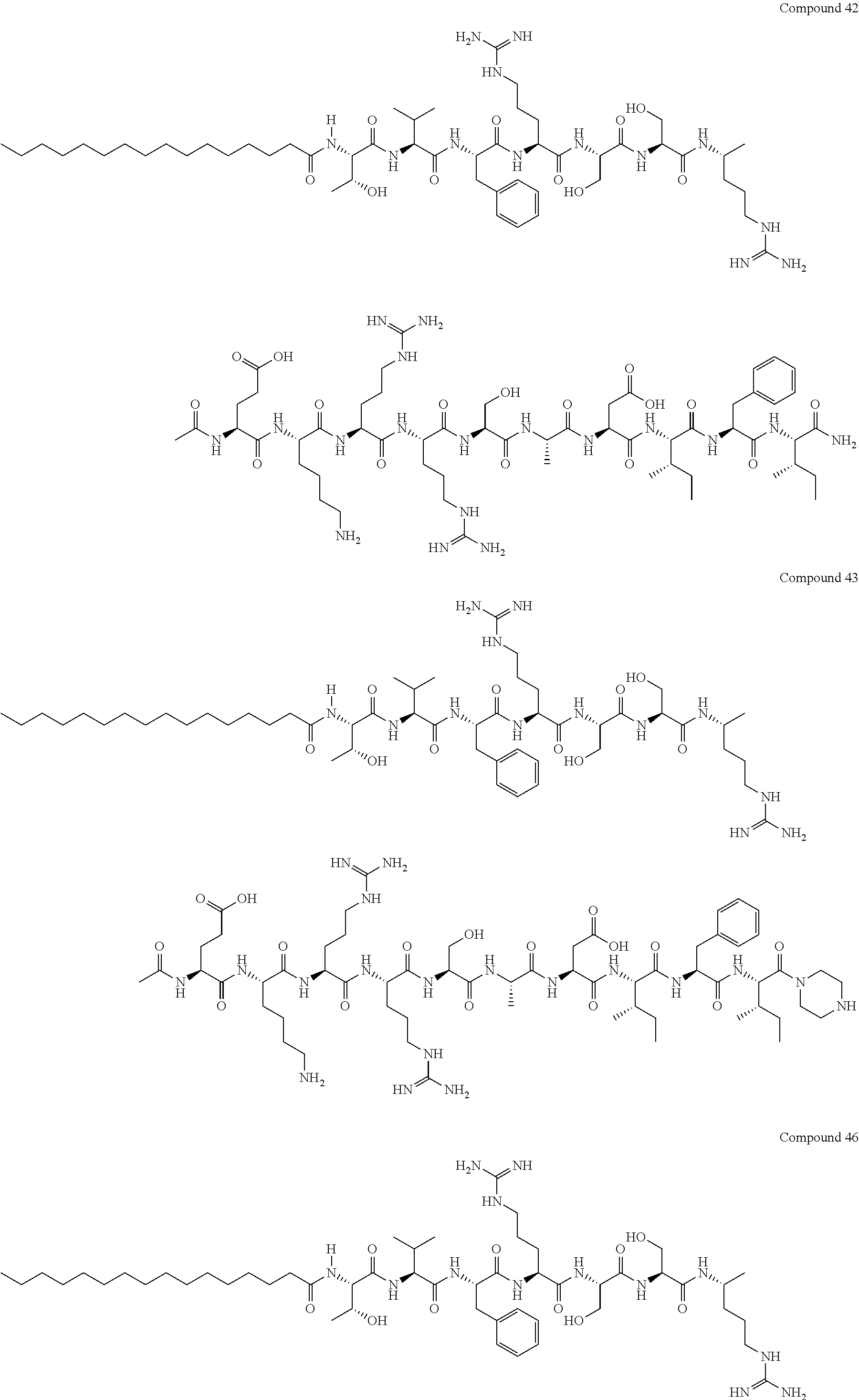

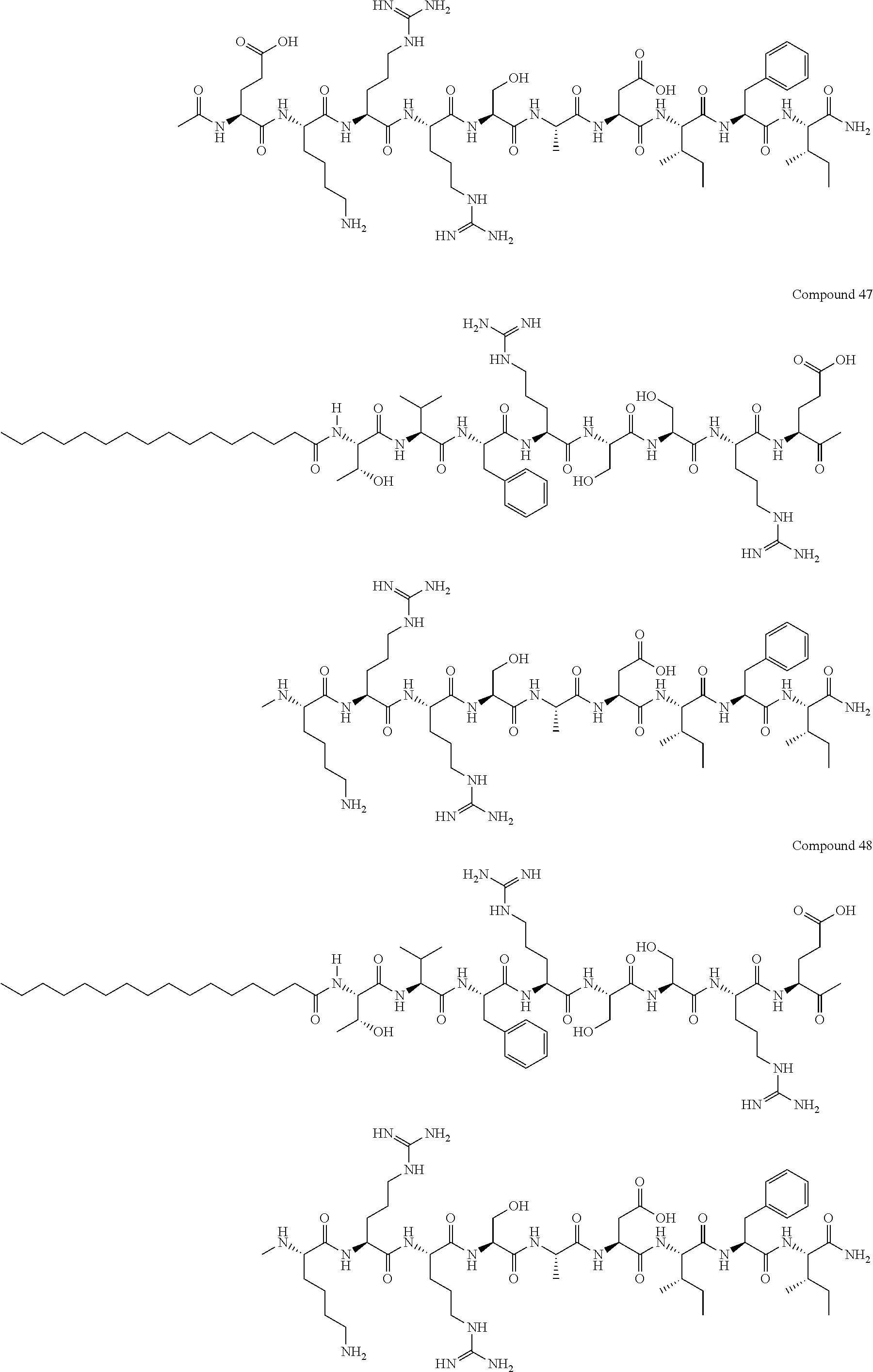

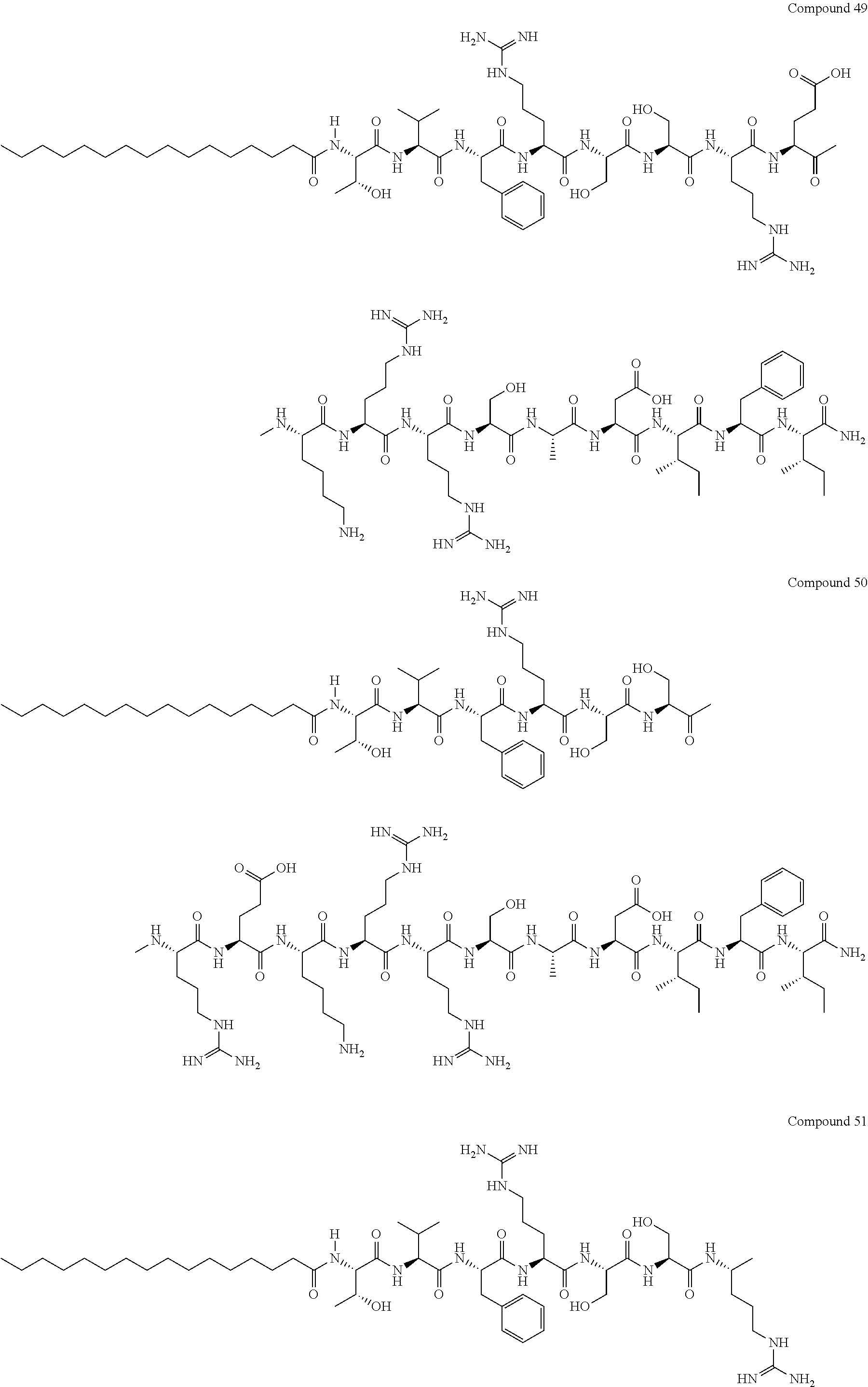

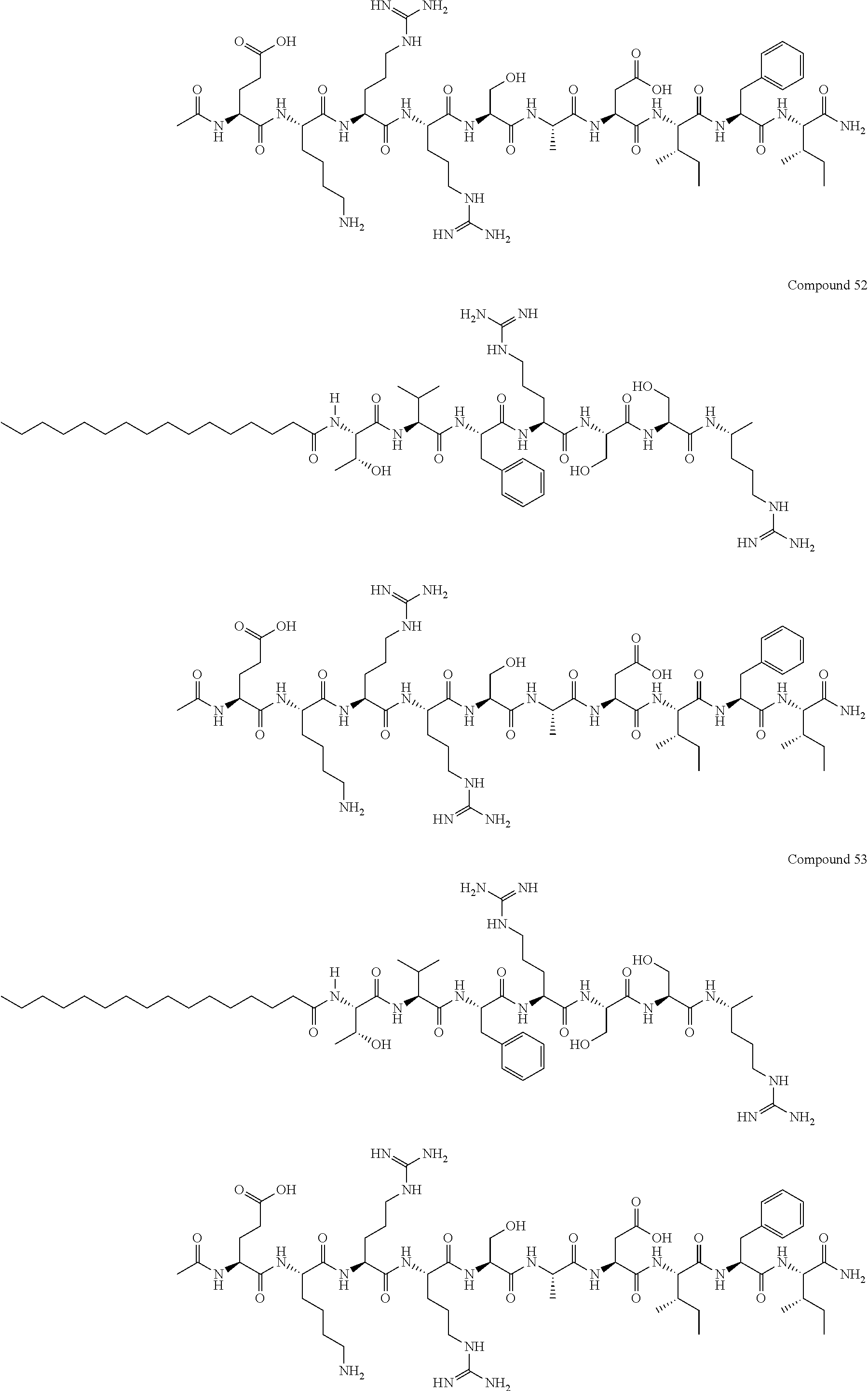

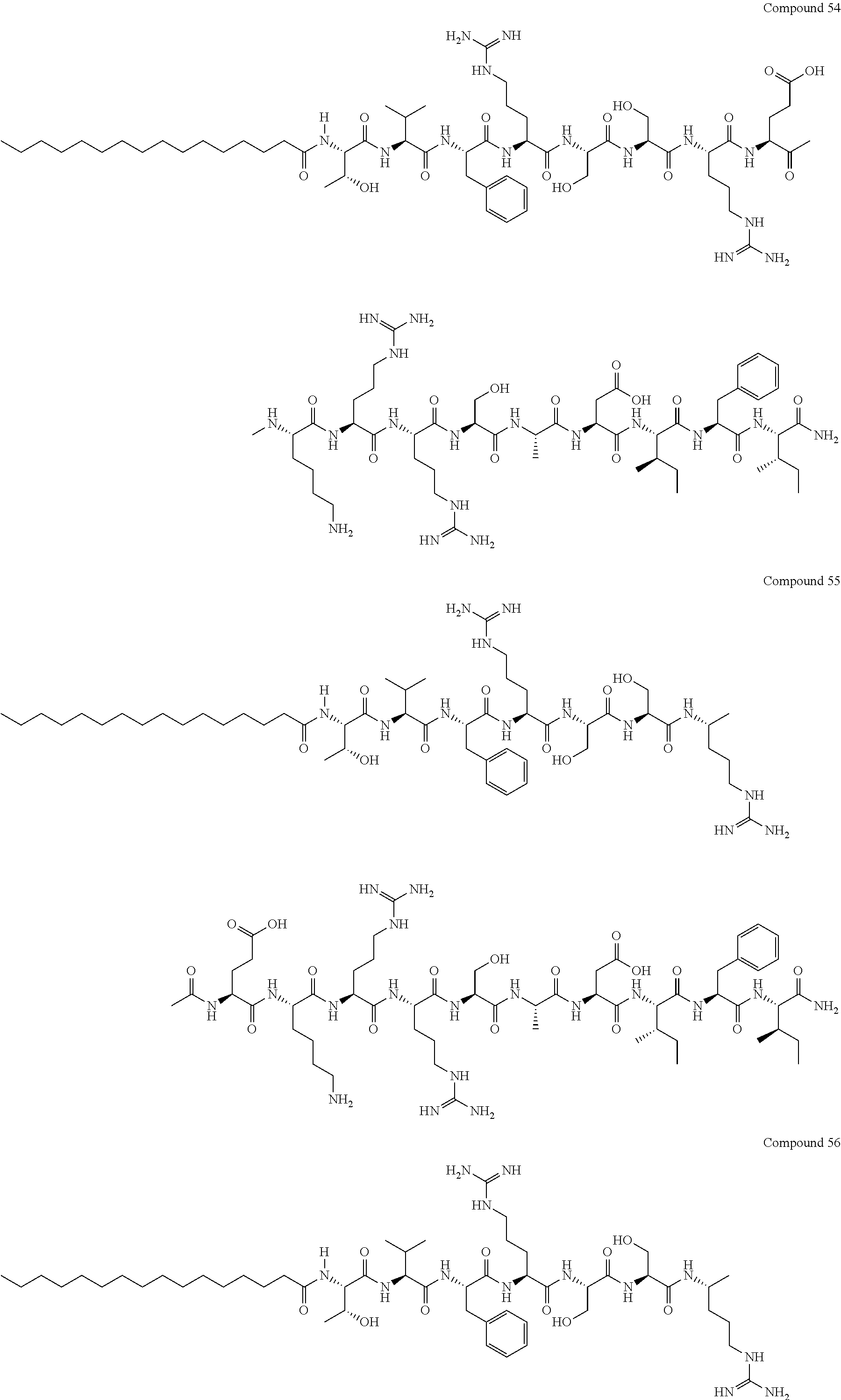

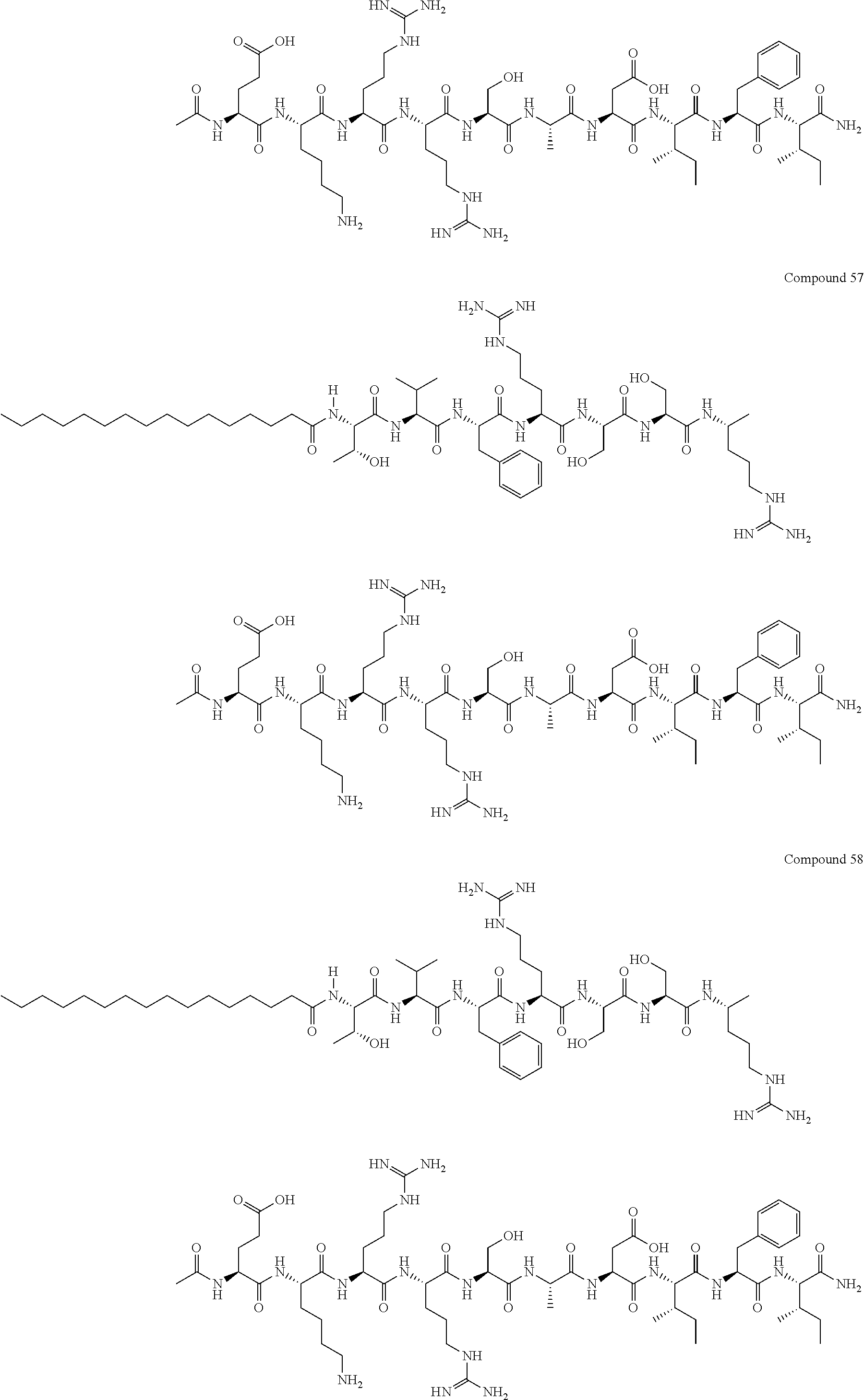

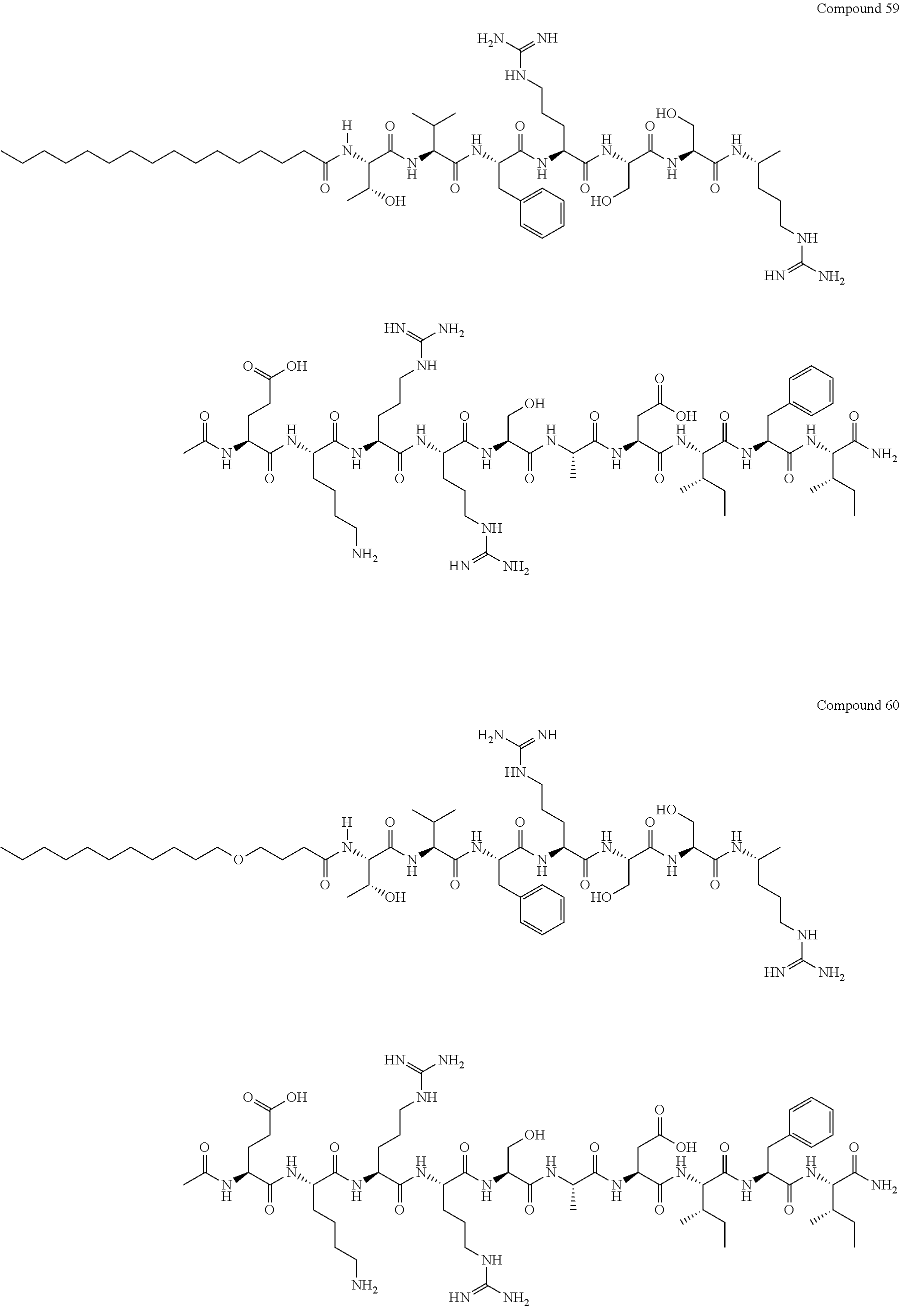

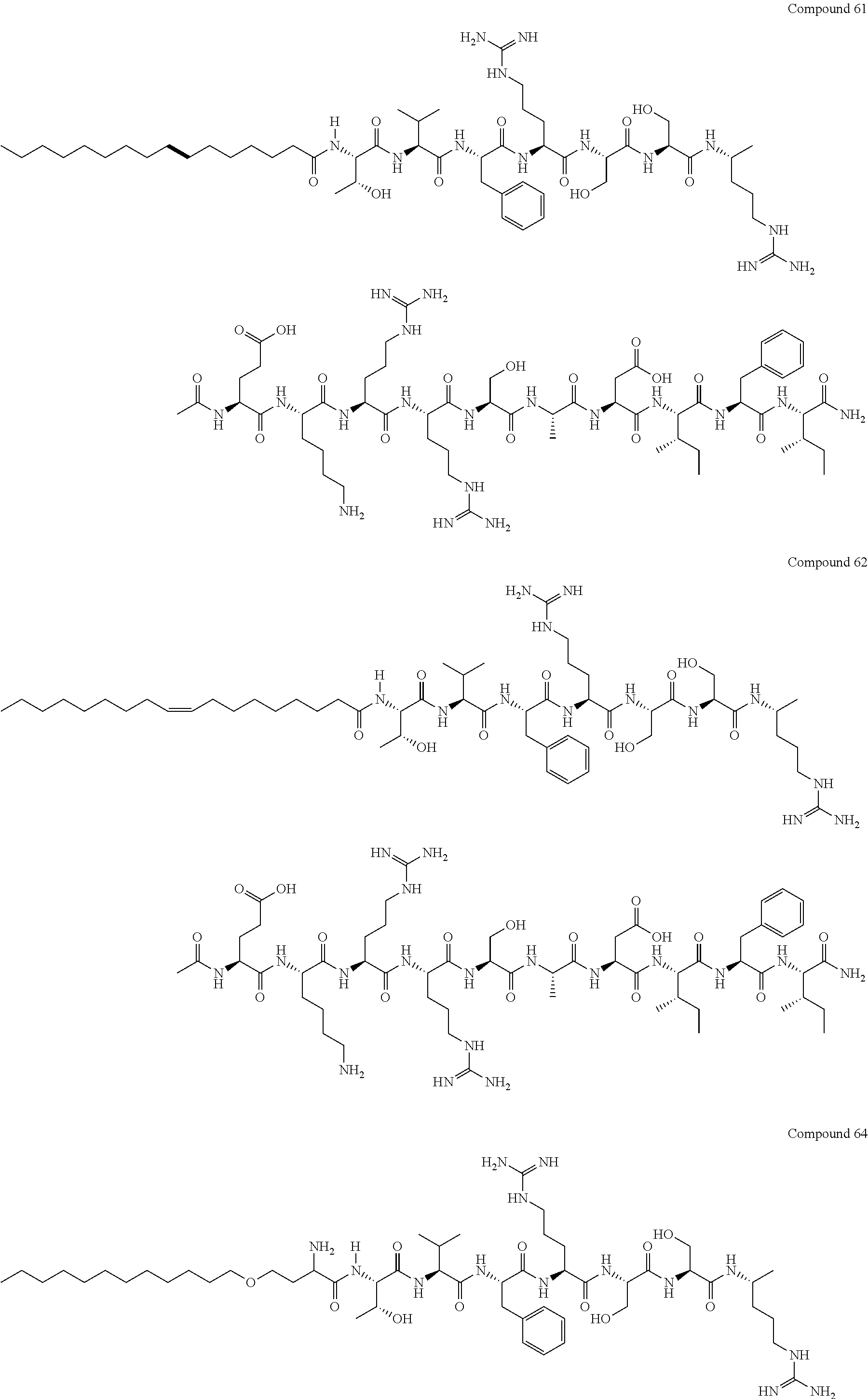

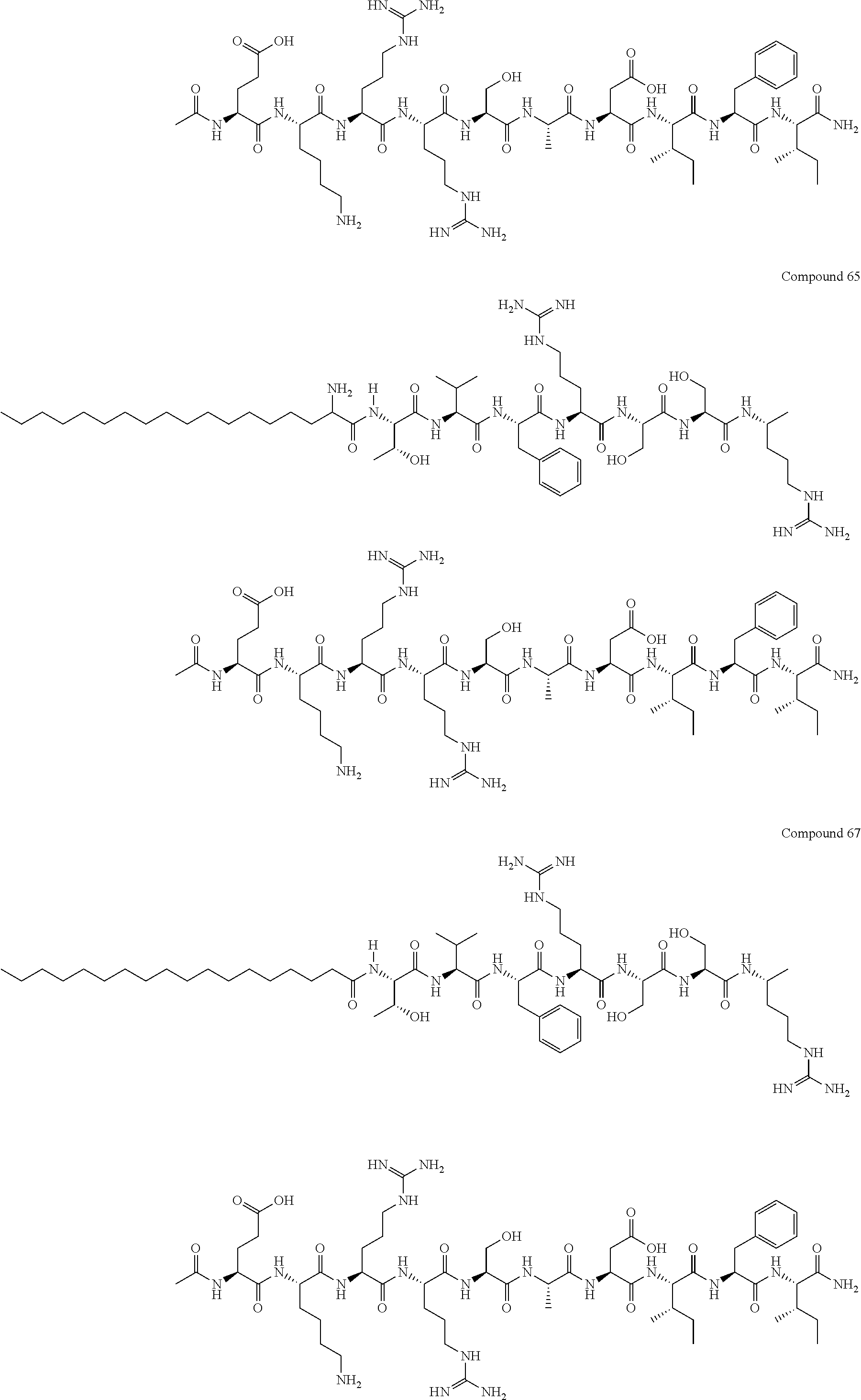

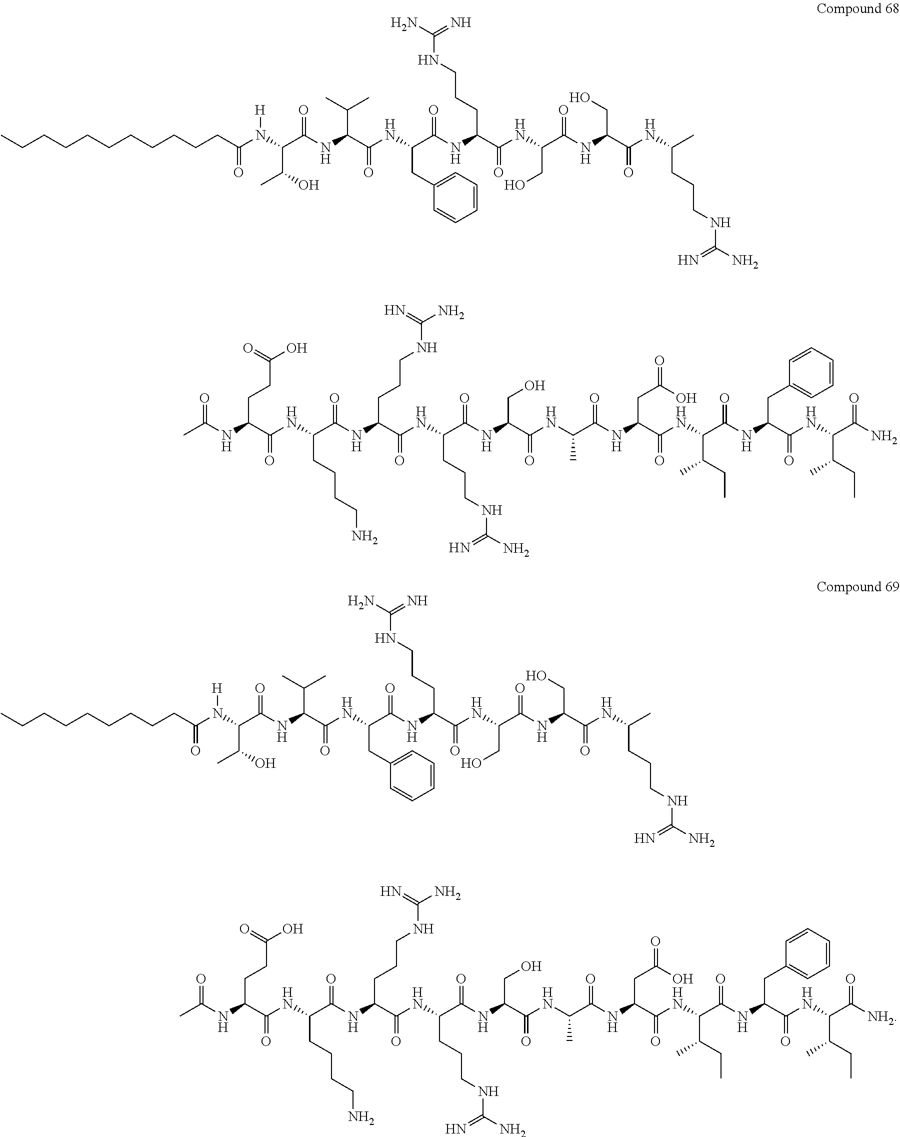

In the above listing of compounds, structures follow the compound number identifier.

In yet another embodiment, a GPCR compound of the invention is selected from one of the following compounds or a pharmaceutically acceptable salt thereof:

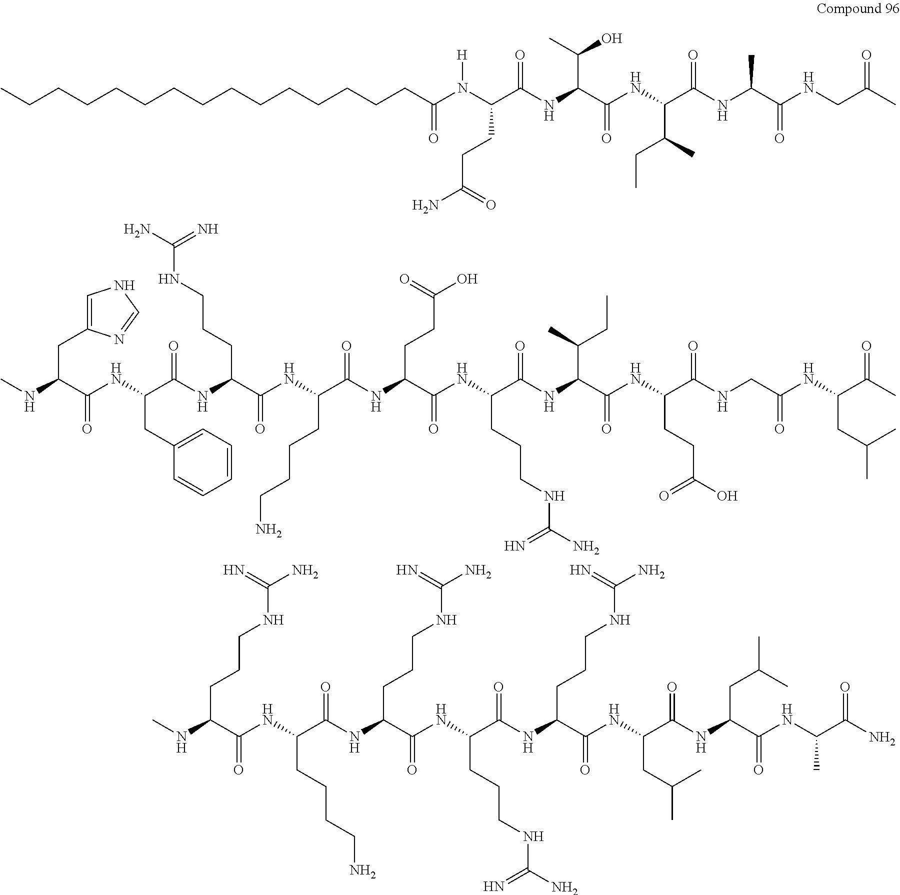

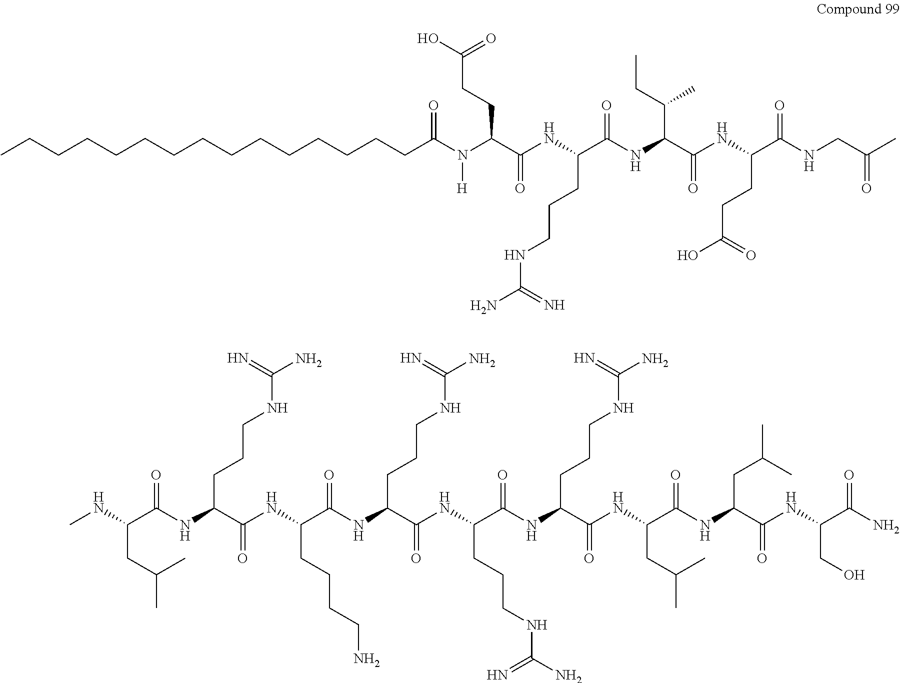

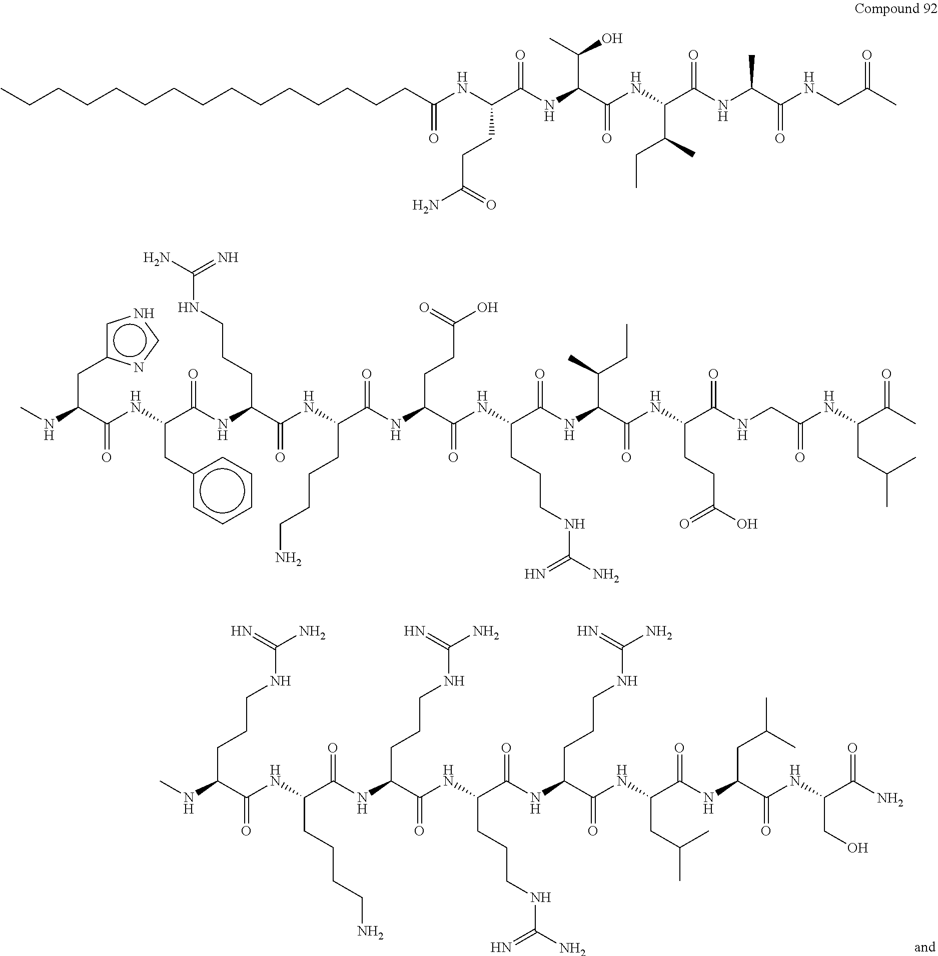

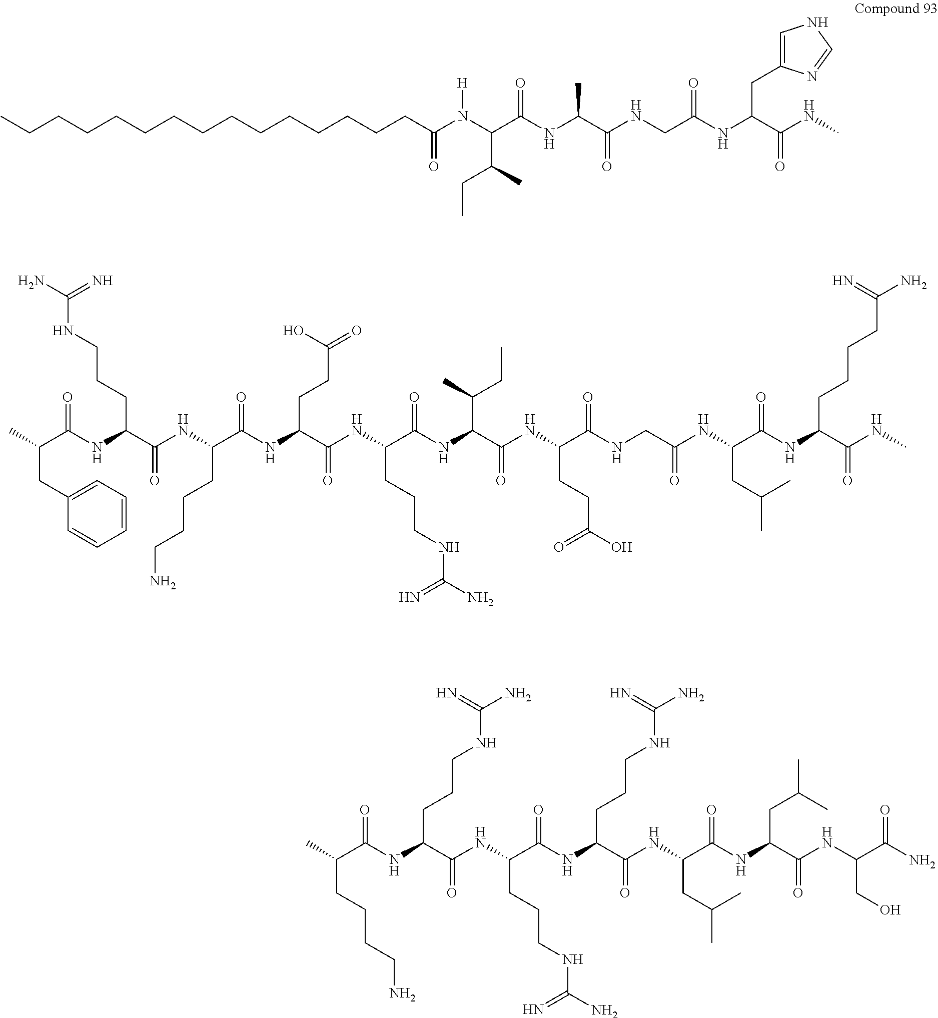

In the above listing of compounds, structures follow the compound identifiers.

“Cycloalkyl” used alone or as part of a larger moiety such as “cycloalkylalkyl” refers to a monocyclic or polycyclic, non-aromatic ring system of 3 to 20 carbon atoms, 3 to 12 carbon atoms, or 3 to 9 carbon atoms, which may be saturated or unsaturated. Examples of cycloalkyl groups include cyclopropyl, cyclobutyl, cyclopentyl, cyclohexyl, cyclohexenyl, cyclohexa-1,3-dienyl, cyclooctyl, cycloheptanyl, norbornyl, adamantyl, and the like.

“Heterocycloalkyl” refers to a saturated or unsaturated, non-aromatic, monocyclic or polycyclic ring system of 3 to 20 atoms, 3 to 12 atoms, or 3 to 8 atoms, containing one to four ring heteroatoms chosen from O, N and S. Examples of heterocyclyl groups include pyrrolidine, piperidine, tetrahydrofuran, tetrahydropyran, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine-1,1-dioxide, tetrahydro-2H-1,2-thiazine-1,1-dioxide, isothiazolidine-1,1-dioxide, pyrrolidin-2-one, piperidin-2-one, piperazin-2-one, and morpholin-2-one, and the like.

“Halogen” and “halo” refer to fluoro, chloro, bromo or iodo.

“Haloalkyl” refers to an alkyl group substituted with one or more halogen atoms. By analogy, “haloalkenyl”, “haloalkynyl”, etc., refers to the group (for example alkenyl or alkynyl) substituted by one or more halogen atomes.

“Cyano” refers to the group —CN.

“Oxo” refers to a divalent ═O group.

“Thioxo” refers to a divalent ═S group.

“Ph” refers to a phenyl group.

“Carbonyl” refers to a divalent —C(O)— group.

“Alkyl” used alone or as part of a larger moiety such as “hydroxyalkyl”, “alkoxyalkyl”, “alkylamine” refers to a straight or branched, saturated aliphatic group having the specified number of carbons, typically having 1 to 12 carbon atoms. More particularly, the aliphatic group may have 1 to 10, 1 to 8, 1 to 6, or 1 to 4 carbon atoms. This term is exemplified by groups such as methyl, ethyl, n-propyl, isopropyl, n-butyl, isobutyl, tert-butyl, n-hexyl, and the like.

“Alkenyl” refers to a straight or branched aliphatic group with at least one double bond. Typically, alkenyl groups have from 2 to 12 carbon atoms, from 2 to 8, from 2 to 6, or from 2 to 4 carbon atoms. Examples of alkenyl groups include ethenyl (—CH═CH₂), n-2-propenyl (allyl, —CH₂CH═CH₂), pentenyl, hexenyl, and the like.

“Alkynyl” refers to a straight or branched aliphatic group having at least 1 site of alkynyl unsaturation. Typically, alkynyl groups contain 2 to 12, 2 to 8, 2 to 6 or 2 to 4 carbon atoms. Examples of alkynyl groups include ethynyl (—C≡CH), propargyl (—CH₂C≡CH), pentynyl, hexynyl, and the like.

“Alkylene” refers to a bivalent saturated straight-chained hydrocarbon, e.g., C₁-C₆ alkylene includes —(CH₂)₆—, —CH₂—CH—(CH₂)₃CH₃, and the like. “Bivalent means that the alkylene group is attached to the remainder of the molecule through two different carbon atoms.

“Alkenylene” refers to an alkylene group with in which one carbon-carbon single bond is replaced with a double bond.

“Alkynylene” refers to an alkylene group with in which one carbon-carbon single bond is replaced with a triple bond.

“Aryl” used alone or as part of a larger moiety as in “aralkyl” refers to an aromatic carbocyclic group of from 6 to 14 carbon atoms having a single ring or multiple condensed rings. The term “aryl” also includes aromatic carbocycle(s) fused to cycloalkyl or heterocycloalkyl groups. Examples of aryl groups include phenyl, benzo[d][1,3]dioxole, naphthyl, phenantrenyl, and the like.

“Aryloxy” refers to an —OAr group, wherein O is an oxygen atom and Ar is an aryl group as defined above.

“Aralkyl” refers to an alkyl having at least one alkyl hydrogen atom replaced with an aryl moiety, such as benzyl, —(CH₂)₂phenyl, —(CH₂)₃phenyl, —CH(phenyl)₂, and the like.

“Alkyl cycloalkyl” refers to an alkyl having at least one alkyl hydrogen atom replaced with a cycloalkyl moiety, such as —CH₂-cyclohexyl, —CH₂-cyclohexenyl, and the like.

“Heteroaryl” used alone or a part of a larger moiety as in “heteroaralkyl” refers to a 5 to 14 membered monocyclic, bicyclic or tricyclic heteroaromatic ring system, containing one to four ring heteroatoms independently selected from nitrogen, oxygen and sulfur. The term “heteroaryl” also includes heteroaromatic ring(s) fused to cycloalkyl or heterocycloalkyl groups. Particular examples of heteroaryl groups include optionally substituted pyridyl, pyrrolyl, pyrimidinyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 1,2,3,4-tetrahydroquinolyl, 1,2,3,4-tetrahydroisoquinolyl, purinyl, pteridinyl, carbazolyl, xanthenyl, benzoquinolyl, and the like.

“Heteroaryloxy” refers to an —OHet group, wherein O is an oxygen atom and Het is a heteroaryl group as defined above.

“Heteroaralkyl” refers to an alkyl having at least one alkyl hydrogen atom replaced with a heteroaryl moiety, such as —CH₂-pyridinyl, —CH₂-pyrimidinyl, and the like.

“Alkoxy” refers to the group —O—R where R is “alkyl”, “cycloalkyl”, “alkenyl”, or “alkynyl”. Examples of alkoxy groups include for example, methoxy, ethoxy, ethenoxy, and the like.

“Alkyl heterocycloalkyl” refers to an alkyl having at least one alkyl hydrogen atom replaced with a heterocycloalkyl moiety, such as —CH₂-morpholino, —CH₂-piperidyl and the like.

“Alkoxycarbonyl” refers to the group —C(O)OR where R is “alkyl”, “alkenyl”, “alkynyl”, “cycloalkyl”, “heterocycloalkyl”, “aryl”, or “heteroaryl”.

“Hydroxyalkyl” and “alkoxyalkyl” are alky groups substituted with hydroxyl and alkoxy, respectively.

“Amino” means —NH₂; “alkylamine” and “dialkylamine” mean —NHR and —NR₂, respectively, wherein R is an alkyl group. “Cycloalkylamine” and “dicycloalkylamine” mean —NHR and —NR₂, respectively, wherein R is a cycloalkyl group. “Cycloalkylalkylamine” means —NHR wherein R is a cycloalkylalkyl group. “[Cycloalkylalkyl][alkyl]amine” means —N(R)₂ wherein one R is cycloalkylalkyl and the other R is alkyl.

Haloalkyl and halocycloalkyl include mono, poly, and perhaloalkyl groups where the halogens are independently selected from fluorine, chlorine, bromine and iodine.

Suitable substituents for “alkyl”, “alkenyl”, “alkynyl”, “cycloalkyl”, “heterocycloalkyl”, “aryl”, or “heteroaryl”, etc., are those which will form a stable compound of the invention. Examples of suitable substituents are those selected from the group consisting of halogen, —CN, —OH, —NH₂, (C₁-C₄)alkyl, (C₁-C₄)haloalkyl, aryl, heteroaryl, (C₃-C₇)cycloalkyl, (5-7 membered) heterocycloalkyl, —NH(C₁-C₆)alkyl, —N((C₁-C₆)alkyl)₂, (C₁-C₆)alkoxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)N((C₁-C₆)alkyl)₂, —N(C₁-C₆)alkylC(S)N((C₁-C₆)alkyl)₂, —CONH(C₁-C₆)alkyl, —OCONH(C₁-C₆)alkyl-CON((C₁-C₆)alkyl)₂, —C(S)(C₁-C₆)alkyl, —S(O)ₚ(C₁-C₆)alkyl, —S(O)ₚNH₂, —S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H. More particularly, the substituents are selected from halogen, —CN, —OH, —NH₂, (C₁-C₄)alkyl, (C₁-C₄)haloalkyl, (C₁-C₄)alkoxy, phenyl, and (C₃-C₇)cycloalkyl. Within the framework of this invention, said “substitution” is also meant to encompass situations where a hydrogen atom is replaced with a deuterium atom. p is an integer with a value of 1 or 2.

Pharmaceutically acceptable salts of the compounds disclosed herein are included in the present invention. For example, an acid salt of a compound containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

Salts of the compounds containing an acidic functional group can be prepared by reacting with a suitable base. Such a pharmaceutically acceptable salt can be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

### Pharmaceutical Compositions

The invention also provides pharmaceutical compositions comprising an effective amount of a compound Formula I (e.g., including any of the formulae herein), or a pharmaceutically acceptable salt of said compound; and a pharmaceutically acceptable carrier. The carrier(s) are “pharmaceuticallyacceptable” in that they are not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), pulmonary, vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the patient therapeutics may be local, so as to be administered at the site of interest. Various techniques can be used for providing the patient compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. In one embodiment, the second therapeutic agent is one or more additional compounds of the invention.

In another embodiment, the second therapeutic agent may be selected from any compound or therapeutic agent known to have or that demonstrates advantageous properties when administered with a compound having the same mechanism of action as the APJ receptor compound of Formula I.

In a particular embodiment, the second therapeutic is an agent useful in the treatment or prevention of a disease or condition selected from, heart diseases (e.g., hypertension and heart failure, such as congestive heart failure), cancer, diabetes, stem cell trafficking, fluid homeostasis, cell proliferation, immune function, obesity, metastatic disease, and HIV infection. In another embodiment, the second therapeutic is an agent useful in the treatment or prevention of a disease or condition selected from hypertension and heart failure, in particular, congestive heart failure.

For example, when the disease or condition is congestive heart failure, the second therapeutic agent can be selected from: ACE inhibitors, beta blockers, vasodilator, calcium channel blockers, loop diuretics, aldosterone antagonists, and angiotensin receptor blockers.

When the disease or condition being treated is hypertension, the second therapeutic agent can be selected from: α-blockers, β-blockers, calcium channel blockers, diuretics, natriuretics, saluretics, centrally acting antihypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts.

A diuretic is, for example, a thiazide derivative selected from amiloride, chlorothiazide, hydrochlorothiazide, methylchlorothiazide, and chlorothalidon.

Centrally acting antihypertensives include clonidine, guanabenz, guanfacine and methyldopa.

ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxentan, and tezosentan and their pharmaceutically acceptable salts.

In one embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy. Preferably, the compound is present in the composition in an amount of from 0.1 to 50 wt. %, more preferably from 1 to 30 wt. %, most preferably from 5 to 20 wt. %.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily treatment dose or one of multiple daily treatment doses (e.g., about 1 to 4 or more times per day). When multiple daily treatment doses are used, the unit dosage form can be the same or different for each dose.

### Methods of Treatment

As used herein the term “subject” and “patient” typically means a human, but can also be an animal in need of treatment, e.g., companion animals (dogs, cats, and the like), farm animals (cows, pigs, horses, sheep, goats, and the like) and laboratory animals (rats, mice, guinea pigs, and the like).

The terms “treat” and “treating” are used interchangeably and include both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The invention also includes methods of treating diseases, disorders or pathological conditions which benefit from modulation of the APJ receptor comprising administering an effective amount of an APJ receptor compound of the invention to a subject in need thereof. Diseases and conditions which can benefit from modulation (inhibition or activation) of the APJ receptor include, but are not limited to, heart diseases (e.g., hypertension and heart failure, such as congestive heart failure), cancer, diabetes, stem cell trafficking, fluid homeostasis, cell proliferation, immune function, obesity, metastatic disease, and HIV infection.

In one embodiment, APJ receptor compounds of the invention are useful as inotropic agents for use in patients with heart failure.

In another embodiment, the APJ receptor compounds of the invention can be administered for treatment of the hypertension.

In another embodiment, the APJ receptor compounds of the invention can be administered for treatment of HIV infection.

In an additional aspect, the APJ receptor compounds of the invention can be administered for treatment of tumor metastases.

In one embodiment, an effective amount of a compound of this invention can range from about 0.005 mg to about 5000 mg per treatment. In more specific embodiments, the range is from about 0.05 mg to about 1000 mg, or from about 0.5 mg to about 500 mg, or from about 5 mg to about 50 mg. Treatment can be administered one or more times per day (for example, once per day, twice per day, three times per day, four times per day, five times per day, etc.). When multiple treatments are used, the amount can be the same or different. It is understood that a treatment can be administered every day, every other day, every 2 days, every 3 days, every 4 days, every 5 days, etc. For example, with every other day administration, a treatment dose can be initiated on Monday with a first subsequent treatment administered on Wednesday, a second subsequent treatment administered on Friday, etc. Treatment is typically administered from one to two times daily. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

Alternatively, the effective amount of a compound of the invention is from about 0.01 mg/kg/day to about 1000 mg/kg/day, from about 0.1 mg/kg/day to about 100 mg/kg/day, from about 0.5 mg/kg/day to about 50 mg/kg/day, or from about 1 mg/kg/day to 10 mg/kg/day.

In another embodiment, any of the above methods of treatment comprises the further step of co-administering to said patient one or more second therapeutic agents. The choice of second therapeutic agent may be made from any second therapeutic agent known to be useful for co-administration with a compound that modulates the APJ receptor. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

### Kits

The present invention also provides kits for use to treat the target disease, disorder or condition. These kits comprise (a) a pharmaceutical composition comprising a compound of Formula I, or a salt thereof, wherein said pharmaceutical composition is in a container; and (b) instructions describing a method of using the pharmaceutical composition to treat the target disease, disorder or condition.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### General Methods for Preparing APJ Receptor Compounds

### Synthesis of Peptides

The peptide component (P) of the compounds of the invention can be synthesized by incorporating orthogonally protected amino acids in a step-wise fashion. Any suitable synthetic methods can be used. Traditional Fmoc or Boc chemistry can be easily adapted to provide the desired peptide component (P) of the compounds of the invention. Fmoc is generally preferred, because the cleavage of the Fmoc protecting group is milder than the acid deprotection required for Boc cleavage, which requires repetitive acidic deprotections that lead to alteration of sensitive residues, and increase acid catalyzed side reactions. (G. B. FIELDS et al. in Int. J. Pept. Protein, 1990, 35, 161).

The peptides can be assembled linearly via Solid Phase Peptide Synthesis (SPPS), can be assembled in solution using modular condensations of protected or unprotected peptide components or a combination of both.

### Solid Phase Peptide Synthesis

For SPPS, an appropriate resin is chosen that will afford the desired moiety on the C-terminus upon cleavage. For example upon cleavage of the linear peptide, a Rink amide resin will provide a primary amide on the C-terminus, whereas a Rink acid resin will provide an acid. Rink acid resins are more labile than Rink amide resins and the protected peptide could also be cleaved and subsequently the free acid activated to react with amines or other nucleophiles. Alternatively, other resins could provide attachment of other moieties prior to acylation, leading to cleavage of an alkylated secondary amide, ester or other desired C-terminal modification. A review of commonly used resins and the functional moiety that results after cleavage can be found in manufacturer literature such as NovaBiochem or Advanced Chemtech catalogues.

Typically a resin is chosen such that after cleavage the C-terminus is an amide bond. Rink amide resin is a resin that results in a C-terminal amide during cleavage. The orthogonally protected Fmoc amino acids are added stepwise using methods well known in literature (Bodansky M. Principles of Peptide synthesis (1993) 318p; Peptide Chemistry, a Practical Textbook (1993); Spinger-Verlag). These procedures could be done manually or by using automated peptide synthesizers.

The process involves activating the acid moiety of a protected amino acid, using activating agents such as HBTU, HATU, PyBop or simple carbodiimides. Often an additive is used to decrease racemization during coupling such as HOBt or HOAt (M. SCHNÖLZER et al., Int. J. Pept. Protein Res., 1992, 40, 180). Manually, the coupling efficiency can be determined photometrically using a ninhydrin assay. If the coupling efficiency is below 98%, a second coupling may be desired. After the second coupling a capping step may be employed to prevent long deletion sequences to form, simplifying the purification of the desired final compound. With automation, second couplings are not commonly required, unless a residue is known to be problematic such as Arginine.

Deprotection of the Fmoc is most commonly accomplished using piperidine (20%) in dimethylformamide (DMF). Alternatively other secondary amines may also be used such as morpholine, diethylamine or piperazine. This reaction is facile and normally is accomplished within 20 minutes using piperidine. After deprotection the resin is washed several times with DMF and DCM prior to coupling with the next residue. This process is repeated, assembling the peptide linearly until the sequence is complete. The final Fmoc is removed, which allows for coupling with the tether moiety.

In a preferred synthesis, the peptide is formed by SPPS accomplished manually or in an automated fashion using a commercially available synthesizer such as the CEM Microwave peptide synthesizer, Rainin Symphony synthesizer, or ABI 433 flow-through synthesizer. Commercially available Rink Amide resin is used for synthesizing the C-terminal amide peptides (Rink, H. Tetrahedron Lett, 28, 4645, 1967). Peptide synthesis reagents (coupling, deprotection agents) are commercially available and include HOBT, HBTU (Novabiochem) as well as DMF, DCM, Piperidine, NMP, and DIEA (Sigma-Aldrich). Suitably protected amino acids for use in solid phase peptide synthesis are commercially available from many sources, including Sigma-Aldrich and CEM Corporation.

For example, a convenient preparation of peptides on a 0.1 mmol or 0.25 mmol scale uses Rink amide solid-phase resin with a substitution of about 0.6 mmol/g. Linear attachment of the amino acids is accomplished on a ABI continuous flow automated synthesizer using 5 eq of orthogonally protected amino acid (AA), and using HBTU/HOBt coupling protocol, (5 eq. of each reagent). In another preferred synthesis, peptides can be synthesized using a microwave instrument using 10 eq of reagents. Deprotection of Fmoc can be accomplished with 20% piperidine in DMF followed by washing with DMF and DCM.

In both cases (i.e., Rink acid and Rink amide resins), final Fmoc deprotection of the N-terminus would leave a free amine after cleavage from the resin unless it is modified prior to cleavage. In the compounds of the invention, tether moieties are attached through amide bonds.

### Solution Phase Synthesis of Peptides

For solution phase synthesis the desired peptide is generally broken down into peptide fragments in units of 2-4 amino acids. The selected unit is dependent on the sequence, the stability of the fragment to racemization, and the ease of assembly. As each amino acid is added, only 1-1.5 eq of the residue is required, versus the 5-10 equivalents of reagent required for SSPS. Preactivated amino acids such as OSu active ester and acid fluorides also can be used, requiring only a base for completion of the reaction.

Coupling times require 1.5-2 hours for each step. Two fragments are condensed in solution, giving a larger fragment that then can be further condensed with additional fragments until the desired sequence is complete. The solution phase protocol uses only 1 eq of each fragment and will use coupling reagents such as carbodiimides (DIC). For racemized prone fragments, PyBop or HBTU/HOBt can be used. Amino acids with Bsmoc/tBu or Fmoc/tBu and Boc/Benzyl protection are equally suitable for use.

When Fmoc is used, the use of 4-(aminomethyl) piperidine or tris(2-aminoethyl)amine as the deblocking agent can avoid undesired side reactions. The resulting Fmoc adduct can be extracted with a phosphate aqueous buffer of pH 5.5 (Organic Process Research & Development 2003, 7, 2837). If Bsmoc is used, no buffer is required, only aqueous extractions are needed. Deprotections using these reagents occur in 30-60 minutes. Deblocking of the Fmoc group on the N-terminal residue provides a free terminal amine that is used for attachment of the tether moiety. In the compounds of the invention, tether moieties are attached through amide bonds to the N-terminal amine.

One advantage of solution phase synthesis is the ability to monitor the compound after every coupling step by mass spectrometry to see that the product is forming. In addition, a simple TLC system could be used to determine completion of reaction.

### Attachment of Tethers

Tethers are attached to the terminal nitrogen of the N-terminal amino acid of the peptide chain using amide bond coupling:

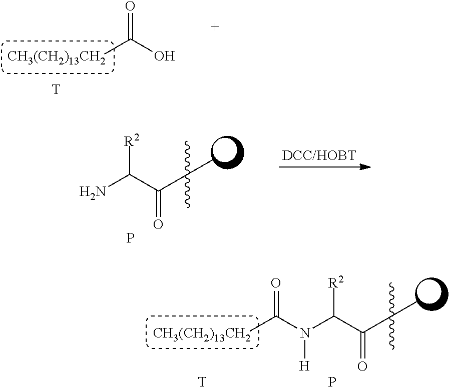

The tether can be attached using solid phase procedures or in solution using an amide bond coupling. After the N-terminus is suitably coupled, the final compound is cleaved from the resin using an acidic cocktail (Peptide Synthesis and Applications, John Howl, Humana Press, 262p, 2005). Typically these cocktails use concentrated trifluoroacetic acid (80-95%) and various scavengers to trap carbocations and prevent side chain reactions. Typical scavengers include isopropylsilanes, thiols, phenols and water. The cocktail mixture is determined by the residues of the peptide. Special care needs to be taken with sensitive residues, such as methionine, aspartic acid, and cysteine. Typical deprotection occurs over 2-5 hours in the cocktail. A preferred deprotection cocktail include the use of triisopropylsilane (TIS), Phenol, thioanisole, dodecanethiol (DDT) and water. Methane sulfonic acid (MSA) may also be used in the cocktail (4.8%). A more preferred cocktail consists of (TFA:MSA:TIS:DDT:Water 82:4.5:4.5:4.5:4.5; 10 mL/0.1 mmol resin).

After deprotection, the resin is removed via filtration, and the final compound is isolated via precipitation from an organic solvent such as diethyl ether, m-tert-butyl ether, or ethyl acetate and the resulting solid collected via filtration or lyophilized to a powder. Purification of the peptide using reverse phase HPLC may be required to achieve sufficient purity. Generally, a gradient of aqueous solvent with an organic solvent will provide sufficient separation from impurities and deletion sequences. Typically 0.1% TFA is used as the aqueous and organic modifier, however, other modifiers such as ammonium acetate can also be used. After purification, the compound is collected, analyzed and fractions of sufficient purity are combined and lyophilized, providing the compound as a solid.

### Amino Acid Reagents

The following commercially available orthogonally protected amino acids used can be used in the synthesis of compounds of the invention: Fmoc-Tyr(tBu)-OH, Fmoc-Ala-OH*H₂O, Fmoc-Arg(Pbf)-OH, Fmoc, Asn(Trt)-OH, Fmoc-Asp(tBu), Fmoc-Cys(tBu)-OH, Fmoc-Glu(tBu)-OH, Fmoc-Glx(Pbf)-OH, Fmoc-Gly-OH, Fmoc-His(Trt)-OH, Fmoc-Leu-OH, Fmoc-Ile-OH, Fmoc, Lys(tBu)-OH, Fmoc-Met-OH, Fmoc-Phe-OH, Fmoc-Ser(tBu)-OH, Fmoc-Thr(tBu)-OH, Fmoc-Typ-OH, and Fmoc-Val-OH. Additional amino acids suitable for incorporation into the compounds of the invention (e.g., D amino acids, substituted amino acids and other protecting group variations) are also commercially available or synthesized by methods known in the art.

### Analytical Methods

The compounds of the invention are analyzed for purity by HPLC using the methods listed below. Purification is achieved by preparative HPLC.

Fast LC/MS Method
Column: Phenomenex Luna C-5 20×30 mm
Flow: 1.0 ml/min
Solvent A: 0.1% TFA in Type I water
Solvent B: 0.1% TFA in Acetonitrile
UV 220 nm
Injection: 20 ul
Gradient 5-95% B (7 minutes); 95-5% B (1 minute); 5% B (4 minutes)

Analytical Purity Method
Column: Phenomenex Luna C-5 20×30 mm
Flow: 1.0 ml/min
Solvent A: 0.1% TFA in Type I water
Solvent B: 0.1% TFA in Acetonitrile
UV: 220 nm
Injection: 20 ul
Gradient: 2-95% B (10 minutes); 95-2% B (2 minutes); 2% B (2 minutes)

Preparative LC/MS Method
Column: Phenomenex Luna C-5 250×150 mm
Flow: 5.0 ml/min
Solvent A: 0.1% TFA in Type I water
Solvent B: 0.1% TFA in Acetonitrile
UV: 220 nm
Injection: 900 ul
Gradient: 35% B (5 minutes); 35-85% B (13 minutes); 85-35% B (0.5 minutes); 35% B (1.5 minutes)

### Synthesis of Compounds

### Compound 12 Pal-TVFRSSREKRRSADIFI-Amide

Compound 12 was synthesized as described above on Rink amide resin at 0.1 mmol scale. Amino acids were coupled sequentially as described above. Following deprotection of the Fmoc group on the N-terminal residue serine, the N-terminal amine was capped with palmitic acid (10 eq.), HBTU (10 eq.) and DIEA (10 eq.) as described above. The pepducin was cleaved from the resin by TFA containing MS, TIS, DDT, and water (82:4.5:4.5:4.5:4.5;10 mL), filtered through a medium frit Büchner full, triturated with ether and the resulting precipitate collected by centrifugation. Crude peptide was taken up in minimum amount of DMSO (˜1 ml) and purified by RP-HPLC as described previously. Fractions with correct MW were pooled and lyophilized and analyzed for purity using Method A. The yield of representative lots is illustrated in the following table.

| Lot # | Yield (mg) |
|---|---|
| 1 | 2.3 |
| 2 | 4.6 |
| 3 | 5.1 |
| 4 | 26.3 |
| 5 | 14.5 |
| 6 | 28 |

### Compound 96 Pal-QTIAGHFRKERIEGLRKRRRLLA-Amide

Compound 96 was synthesized as described for Compound 12. The yield of representative lots is illustrated in the following table.

| Lot # | Yield (mg) |
|---|---|
| 1 | 3.7 |
| 2 | 20 |
| 3 | 9.3 |

### Compound 11 Pal-FRSSREKRRSADIFI-Amide

Compound 11 was synthesized as described for Compound 12. The yield of representative lots is illustrated in the following table.

| Lot # | Yield (mg) |
|---|---|
| 1 | 4.5 |
| 2 | 9 |

Additional compounds that were synthesized following the above-described method are listed in Table 6.

TABLE 6

| | | MS | MS | |
|---|---|---|---|---|
| Compound | Loop | Theoretical | Observed | MW |
| Compound 1 | i1 | 653.8 | 653.8 | 1958.355 |
| Compound 2 | i1 | 902.1 | 902.5 | 1802.169 |
| Compound 3 | i1 | 572.7 | 572.9 | 1715.092 |
| Compound 4 | i1 | 543.5 | 543.3 | 1628.014 |
| Compound 5 | i1 | 735.9 | 735.8 | 2204.66 |
| Compound 6 | i1 | 671.8 | 671.5 | 1341.603 |
| Compound 7 | i1 | 527.9 | 628 | 1383.639 |
| Compound 8 | i1 | 866.7 | 867 | 1732.039 |
| Compound 9 | i1 | 793.2 | 793.4 | 1584.865 |
| Compound 10 | i1 | 1429.68 | 1429.7 | 1428.68 |
| Compound 11 | i1 | 1053.9 | 1053.7 | 2105.529 |
| Compound 12 | i1 | 769.5 | 769.3 | 2305.763 |
| Compound 13 | i1 | 967.1 | 967.1 | 1932.274 |
| Compound 14 | i1 | 758.4 | 758 | 1514.857 |
| Compound 15 | i1 | 684.8 | 684.5 | 1367.683 |
| Compound 16 | i1 | 616.06 | 616 | 1845.197 |
| Compound 17 | i1 | 639.8 | 639.6 | 1916.275 |
| Compound 18 | i1 | 649.14 | 648.8 | 1944.328 |
| Compound 19 | i1 | 760.2 | 760.3 | 2277.71 |
| Compound 20 | i1 | 607.47 | 607.3 | 2425.912 |
| Compound 21 | i1 | 759.56 | 759.35 | 2275.738 |
| Compound 22 | i1 | 760.2 | 760.3 | 2277.71 |
| Compound 23 | i1 | 744.2 | 744 | 2229.667 |
| Compound 24 | i1 | 741.2 | 741.2 | 2220.656 |
| Compound 25 | i1 | 634.8 | 634.6 | 1901.3 |
| Compound 26 | i1 | | | 2521.92 |
| Compound 27 | i1 | 573.5 | 573.3 | 2289.764 |
| Compound 28 | i1 | 573.5 | 573.35 | 2289.764 |
| Compound 29 | i1 | 563.2 | 563.05 | 2248.669 |
| Compound 30 | i1 | 740.8 | 741.45 | 2248.666 |
| Compound 31 | i1 | 741.4 | 741.5 | 2220.656 |
| Compound 32 | i1 | 764.2 | 764.1 | 2289.764 |
| Compound 33 | i1 | 625.4 | 625.3 | 1873.247 |
| Compound 34 | i1 | 654.4 | 654.3 | 1960.324 |
| Compound 35 | i1 | 556.2 | 556 | 2220.656 |
| Compound 36 | i1 | 779.5 | 779.6 | 2335.788 |
| Compound 37 | i1 | 754.6 | 754.9 | 2261.754 |
| Compound 38 | i1 | 566.9 | 566.8 | 2263.684 |
| Compound 39 | i1 | 744.2 | 744.1 | 2229.667 |
| Compound 40 | i1 | 566.9 | 566.5 | 2263.684 |
| Compound 41 | i1 | 562.9 | 562.6 | 2247.727 |
| Compound 42 | i1 | 577.7 | 577.3 | 2306.748 |
| Compound 44 | i1 | 577.4 | 577.1 | 2305.763 |
| Compound 45 | i1 | 577.4 | 577 | 2305.763 |
| Compound 46 | i1 | 577.4 | 577 | 2305.763 |
| Compound 47 | i1 | 577.4 | 577 | 2305.763 |
| Compound 48 | i1 | 577.4 | 577.2 | 2305.763 |
| Compound 49 | i1 | 577.4 | 577.1 | 2305.763 |
| Compound 50 | i1 | 577.4 | 577.1 | 2305.763 |
| Compound 51 | i1 | 577.4 | 577 | 2305.763 |
| Compound 52 | i1 | 577.4 | 577.1 | 2305.763 |
| Compound 53 | i1 | 577.4 | 576.9 | 2305.763 |
| Compound 54 | i1 | 577.4 | 577 | 2305.763 |
| Compound 55 | i1 | 577.4 | 577.1 | 2305.763 |
| Compound 56 | i1 | 577.4 | 577.1 | 2305.763 |
| Compound 57 | i1 | 577.4 | 577 | 2305.763 |
| Compound 58 | i1 | 577.4 | 577 | 2305.763 |
| Compound 59 | i1 | 577.4 | 577 | 2305.763 |
| Compound 60 | i1 | 770.2 | 769.8 | 2307.736 |
| Compound 61 | i1 | 778.2 | 777.6 | 2331.801 |
| Compound 62 | i1 | 778.2 | 777.8 | 2331.801 |
| Compound 63 | i1 | 581.4 | 581 | 2321.763 |
| Compound 64 | i1 | 585.2 | 584.7 | 2336.778 |
| Compound 65 | i1 | 588.2 | 587.8 | 2348.831 |
| Compound 67 | i1 | 778.9 | 778.4 | 2333.817 |
| Compound 68 | i1 | 750.9 | 750.3 | 2249.657 |
| Compound 69 | i1 | 741.5 | 741 | 2221.604 |
| Compound 75 | i2 | 902.1 | 902 | 1802.262 |
| Compound 76 | i2 | 951.7 | 951.7 | 1901.393 |
| Compound 77 | i2 | 1008.3 | 1008.1 | 2014.55 |
| Compound 78 | i2 | 696.2 | 696 | 2085.628 |
| Compound 79 | i2 | 733.9 | 733.3 | 2198.786 |
| Compound 80 | i2 | 695.4 | 695.1 | 1388.79 |
| Compound 81 | i2 | 617.3 | 617 | 1232.604 |
| Compound 82 | i2 | 865.13 | 865 | 1728.263 |
| Compound 83 | i2 | 830.1 | 830 | 1658.133 |
| Compound 84 | i2 | 751.5 | 751.5 | 1501.947 |
| Compound 85 | i2 | 879.6 | 879.4 | 1757.264 |
| Compound 86 | i2 | 615.8 | 615.5 | 1844.341 |
| Compound 87 | i3 | 706.7833333 | 707.1 | 2117.35 |
| Compound 88 | i3 | 749.614 | 749.7 | 2245.842 |
| Compound 89 | i3 | 863.7326667 | | 2588.198 |
| Compound 90 | i3 | 768.9616667 | | 2303.885 |
| Compound 91 | i3 | 674.1756667 | | 2019.527 |
| Compound 92 | i3 | 765.225 | 765.7 | 3056.9 |
| Compound 93 | i3 | 944.1616667 | 944 | 2829.485 |
| Compound 94 | i3 | 882.75 | 882.5 | 2645.25 |
| Compound 95 | i3 | 536.0233333 | 535.8 | 1605.07 |
| Compound 96 | i3 | 1013.976667 | 1015.1 | 3038.93 |
| Compound 97 | i3 | 812.6866667 | 812.4 | 2435.06 |
| Compound 98 | i3 | 864.0466667 | 864.35 | 2589.14 |
| Compound 99 | i3 | 760.5946667 | 760 | 2278.784 |
| Compound 100 | i3 | 707.2066667 | 707.25 | 2118.62 |
| Compound 101 | i3 | 565.3626667 | 565.45 | 1693.088 |
| Compound 102 | i3 | 877.4166667 | 877.3 | 2629.25 |
| Compound 103 | i3 | 925.4596667 | 925.4 | 2773.379 |
| Compound 104 | i3 | 954.4853333 | 954.3 | 2860.456 |
| Compound 105 | i3 | 906.4423333 | 906.3 | 2716.327 |
| Compound 106 | i3 | 973.5023333 | 973.2 | 2917.507 |
| Compound 107 | i3 | 780.70375 | 780.65 | 3118.815 |
| Compound 108 | i3 | 790.46275 | 790.35 | 3157.851 |

### Methods of Screening

### Functional Assays

Functional assays suitable for use in detecting and characterizing GPCR signaling include Gene Reporter Assays and Calcium Flux assays, cAMP and kinase activation assays. Several suitable assays are described in detail below.

### Gene Reporter Assays

Cells expressing the APJ receptor can be transiently or stably transfected with a reporter gene plasmid construct containing an enhancer element which responds to activation of a second messenger signaling pathway or pathways, thereby controlling transcription of a cDNA encoding a detectable reporter protein. APJ expression can be the result of endogenous expression on a cell line or cell type or the result of stable or transient transfection of DNA encoding the receptor of interest into a cell line by means commonly used in the art. Immortalized cell lines or primary cell cultures can be used.

If the activated pathway is stimulatory (e.g., Gs or Gq), agonist activity results in activation of transcription factors, in turn causing an increase in reporter gene transcription, detectable by an increase in reporter activity. To test for agonist or inverse agonist activity, cells expressing the APJ receptor and the reporter gene construct can be challenged by the test compound for a predetermined period of time (e.g., 2-12 hours, typically 4 hours). Cells can then be assessed for levels of reporter gene product. Inverse agonists will suppress levels of reporter to below basal levels in a dose dependent manner. To test for antagonist or inhibitory activity through a stimulatory pathway, cells expressing both the APJ receptor and the reporter gene construct can be activated by a receptor agonist to increase gene reporter product levels. Treatment with antagonists will counter the effect of agonist stimulation in a dose- and receptor-dependent manner.

To test for agonist activity on receptor signaling through an inhibitory pathway (eg, Gi, which couples to APJ), cells can be treated with a systematic activator (e.g., forskolin) to increase levels of reporter gene product. Activation of Gi by treatment with receptor agonist will inhibit this expression by inhibiting adenylyl cyclase. To screen for antagonist activity, test compounds can be assessed for the ability to counter agonist inhibition of adenylyl cyclase, resulting in increase reporter transcription.

Alternatively, a plasmid construct expressing the promiscuous G-protein Gal6 can be used to obtain a positive signal from a GPCR which normally couples to an inhibitory G-protein. Co-expression of the chimeric G-protein Gaq/Gai5 (Coward et al. Analytical Biochemistry 270, 242-248 (1999)) allows coupling to Gi-coupled receptors and conversion of second messenger signaling from the inhibitory Gi pathway to the stimulatory Gq pathway. Agonist and antagonist assessment in these systems is the same as the stimulatory pathways. Well-to-well variation caused by such factors as transfection efficiency, unequal plating of cells, and cell survival rates can be normalized in transient transfection assays by co-transfecting a constitutively expressing reporter gene with a non-interfering signal independent of the regulated reporter.

### Calcium Flux Assay

Calcium Flux Assay is one of the most popular cell-based GPCR functional assays. It most often uses calcium sensing fluorescent dyes such as fura2 AM, fluo-4 and Calcium-4 to measure changes in intracellular calcium concentration. It is used mainly to detect GPCR signaling via Gαq subunit. Activation of these Gq-coupled GPCRs leads to activation of phospholipase C, which subsequently leads to increase in inositol phosphate production. IP3 receptors on endoplasmic reticulum sense the change then release calcium into cytoplasm. Intracellular calcium binding to the fluorescent dyes can be detected by instruments that quantify fluorescent intensities, such as FLIPR Tetra, Flexstation (MDS) and FDSS (Hamamatsu). In additional to assess Gq-couple receptor signaling, calcium flux assay can also be used to study Gs and Gi couple receptors by co-expressing CNG (cycic nucleotide gated calcium channel) or chimeric G-proteins (Gqi5, Gsi5 for example). Activation of some Gi-coupled receptors can also be detected by calcium flux assay via Gβγ mediated phospholipase C activation.

### APJ Testing

An example of the use of the calcium flux assay can be assessing Apelin activation of APJ receptors in Molt3 human cell lines or in Rat RBL cells stably transfected with APJ. Cells can be seeded into 96-well black plates with clear bottom at 200K/well in Hank's balanced salt solution with 20 mM HEPES, 0.1% BSA. After dye loaded by incubating in Calcium-4 dye at room temperature for 1 hour, cell plates can be placed in Flexstation 3. The addition of test compound or reference antagonists can be done either by manual pipetting or by liquid handling on Flexstation. The latter allows the assessment of agonist activity of the test compound. After incubation of 15 minutes at 37° C., Apelin can be added on Flexstation and receptor activation can be assessed by measuring changes in fluorescent intensity. This mode of assay also allows the detection of agonists and agonistic modulators of APJ activity.

HTRF cAMP Assay and IP-One Assay (Cisbio)

HTRF (homogeneous time resolved fluorescence) is a technology developed by Cisbio Bioassays based on TR-FRET (time-resolved fluorescence resonance energy transfer). Cisbio Bioassays has developed a wide selection of HTRF-based assays compatible with whole cells, thereby enabling functional assays to run under more physiological conditions. The IP-One assays are competitive immunoassays using cryptate-labeled anti-IPI monoclonal antibody and d2-labeled IP1. IP1 is a relatively stable downstream metabolite of IP3, and accumulates in cells following Gq receptor activation.

cAMP kits based on a competitive immunoassay using cryptate-labeled anti-cAMP antibody and d2-labeled cAMP were used to assay the effects of APJ compounds of the present invention. This assay measures the increase in intracellular cAMP upon Gs-coupled receptor activation as well as decrease in forskolin (or a more soluble version of forskolin—NKH477) stimulated increase in cAMP upon Gi-coupled receptor activation. For example, treatment of HEK cells stably expressing the Gi-coupled receptor APJ with its endogenous ligand Apelin inhibited NKH477 stimulated increase in cAMP with an EC₅₀ of 5 e-10 M.

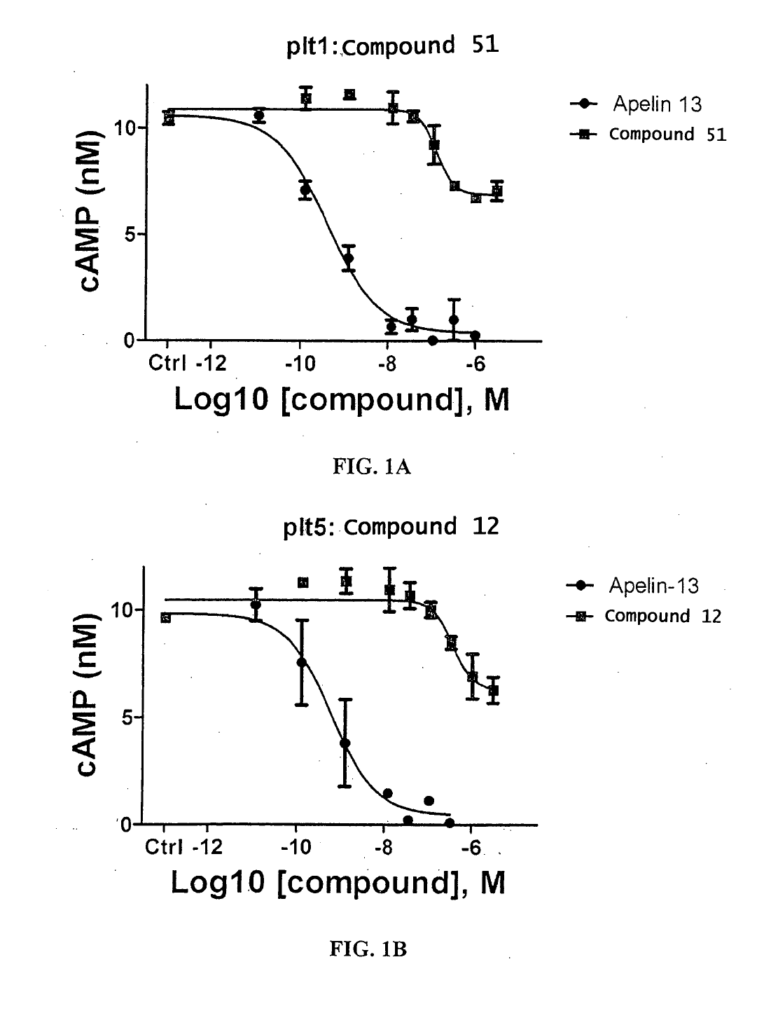

Representative data for this assay are described for Compound 51 and Compound 12 in FIGS. 1A and 1B, respectively. Further testing of compounds was conducted and the results are set forth in Table 7. For the data in Table 7 EC50 values from: 1 nM to 500 nM are designated as *****; 501 nM to 1000 nM are designated as ****; 1001 nM to 5000 nM are designated as ***; 5001 nM to 10,000 nM are designated as **; and greater than 10,000 nM are designated *.

TABLE 7

| Compound | Loop | EC₅₀ value (nM), SEM |
|---|---|---|
| Compound 1 | i1 | ***** |
| Compound 2 | i1 | ***** |
| Compound 3 | i1 | * |
| Compound 4 | i1 | * |
| Compound 5 | i1 | ***** |
| Compound 6 | i1 | * |
| Compound 7 | i1 | * |
| Compound 8 | i1 | * |
| Compound 9 | i1 | * |
| Compound 10 | i1 | * |
| Compound 11 | i1 | ***** |
| Compound 12 | i1 | ***** |
| Compound 13 | i1 | * |
| Compound 14 | i1 | * |
| Compound 15 | i1 | * |
| Compound 16 | i1 | * |
| Compound 17 | i1 | * |
| Compound 18 | i1 | * |
| Compound 19 | i1 | ***** |
| Compound 20 | i1 | **** |
| Compound 21 | i1 | ***** |
| Compound 22 | i1 | ***** |
| Compound 24 | i1 | *** |
| Compound 25 | i1 | *** |
| Compound 26 | i1 | **** |
| Compound 27 | i1 | **** |
| Compound 28 | i1 | ***** |
| Compound 32 | i1 | ***** |
| Compound 33 | i1 | * |
| Compound 36 | i1 | *** |
| Compound 38 | i1 | ***** |
| Compound 39 | i1 | ***** |
| Compound 40 | i1 | **** |
| Compound 41 | i1 | ***** |
| Compound 42 | i1 | *** |
| Compound 44 | i1 | * |
| Compound 45 | i1 | * |
| Compound 46 | i1 | ***** |
| Compound 47 | i1 | ***** |
| Compound 48 | i1 | ***** |
| Compound 49 | i1 | ***** |
| Compound 50 | i1 | ***** |
| Compound 51 | i1 | ***** |
| Compound 52 | i1 | ***** |
| Compound 53 | i1 | ***** |
| Compound 54 | i1 | ***** |
| Compound 55 | i1 | ***** |
| Compound 56 | i1 | **** |
| Compound 57 | i1 | **** |
| Compound 58 | i1 | ***** |
| Compound 59 | i1 | ***** |
| Compound 60 | i1 | ***** |
| Compound 75 | i2 | * |
| Compound 76 | i2 | * |
| Compound 77 | i2 | * |
| Compound 78 | i2 | * |
| Compound 79 | i2 | * |
| Compound 80 | i2 | * |
| Compound 81 | i2 | * |
| Compound 82 | i2 | * |
| Compound 83 | i2 | * |
| Compound 84 | i2 | * |
| Compound 85 | i2 | * |
| Compound 86 | i2 | * |
| Compound 87 | i3 | *** |
| Compound 88 | i3 | *** |
| Compound 89 | i3 | *** |
| Compound 90 | i3 | *** |
| Compound 91 | i3 | ***** |
| Compound 92 | i3 | ***** |
| Compound 93 | i3 | ***** |
| Compound 94 | i3 | * |
| Compound 95 | i3 | * |
| Compound 96 | i3 | ***** |
| Compound 97 | i3 | * |
| Compound 98 | i3 | * |
| Compound 99 | i3 | * |
| Compound 100 | i3 | * |
| Compound 101 | i3 | * |
| Compound 102 | i3 | * |
| Compound 103 | i3 | * |
| Compound 104 | i3 | * |
| Compound 105 | i3 | * |

### AlphaScreen Cellular Linase Assays.

GPCR activation results in modulation of downstream kinase systems and is often used to probe GPCR function and regulation. TGR Bioscience and PerkinElmer have developed Surefire cellular kinase assay kits that are HTS capable and useful in screening kinase regulation. Such kits enable the monitoring of Gi regulated downstream kinases like ERK1/2. The assay allows the measurement of increases in ERK1/2 kinase phosphorylation upon Gi coupled receptor (e.g., APJ) activation and this signal in turn can be used to assay Gi coupled receptor modulator. Similar kits are also availibel to assay other pathway dependent siganlling kinases such as MAP and BAD.

### IN VIVO Assays

The G-protein coupled receptor APJ is important in several therapeutic areas including heart failure, hypertension, HIV infection, and oncology. APJ compounds of the present invention (agonists, antagonists, modulators) can be assessed using suitable in vivo models. Such in vivo models include rodent models of heart failure and hypertension or evaluation of cardiac contractility in isolated perfused hearts.

The spontaneous hypertensive rat (SHR) is an especially useful acute rat models of chronic ventricular pressure overload whereas the mean arterial pressure in male wt Wistar or SHR rats can be assessed via femoral cannulation of in the carotid via a blood pressure transducer (Regul. Pept. 2001: 99:87). In addition, isolated rat heart preps have been used to demonstrate the inotropic effects of apelin and therefor could be used to evaluate APJ agonists, antagonists, or modulators (Circ. Res 2002; 91(5):434-40) For a more chronic model of heart failure, a useful exemplary model is the mouse with with aortic banding (Circ Res. 2007: 101:e32-e42).

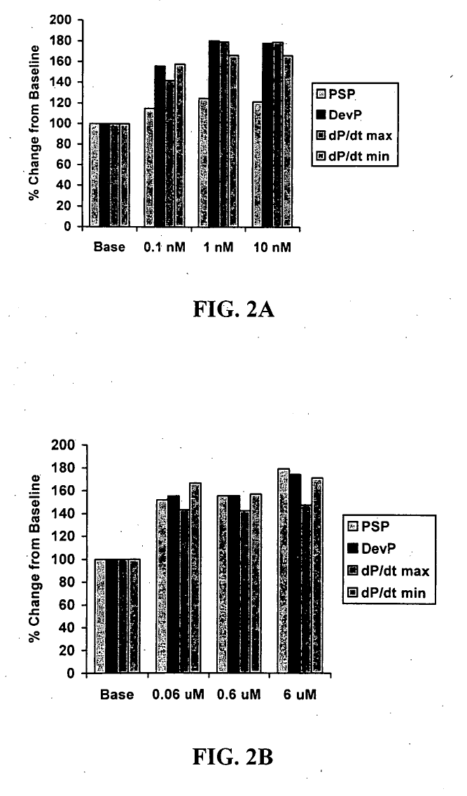

Mouse and rat Langendorff heart preparations were used to characterize the direct cardiac effects apelin and APJ pepducins have on the target tissue (Szokodi, Circ. Res 2002: 91 434-440). Hearts were perfused with (in mM) 118 NaCl, 4.7 KCl, 1.2 KH2PO4, 1.5 CaCl₂, 1.2 MgCl₂, 23 NaHCO₃, and 10.0 dextrose, gassed with 95% O₂-5% CO₂ and adjusted to a pH of 7.4. A pressure-sensing balloon catheter was inserted in the LV cavity to record changes in the developed pressure. Heart function was assessed by measuring standard parameters such as heart rate, mean peak systolic pressure, mean end diastolic pressure, developed pressure, dP/dt max, and dP/dt min. An increase in developed pressure is consistent with the known inotropic effect of the endogenous ligand for APJ, apelin. FIG. 2A illustrates the effect of the endogenous ligand apelin-13 on mouse heart function. The increase in developed pressure at 15 minutes is consistent with the known inotropic effects of the natural APJ ligand apelin. Likewise, FIG. 2B shows that Compound 12 demonstrates inotropic activity in the functioning heart tissue, consistent with the agonist effects at the APJ receptor.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A compound represented by Formula I:
T-L-P,
or pharmaceutically acceptable salts thereof, wherein:
P is a peptide comprising at least three contiguous amino-acid residues of an intracellular i1, i2, i3 loop or an intracellular i4 domain of the APJ receptor;
L is a linking moiety represented by C(O) and bonded to P at an N terminal nitrogen of an N-terminal amino-acid residue;
and T is a lipophilic tether moiety bonded to L, where the C-terminal amino acid residue of P is optionally functionalized.

2. The compound of claim 1, wherein P comprises at least six contiguous amino acid residues.

3. The compound of claim 1, wherein P is derived from the i1 loop and is represented by the following structural formula or pharmaceutically acceptable salts thereof,
X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-R₁,
wherein
X₁ is absent or a threonine or alanine residue;
X₂ is absent or a valine or alanine residue;
X₃ is absent or a phenylalanine or alanine residue;
X₄ is absent or an arginine, tryptophan, valine or alanine residue;
X₅ is absent or a serine or alanine residue;
X₆ is absent or a serine, glutamic acid or alanine residue;
X₇ is absent or an arginine or alanine residue;
X₈ is absent or a glutamic acid, alanine or proline residue;
X₉ is absent or a lysine, alanine, proline, glutamic acid, D-2,3-diaminionpropionic acid, or D-ornithine;
X₁₀ is absent or an arginine, alanine or lysine residue;
X₁₁ is absent or an arginine or alanine residue;
X₁₂ is absent or a serine, aspartic acid, alanine or arginine residue;
X₁₃ is absent or an alanine or serine residue;
X₁₄ is absent or an aspartic acid or alanine residue;
X₁₅ is absent or an isoleucine, alanine or aspartic acid residue;
X₁₆ is absent or a phenylalanine, valine, alanine or isoleucine residue;
X₁₇ is absent or an isoleucine, alanine or phenylalanine residue;
X₁₈ is absent or an alanine or isoleucine residue;
X₁₉ is absent or a serine residue;
provided that at least five of X₁-X₁₉ are present;
R₁ is OR₂ or N(R₂)₂;
each R₂ is independently hydrogen or (C₁-C₁₀)alkyl; and
from 0 to 5 amino acid residues are present in the D configuration.

4. The compound of claim 3, wherein at least four of X₇, X₈, X₉, X₁₀, X₁₁, X₁₂, X₁₃ and X₁₄ are present.

5. The compound of claim 4, wherein
X₇ is an arginine or alanine;
X₈ is a glutamic acid, alanine or proline;
X₉ is a lysine, alanine, proline, glutamic acid, D-2,3-diaminionpropionic acid, or D-ornithine; and
X₁₀ is an arginine, alanine or lysine.

6. The compound of claim 5, wherein at least one of X₇, X₈, X₉, or X₁₀ is alanine.

7. The compound of claim 4, wherein
X₁₁ is an arginine or alanine;
X₁₂ is a serine, aspartic acid, alanine or arginine;
X₁₃ is an alanine or serine; and
X₁₄ is an aspartic acid or alanine.

8. The compound of claim 7, wherein at least one of X₁₁, X₁₂, X₁₃ or X₁₄ is alanine.

9. The compound of claim 4, wherein
X₇ is an arginine or alanine;
X₈ is a glutamic acid, alanine or proline;
X₉ is a lysine, alanine, proline, glutamic acid, D-2,3-diaminionpropionic acid, or D-ornithine;
X₁₀ is an arginine, alanine or lysine;
X₁₁ is an arginine or alanine;
X₁₂ is a serine, aspartic acid, alanine or arginine;
X₁₃ is an alanine or serine; and
X₁₄ is an aspartic acid or alanine.

10. The compound of claim 9, wherein at least one of X₇, X₈, X₉, X₁₀, X₁₁, X₁₂, X₁₃ or X₁₄ is alanine.

11. The compound of claim 9, wherein
X₇ is an arginine;
X₈ is a glutamic acid;
X₉ is a lysine; and
X₁₀ is an arginine.

12. The compound of claim 9, wherein
X₁₁ is an arginine;
X₁₂ is a serine;
X₁₃ is an alanine; and
X₁₄ is an aspartic acid.

13. The compound of claim 3, selected from: 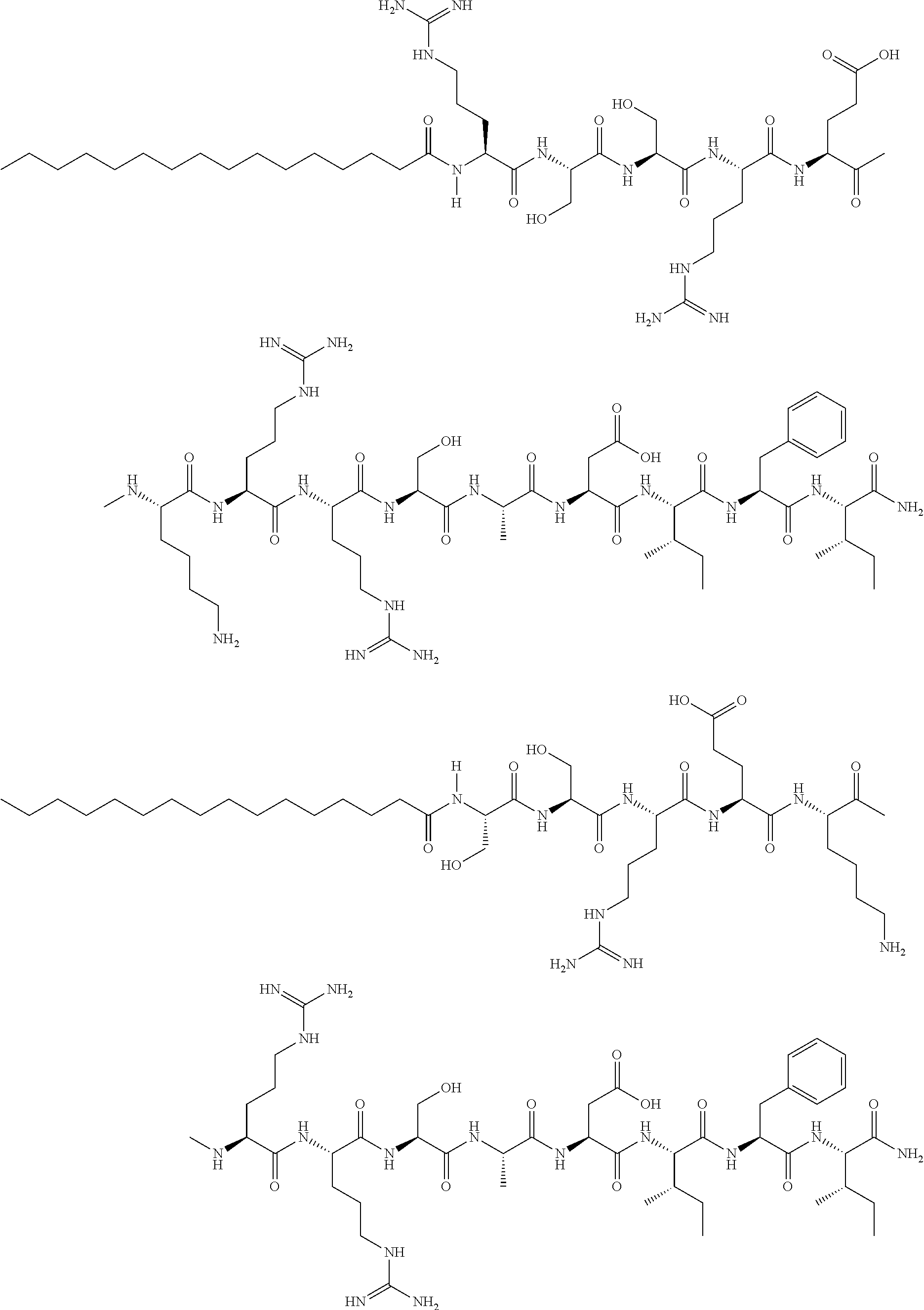 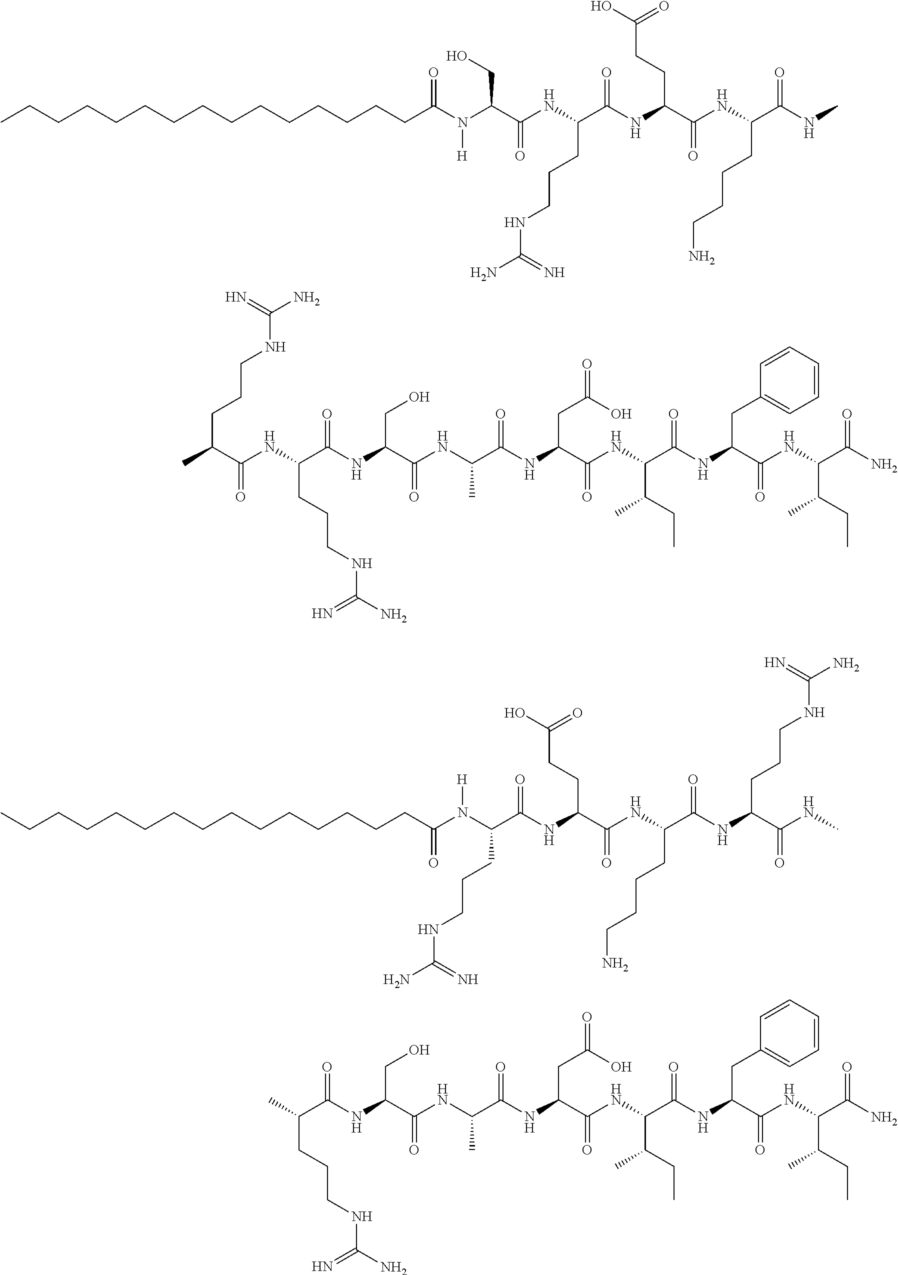 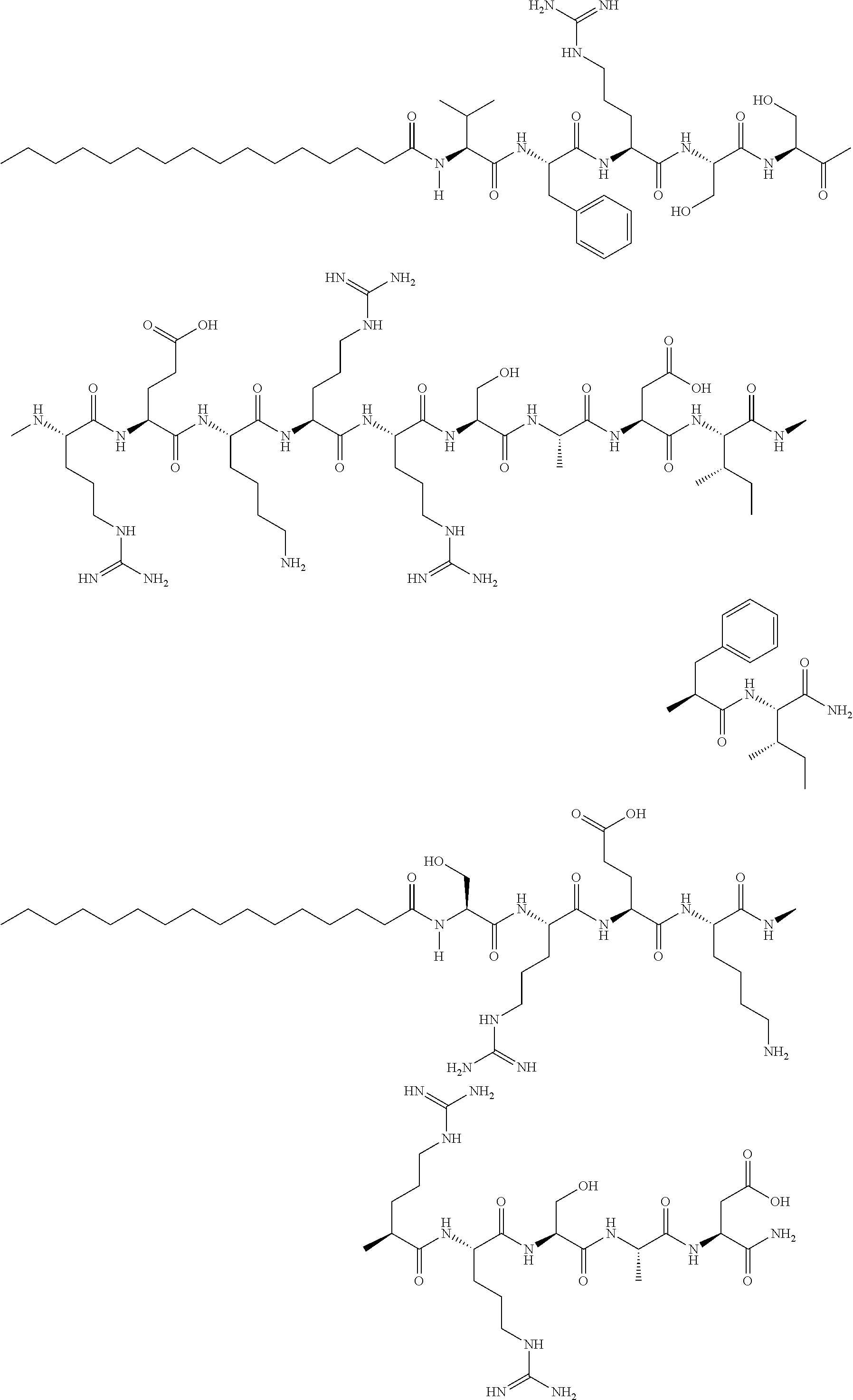 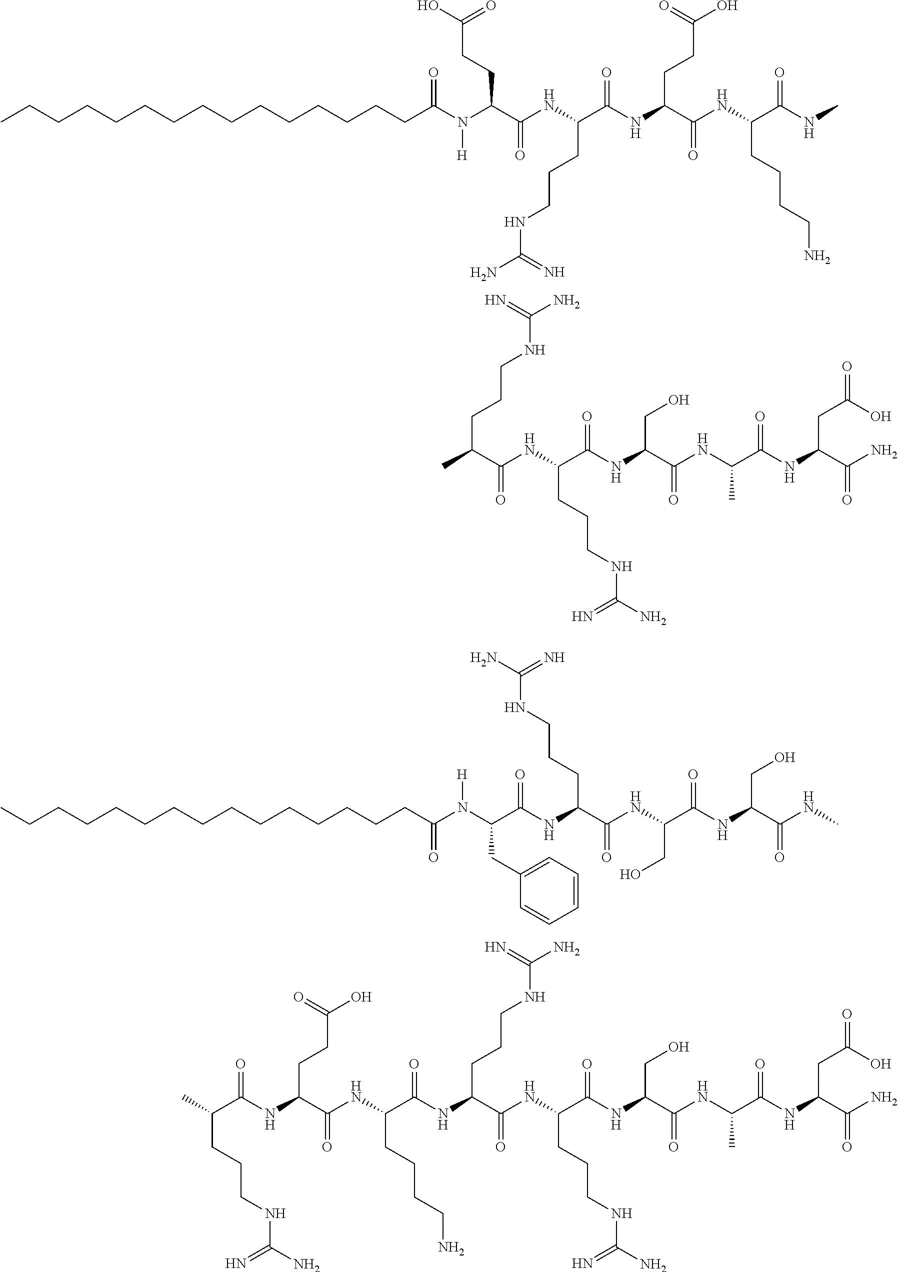 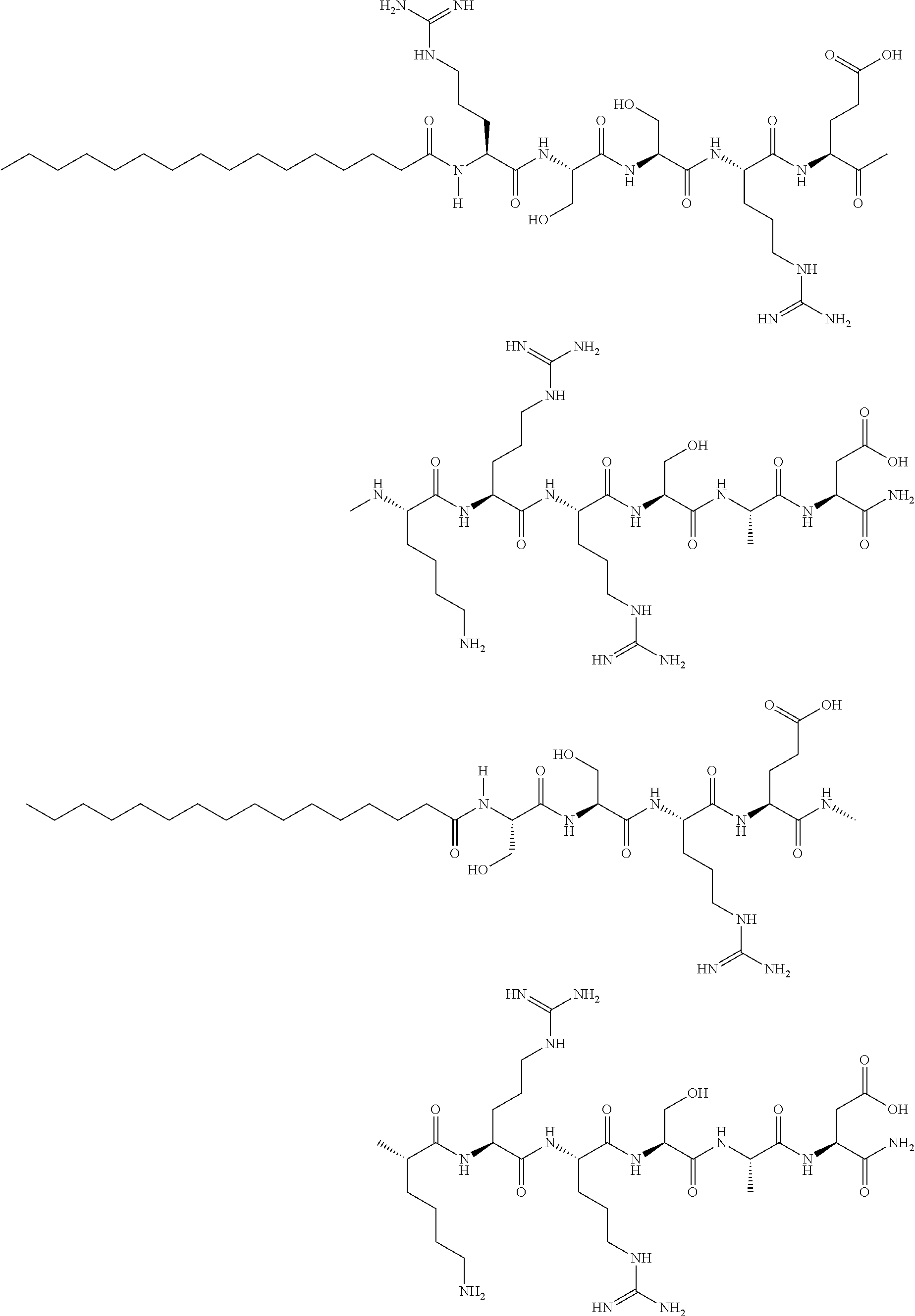 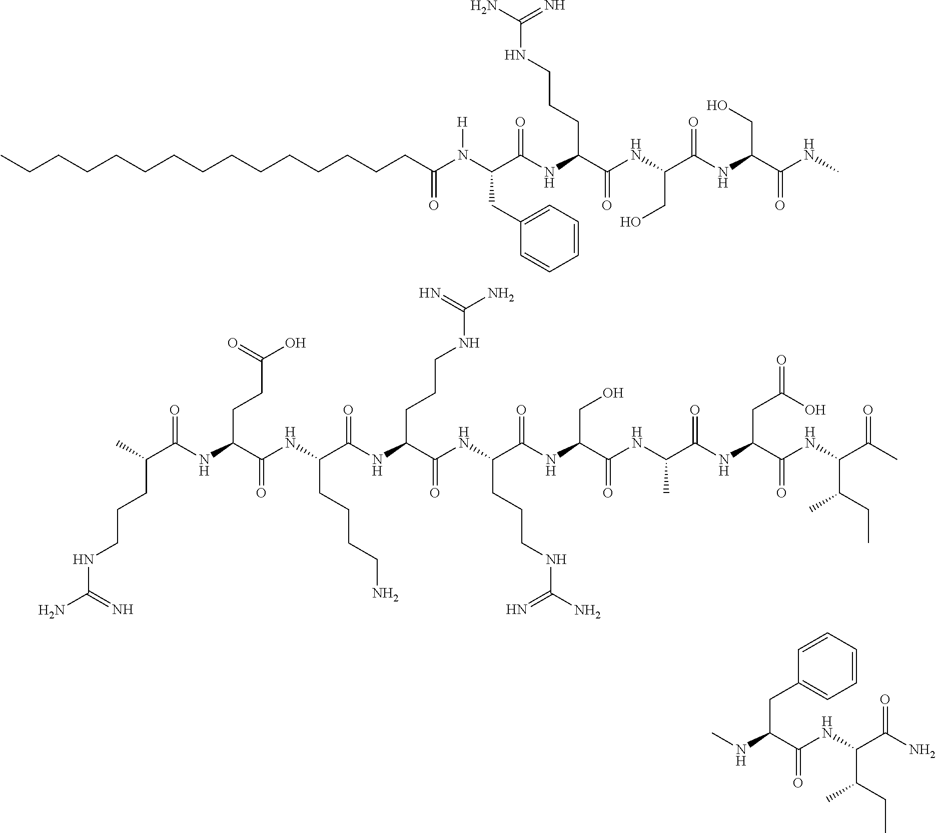 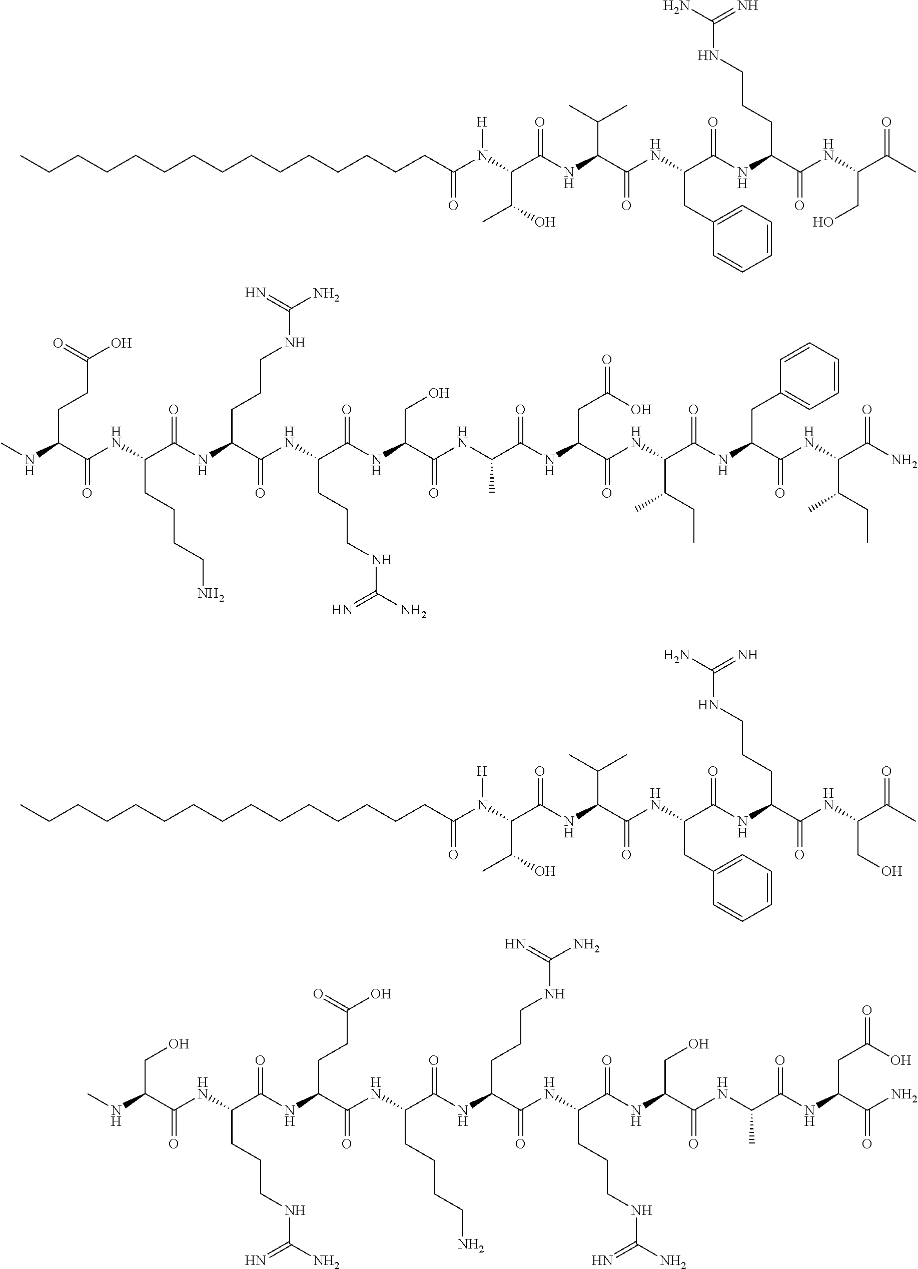 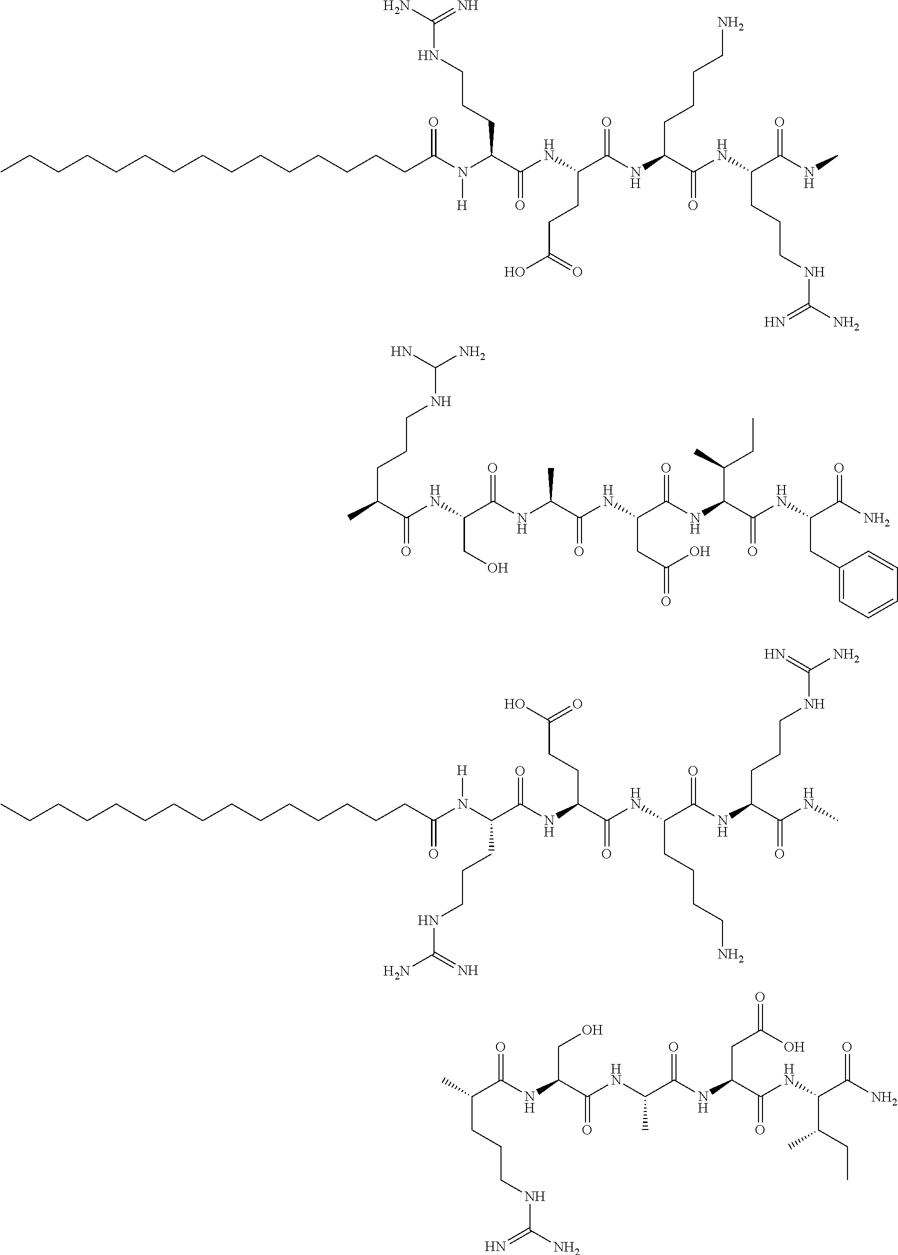 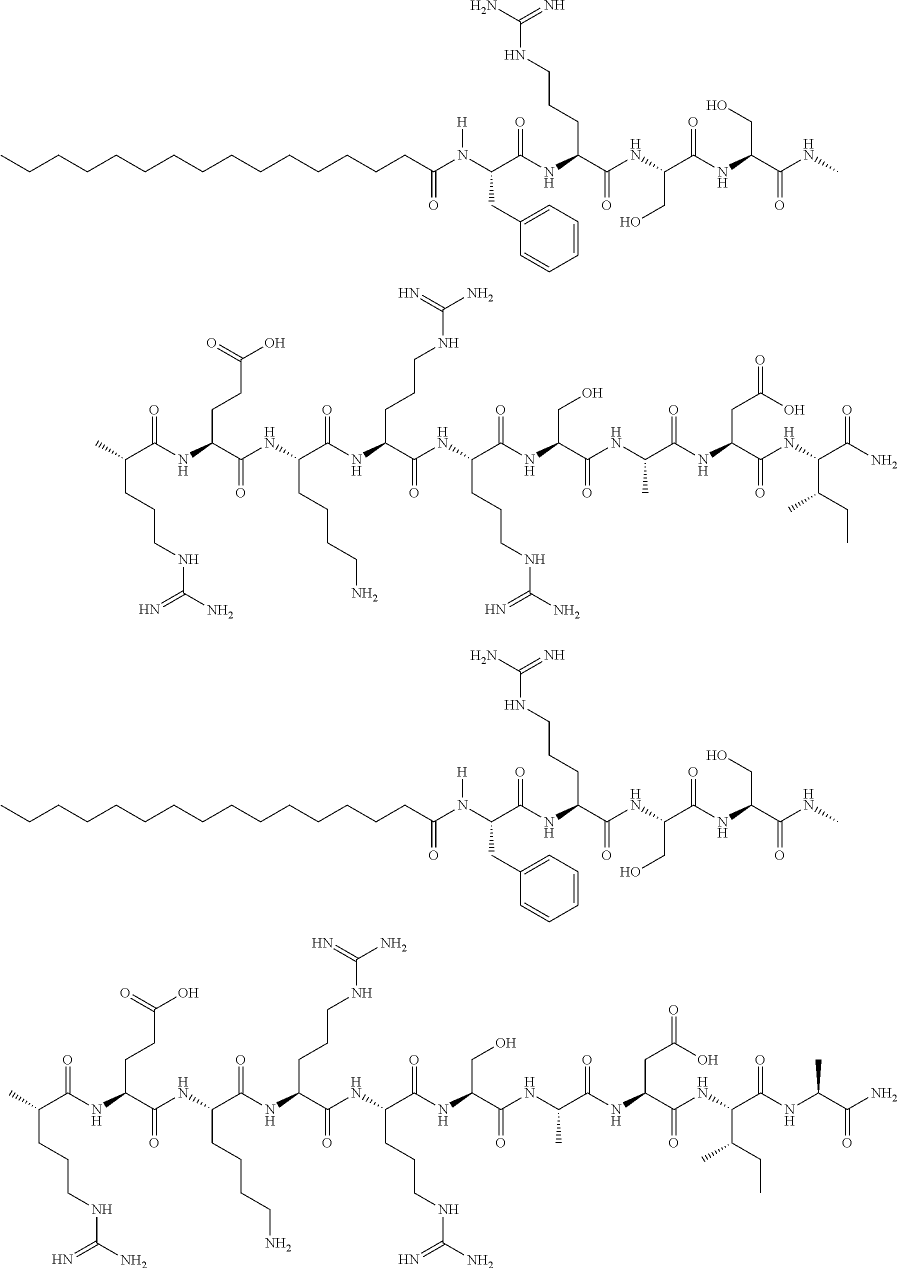 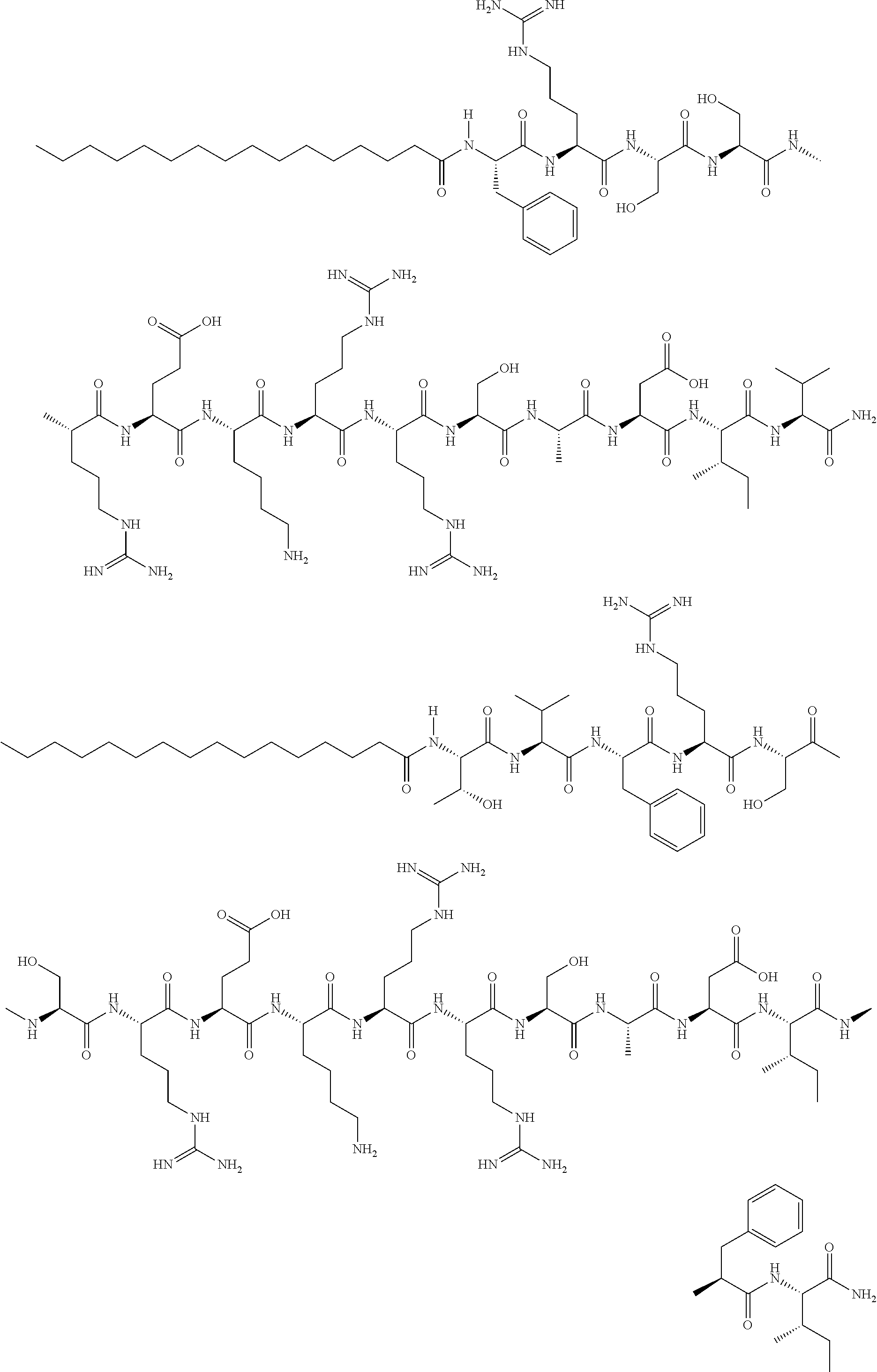 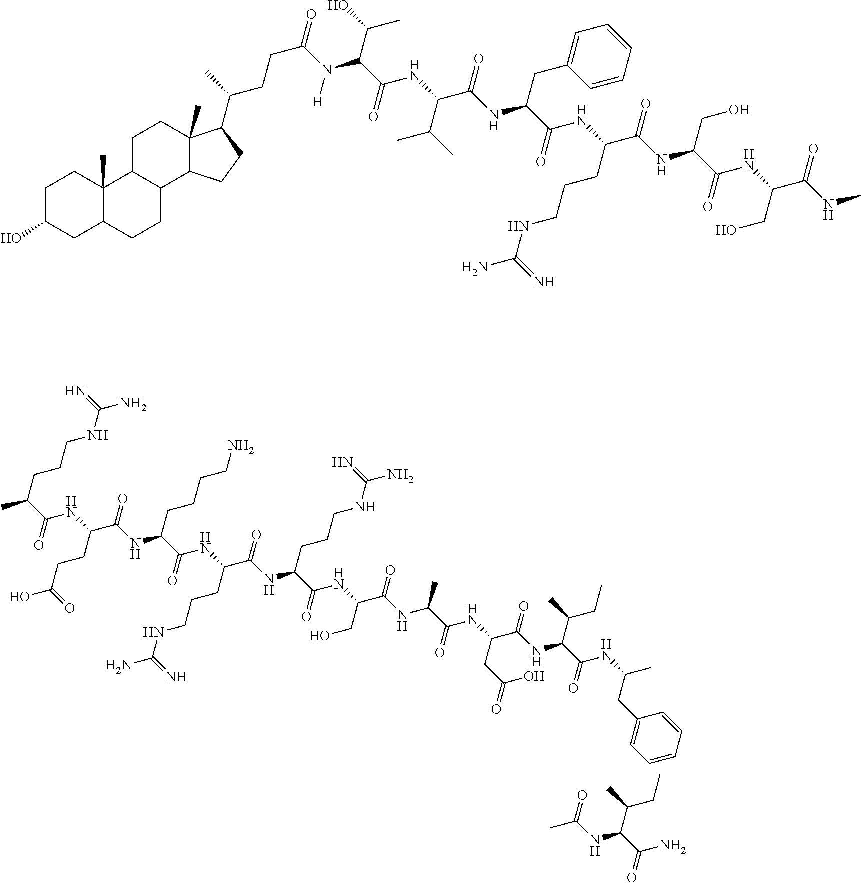 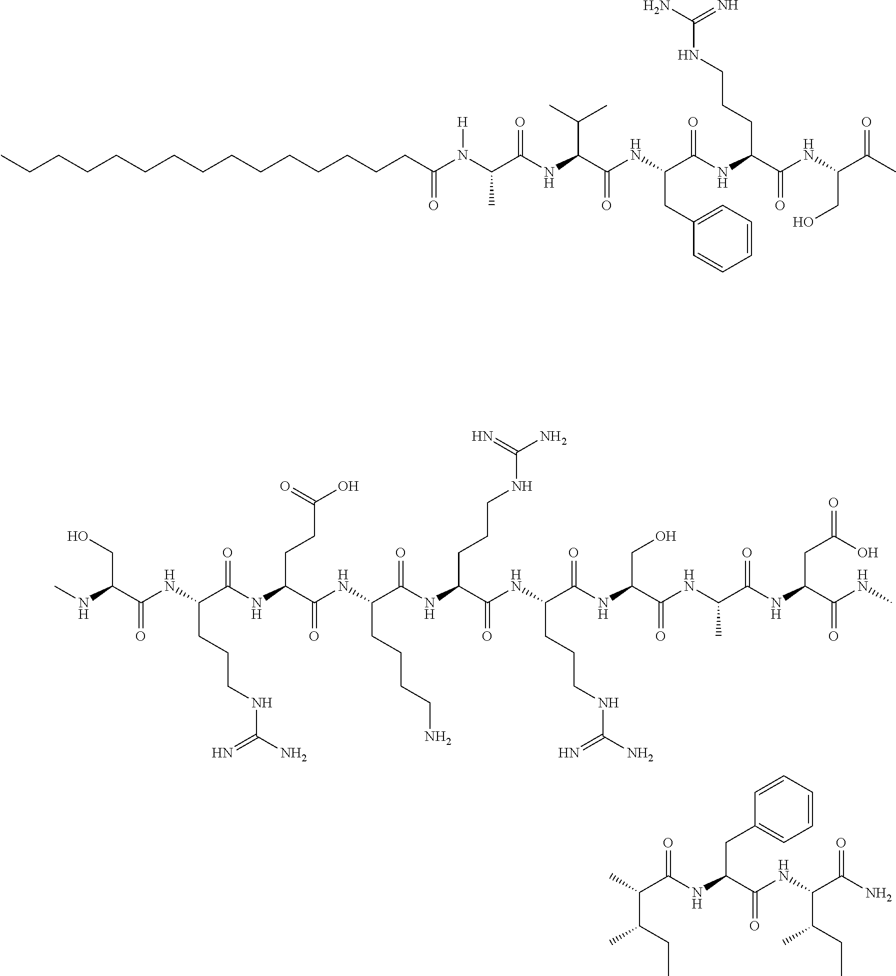 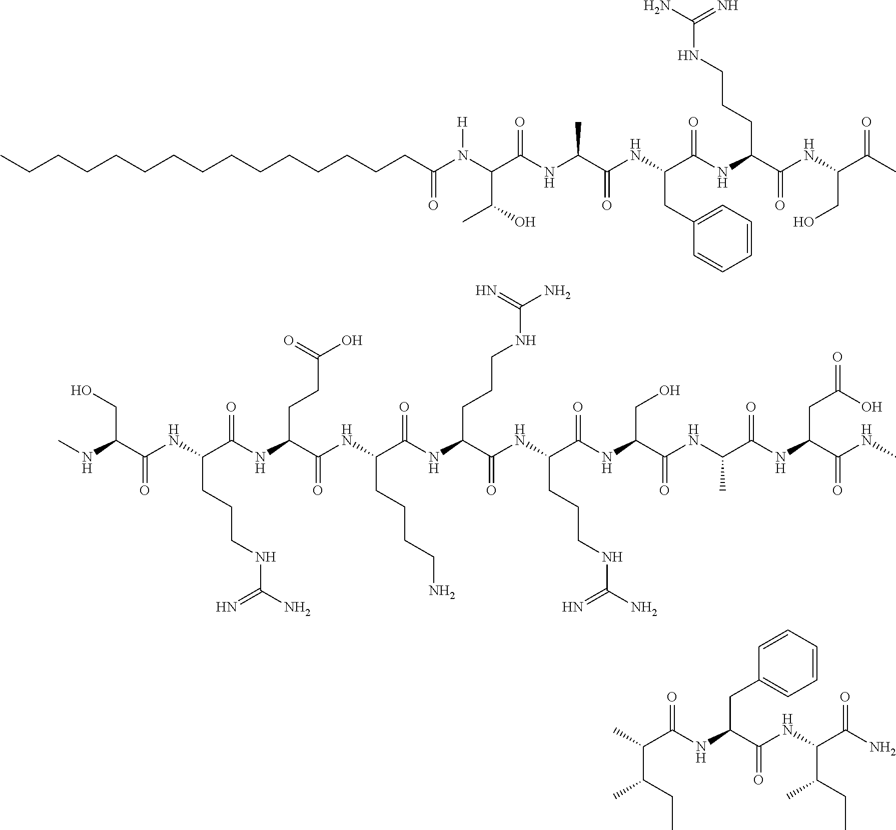 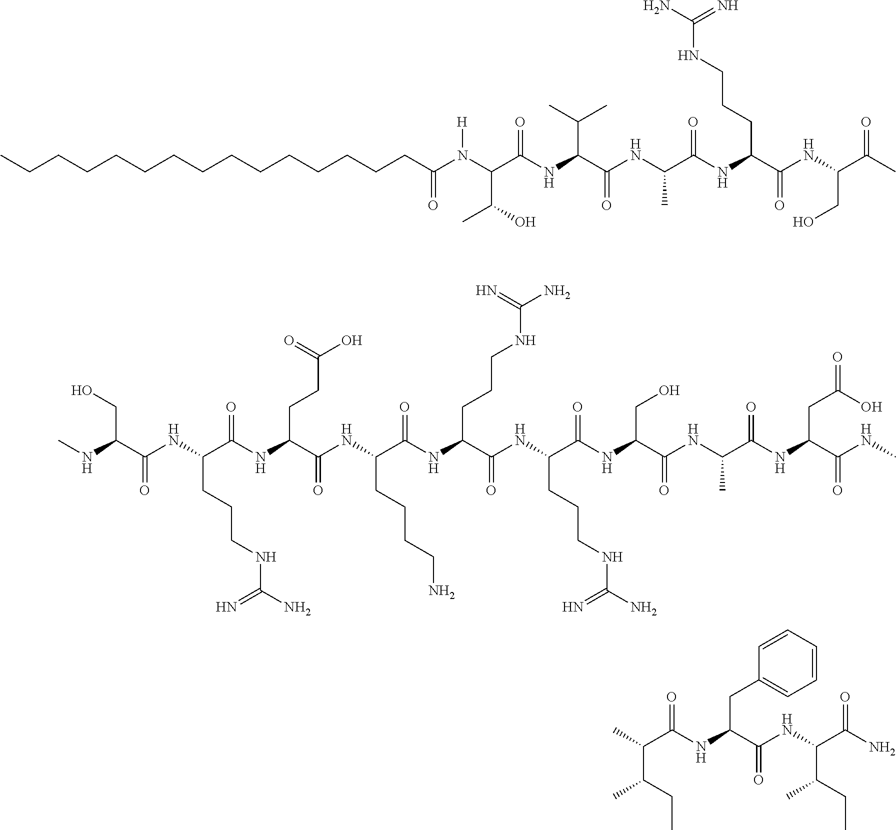 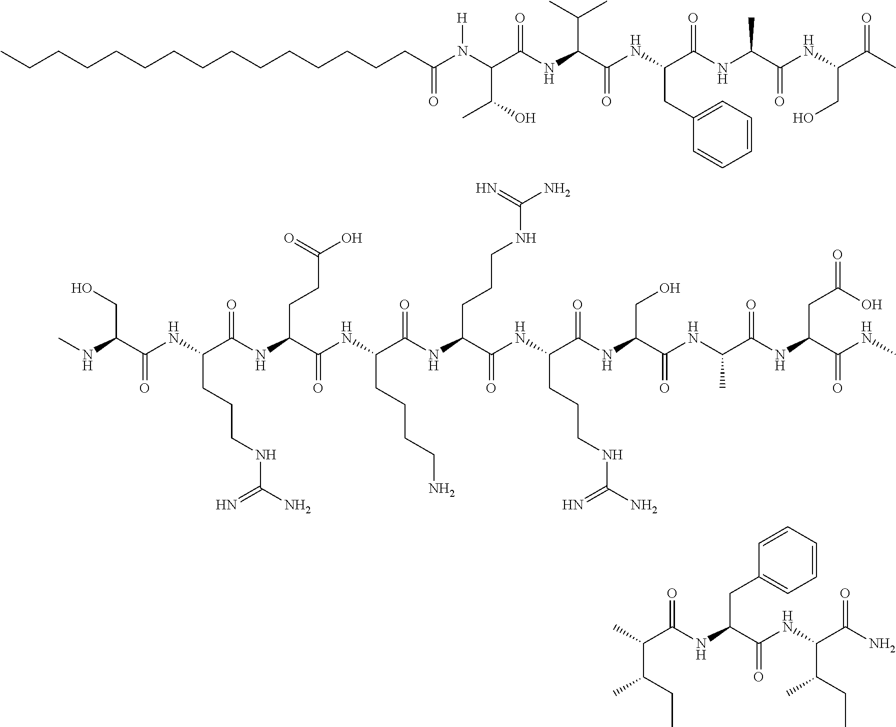 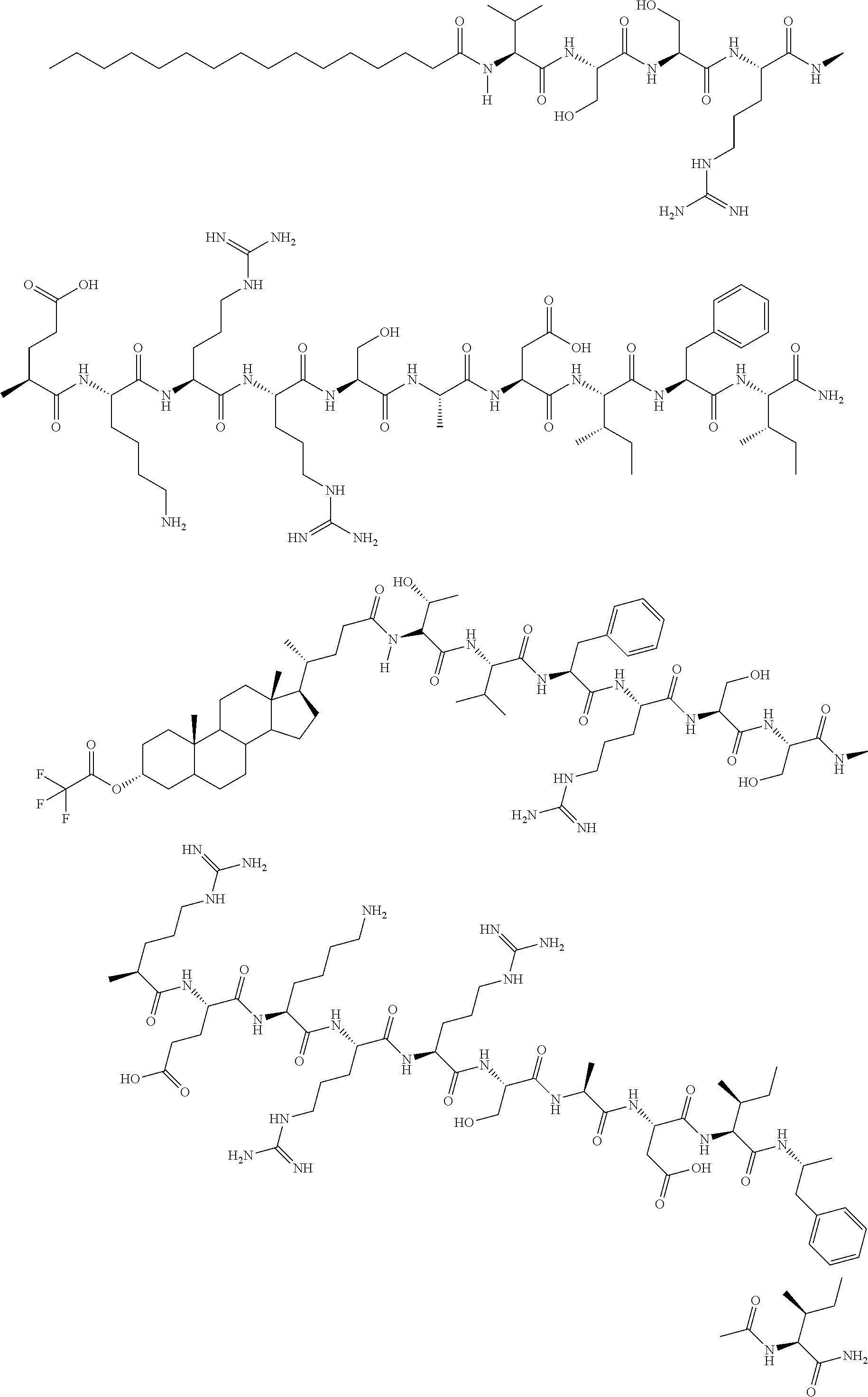 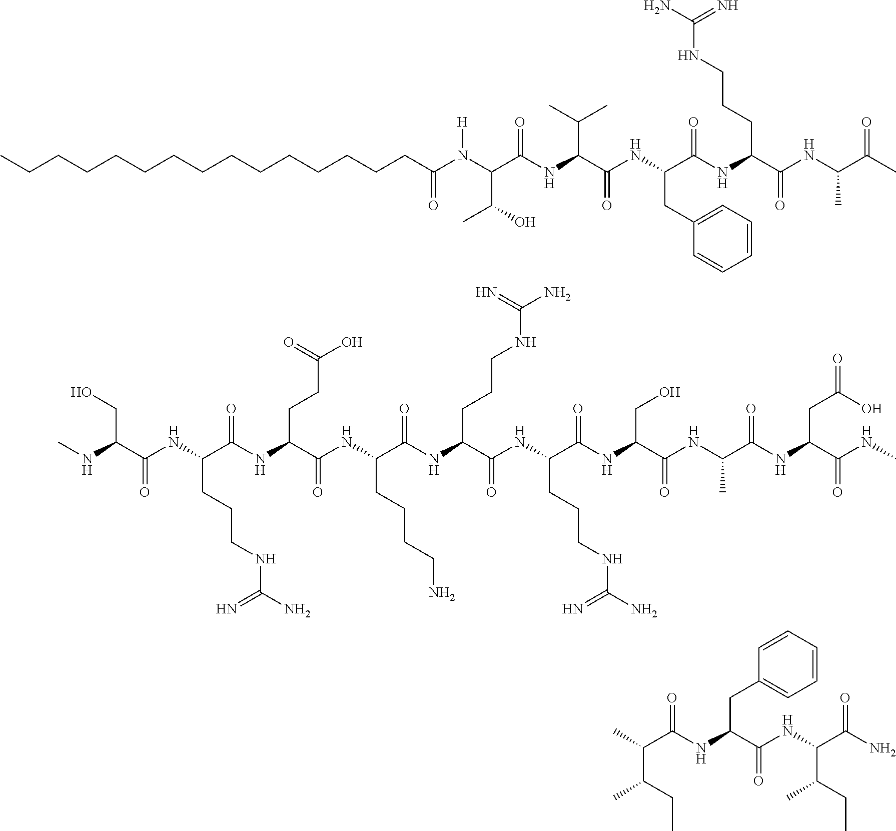 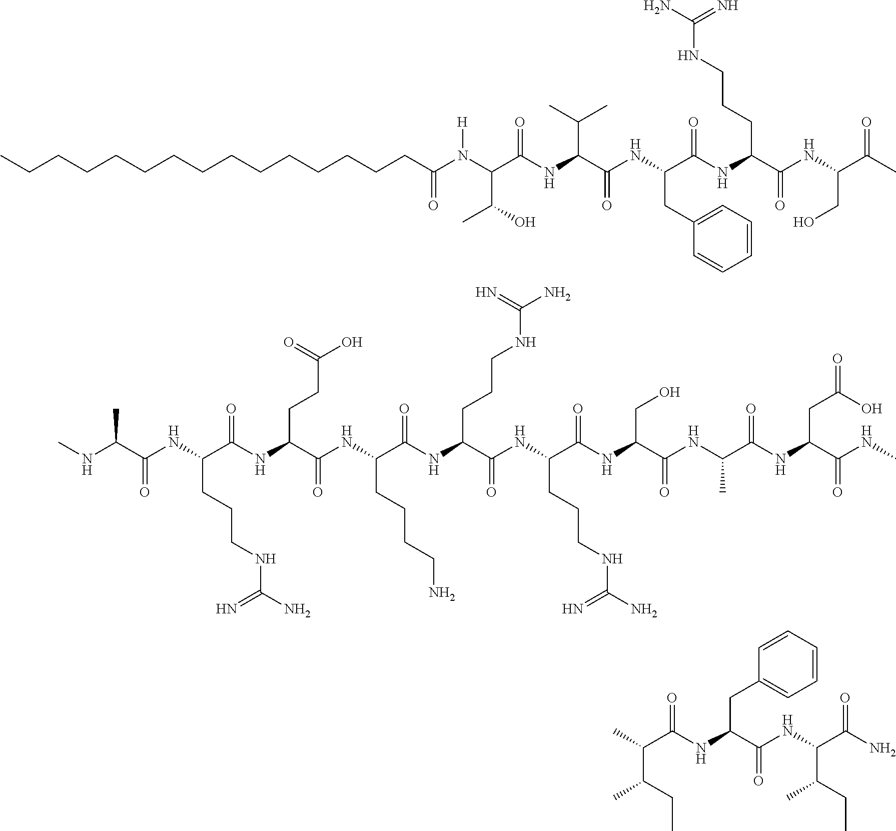 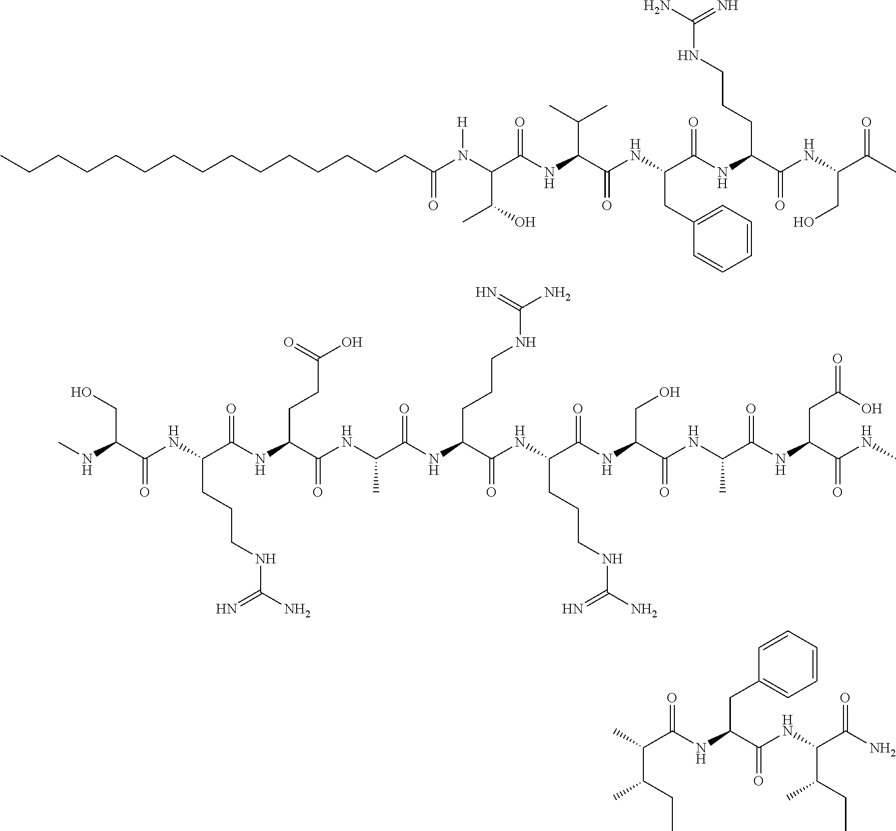 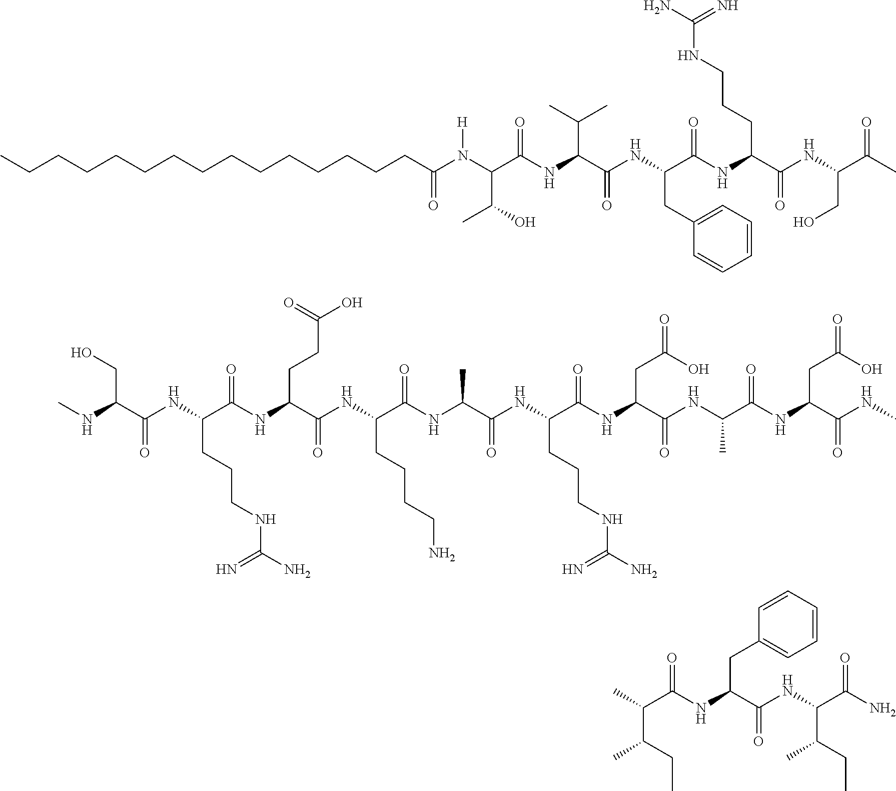 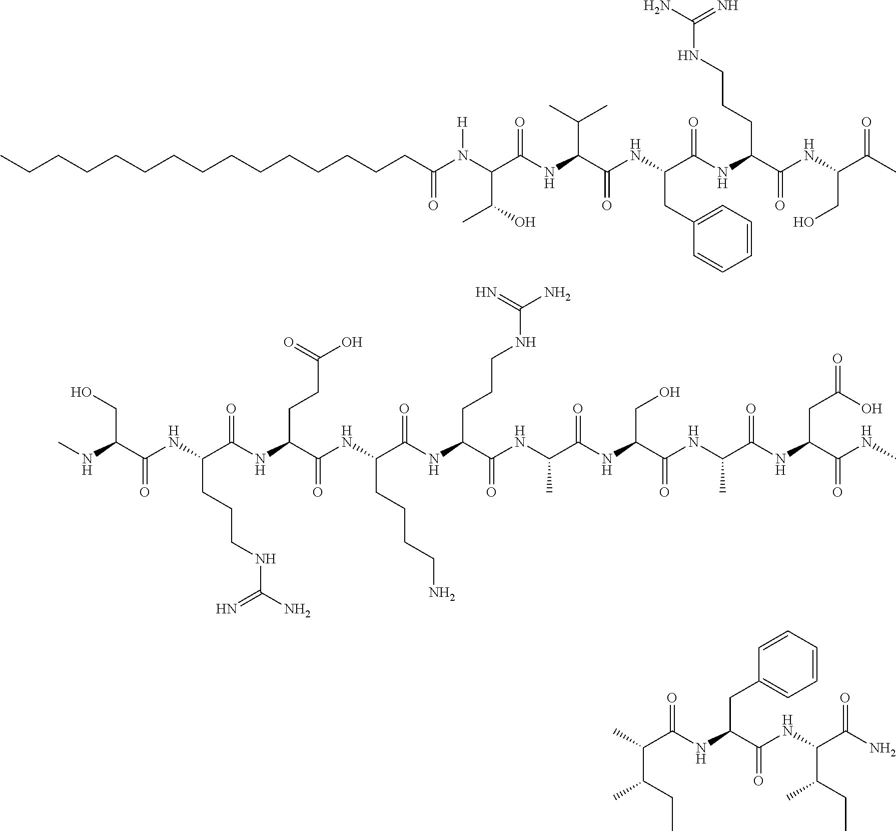 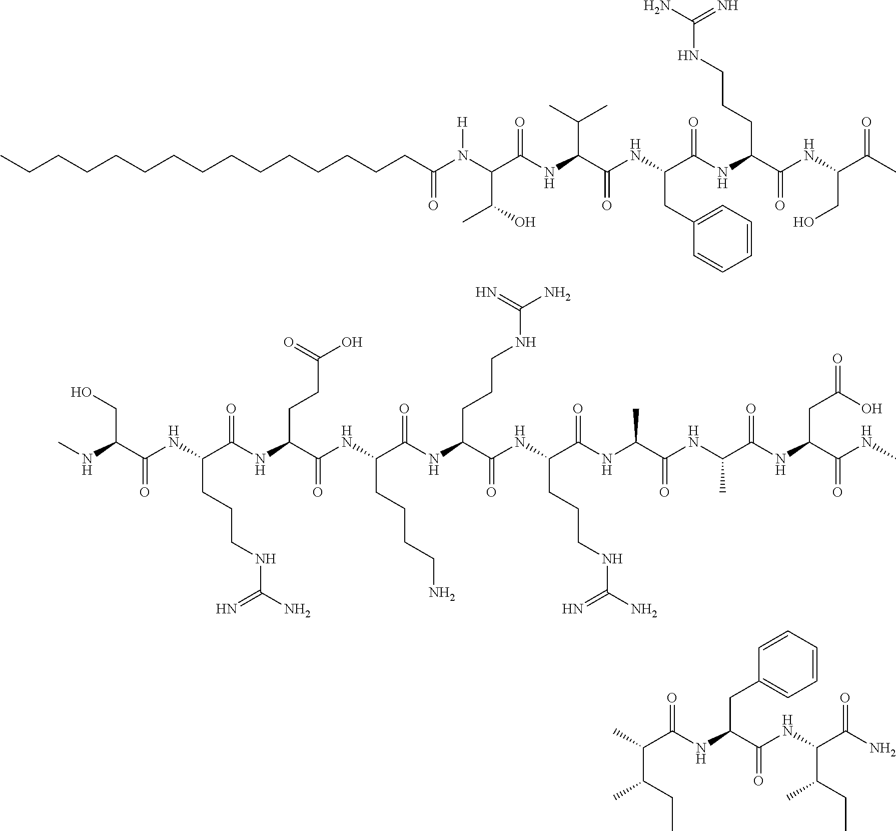 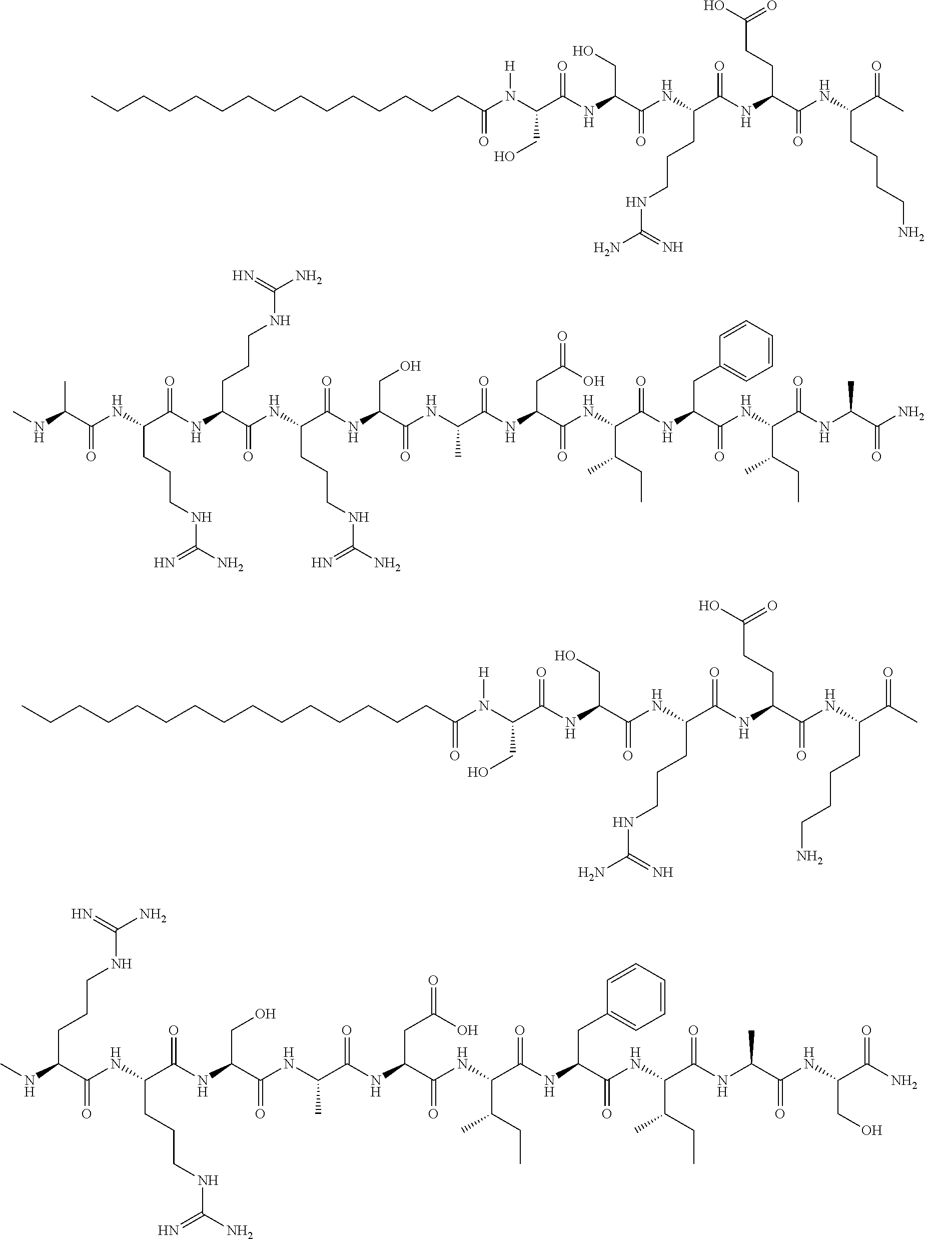 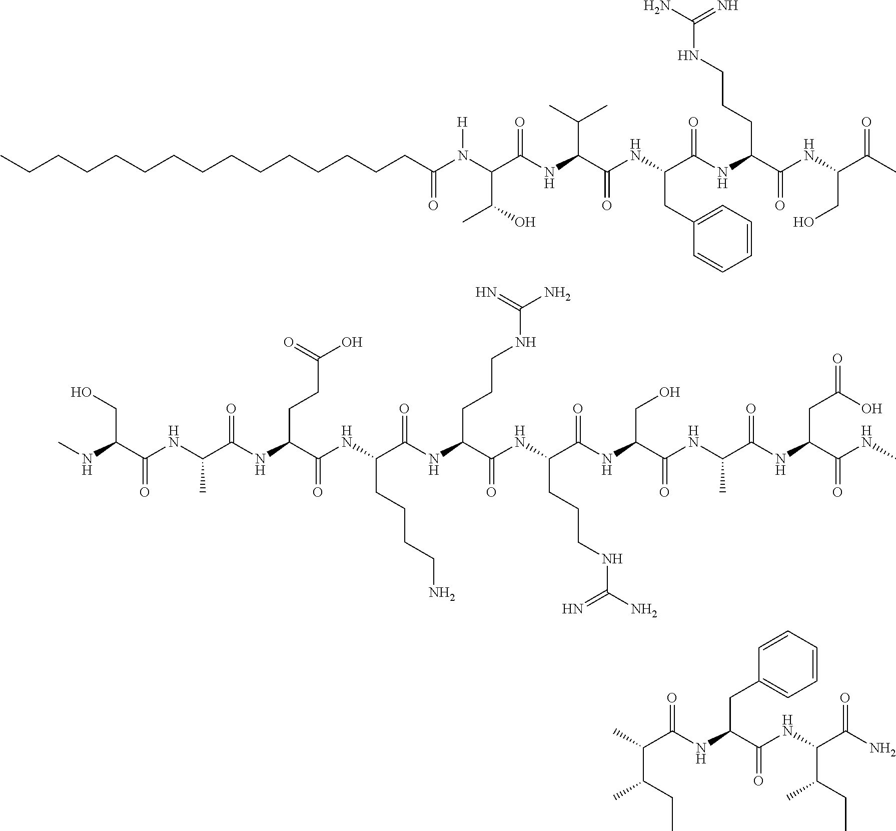 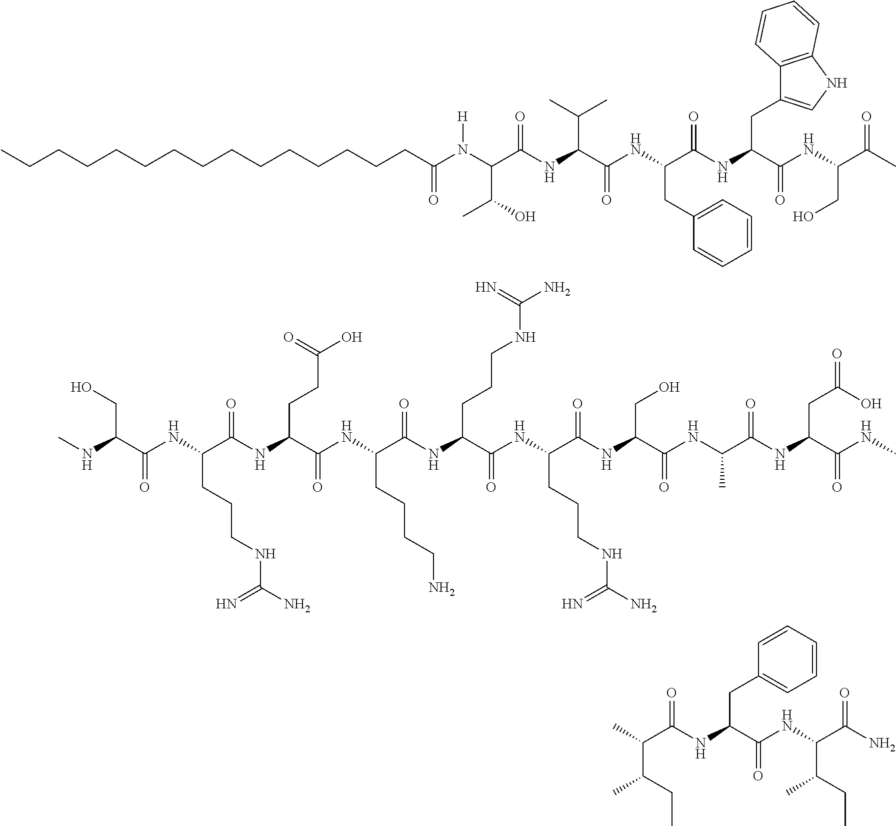 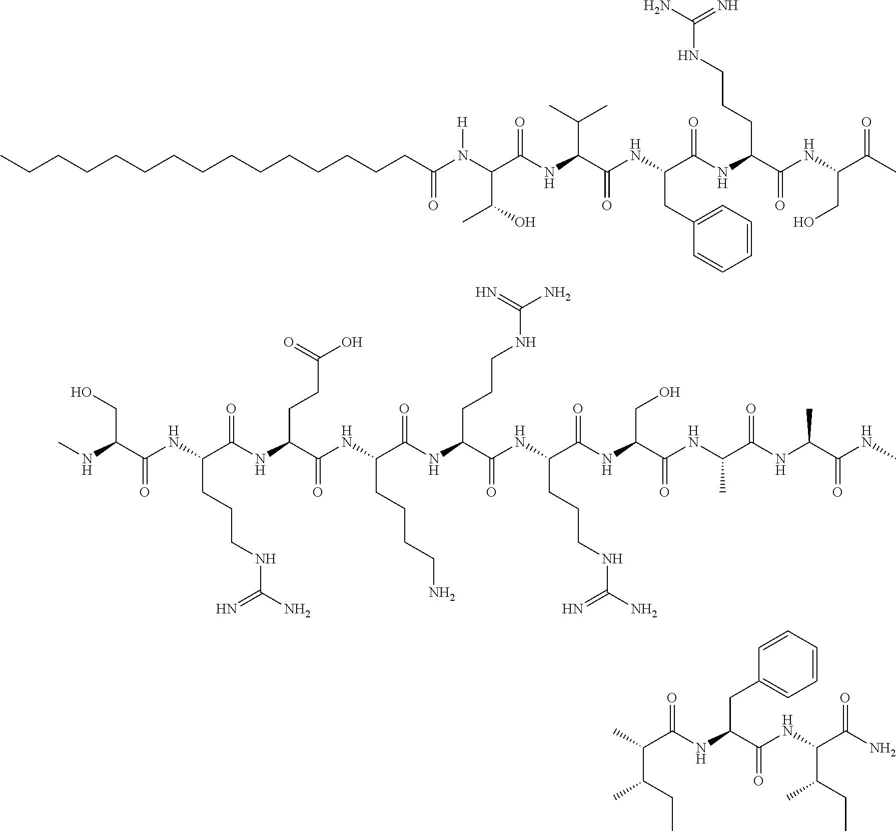 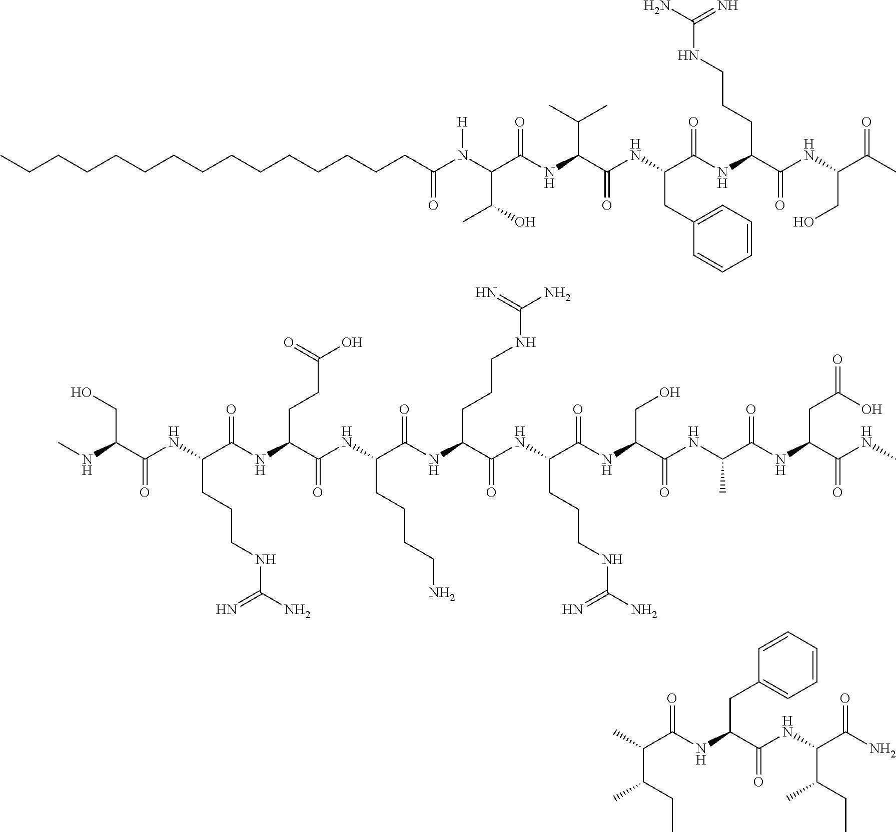 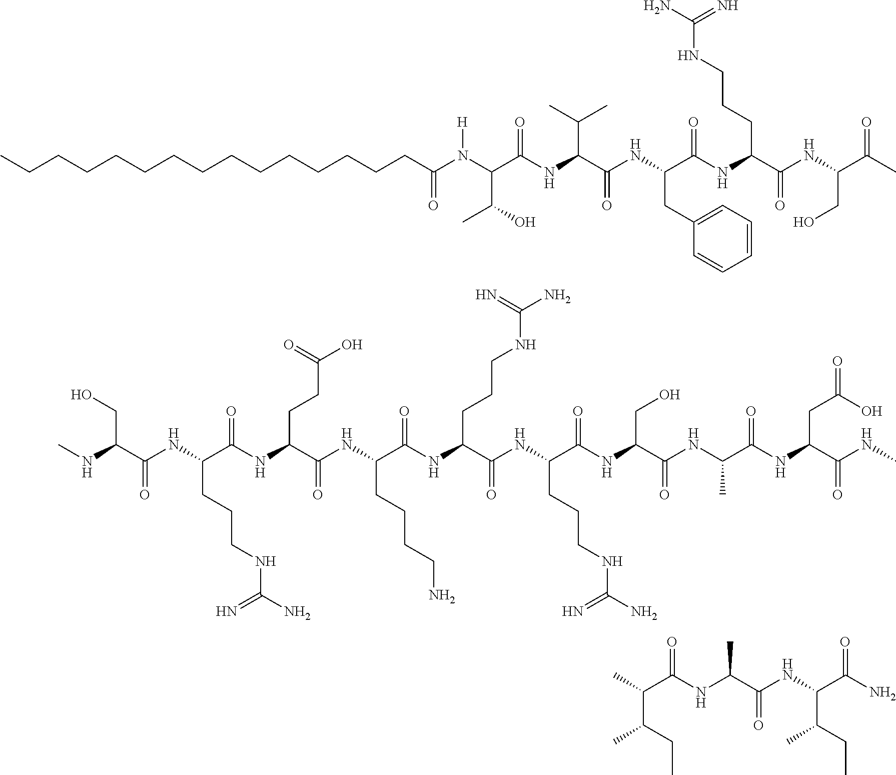 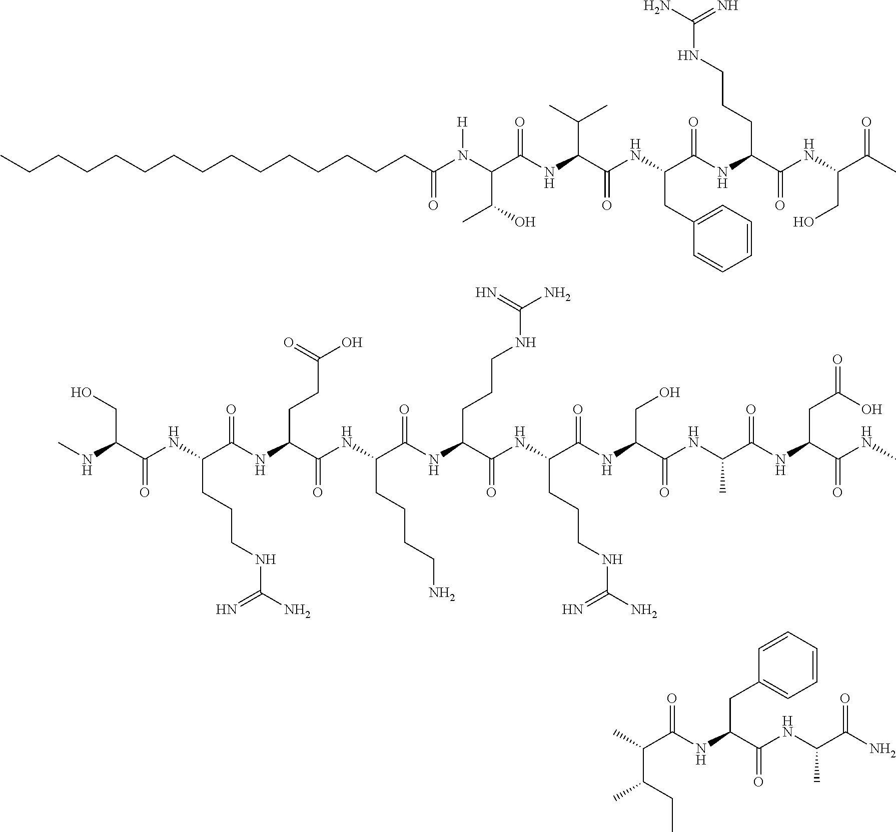 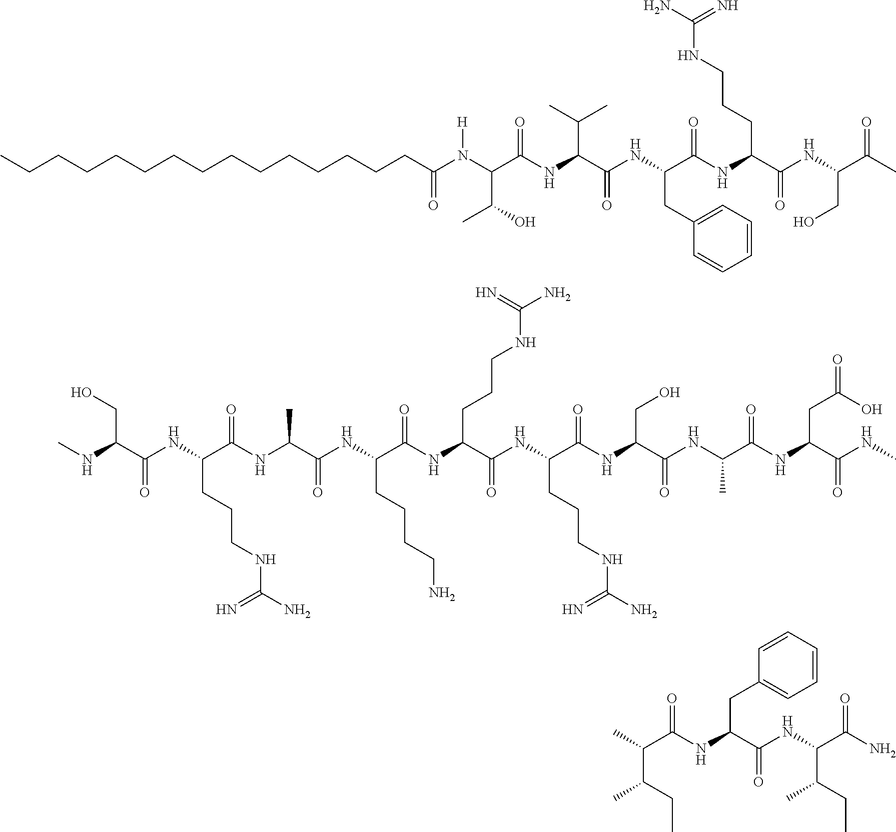 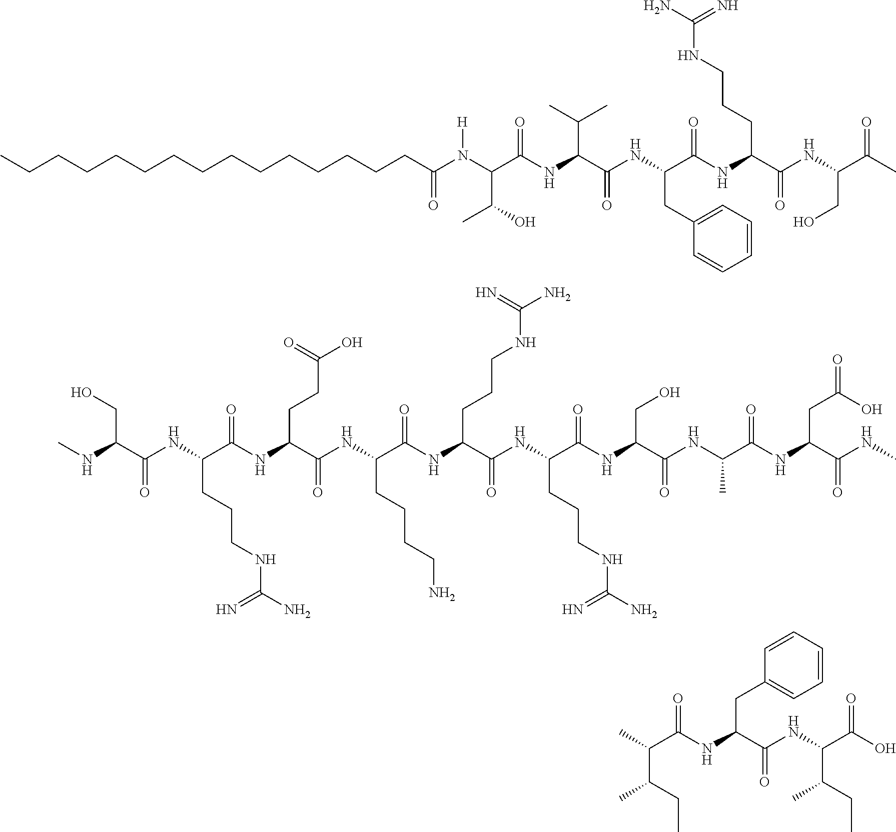 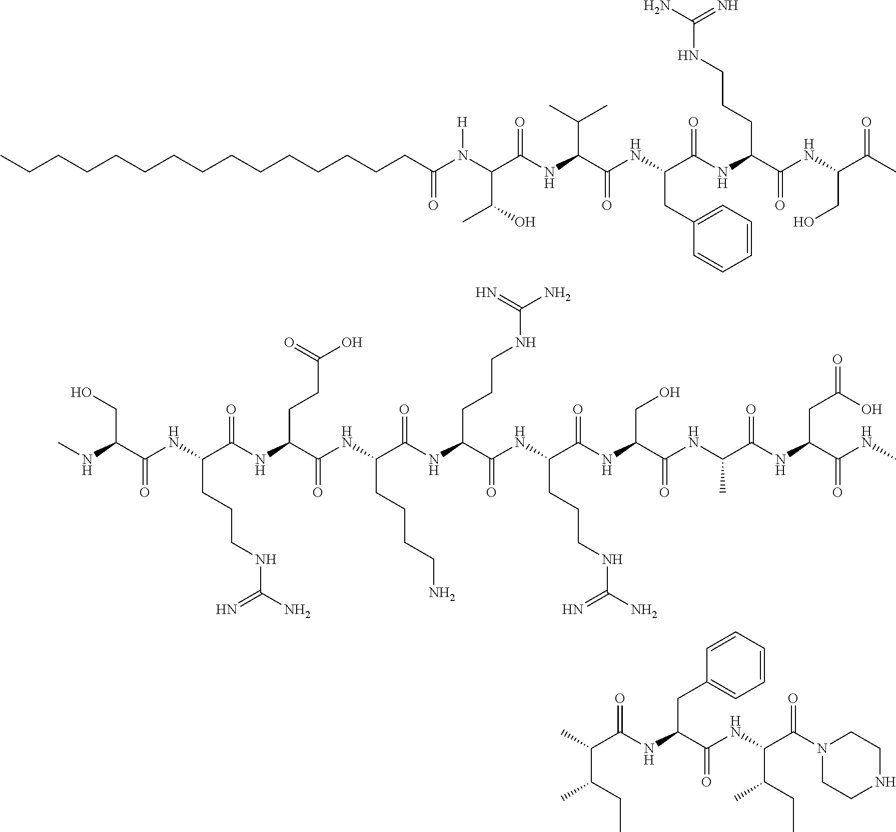 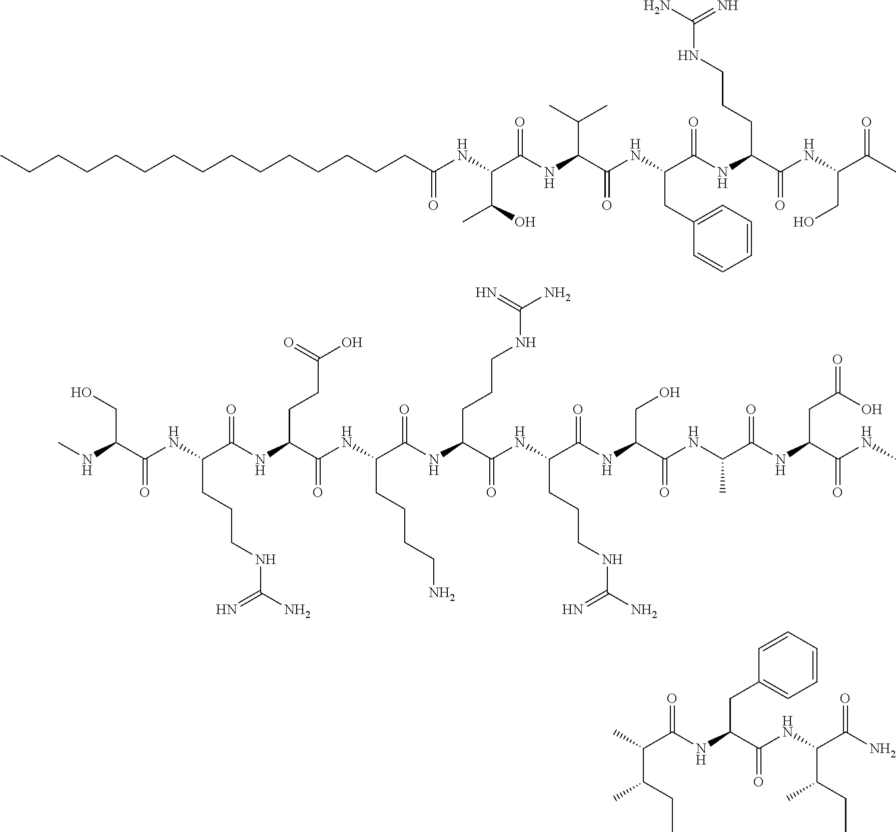 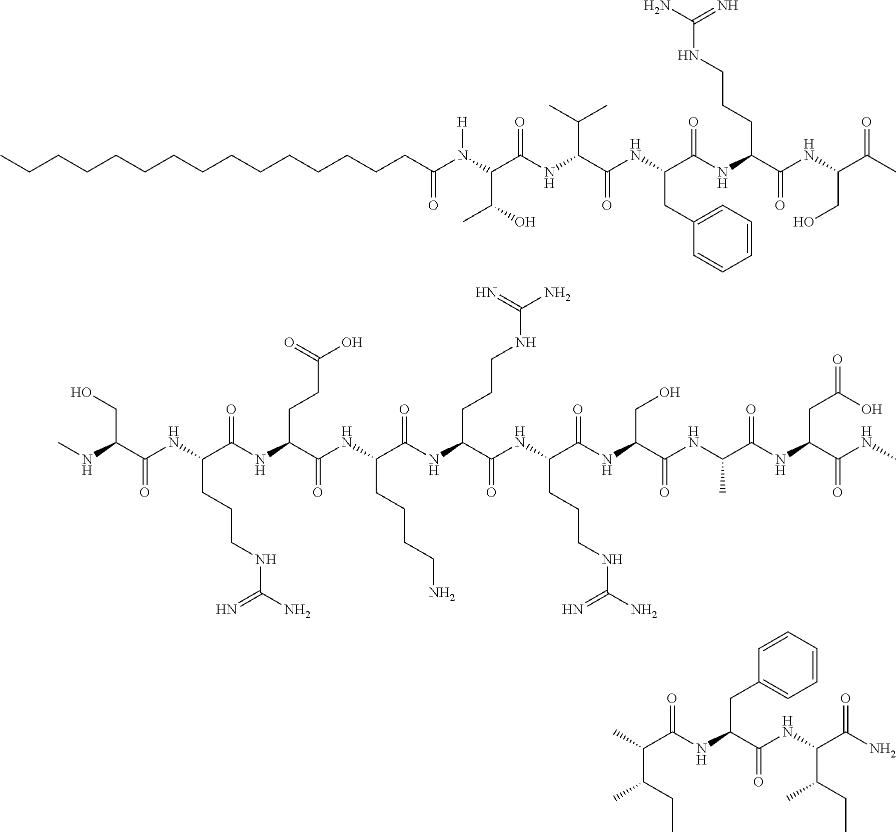 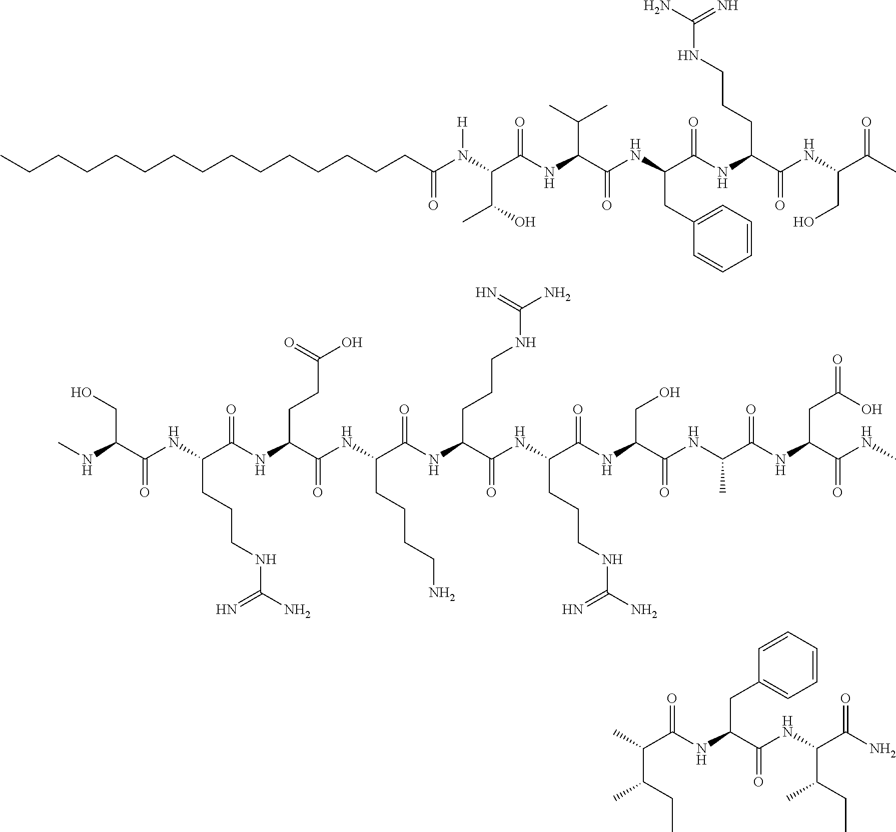 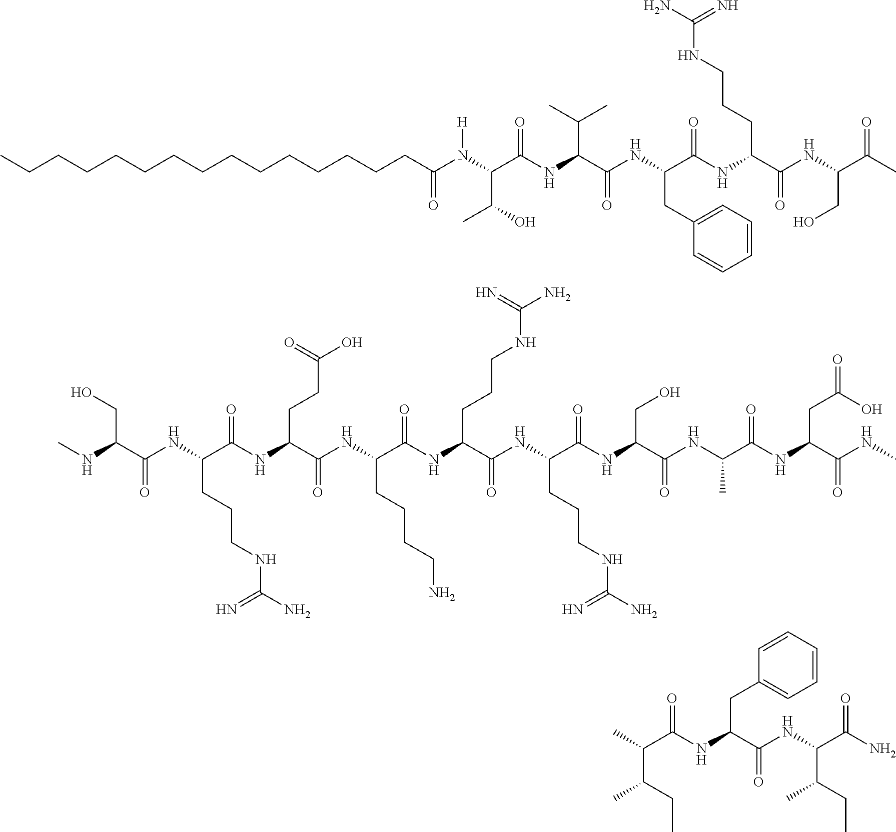 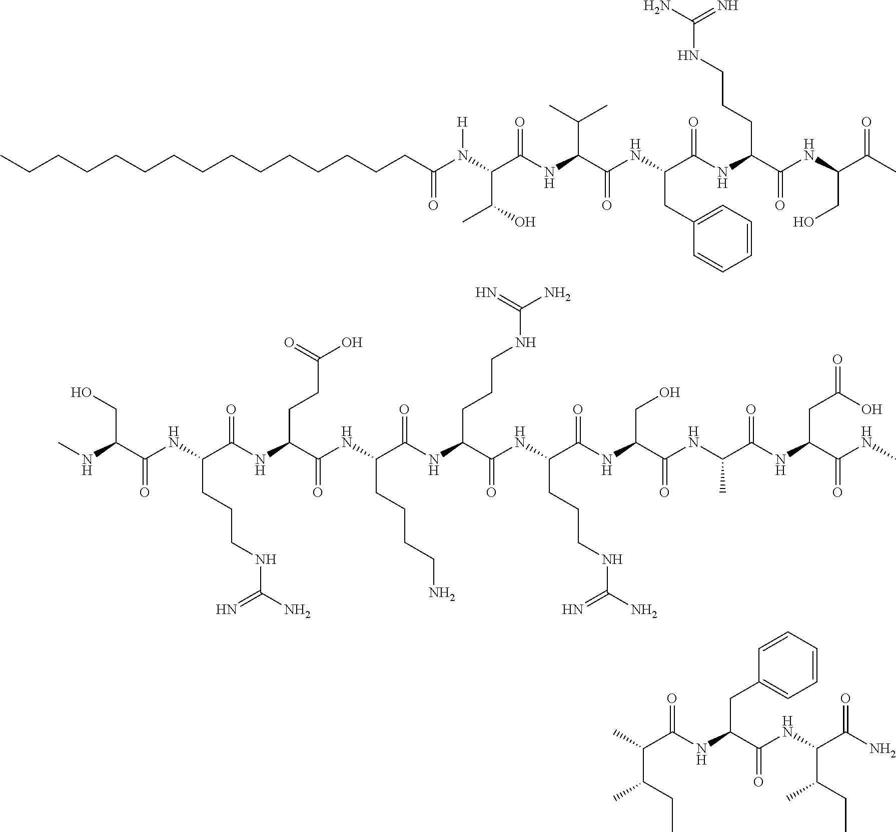 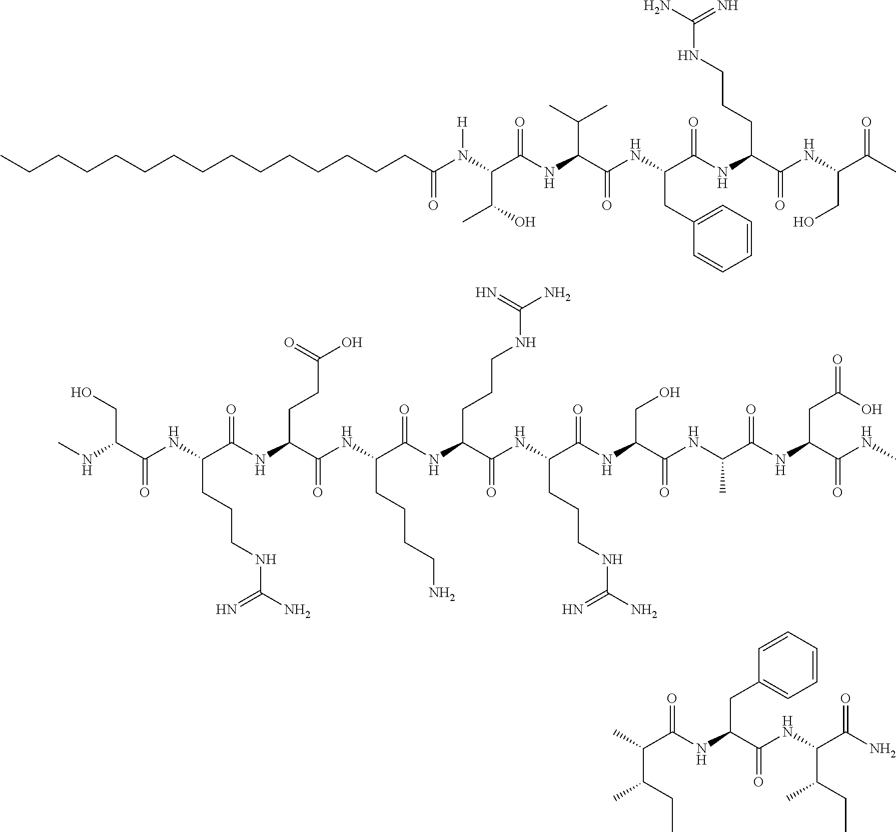 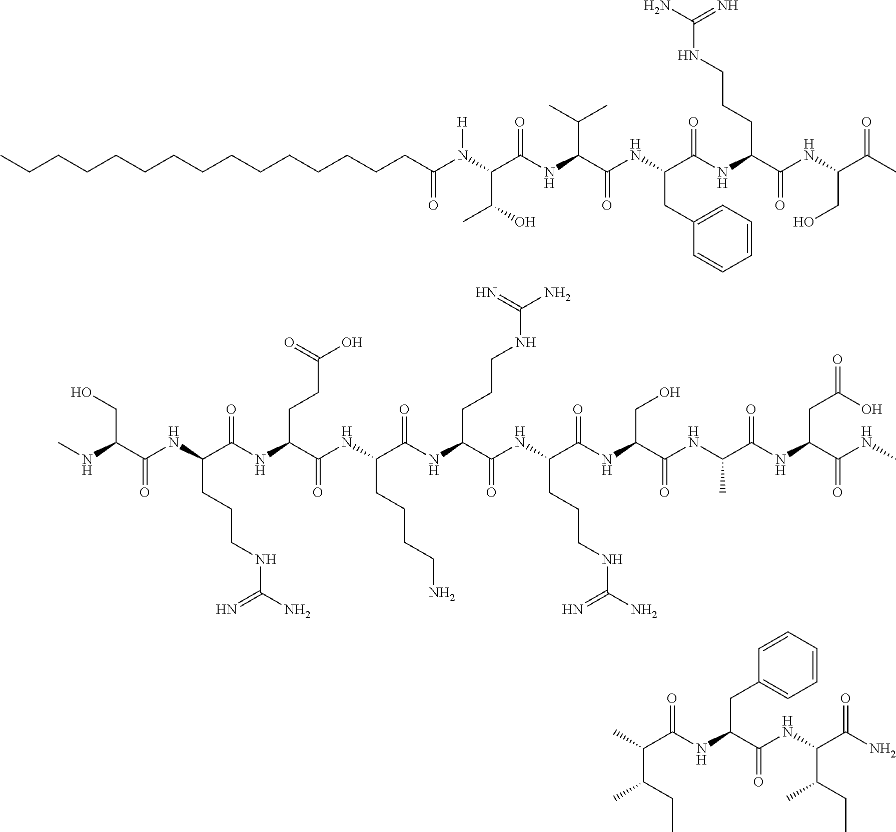 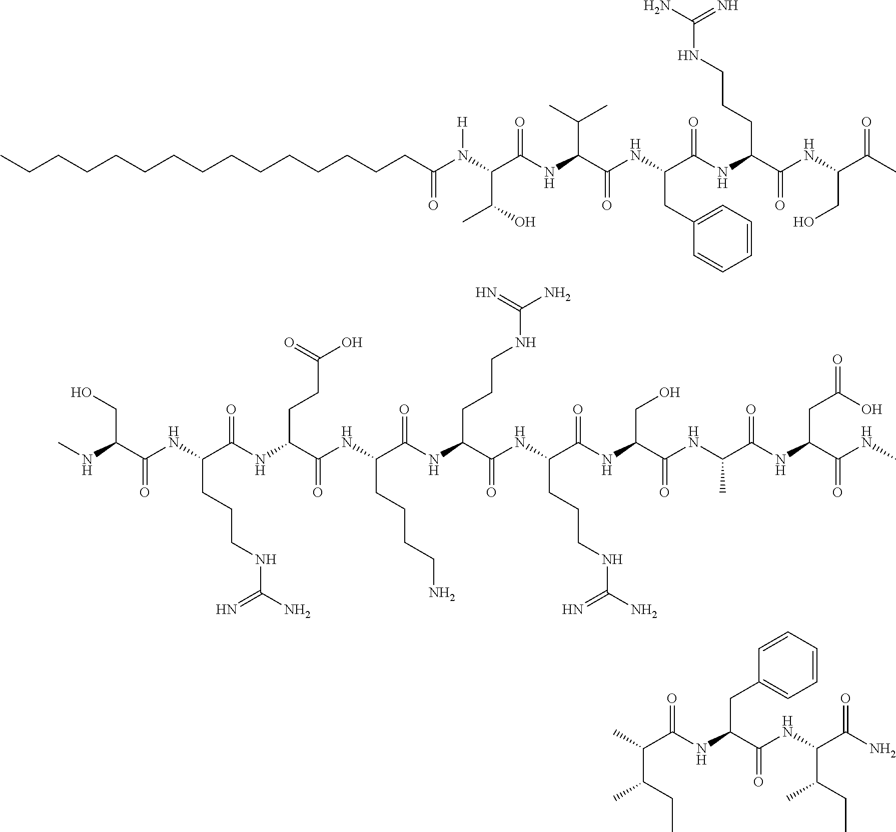 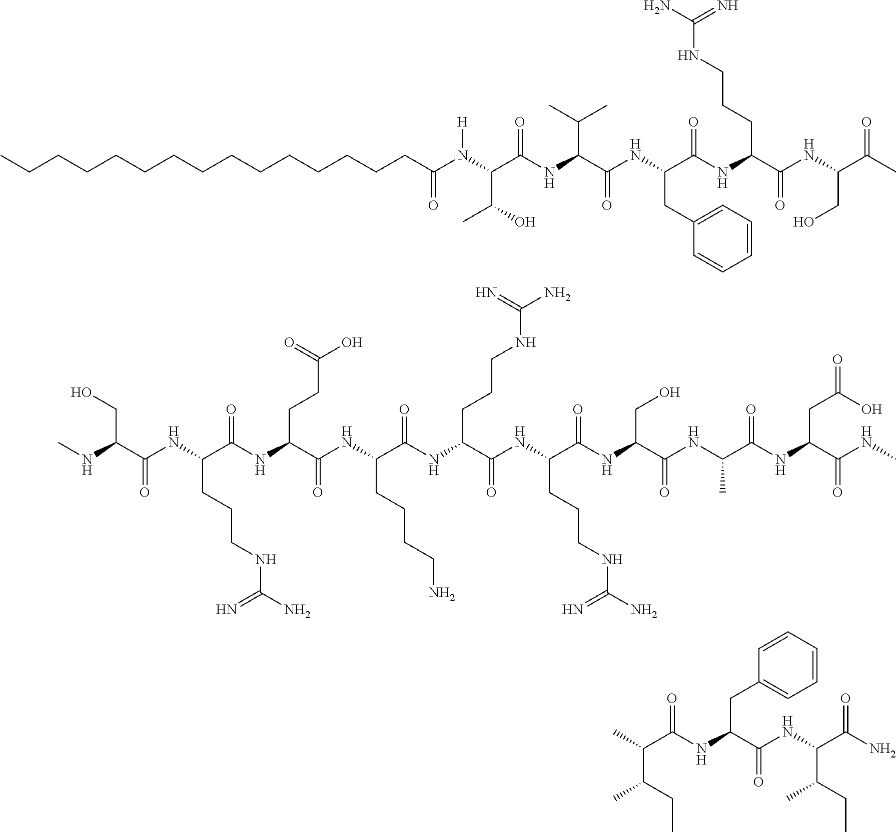 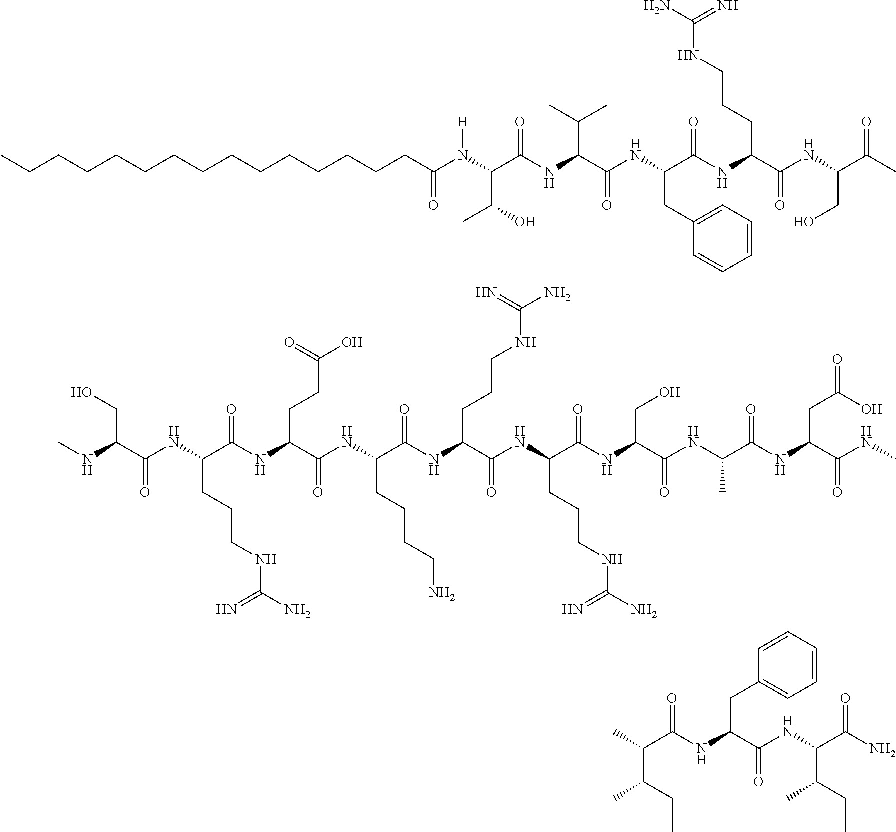 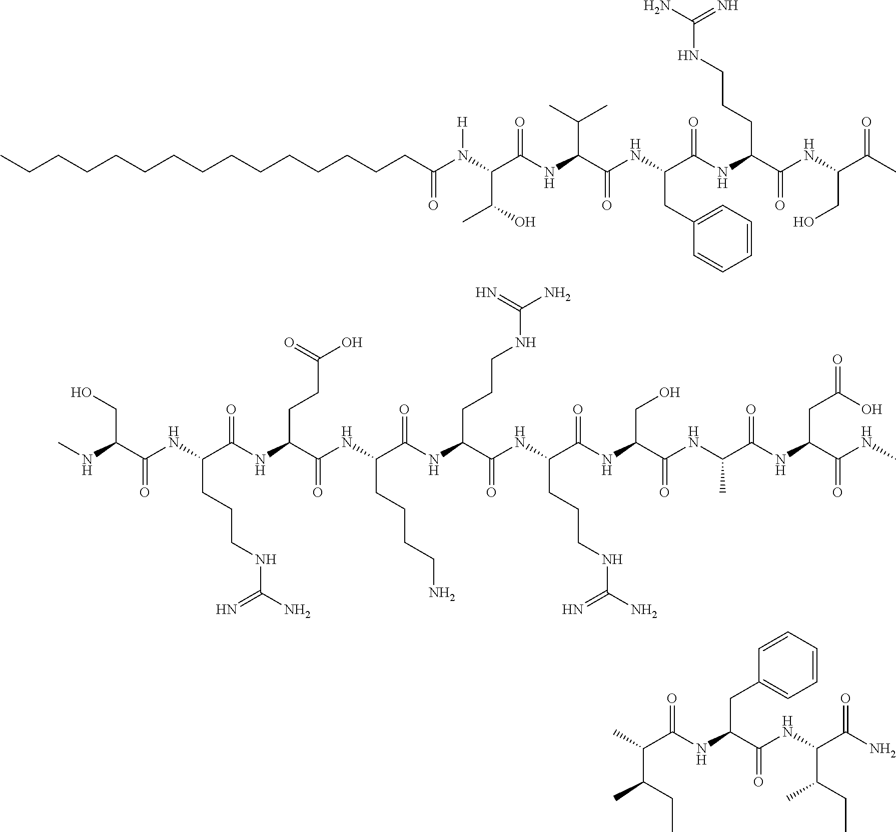 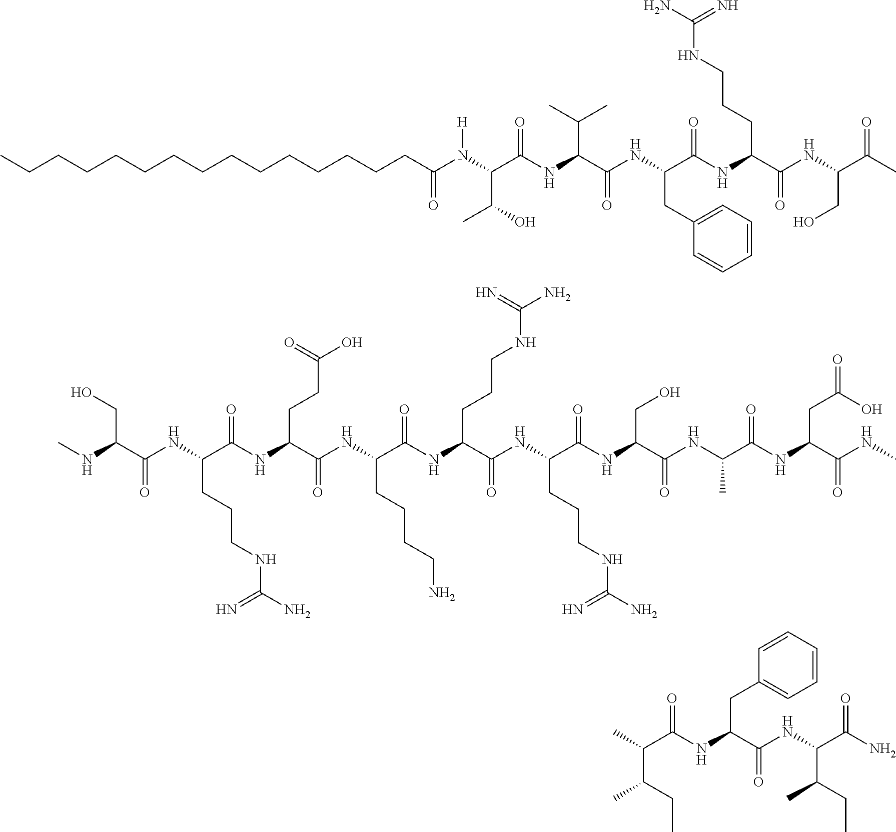 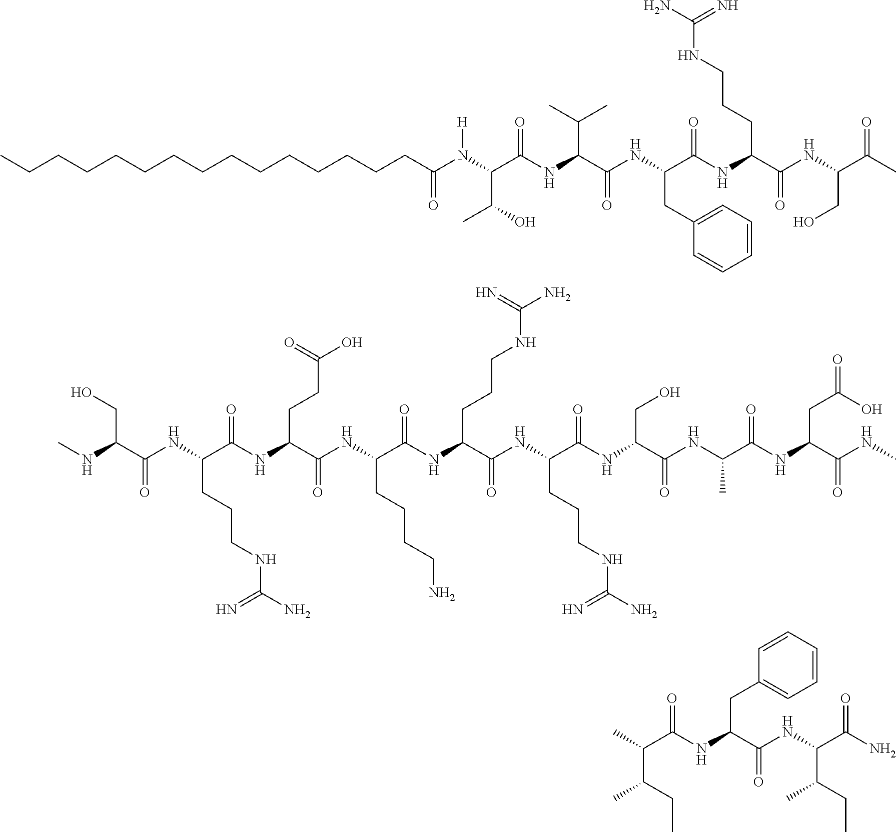 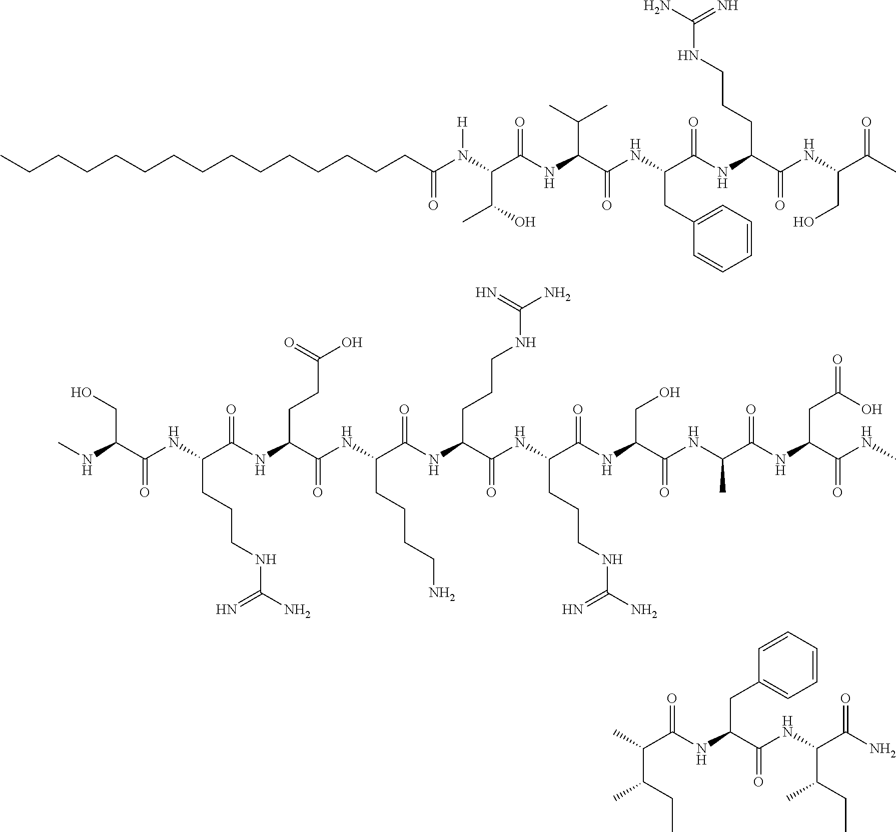 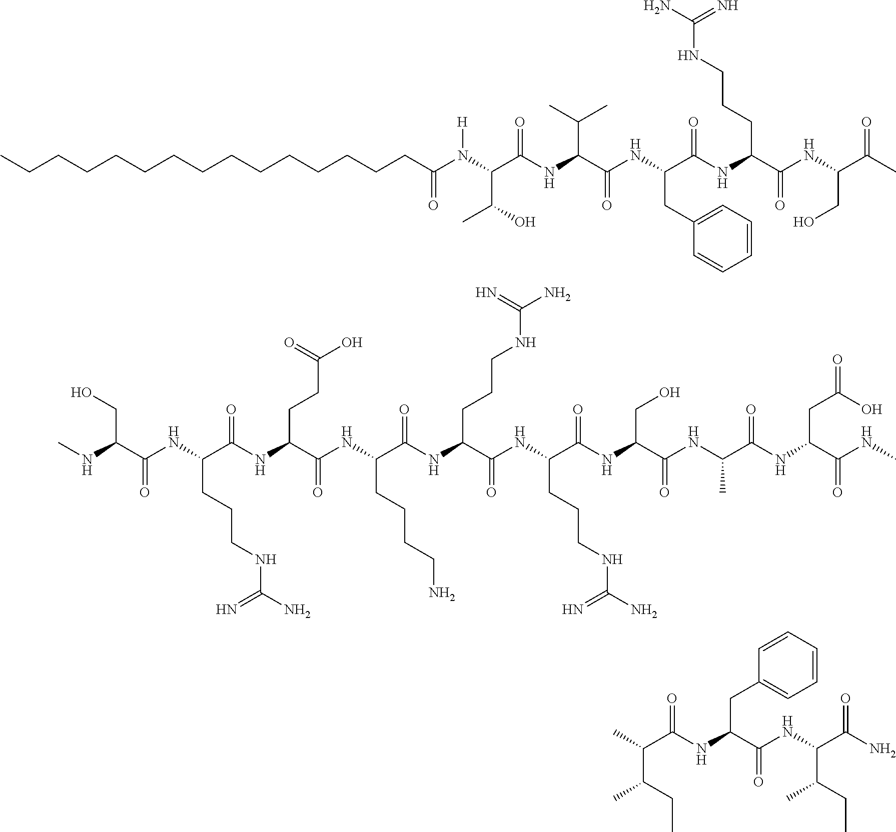 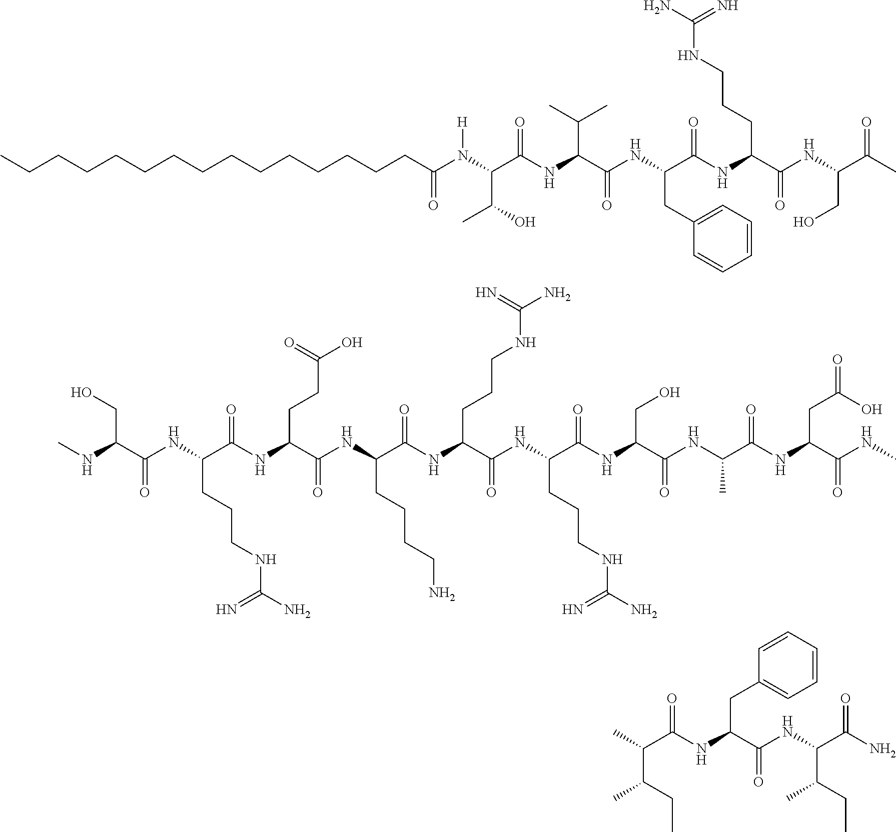 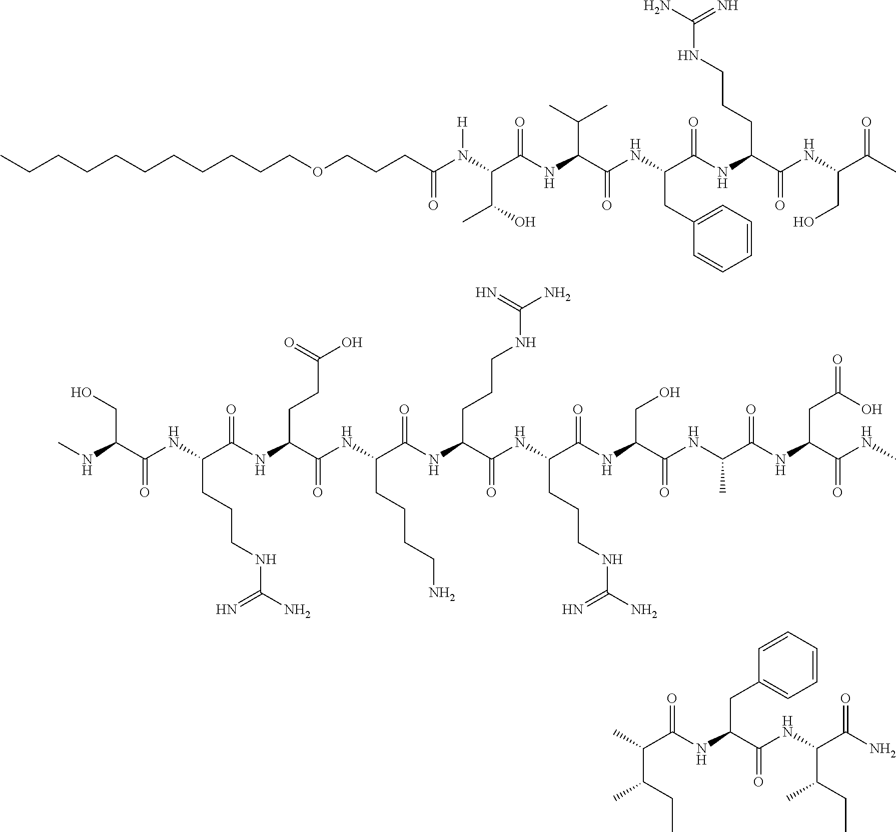 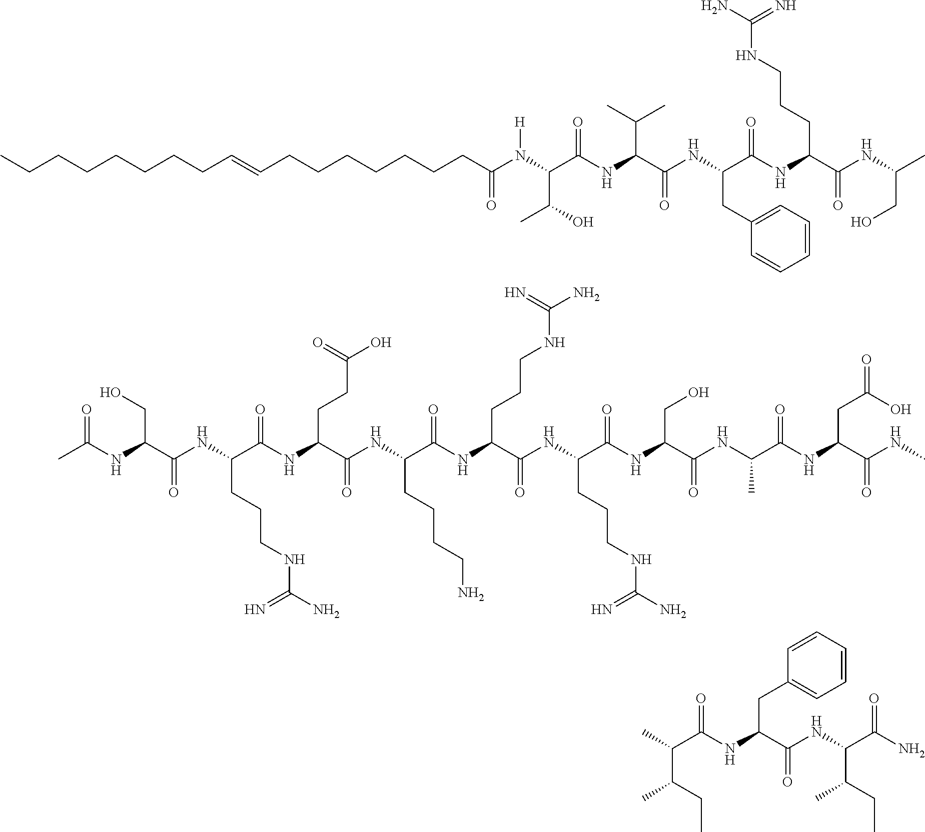 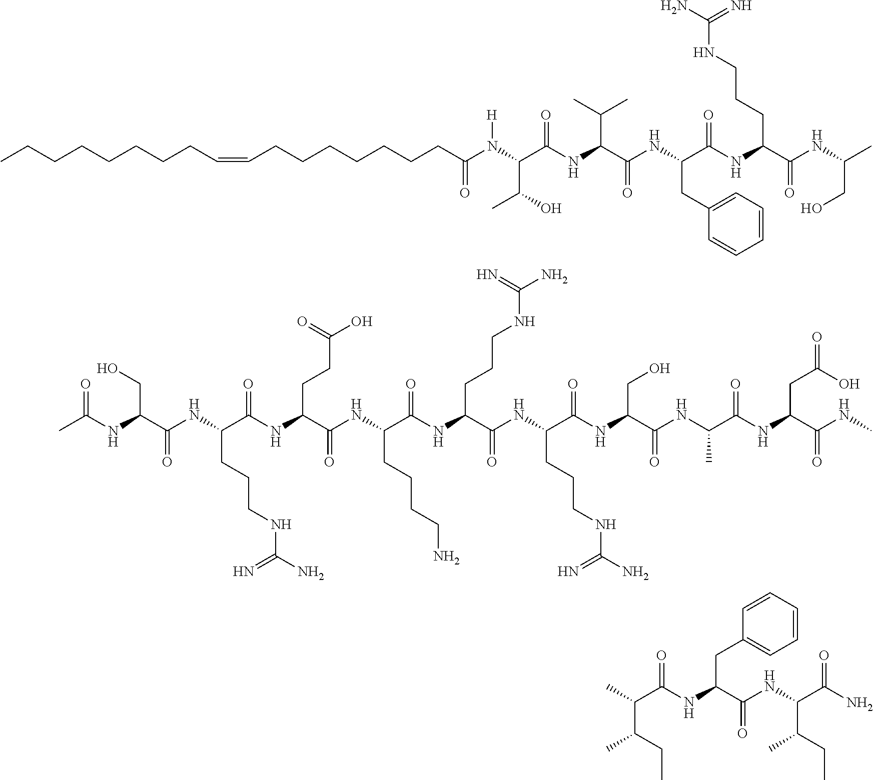 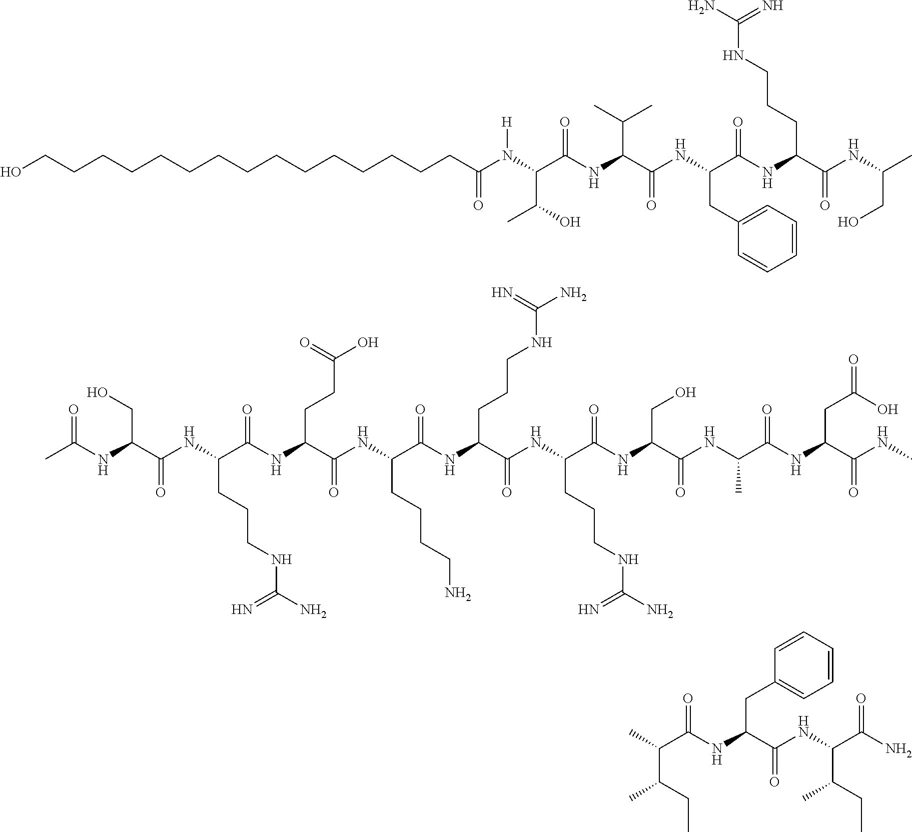 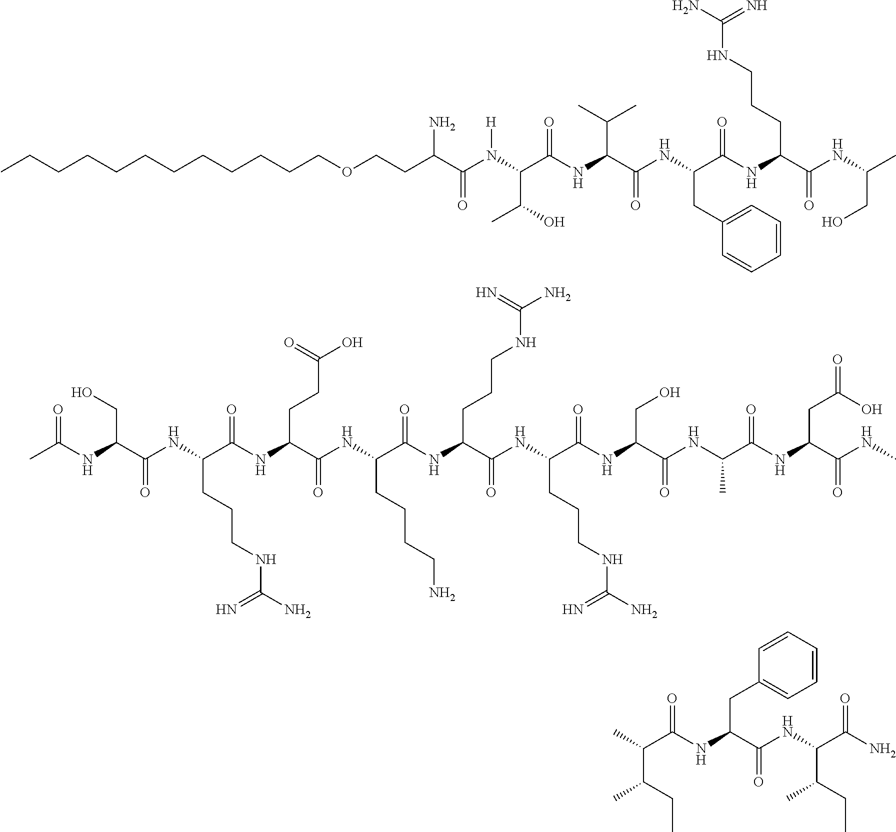 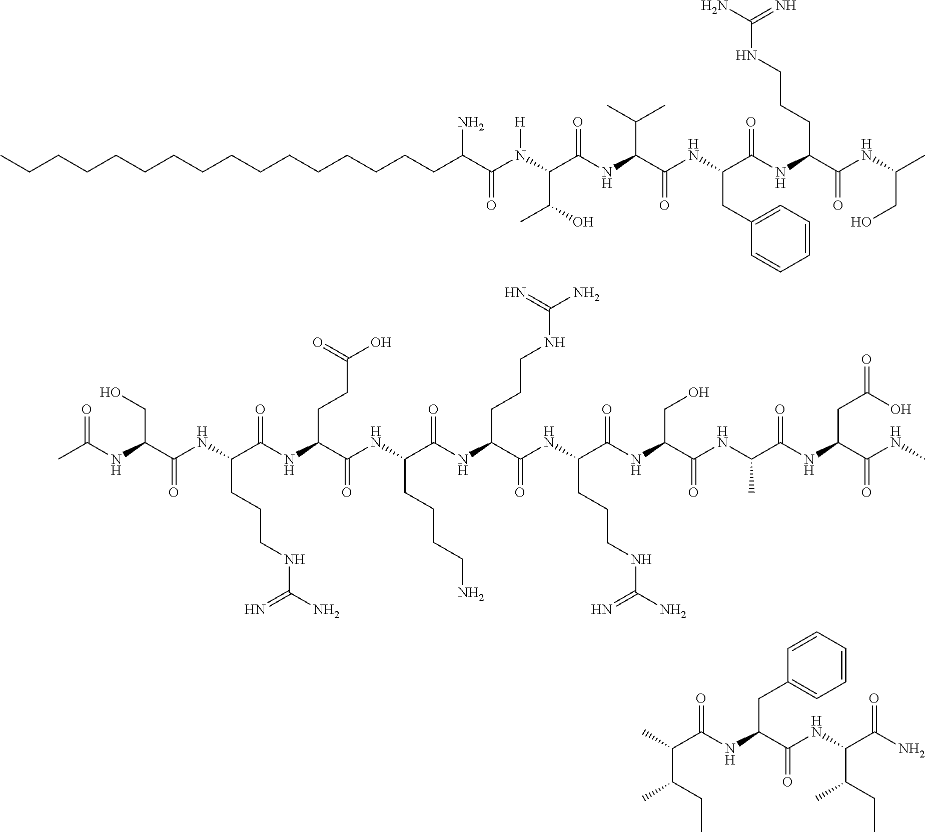 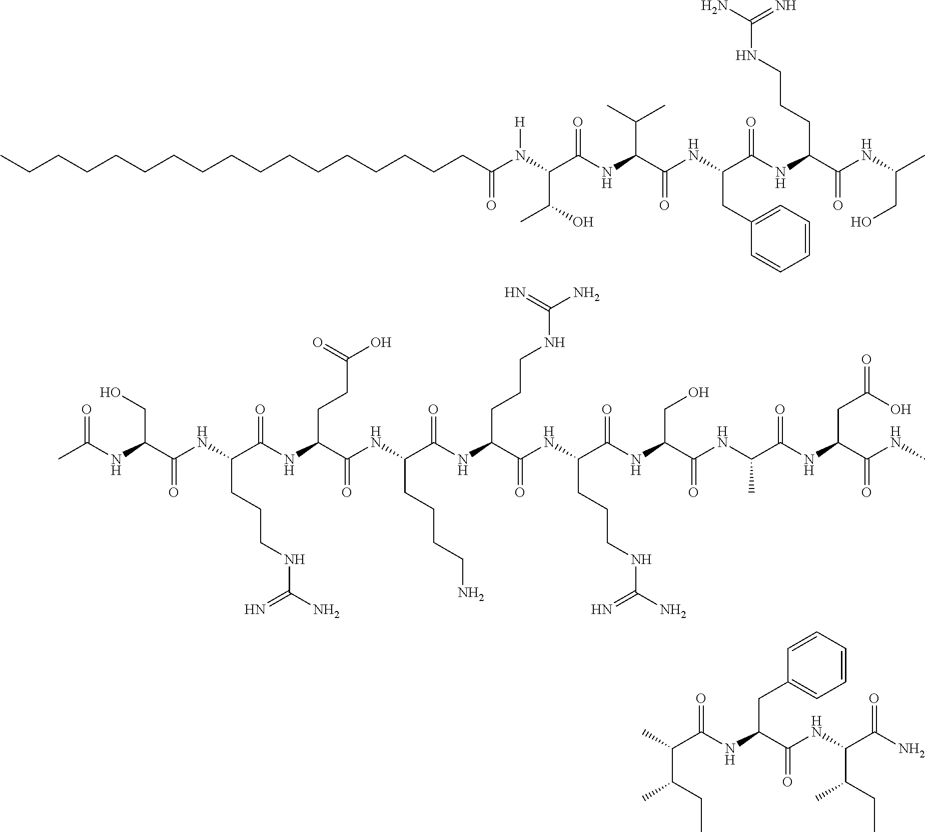 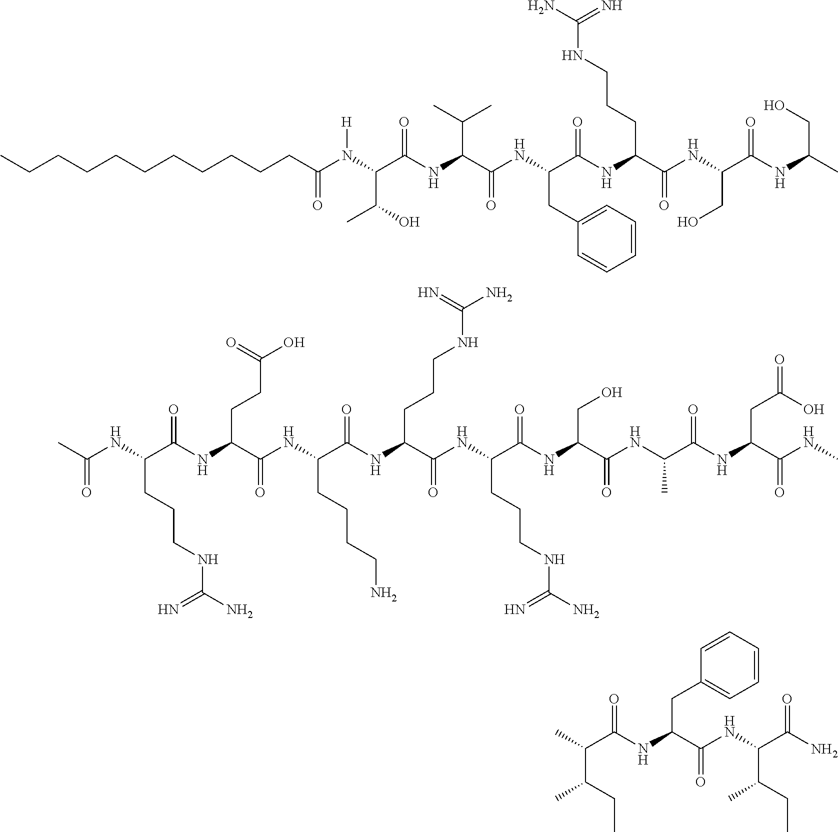 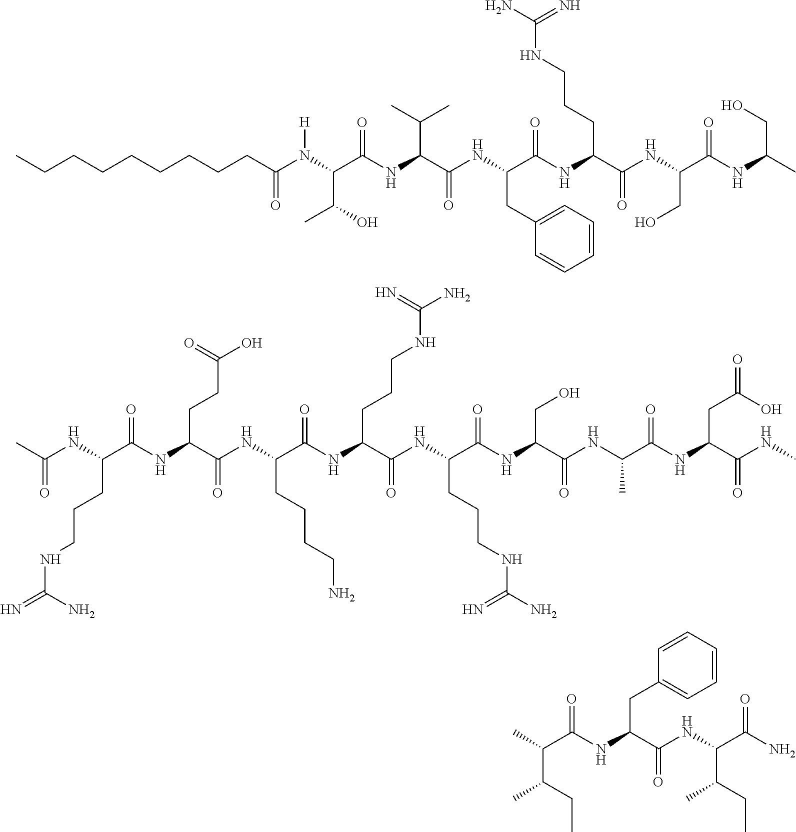
or a pharmaceutically acceptable salt of any of the foregoing.

14. The compound of claim 1, wherein P comprises at least three contiguous amino acid residues of the i1 loop.

15. The compound of claim 14, wherein P is derived from the following sequence:

| | |
|---|---|
| TVFRSSREKRRSADIFI. | (SEQ ID NO: 1) |

16. The compound of claim 15, wherein P is a sequence selected from:

| | |
|---|---|
| TVFRSSREKRRSADIFI; | (SEQ ID NO: 1) |
| AVFRSSREKRRSADIFI; | (SEQ ID NO: 2) |
| TAFRSSREKRRSADIFI; | (SEQ ID NO: 3) |
| TVARSSREKRRSADIFI; | (SEQ ID NO: 4) |
| TVFASSREKRRSADIFI; | (SEQ ID NO: 5) |
| TVFRASREKRRSADIFI; | (SEQ ID NO: 6) |
| TVFRSAREKRRSADIFI; | (SEQ ID NO: 7) |
| TVFRSSAEKRRSADIFI; | (SEQ ID NO: 8) |
| TVFRSSRAKRRSADIFI; | (SEQ ID NO: 9) |
| TVFRSSREARRSADIFI; | (SEQ ID NO: 10) |
| TVFRSSREKARDADIFI; | (SEQ ID NO: 11) |
| TVFRSSREKRASADIFI; | (SEQ ID NO: 12) |
| TVFRSSREKRRAADIFI; | (SEQ ID NO: 13) |
| TVFRSSREKRRSAAIFI; | (SEQ ID NO: 14) |
| TVFRSSREKRRSADAFI; | (SEQ ID NO: 15) |
| TVFRSSREKRRSADIAI; | (SEQ ID NO: 16) |
| TVFRSSREKRRSADIFA; | (SEQ ID NO: 17) |
| tVFRSSREKRRSADIFI; | (SEQ ID NO: 18) |
| TvFRSSREKRRSADIFI; | (SEQ ID NO: 19) |
| TVfRSSREKRRSADIFI; | (SEQ ID NO: 20) |
| TVFrSSREKRRSADIFI; | (SEQ ID NO: 21) |
| TVFRsSREKRRSADIFI; | (SEQ ID NO: 22) |
| TVFRSsREKRRSADIFI; | (SEQ ID NO: 23) |
| TVFRSSrEKRRSADIFI; | (SEQ ID NO: 24) |
| TVFRSSReKRRSADIFI; | (SEQ ID NO: 25) |
| TVFRSSREKrRSADIFI; | (SEQ ID NO: 26) |
| TVFRSSREKRrSADIFI; | (SEQ ID NO: 27) |
| TVFRSSREKRRSADiFI; | (SEQ ID NO: 28) |
| TVFRSSREKRRSADIFi; | (SEQ ID NO: 29) |
| TVFRSSREKRRsADIFI; | (SEQ ID NO: 30) |
| TVFRSSREKRRSaDIFI; | (SEQ ID NO: 31) |
| TVFRSSREKRRSAdIFI; | (SEQ ID NO: 32) |
| TVFRSSREkRRSADIFI; | (SEQ ID NO: 33) |
| TVFWSSREKRRSADIFI; | (SEQ ID NO: 34) |
| VFRSSREKRRSADIFI; | (SEQ ID NO: 35) |
| FRSSREKRRSADIFI; | (SEQ ID NO: 36) |
| SSREKRRSADIFIAS; | (SEQ ID NO: 37) |
| RSSREKRRSADIFI; | (SEQ ID NO: 38) |
| FRSSREKRRSADIA; | (SEQ ID NO: 39) |
| FRSSREKRRSADIV; | (SEQ ID NO: 40) |
| TVFRSSREKRRSAD; | (SEQ ID NO: 41) |
| SSREKRRSADIFIA; | (SEQ ID NO: 42) |
| VSSREKRRSADIFI; | (SEQ ID NO: 43) |
| SSREKRRSADIFI; | (SEQ ID NO: 44) |
| FRSSREKRRSADI; | (SEQ ID NO: 45) |
| FRSSREKRRSAD; | (SEQ ID NO: 46) |
| SREKRRSADIFI; | (SEQ ID NO: 47) |
| REKRRSADIFI; | (SEQ ID NO: 48) |
| RSSREKRRSAD; | (SEQ ID NO: 49) |
| REKRRSADIF; | (SEQ ID NO: 50) |
| SSREKRRSAD; | (SEQ ID NO: 51) |
| REKRRSADI; | (SEQ ID NO: 52) |
| SREKRRSAD; | (SEQ ID NO: 53) |
| EREKRRSAD; | (SEQ ID NO: 54) |
| and | |
| REKRRSAD. | (SEQ ID NO: 55) |

17. The compound of claim 1, wherein is P derived from the i2 loop and is represented by the following structural formula or a pharmaceutically acceptable salt thereof,
Y₁-Y₂-Y₃-Y₄-Y₅-Y₆-Y₇-Y₈-Y₉-Y₁₀-Y₁₁-Y₁₂-Y₁₃-Y₁₄-Y₁₅-Y₁₆-Y₁₇-Y₁₈-Y₁₉-Y₂₀-Y₂₁Y₂₂-R₁
wherein:
Y₁ is absent or an aspartic acid residue;
Y₂ is absent or an arginine residue;
Y₃ is absent or a tyrosine residue;
Y₄ is absent or a leucine residue;
Y₅ is absent or an alanine residue;
Y₆ is absent or an isoleucine residue;
Y₇ is absent or a valine residue;
Y₈ is absent or an arginine residue;
Y₉ is absent or a proline residue;
Y₁₀ is absent or a valine residue;
Y₁₁ is absent or an alanine residue;
Y₁₂ is absent or an asparagine residue;
Y₁₃ is absent or an alanine residue;
Y₁₄ is absent or an arginine residue;
Y₁₅ is absent or a leucine residue;
Y₁₆ is absent or an arginine residue;
Y₁₇ is absent or a leucine residue;
Y₁₈ is absent or an arginine or leucine residue;
Y₁₉ is absent or a valine or leucine residue;
Y₂₀ is absent or a serine;
Y₂₁ is absent or a glycine residue; and
Y₂₂ is absent or an alanine, provided that at least five of Y₁-Y₂₂ are present;
R₁ is OR₂ or N(R₂)₂;
each R₂ is independently hydrogen or (C₁-C₁₀)alkyl; and
from 0 to 5 amino acid residues are present in the D configuration.

18. The compound of claim 17, wherein at least three of Y₈, Y₉, Y₁₀, Y₁₁, Y₁₂, Y₁₃, and Y₁₄ are present.

19. The compound of claim 18, wherein
Y₈ is an arginine residue;
Y₉ is a proline residue;
Y₁₀ is a valine residue; and
Y₁₁ is an alanine residue;

20. The compound of claim 18, wherein
Y₁₂ is an asparagine residue;
Y₁₃ is an alanine residue; and
Y₁₄ is an arginine residue; and
Y₁₅ is a leucine residue.

21. The compound of claim 17, selected from: 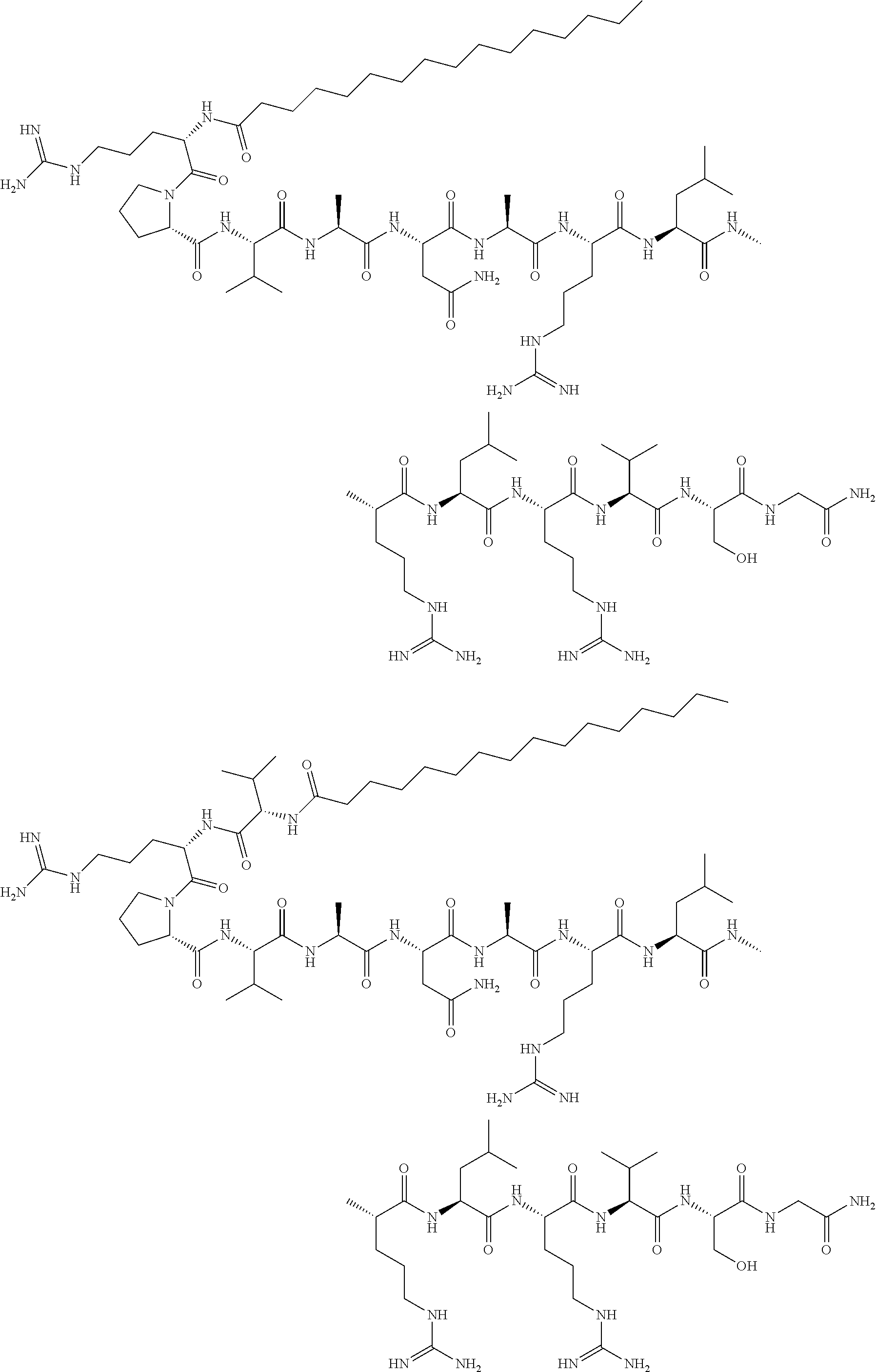 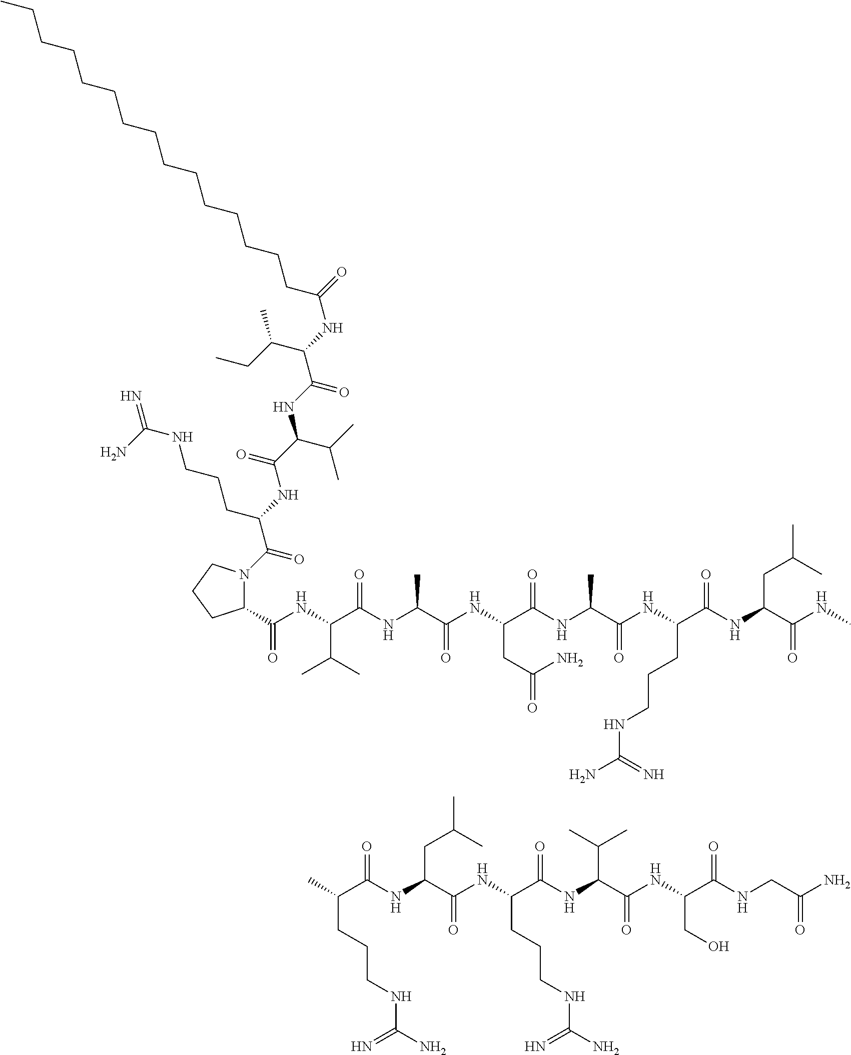 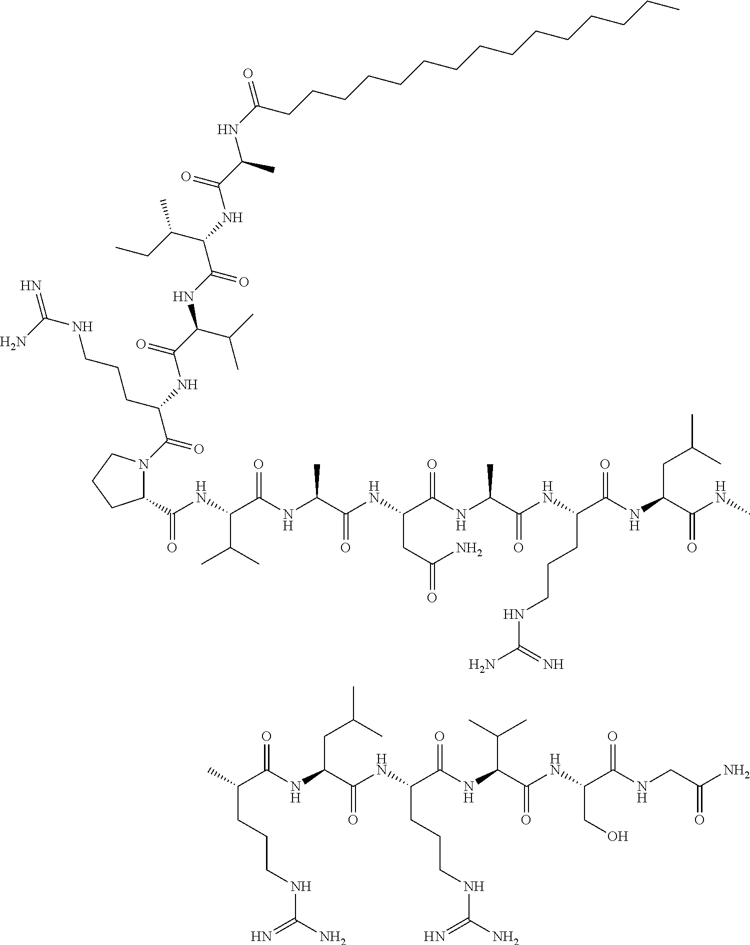 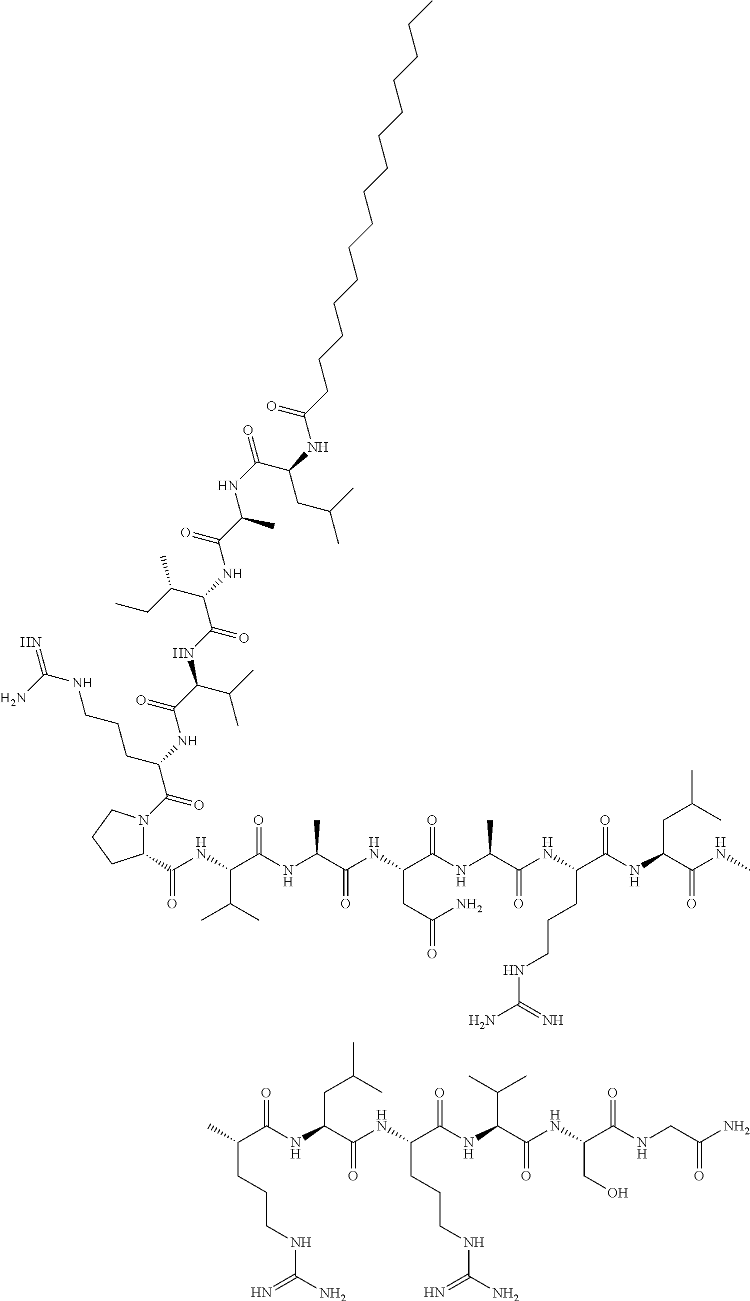 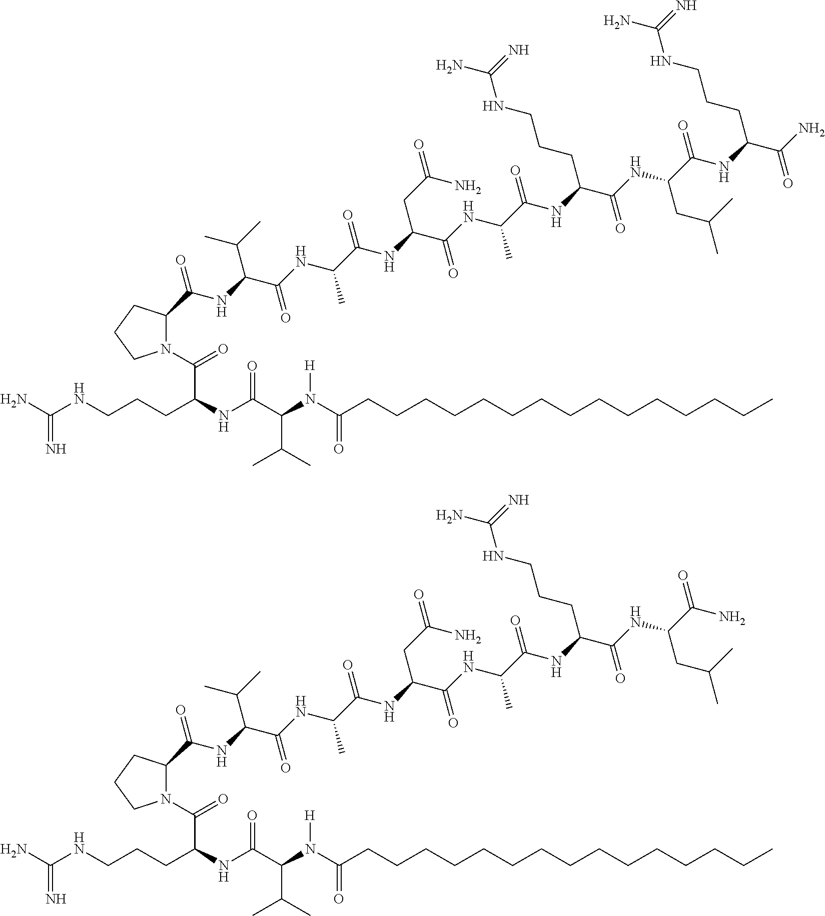 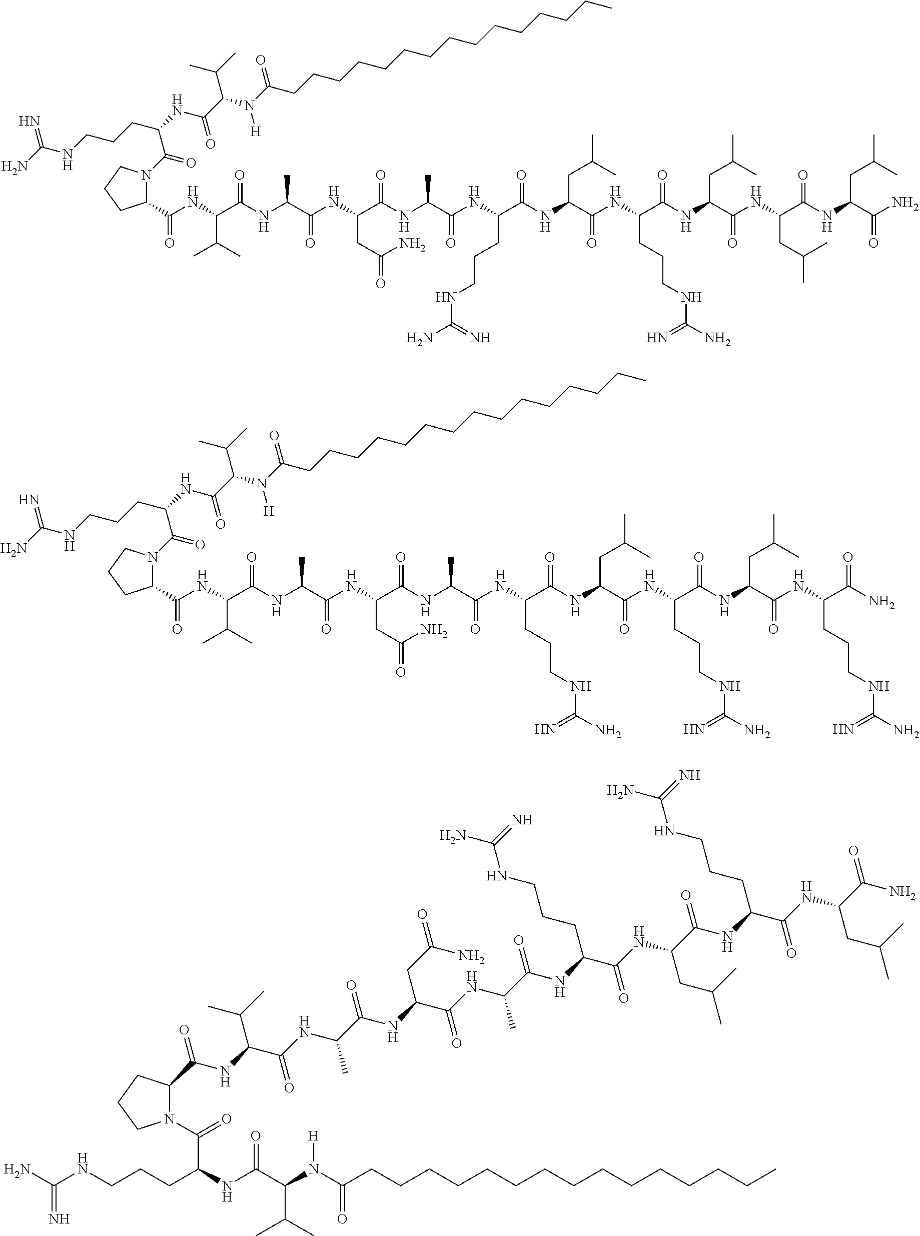 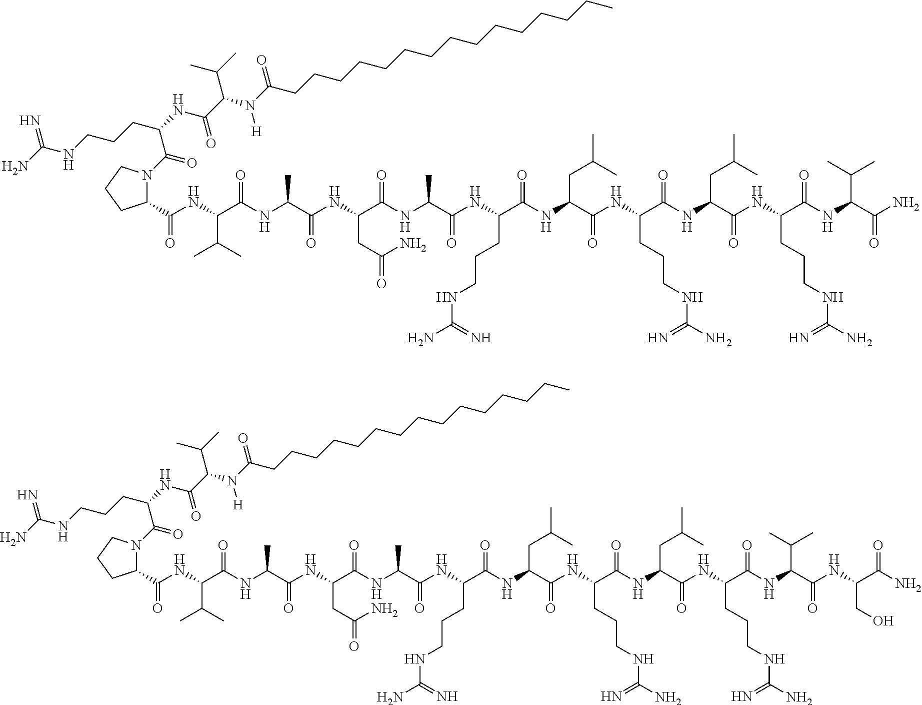
or a pharmaceutically acceptable salt of any of the foregoing.

22. The compound of claim 1, wherein P comprises at least three contiguous amino acid residues of the i2 loop.

23. The compound of claim 22, wherein P is derived from the following sequence:

| | |
|---|---|
| DRYLAIVRPVANARLRLRVSGA. | (SEQ ID NO: 56) |

24. The compound of claim 23, wherein P is a sequence selected from:

| | |
|---|---|
| DRYLAIVRPVANARLRLRVSGA; | (SEQ ID NO: 56) |
| LAIVRPVANARLRLRVSG; | (SEQ ID NO: 57) |
| AIVRPVANARLRLRVSG; | (SEQ ID NO: 58) |
| IVRPVANARLRLRVSG; | (SEQ ID NO: 59) |
| VRPVANARLRLRVSG; | (SEQ ID NO: 60) |
| RPVANARLRLRVSG; | (SEQ ID NO: 61) |
| VRPVANARLRLRVS; | (SEQ ID NO: 62) |
| AIVRPVANARLRL; | (SEQ ID NO: 63) |
| RPVANARLRLRVS; | (SEQ ID NO: 64) |
| VRPVANARLRLLL; | (SEQ ID NO: 65) |
| VRPVANARLRLRV; | (SEQ ID NO: 66) |
| RPVANARLRLRV; | (SEQ ID NO: 67) |
| VRPVANARLRLR; | (SEQ ID NO: 68) |
| RPVANARLRLR; | (SEQ ID NO: 69) |
| VRPVANARLRL; | (SEQ ID NO: 70) |
| RPVANARLRL; | (SEQ ID NO: 71) |
| VRPVANARLR; | (SEQ ID NO: 72) |
| and | |
| VRPVANARL. | (SEQ ID NO: 73) |

25. The compound of claim 1, wherein P derived from the i3 loop and is represented by the following structural formula or a pharmaceutically acceptable salt thereof,
P is Z₁-Z₂-Z₃-Z₄-Z₅-Z₆-Z₇-Z₈-Z₉-Z₁₀-Z₁₁-Z₁₂-Z₁₃-Z₁₄-Z₁₅-Z₁₆-Z₁₇-Z₁₈-Z₁₉-Z₂₀-Z₂₁-Z₂₂-Z₂₃-Z₂₄-Z₂₅-Z₂₆-Z₂₇-Z₂₈-Z₂₉-Z₃₀-R₁,
wherein:
Z₁ is absent or an isoleucine residue;
Z₂ is absent or an alanine residue;
Z₃ is absent or a glutamine or glycine residue;
Z₄ is absent or a threonine or serine residue;
Z₅ is absent or a isoleucine or glycine residue;
Z₆ is absent or an alanine or serine residue;
Z₇ is absent or a glycine residue;
Z₈ is absent or a histidine residue;
Z₉ is absent or a phenylalanine residue;
Z₁₀ is absent or an arginine residue;
Z₁₁ is absent or a lysine residue;
Z₁₂ is absent or a glutamic acid residue;
Z₁₃ is absent or an arginine residue;
Z₁₄ is absent or an isoleucine residue;
Z₁₅ is absent or a glutamic acid or glycine residue;
Z₁₆ is absent or a glycine residue;
Z₁₇ is absent or a leucine residue;
Z₁₈ is absent or an arginine or glycine residue;
Z₁₉ is absent or lysine residue;
Z₂₀ is absent or an arginine residue;
Z₂₁ is absent or an arginine residue;
Z₂₂ is absent or an arginine residue;
Z₂₃ is absent or a leucine residue;
Z₂₄ is absent or a leucine residue;
Z₂₅ is absent or a serine, alanine, phenylalanine or tryptophan residue;
Z₂₆ is absent or an isoleucine residue;
Z₂₇ is absent or an isoleucine residue;
Z₂₈ is absent or a valine residue;
Z₂₉ is absent or a valine residue; and
Z₃₀ is absent or a leucine residue; provided that at least five of Z₁-Z₃₀ are present; and
R₁ is OR₂ or N(R₂)₂;
each R₂ is independently hydrogen or (C₁-C₁₀)alkyl; and
from 0 to 5 amino acid residues are present in the D configuration.

26. The compound of claim 25, wherein at least four of Z₁₂, Z₁₃, Z₁₄, Z₁₅, Z₁₆, Z₁₇, Z₁₈, and Z₁₉ are present.

27. The compound of claim 26, wherein:
Z₁₂ is a glutamic acid residue;
Z₁₃ is an arginine residue;
Z₁₄ is an isoleucine residue; and
Z₁₅ is a glutamic acid or glycine residue.

28. The compound of claim 26, wherein:
Z₁₆ is a glycine residue;
Z₁₇ is a leucine residue;
Z₁₈ is a arginine residue or glycine residue; and
Z₁₉ is a lysine residue.

29. The compound of claim 25 ,wherein at least four of Z₂₁, Z₂₂, Z₂₃, Z₂₄, and Z₂₅ are present.

30. The compound of claim 29, wherein
Z₂₁ is an arginine residue;
Z₂₂ is an arginine residue;
Z₂₃ is a leucine residue;
Z₂₄ is a leucine residue; and
Z₂₅ is a serine residue or an alanine, phenylalanine or tryptophan residue.

31. The compound of claim 30 wherein Z₂₅ is an alanine residue.

32. The compound of claim 25, wherein Z₃, Z₄, Z₅, and Z₆ are present.

33. The compound of claim 32, wherein
Z₃ is a glutamine residue;
Z₄ is a threonine residue;
Z₅ is an isoleucine residue; and
Z₆ is an alanine residue.

34. The compound of claim 32, wherein
Z₃ is a glycine residue;
Z₄ is a serine residue;
Z₅ is a glycine residue; and
Z₆ is a serine residue.

35. The compound of claim 25, selected from: 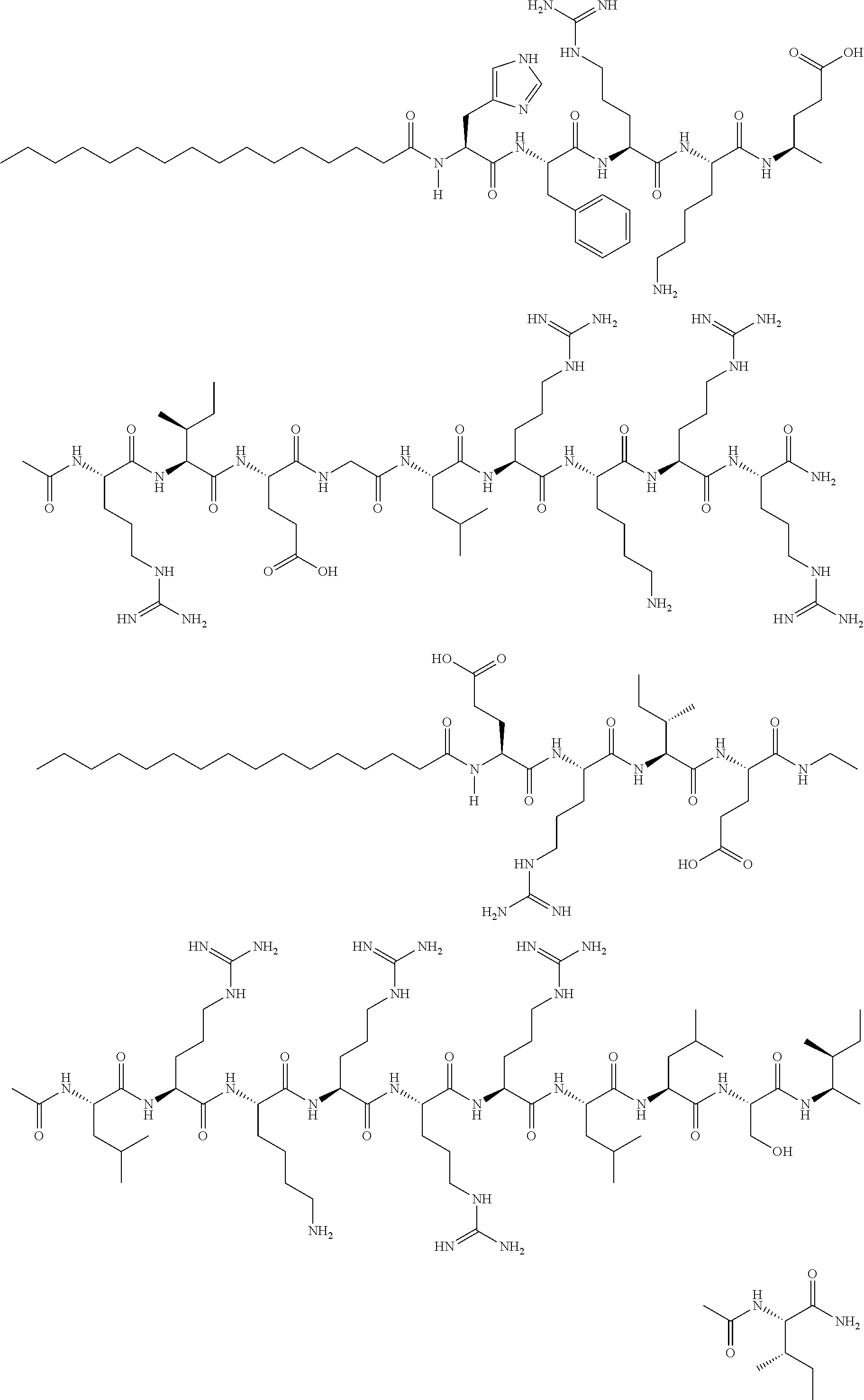 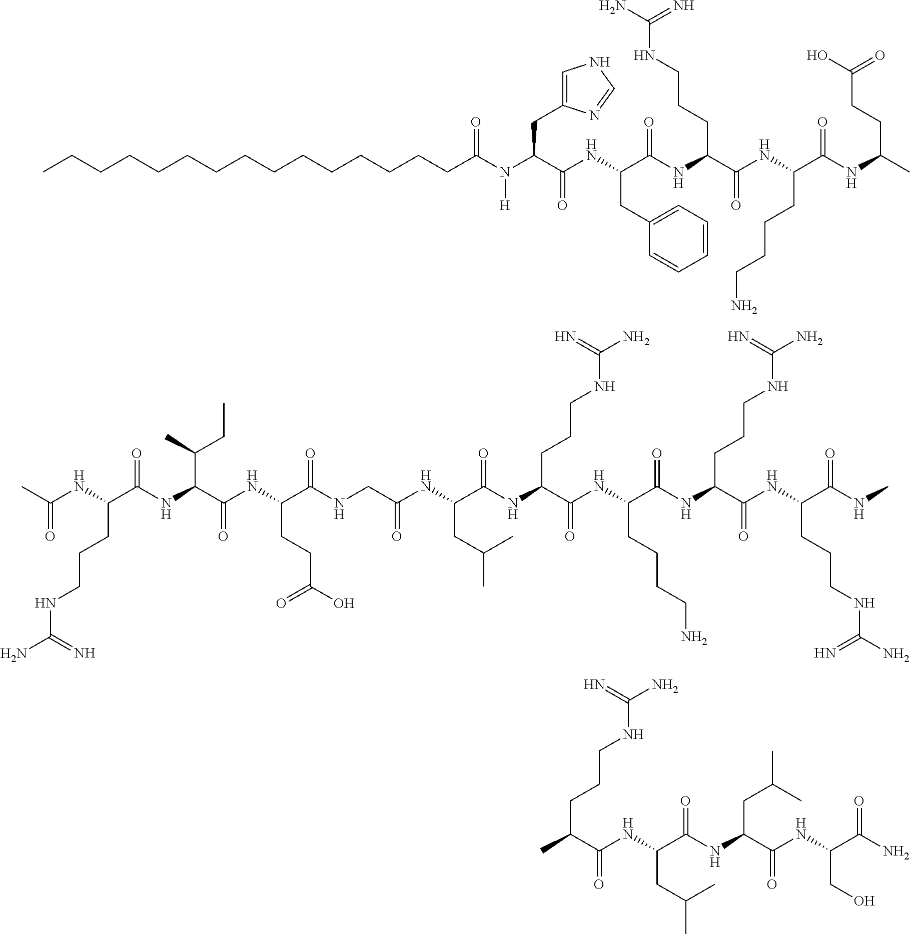 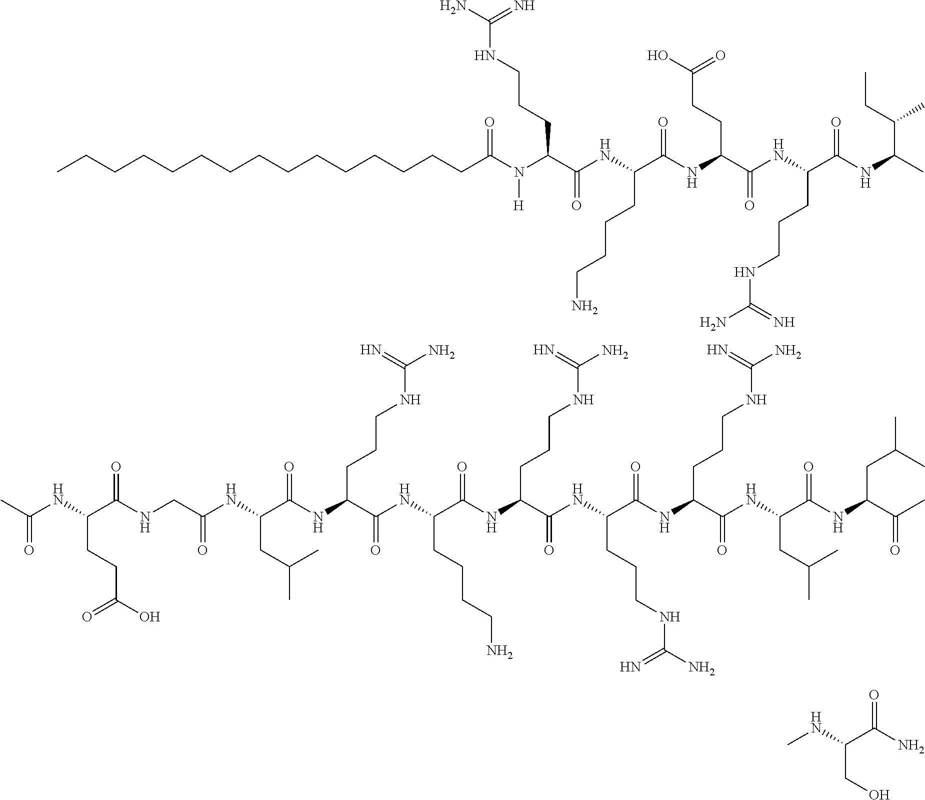 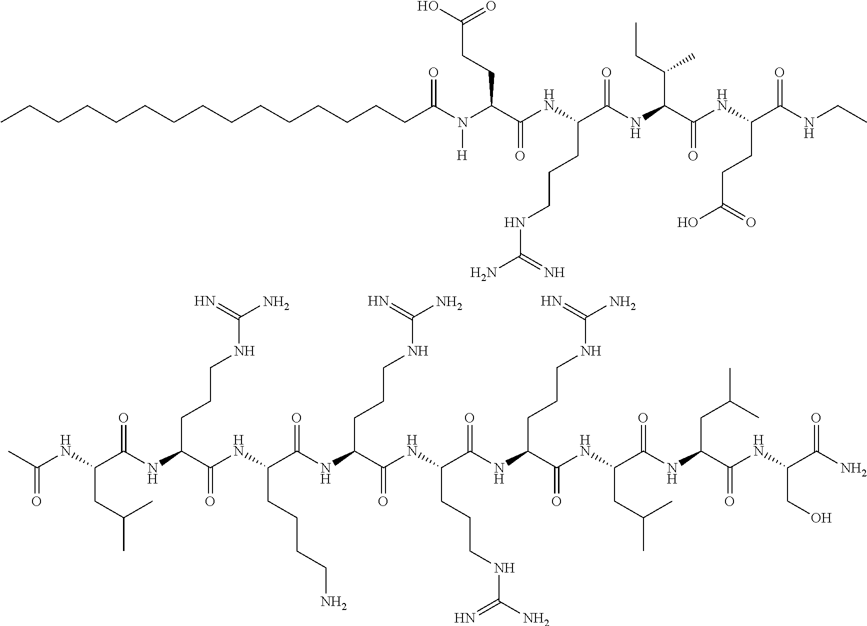 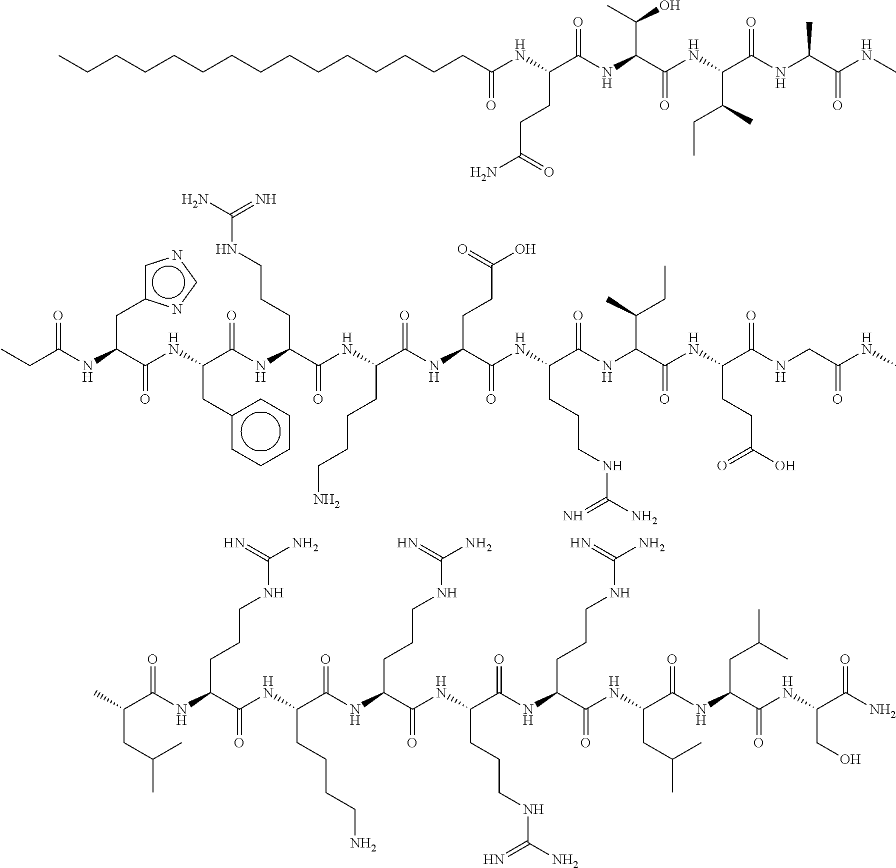 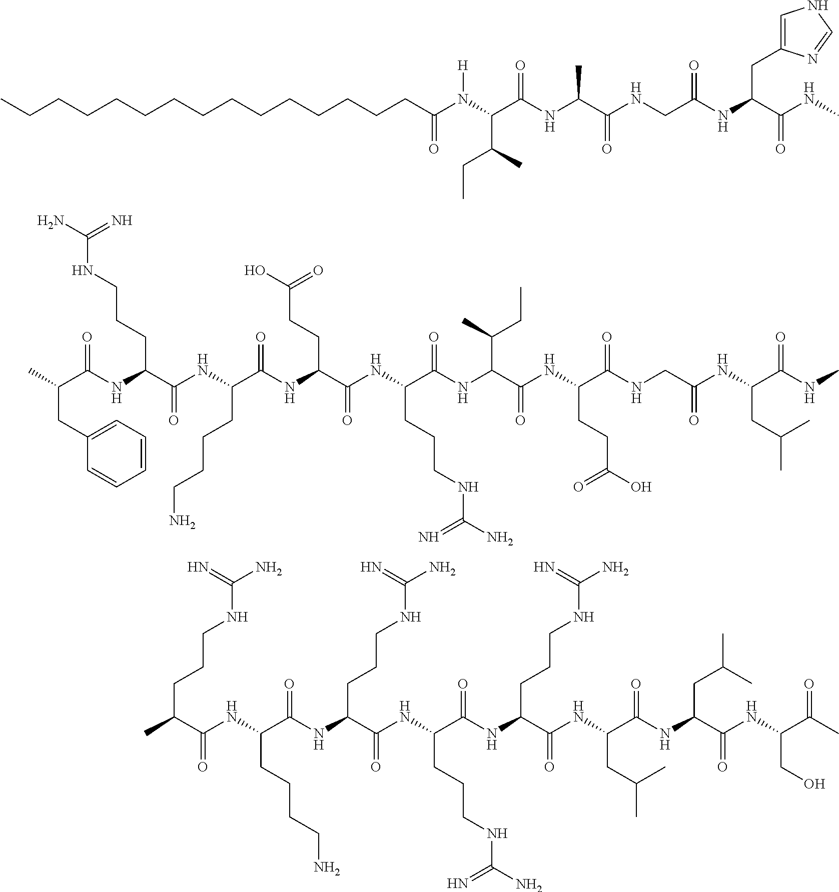 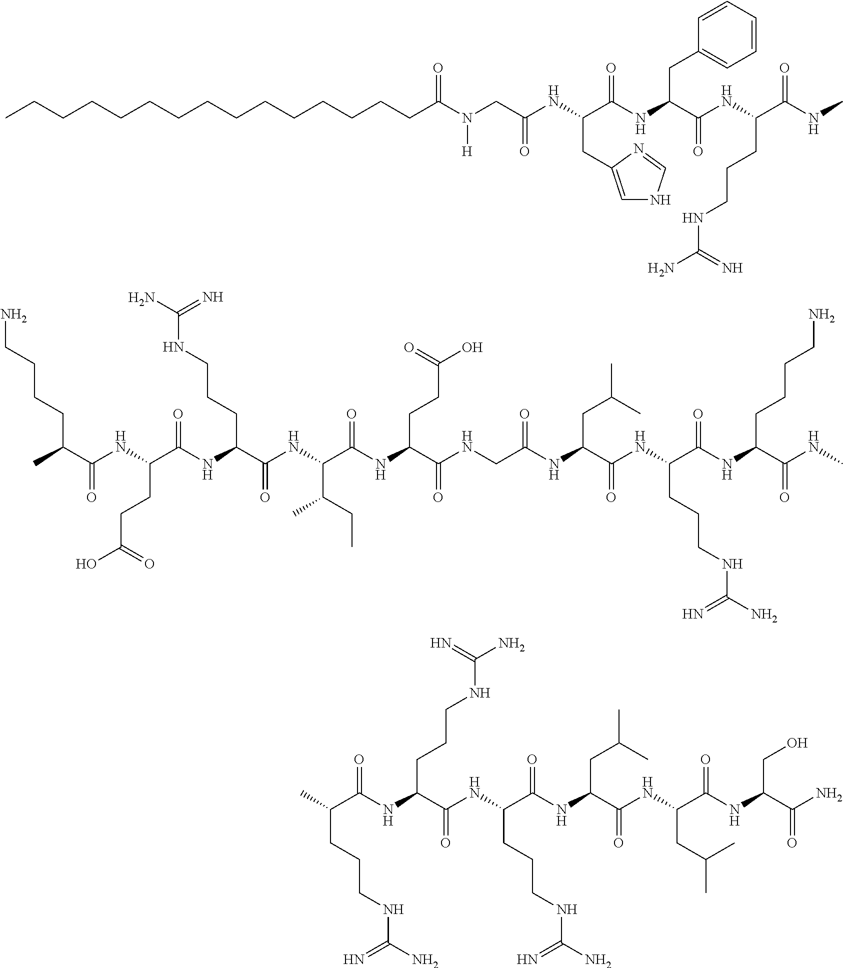 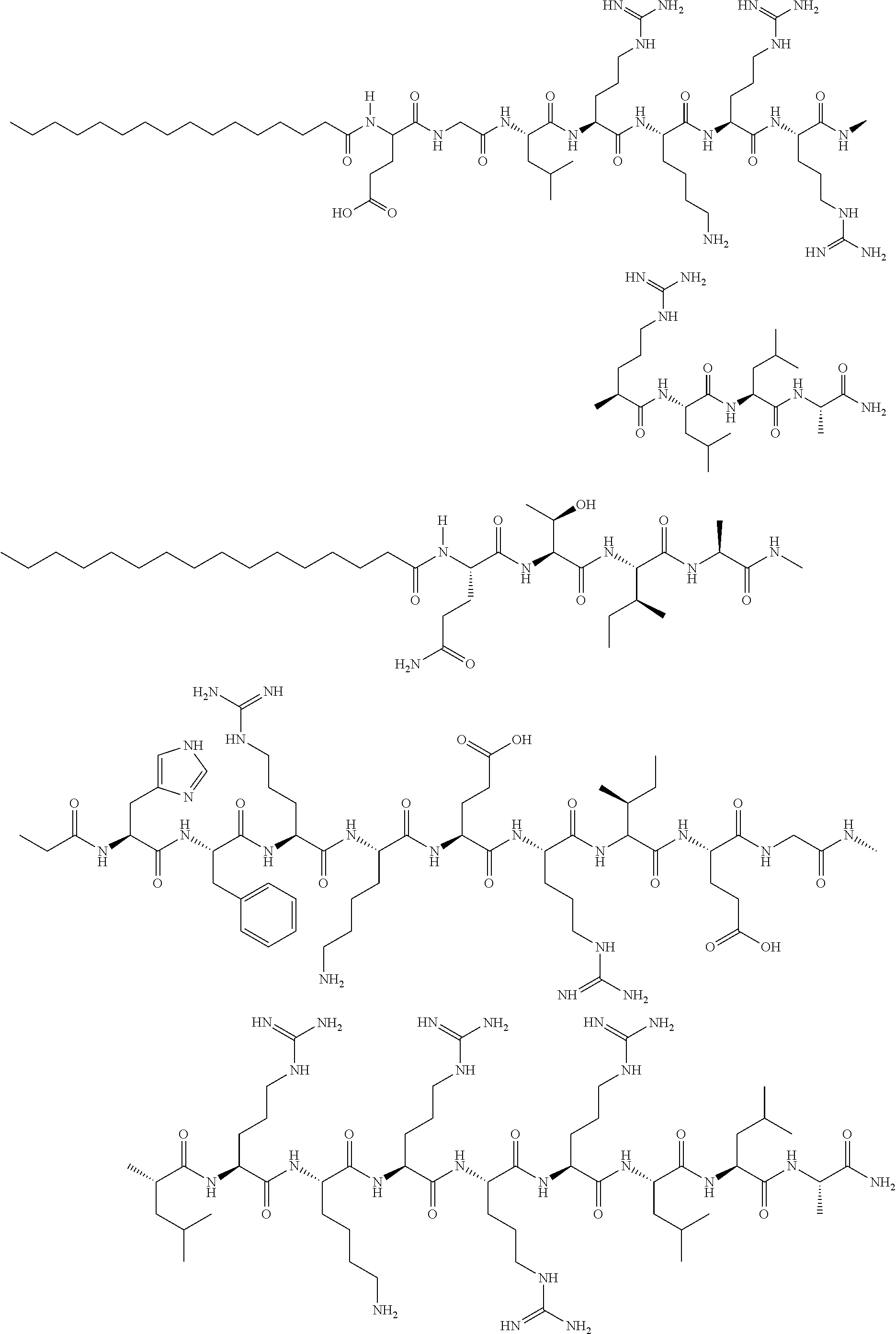 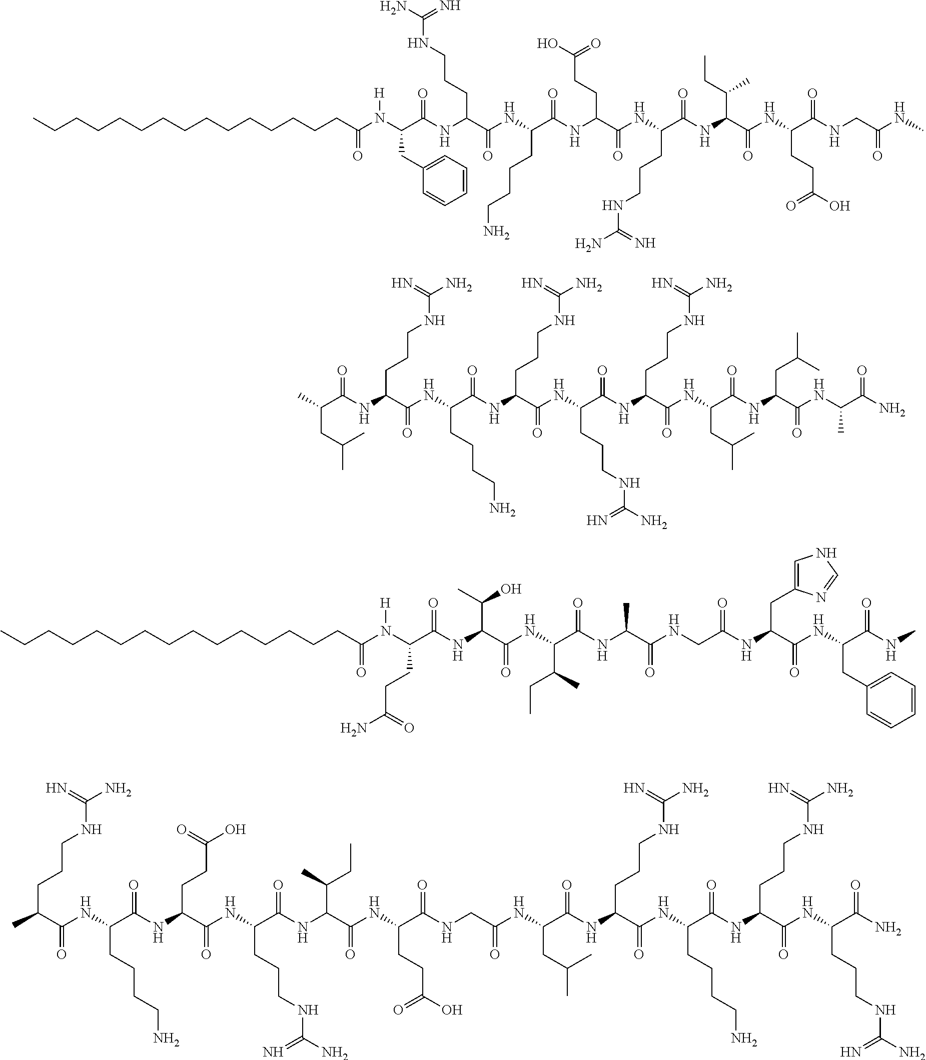 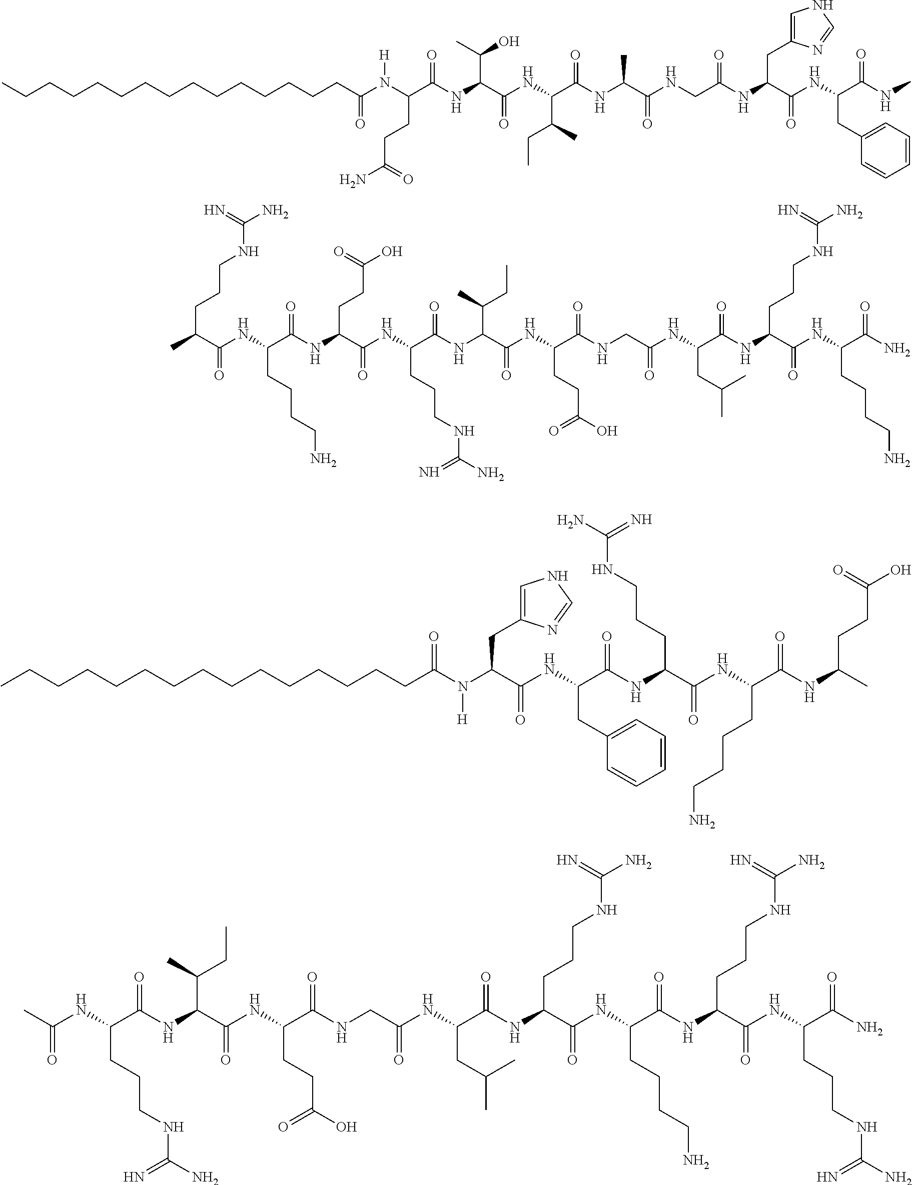 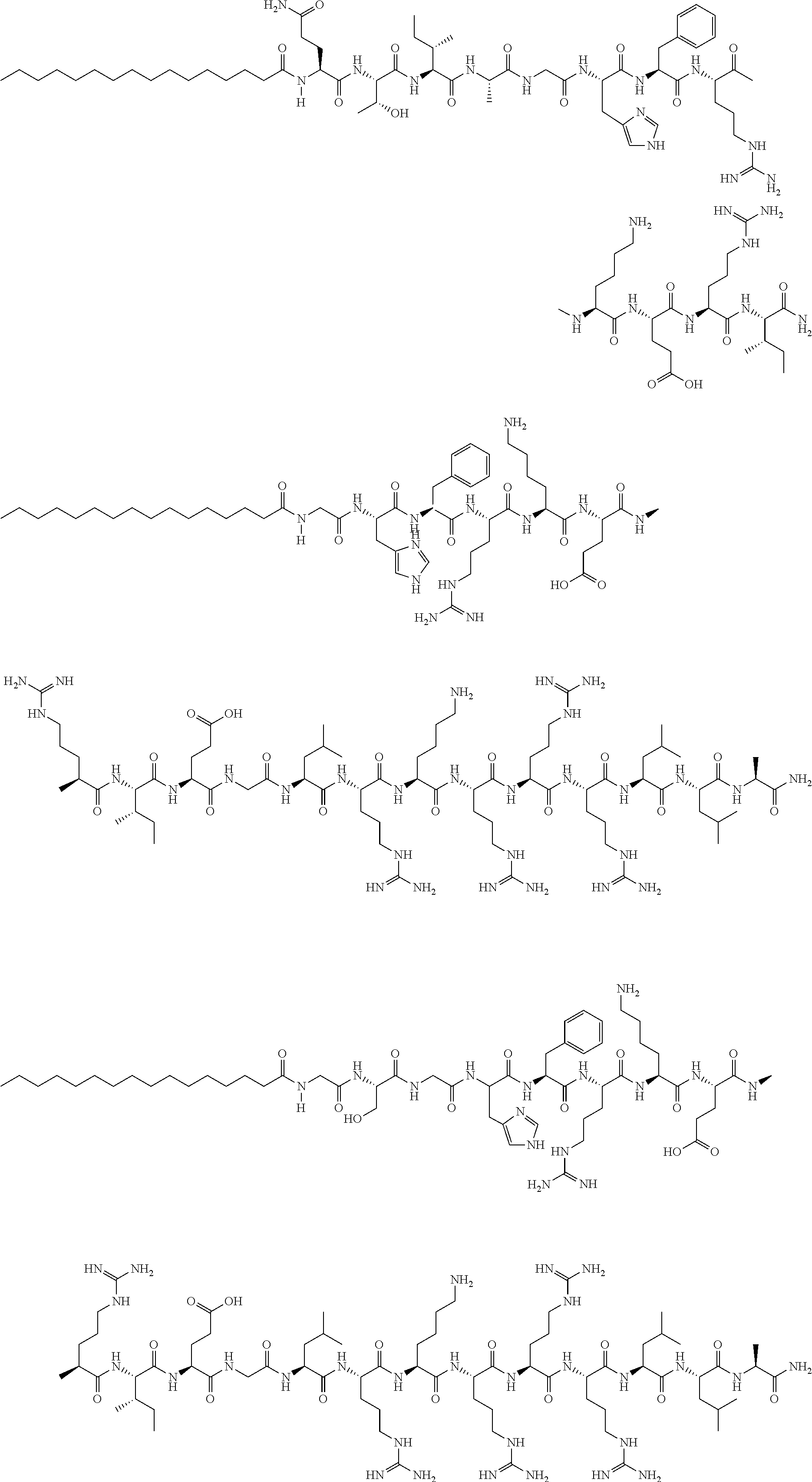 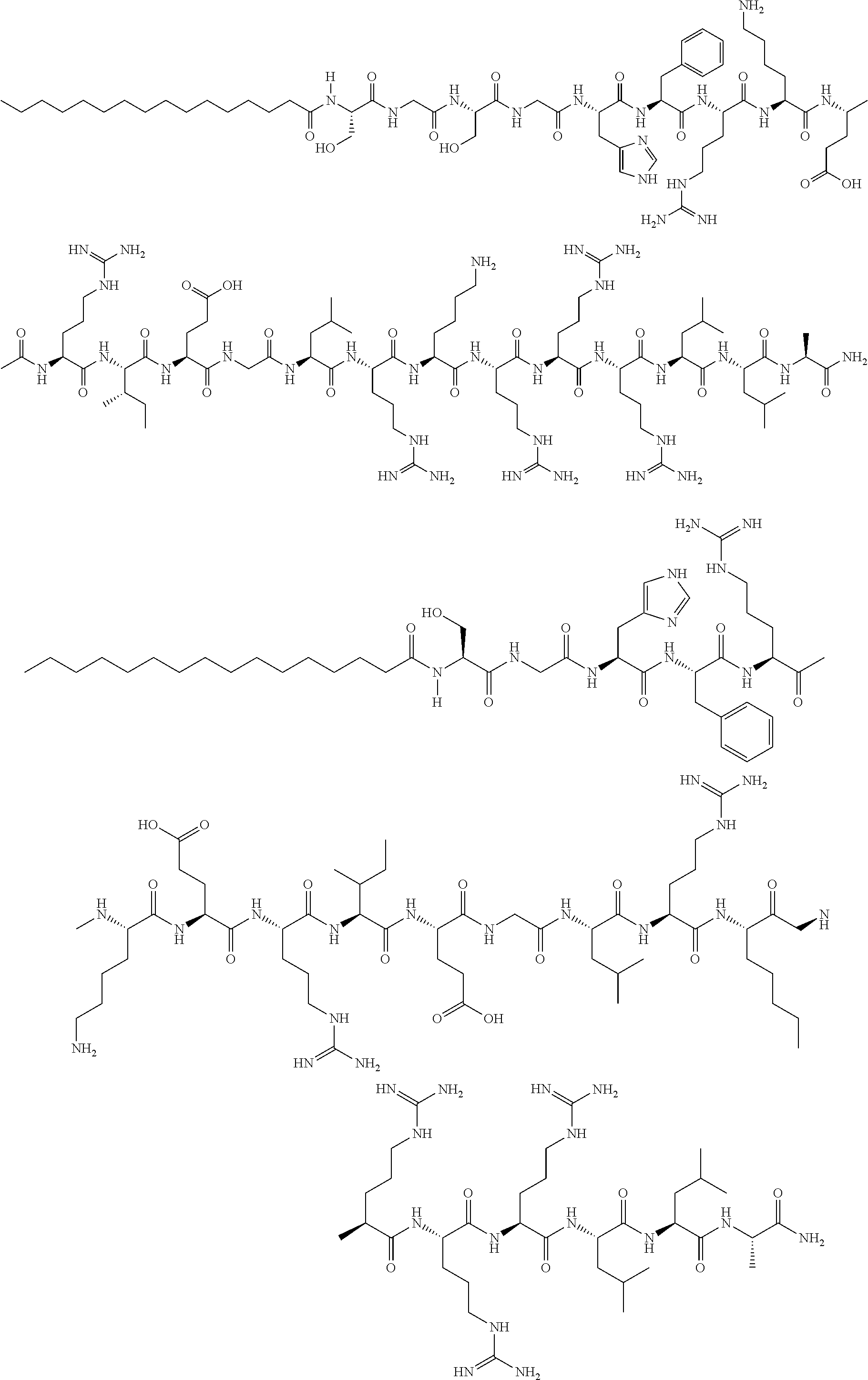 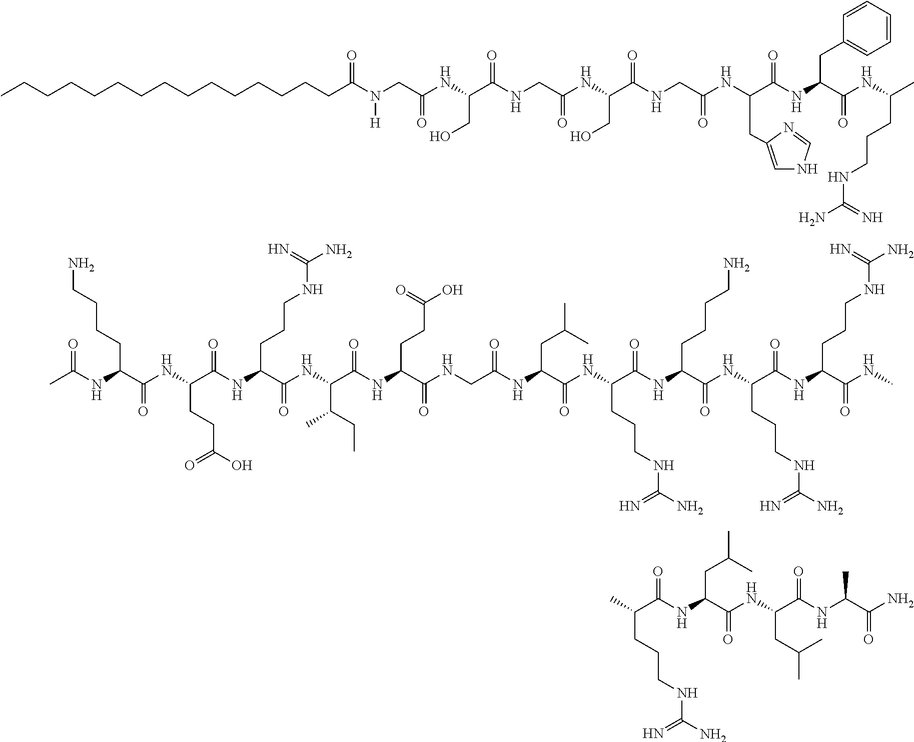 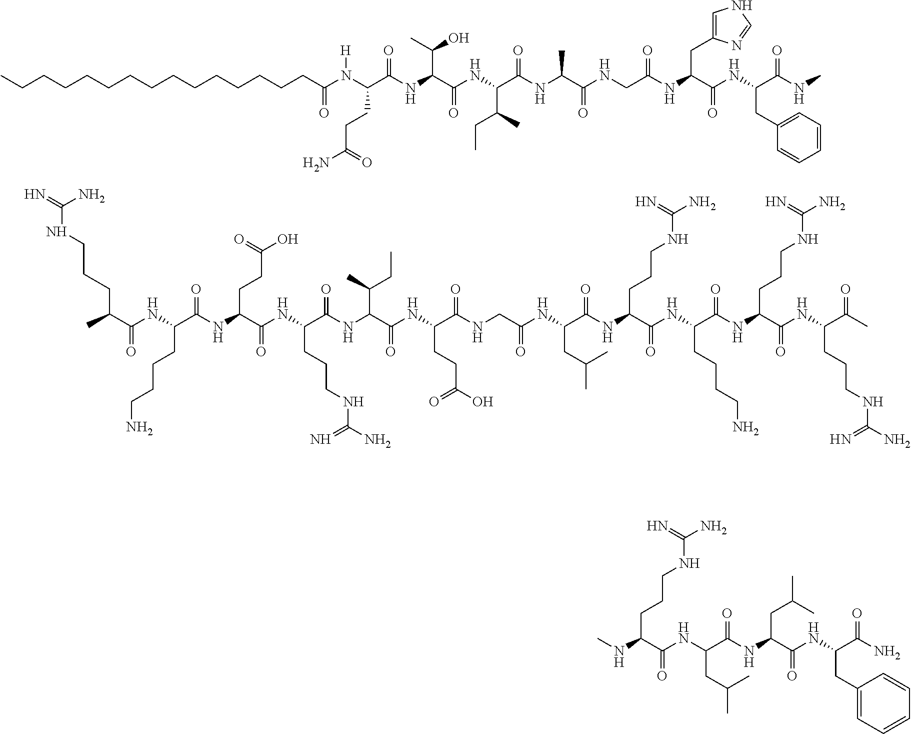 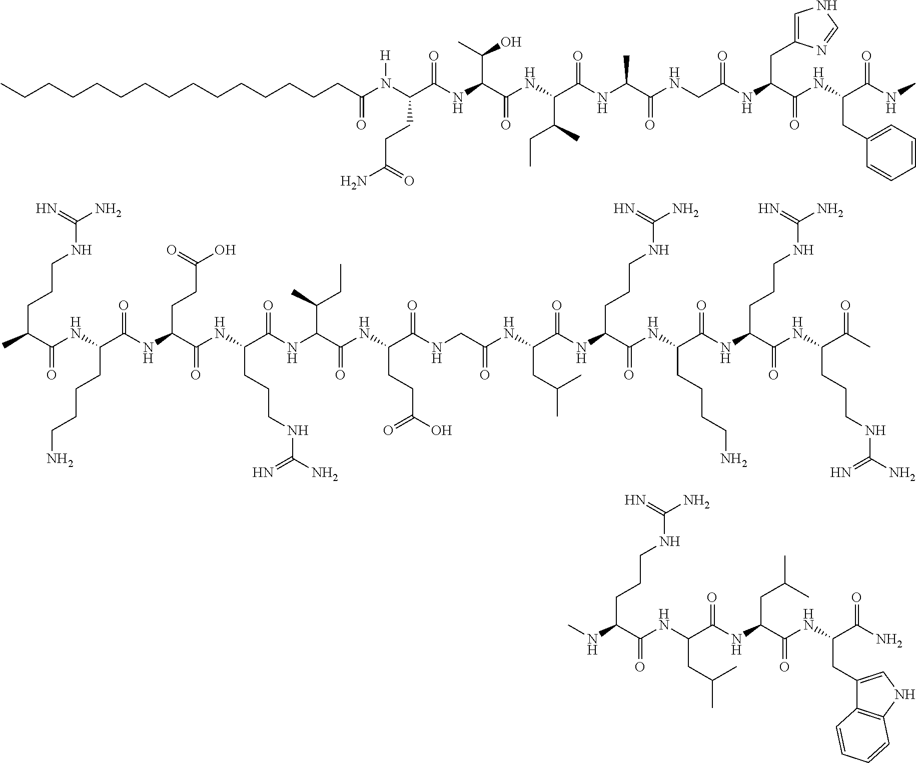
or a pharmaceutically acceptable salt of any of the foregoing.

36. The compound of claim 1, wherein P comprises at least three contiguous amino acid residues of the i3 loop.

37. The compound of claim 36, wherein P is derived from the following sequence:

| | |
|---|---|
| IAQTIAGHFRKERIEGLRKRRRLLSIIVVL. | (SEQ ID NO: 74) |

38. The compound of claim 37, wherein P is a sequence selected from:

| | |
|---|---|
| IAQTIAGHFRKERIEGLRKRRRLLSIIVVL; | (SEQ ID NO: 74) |
| QTIAGHFRKERIEGLRKRRRLLS; | (SEQ ID NO: 75) |
| QTIAGHFRKERIEGLRKRRRLLA; | (SEQ ID NO: 76) |
| QTIAGHFRKERIEGLRKRRRLLF; | (SEQ ID NO: 77) |
| QTIAGHFRKERIEGLRKRRRLLW; | (SEQ ID NO: 78) |
| GSGSGHFRKERIEGLRKRRRLLA; | (SEQ ID NO: 79) |
| SGSGHFRKERIEGLRKRRRLLA; | (SEQ ID NO: 80) |
| IAGHFRKERIEGLRKRRRLLS; | (SEQ ID NO: 81) |
| QTIAGHFRKERIEGLRKRRRL; | (SEQ ID NO: 82) |
| GSGHFRKERIEGLRKRRRLLA; | (SEQ ID NO: 83) |
| SGHFRKERIEGLRKRRRLLA; | (SEQ ID NO: 84) |
| GHFRKERIEGLRKRRRLLS; | (SEQ ID NO: 85) |
| QTIAGHFRKERIEGLRKRR; | (SEQ ID NO: 86) |
| GHFRKERIEGLRKRRRLLA; | (SEQ ID NO: 87) |
| HFRKERIEGLRKRRRLLS; | (SEQ ID NO: 88) |
| QTIAGHFRKERIEGLRK; | (SEQ ID NO: 89) |
| FRKERIEGLRKRRRLLA; | (SEQ ID NO: 90) |
| RKERIEGLRKRRRLLS; | (SEQ ID NO: 91) |
| ERIEGLRKRRRLLSII; | (SEQ ID NO: 92) |
| HFRKERIEGLRKRRRL; | (SEQ ID NO: 93) |
| FRKERIGGKRRRLLA; | (SEQ ID NO: 94) |
| ERIEGLRKRRRLLS; | (SEQ ID NO: 95) |
| HFRKERIEGLRKRR; | (SEQ ID NO: 96) |
| QTIAGHFRKERI; | (SEQ ID NO: 97) |
| EGLRKRRRLLA; | (SEQ ID NO: 98) |
| and | |
| QTIAGHFRKER. | (SEQ ID NO: 99) |

39. The compound of claim 1, wherein P derived from the i4 domain and is represented by the following structural formula or a pharmaceutically acceptable salt thereof,
M₁-M₂-M₃-M₄-M₅-M₆-M₇-M₈-M₉-M₁₀-M₁₁-M₁₂-M₁₃-M₁₄-R₁,
wherein:
M₁ is absent or a phenylalanine residue;
M₂ is absent or a phenylalanine residue;
M₃ is absent or an aspartic acid residue;
M₄ is absent or a proline residue;
M₅ is absent or an arginine residue;
M₆ is absent or a phenylalanine residue;
M₇ is absent or an arginine residue;
M₈ is absent or a glutamine residue;
M₉ is absent or an alanine residue;
M₁₀ is absent or a serine residue;
M₁₁ is absent or a threonine residue;
M₁₂ is absent or a serine residue;
M₁₃ is absent or a methionine residue; and
M₁₄ is absent or a leucine residue; provided that at least five of M₁-M₁₄ are present;
R₁ is OR₂ or N(R₂)₂;
each R₂ is independently hydrogen or (C₁-C₁₀)alkyl; and

40. The compound of claim 39, wherein
M₃ is an aspartic acid residue;
M₄ is a proline residue;
M₅ is an arginine residue; and
M₆ is a phenylalanine residue.

41. The compound of claim 1, wherein P comprises at least three contiguous amino acid residues of the i4 domain.

42. The compound of claim 41, wherein P is derived from the following sequence:
(SEQ ID NO: 100)
FFDPRFRQACTSMLCCGQSRCAGTSHSSSGEKSASYSSGHSQGP
GPNMGKGGEQMHEKSIPYSQETLVVD.

43. The compound of claim 42, wherein P is a sequence selected from

| | |
|---|---|
| FFDPRFRQASTSML; | (SEQ ID NO: 101) |
| FDPRFRQASTSML; | (SEQ ID NO: 102) |
| and | |
| DPRFRQASTSML. | (SEQ ID NO: 103) |

44. The compound of claim 1, wherein T is an optionally substituted (C₆-C₃₀)alkyl, (C₆-C₃₀)alkenyl, (C₆-C₃₀)alkynyl, wherein 0-3 carbon atoms are replaced with oxygen, sulfur, nitrogen or a combination thereof.

45. The compound of claim 44, wherein T is selected from the group consisting of: CH₃(CH₂)_{16,} CH₃(CH₂)₁₅, CH₃(CH₂)_{14,} CH₃(CH₂)₁₃, CH₃(CH₂)₁₂, CH₃(CH₂)₁₁, CH₃(CH₂)₁₀, CH₃(CH₂)_{9,} CH₃(CH₂)_{8,} CH₃(CH₂)₉OPh-, CH₃(CH₂)₆C═C(CH₂)_{6,} CH₃(CH₂)₁₁O(CH₂)_{3,} and CH₃(CH₂)₉O(CH₂)₂.

46. The compound of claim 1, wherein T is a fatty acid derivative.

47. The compound of claim 46, wherein the fatty acid is selected from the group consisting of: butyric acid, caproic acid, caprylic acid, capric acid, lauric acid, myristic acid, palmitic acid, stearic acid, arachidic acid, behenic acid, lignoceric acid, myristoleic acid, palmitoleic acid, oleic acid, linoleic acid, a-linolenic acid, arachidonic acid, eicosapentaenoic acid, erucic acid, docosahexaenoic acid.

48. The compound of claim 1, wherein T is a bile acid derivative.

49. The compound of claim 48, wherein the bile acid is selected from the group consisting of: lithocholic acid, chenodeoxycholic acid, deoxycholic acid, cholanic acid, cholic acid, ursocholic acid, ursodeoxycholic acid, isoursodeoxycholic acid, lagodeoxycholic acid, dehydrocholic acid, hyocholic acid, and hyodeoxycholic acid.

50. The compound of claim 1, wherein T is selected from sterols; progestagens; glucocorticoids; mineralcorticoids; androgens; and estrogens.

51. The compound of claim 1, wherein T is selected from:

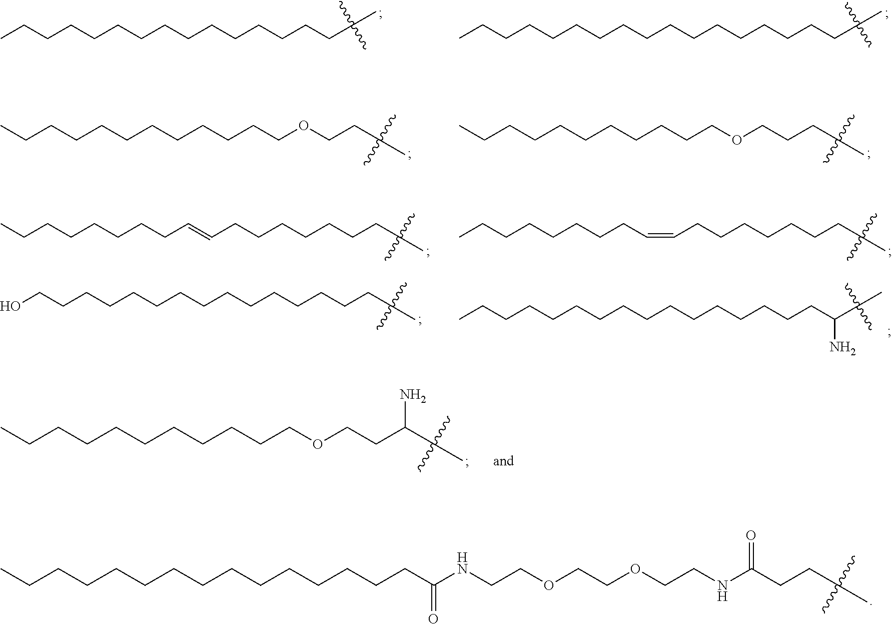

52. The compound of claim 1, wherein TL is selected from:
CH₃(CH₂)₁₅—C(O);
CH₃(CH₂)₁₃—C(O);
CH₃(CH₂)₉O(CH₂)₂C(O);
CH₃(CH₂)₁₀O(CH₂)₂C(O);
CH₃(CH₂)₆C═C(CH₂)₆—C(O);
LCA-C(O); and
CH₃(CH₂)₉OPh-C(O) wherein

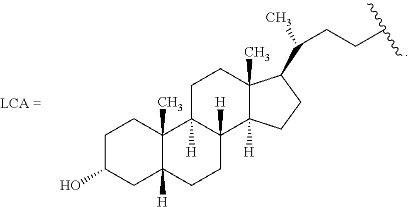

53. A method of treating heart disease, cancer, diabetes, stem cell trafficking, fluid homeostasis, cell proliferation, immune function, obesity, metastatic disease, and HIV infection in a patient in need thereof comprising administering to said patient and effective amount of a compound of claim 1.

54. The method of claim 53, wherein the heart disease is selected from: hypertension and heart failure.

55. The method of claim 54, wherein the heart failure is congestive heart failure.

56. A pharmaceutical composition, comprising a compound of claim 1 and a pharmaceutically acceptable carrier.
